SEQUENCE LISTING (10 sequences)

SEQ ID NO: 1 - protein, 7 aa, synthetic construct
GYSITDD

SEQ ID NO: 2 - protein, 8 aa, synthetic construct
IDPEDGET

SEQ ID NO: 3 - protein, 14 aa, synthetic construct
ARSLDSTWIYPFAY

SEQ ID NO: 4 - protein, 12 aa, synthetic construct
QSLLRRGNQKNH

SEQ ID NO: 5 - protein, 9 aa, synthetic construct
LQSYITPPT

SEQ ID NO: 6 - protein, 121 aa, synthetic construct
QVQLVQSGAEVKKPGSSVKVSCKASGYSITDDLIHWVRQAPGQGLEWMGWIDPEDGETNYAQKFQGRVTLTADTSTSTAYMELSSLRSEDTAVYYCARSLDSTWIYPFAYWGQGTLVTVSS

SEQ ID NO: 7 - protein, 113 aa, synthetic construct
DIVMTQSPDSLAVSLGERATINCKSSQSLLRRGNQKNHLAWYQQKPGQPPKLLIYDASTGQSGVPDRFSGSGSGTDFTLTISSLQAEDVAVYYCLQSYITPPTFGAGTKVEIK

SEQ ID NO: 8 - protein, 451 aa, synthetic construct
QVQLVQSGAEVKKPGSSVKVSCKASGYSITDDLIHWVRQAPGQGLEWMGWIDPEDGETNYAQKFQGRVTLTADTSTSTAYMELSSLRSEDTAVYYCARSLDSTWIYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 220 aa, synthetic construct
DIVMTQSPDSLAVSLGERATINCKSSQSLLRRGNQKNHLAWYQQKPGQPPKLLIYDASTGQSGVPDRFSGSGSGTDFTLTISSLQAEDVAVYYCLQSYITPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 747 bp, synthetic construct
  source 1..747: mol_type = other DNA
caggtccaactggtgcagtccggtgccgaagttaaaaaacctgggtcttccgttaaagtttcttgcaaagcctctggctacagcatcaccgatgacctgattcactgggtccgtcaggctccaggtcaaggtctggaatggatgggttggatcgatccagaagacggtgaaaccaactatgcccagaaattccagggtcgtgtaaccctgaccgccgacacctccacctctaccgcctacatggagttaagtagcctgcgttcagaggataccgcagtgtactactgcgctcgttcactggactccacctggatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtcgcggtaatcagaaaaaccaccttgcatggtatcagcagaaaccaggtcagccaccaaaattactgatctatgacgcatctaccggtcaaagcggtgtcccagatcgtttcagcggttccggctccggtactgacttcaccctgaccatctcttcccttcaggccgaagatgtggccgtgtattactgcctgcagagctacatcaccccacctactttcggtgctggtactaaagttgaaatcaaa

(19) United States
(12) Patent Application Publication
(10) Pub. No.: US 2026/0274942 A1
(43) Pub. Date: Sep. 17, 2026

(54) ANTI-IL-2 ANTIBODY COMBINATIONS AND METHODS OF USE THEREOF

(71) Applicant: AULOS BIOSCIENCE, INC., Larkspur, CA (US)

(72) Inventors: Inbar AMIT, Mevasert Zion (IL); Timothy WYANT, Bellingham, MA (US); Yanay OFRAN, Tel Aviv (IL); James Robert VASSELLI, San Francisco, CA (US); Aron Marc KNICKERBOCKER, Tiburon, CA (US)

(73) Assignee: AULOS BIOSCIENCE, INC., Larkspur, CA (US)

(21) Appl. No.: 19/691,816

(22) Filed: May 29, 2026

Related U.S. Application Data
(63) Continuation-in-part of application No. 19/381,194, filed on Nov. 6, 2025.
Application No. 19/381,194 is a continuation-in-part of application No. 19/345,303, filed on Sep. 30, 2025.
Application No. 19/345,303 is a continuation-in-part of application No. 18/431,983, filed on Feb. 4, 2024.
Application No. 18/431,983 is a continuation-in-part of application No. 18/404,093, filed on Jan. 4, 2024.
Application No. 18/404,093 is a continuation-in-part of application No. PCT/US2023/079221, filed on Nov. 9, 2023.
(60) Provisional application No. 63/383,086, filed on Nov. 10, 2022.
(60) Provisional application No. 63/503,481, filed on May 21, 2023.
(60) Provisional application No. 63/503,977, filed on May 24, 2023.
(60) Provisional application No. 63/589,659, filed on Oct. 12, 2023.

(51) Int. Cl. C07K 16/24; A61P 35/00; C07K 16/28

(52) U.S. Cl. CPC C07K 16/246; A61P 35/00; C07K 16/2818; C07K 16/2827; C07K 2317/21

(57) ABSTRACT

Described herein are therapeutic methods for subjects having an unresectable locally advanced or metastatic cancer. The method includes administration of engineered anti-IL-2 antibodies in combination with an initial low dose of IL-2 and further include administration of at least one booster low doses of IL-2. Also described herein are combination therapies comprising engineered anti-IL-2 antibodies in combination with an immune checkpoint inhibitor such as nivolumab and an initial low dose of IL-2 and related therapeutic methods for subjects having an unresectable locally advanced or metastatic cancer, for example but not limited to cutaneous melanoma.

### CROSS-REFERENCE TO RELATED APPLICATIONS

This application is a Continuation-in-Part Application of U.S. application Ser. No. 19/381,194 filed Nov. 6, 2025, which is a Continuation-in-Part of U.S. application Ser. No. 19/345,303 filed Sep. 30, 2025, which is a Continuation-in-Part Application of U.S. application Ser. No. 18/431,983 filed Feb. 4, 2024, which is a Continuation-in-Part Application of U.S. application Ser. No. 18/404,093 filed Jan. 4, 2024, which is a Continuation-in-Part Application of PCT International Application No. PCT/US23/79221, International Filing Date Nov. 9, 2023, claiming the benefit of priority of U.S. Provisional Application No. 63/589,659 filed Oct. 12, 2023, U.S. Provisional Application No. 63/503,977 filed May 24, 2023, U.S. Provisional Application No. 63/503,481 filed May 21, 2023, and U.S. Provisional Application No. 63/383,086 filed Nov. 10, 2022, which are all hereby incorporated by reference in their entirety.

### SEQUENCE LISTING STATEMENT

The instant application contains a Sequence Listing which has been submitted electronically in XML format and is hereby incorporated by reference in its entirety. The XML formatted sequence listing, created on Oct. 28, 2025, is named P-621284-US5_SL_280CT25.XML and is 11,360 bytes in size.

### FIELD OF THE INVENTION

The disclosure relates in general to the field of cancer treatment. In some embodiments, the present disclosure describes a combination comprising engineered anti-IL-2 antibodies and an immune checkpoint inhibitor such as nivolumab and the use of the combination for treating cancers, including solid tumors such as unresectable locally advanced or metastatic tumors, for example but not limited to cutaneous melanomas. The present disclosure also describes the methods of use of engineered anti-IL-2 antibodies, a loading dose of IL-2, and one or more booster doses of IL-2 for treating cancers, including solid tumors such as unresectable locally advanced or metastatic tumors.

### BACKGROUND

Interleukin-2 (IL-2), a potent T-cell-stimulating cytokine, was the first U.S. Food and Drug Administration (FDA)-approved immunotherapeutic effective in treating cancers such as metastatic melanoma and renal cell carcinoma (RCC). However, the need for high doses and frequent administration of IL-2 to induce therapeutic effects has led to severe side and off-target effects.

IL-2 signaling has two opposite effects. IL-2 can enhance immune response by activation of effector cells and induce their proliferation. Alternatively, IL-2 can tune down immune response by activation and proliferation of CD4+ regulatory T (Treg) cells. To facilitate these functions, IL-2 mediates its effect by binding to two forms of IL-2 receptor: i) trimeric receptors made up of IL-2Rα (CD25), IL-2Rβ (CD122), and a common IL-2Rγ (γc, CD132) chains, or ii) a dimeric receptor that consists of only the IL-2Rβ and IL-2Rγ subunits. Both the dimeric and trimeric receptors are able to transmit IL-2 binding signaling via the STAT5 pathway. However, IL-2 binds the αβγ receptor trimer at 100-fold tighter than the βγ receptor dimer. It has been demonstrated that the binding affinity of hIL-2 to the αβγ trimer is approximately 10 pM, whereas the hIL-2 affinity to the βγ dimer is 1 nM.

AU-007, also known as imneskibart, is a human IgG1 monoclonal antibody (mAb) that binds to IL-2 with pM affinity on its CD25 binding epitope and completely inhibits its binding to CD25, without hindering its binding to CD132/CD122.

AU-007-bound IL-2 cannot bind trimeric (CD25, CD122, CD132) IL-2 receptors (IL-2R) on regulatory T cells (Tregs), vascular endothelium, or eosinophils, but IL-2's binding to dimeric (CD122, CD132) IL-2R on effector T (T eff) and NK cells is unhindered. AU-007 thus redirects IL-2 towards T eff and NK cell activation, while diminishing Treg activation and vascular leak, and redirects IL-2 generated from T eff cell expansion, converting a Treg-mediated autoinhibitory loop into an immune stimulating loop, driving tumor killing.

AU-007 bound IL-2 prolongs the T_{1/2} of IL-2, allowing endogenous IL-2 or low dose aldesleukin to initiate an anti-tumor response. AU-007 monotherapy at doses up to 12 mg/kg of a subject's weight once every 2 weeks (Q2W) is safe and well tolerated, with initial signs of immune modulation consistent with AU-007's mechanism of action. More work is needed to ascertain how to optimize the benefits of treatment with AU-007 in combination with IL-2 in patients.

When melanoma is diagnosed in its early stages, surgical resection of the lesion is associated with a favorable prognosis. However, for locally advanced and metastatic disease, surgery is not sufficient. The 5-year survival drops from 99% to 20% when distant metastases are present.

Melanoma is one of the most sensitive tumors to immune modulation. Immune checkpoint inhibitors (ICIs) against programmed death-1 (PD-1) have dramatically changed the management of both unresectable and metastatic melanoma as well as those at high risk for recurrence after resection. Unfortunately, primary and secondary resistance and the absence of predictive markers of response are challenging problems with ICI therapy. New therapies are needed for treating melanoma.

### SUMMARY

In one aspect, disclosed herein is a method of treating an unresectable locally advanced or metastatic solid cancer in a subject, said method comprising administering to said subject multiple doses of an anti-IL-2 antibody or a pharmaceutical composition thereof, a loading dose of IL-2 or a pharmaceutical composition thereof, and at least one booster dose of IL-2 or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; wherein said loading dose and booster dose of IL-2 are subcutaneously administered at a dose of between about 15,000 IU/kg of said subject's body weight-500,000 IU/kg of said subject's body weight; wherein said pharmaceutical composition further comprises a pharmaceutically acceptable carrier, thereby treating said unresectable locally advanced or metastatic cancer in said subject.

In a related aspect, a booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof.

In another aspect, disclosed herein is a combination therapy comprising an anti-IL-2 antibody or a pharmaceutical composition thereof, a loading dose of IL-2 or a pharmaceutical composition thereof, and nivolumab or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; wherein said anti-IL-2 antibody is formulated for administration at a dose of 9 mg/kg of a subject's body weight, the loading dose of IL-2 is formulated for subcutaneous administration at a dose of 135,000 IU/kg of a subject's body weight, and said nivolumab is formulated for administration at a dose of 480 mg or 240 mg; and wherein said pharmaceutical composition further comprises a pharmaceutically acceptable carrier.

In another aspect, disclosed herein is a method of treating an unresectable locally advanced or metastatic solid cancer in a subject, said method comprising administering to said subject an anti-IL-2 antibody at a dose of 9 mg/kg of said subject's body weight or a pharmaceutical composition thereof, a loading dose of IL-2 at a dose of 135,000 IU/kg of said subject's body weight or a pharmaceutical composition thereof, and nivolumab at a dose of 480 mg or 240 mg or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; and wherein said pharmaceutical composition further comprises a pharmaceutically acceptable carrier, thereby treating said unresectable locally advanced or metastatic cancer in said subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure of engineered anti-IL-2 antibody and combination therapies comprising the anti-IL-2 antibody, both as to their generation and method of use, together with objects, features, and advantages thereof, may best be understood by reference to the following detailed description when read with the accompanying drawings in which:

FIG. 1 graphically presents the administration and dosage schemes of AU-007 Q2W monotherapy (left—1A), AU-007+a single IL-2 Loading Dose (center—1B), and combination therapy of AU-007 Q2W+IL-2 Q2W (right—1C). When administered, recombinant human IL-2 (aldesleukin) was administered subcutaneously, at much lower doses and much less frequently than the approved regimen of intravenously administered aldesleukin. The terms “3+3” and “1+2” refer to the size of the cohort at a given dose level. For 3+3, the first 3 enrolled patients were administered AU-007 at the dose level that is noted. If no dose-limiting toxicities (DLTs) are seen, then administration was escalated to the next highest dose level. However, if any of the first 3 patients had a dose-limiting toxicity that was drug-related, then an additional three more patients would have received treatment at that same dose level to see if they have DLTs before escalating. 1+2 follows the same principle—start with one patient, if DLTs are observed, add two more, or if no DLTs are observed in the first single patient, then dosages can be escalated.

FIG. 2 provides a timeline of the treatment with AU-007 in combination with single-dose aldesleukin and optional aldesleukin boost administration and tumor assessment. AU-007 is administered once every 2 weeks (Day 1, 15, 29, 43—see black arrow); Aldesleukin is administered as a loading dose once in Cycle 1 (Day 1—see ellipse). Tumor evaluation (black diamond) using Response Evaluation Criteria In Solid Tumors (RECIST) evaluation of CT scan, MRI, PET scan, or ultrasound to measure existing tumor size and assess for any new tumors is carried out at the end of each cycle. Biopsy (4-point star) is conducted to evaluate immune-modulating effect of drug within tumor by evaluating immune cell infiltration. The Dose-Limiting Toxicity (DLT) period lasts 4 weeks and informs dose escalation decisions. The aldesleukin boost administration is administered in patients who are tolerating treatment and are clinically stable at the discretion of the Investigator and Sponsor based on the following: (1) with each cycle (Q8W, Day 1 of cycle) until objective tumor shrinkage is observed on radiologic imaging or physical exam or (2) with objective signs of worsening tumor growth kinetics: e.g., previously shrinking tumors becoming stable, or increase in tumor markers, tumor growth, or appearance of new tumor growth in a tumor that had previously been stable or had decreased in size.

FIGS. 3A-3B show differences in peripheral cell population (Treg, NK, CD8+ cell populations, and the CD8/Treg ratio) measured on Day 15 (FIG. 3A) or Day 44 (FIG. 3B) across all cohorts dosed with any AU-007 (e.g., 4.5 mg/kg AU-007) and escalating doses of subcutaneous aldesleukin (15,000 IU/kg, 45,000 IU/kg, 135,000 IU/kg, or 270,000 IU/kg).

FIG. 4 shows a comparison of progression-free survival (PFS) between patients with less than 43% reduction in Tregs vs. greater than 43% Tregs reduction, where 43% reduction is the median Treg reduction.

FIG. 5 shows the dose response effect of subcutaneous aldesleukin administration and the concentration of circulating IFN-γ.

FIG. 6 shows dose response effect of IV AU-007 administration and the concentration of circulating IFN-γ. On Day 1 (0-24 hours), IFN-γ from blood at 5 different timepoints (pre-dose, 2 hours, 6 hours, 24 hours and 48 hours post dose) was measured; on Day 15 (360 hours), IFN-γ from blood at 3 different timepoints (pre-dose, end of infusion, and 6 hours post-infusion) was measured; on Day 29 (700 hours), IFN-γ from blood at 2 different timepoints (pre-dose and end of infusion) was measured.

FIG. 7 shows the relative risk of progression as a function of IL-2 dose (left hand side) and AU-007 dose (right hand side) for renal cell carcinoma (RCC) (middle row), melanoma (bottom row), and other cancers (top row).

FIG. 8 AU-007 PK and IL-2 Coverage Calculated on a Mole to Mole Ratio

FIGS. 9A-9B show the best response in melanoma patients in Phase 1 and Phase 2 receiving AU-007 Q2W+Single SC Aldesleukin (Arm B regimen; FIG. 9A) or AU-007 Q2W+SC Aldesleukin Q2W (Arm C regimen; FIG. 9B). In the Arm B regimen, AU-007 was administered IV at 4.5 mg/kg and in the Arm C regimen, AU-007 was administered IV at 4.5 or 9 mg/kg. For both arms, Aldesleukin was administered SC at a dose of 15 IU/kg, 45 IU/kg, 135 IU/kg, or 270K IU/kg.

FIGS. 10A-10B show the percentage change overtime in sum of diameters in target lesions vs. baseline in Melanoma Patients in Phase 1 and Phase 2 patients receiving AU-007 Q2W+Single SC Aldesleukin loading dose (Arm B Regimen; FIG. 10A) or AU-007 Q2W+SC Aldesleukin Q2W (Arm C regimen; FIG. 10B). In the Arm B regimen, AU-007 was administered IV at 4.5 mg/kg and in the Arm C regimen, AU-007 was administered IV at 4.5 or 9 mg/kg. For both arms, Aldesleukin was administered SC at a dose of 15 IU/kg, 45 IU/kg, 135 IU/kg, or 270K IU/kg.

FIGS. 11A-11B show the best response in RCC patients in Phase 1 and Phase 2 receiving the AU-007 Q2W+Single SC Aldesleukin (Arm B regimen; FIG. 11A) or AU-007 Q2W+SC Aldesleukin Q2W (Arm C regimen; FIG. 11B).

FIG. 12 shows the percentage change over time in sum of diameters in target lesions vs. baseline in RCC Patients in Phase 1 and Phase 2 receiving AU-007 Q2W+SC aldesleukin loading dose (Arm B Dosing Regimen).

FIG. 13 shows the probability of Progression-Free Survival (PFS) for all Phase 1 and 2 patients with all tumor types, comparing Arm B Regimen vs. Arm C Regimen.

FIGS. 14A and 14B show Treg fold change vs. baseline by Week 8 (FIG. 14A) and Week 16 (FIG. 14B) progression status. The boxplots summarize maximum Treg decrease for each arm and progression status group showing median (line in each box) with lower and upper box boundaries corresponding to the 25th and 75th percentile.

FIGS. 15A-15C show the percent change in Tregs (FIG. 15A) and CD8 Cells (FIG. 15B), and the CD8/Treg ratio (FIG. 15C) normalized to baseline in Arm 2B vs. Arm 2C regimens.

FIGS. 16A-16C show tumor diameter data over time in 2 patients (Patient AU08-0066, FIG. 16A; Patient US01-0098, FIG. 16B; and Patient AU03-0084, FIG. 16C) after receiving 9 mg/kg IV AU-007 Q2W and 135,000 IU/kg SC aldesleukin (IL-2) loading dose. Patient AU08-0066 received a booster dose of aldesleukin on Cycle 4 Day 1 (C4D1; 24 weeks since treatment initiation). Patient US01-0098 received two booster doses of aldesleukin on Cycle 2 Day 1 (C2D1; 8 weeks since treatment initiation); and on Cycle 3 Day 1 (C3D1; 16 weeks since treatment initiation). Patient AU03-0084 received an IL-2 boost of 135,000 IU/kg on Cycle 2 Day 1 (C2D1; 8 weeks since treatment initiation).

FIG. 17 provides a timeline of the treatment with AU-007 in combination with single-dose aldesleukin and nivolumab and optional aldesleukin boost administration and tumor assessment. Cycles are 8 weeks (56 days). AU-007 is administered 4 times in a cycle, i.e., 9 mg/kg subject's body weight once every 2 weeks, (Day 1, 15, 29, 43—see black arrow); nivolumab is administered 2 times in a cycle, i.e., 480 mg once every 4 weeks (Day 1 and 29—see the black triangle); Aldesleukin is administered at 135,000 IU/kg subject's body weight as a loading dose once in Cycle 1 (Day 1—see ellipse). In an alternate embodiment, nivolumab is administered 4 times in a cycle (not shown), i.e., 240 mg once every 2 weeks (Day 1, 15, 29, and 43).

Tumor evaluation (black diamond) using RECIST evaluation of CT scan, MRI, PET scan, or ultrasound to measure existing tumor size and assess for any new tumors is carried out at the end of each cycle. Biopsy (4-point star) is conducted to evaluate immune-modulating effect of drug within tumor by evaluating immune cell infiltration. The Dose-Limiting Toxicity (DLT) period lasts 4 weeks and informs dose escalation decisions. The aldesleukin boost administration (not shown) is administered in patients who are tolerating treatment and are clinically stable at the discretion of the Investigator and Sponsor based on the following: (1) with each cycle (Q8W, Day 1 of cycle) until objective tumor shrinkage is observed on radiologic imaging or physical exam or (2) with objective signs of worsening (increased) tumor growth kinetics: e.g., previously shrinking tumors becoming stable, increase in tumor markers, tumor growth, or appearance of new tumor growth in a tumor that had previously been stable or had decreased in size. If a patient continues to subsequent cycles, they begin with AU-007 and nivolumab on Cycle 2 Day 1, then AU-007 administered with nivolumab on Days 15, 29, and 43 of Cycle 2; and so on.

FIG. 18 presents AU-007 Monotherapy: (Arm 1A) Treatment Duration and Best Response., as of Oct. 13, 2023.

FIGS. 19A and 19B present AU-007+IL-2 (Arm 1B (AU-007+1 Loading Dose Aldesleukin—FIG. 19A); Arm 1C (AU-007+Aldesleukin (Q2W)—FIG. 19B)) Treatment Duration and Best Response, as of October 2023.

FIGS. 20A, 20B, and 20C present Safety Data. FIG. 20A presents the Safety Data for Arm 1A (first 4 cohorts) AU-007 Monotherapy: Mild Toxicity Profile. FIG. 20B presents the population statistics for all three Arms (1A, 1B, and 1C) as of Oct. 13, 2023. * All Grade 3/4 drug-related AEs were transient (3-7 days) lymphopenia. ** A single drug-related SAE of transient (~12 hours) Grade 2 CRS occurred in a patient with cutaneous squamous carcinoma receiving AU-007+Q2W 135K IU/kg aldesleukin. The patient became symptomatic with fever and mild hypotension starting 6 hours after receiving the initial aldesleukin dose. The patient had a pre-treatment pneumonia with RUL consolidation treated with oral antibiotics. The patient continued therapy with mild symptoms on receiving the second doses of AU-007+aldesleukin. FIG. 20C presents a chart detailing Drug-Related Adverse Events for all three Arms (1A, 1B, and 1C) as of Oct. 13, 2023.

FIGS. 21A and 21B present Arm 1B AU-007+Proleukin® (aldesleukin): Mild Toxicity Profile of Drug Related Adverse Events (AEs). In FIG. 21B, dMMR indicates mismatch repair deficient.

FIGS. 22A, 22B, and 22C present AU-007 Objective Response: Waterfall Plot showing the ongoing results for patients within the study suffering from different cancers, as of Oct. 13, 2023. Status of tumors and subject participation within the trial, AU-007 Monotherapy (Arm 1A): Best % Change vs. Baseline, is presented in FIG. 22A. FIG. 22B shows AU-007+Aldesleukin: Best % Change vs. Baseline for all response evaluable patients who received AU-007+aldesleukin. ** Patient had a new brain lesion stabilized with radiation. FIG. 22C presents the data for AU-007+Aldesleukin: Best % Change vs. Baseline Immune Sensitive Tumors (G.I. Cancers Excluded). This includes all response evaluable patients with non-G.I. cancer who received AU-007+aldesleukin.

FIGS. 23A and 23B present AU-007 Spider Plot (FIG. 23A) and AU-007+Aldesleukin (IL-2) (FIG. 23B): Percent (%) Tumor Changes Over Time. FIG. 23B includes all response evaluable patients who received AU-007+aldesleukin as of Oct. 13, 2023.

FIGS. 24A-24C presents tumor assessments by computed tomography scans (baseline and 8-week scans) from melanoma patient, wherein the cancer did not respond to checkpoint inhibitors anti-PD-1 and/or CTLA4.

FIG. 25 presents graphs of AU-007 concentration over time. The left-side figure is an expansion of the first 60 hours demonstrating the Tmax and C-max. The right-side figure represents the data set as of June 2023. Note that not all cohorts have complete data as this data is currently being acquired. Overall, the data show AU007 demonstrates typical IgG1 therapeutic characteristics.

FIGS. 26A and 26B show that AU-007 pharmacodynamic data demonstrating AU-007 continues to decrease peripheral blood circulating Tregs (as measured by flow cytometry) over time (days) following administration of AU-007. Percent change in the absolute number of circulating regulatory T cells. Regulatory T cells were defined as CD3+CD4+CD25+CD127lo of the CD45+ cells. Consistent with the mechanism of action of inhibiting IL-2 from interacting with the trimeric receptor, regulatory T cells decreased in the peripheral circulation. This was observed in both the monotherapy arm and the arms which also included Proleukin® and was consistent among patients. FIG. 26 presents the data per individual patient (receiving AU-007+/− at least one dose of Proleukin®—See Key in FIG. 27) and FIG. 28B presents the data per dosage group (AU-007 only). The values represent the change from baseline of the absolute (abs) cell counts.

FIG. 27 presents average percent change/days in peripheral blood Tregs for cohorts receiving at least one dose of Proleukin® (aldesleukin) along with 4.5 mg/kg of AU-007. The values represent the change from baseline of the absolute (abs) cell counts. Data points are continued to be collected.

FIGS. 28A and 28B show changes in absolute peripheral blood CD8 cells over time (days) following administration of AU-007. FIG. 28A presents the data per individual patient and FIG. 28B presents the data per dosage group. The values represent the change from baseline of the absolute (abs) cell counts. Data collection is ongoing.

FIG. 29 shows average percent change/day in peripheral blood CD8 for cohorts receiving Proleukin® (aldesleukin) along with administration of AU-007. The values represent the change from baseline of the absolute (abs) cell counts. Data collection is ongoing.

FIGS. 30A and 30B show changes in absolute peripheral blood NK cells over time (days) following administration of AU-007. FIG. 30A presents the data per individual patient and FIG. 52B presents the data per dosage group. The values represent the change from baseline of the absolute (abs) cell counts. Data collection is ongoing.

FIG. 31 shows change in peripheral blood NK cells for cohorts receiving Proleukin® (aldesleukin) over time (days) following administration of AU-007. The values represent the change from baseline of the absolute (abs) cell counts. Data as of September 2023

FIGS. 32A and 32B show absolute numbers of eosinophils over time (days) over an extended time period following administration of AU-007. FIG. 32A Individual Peripheral Blood Eosinophil Counts in AU-007-Only Cohorts. FIG. 32B Individual Peripheral Blood Eosinophil Counts in AU-007+Proleukin® Cohorts. FIGS. 32A and 32B show changes over time in the circulating number of eosinophils. FIG. 32A are the cohorts receiving only AU-007 monotherapy and FIG. 32B are cohorts receiving AU-007 with at least 1 dose of Proleukin®. All but one patient in the AU-007 monotherapy and AU-007 with Proleukin® arms demonstrated a decrease or no change in the circulating levels of eosinophils. A patient in the 9 mg/kg cohort had severe seasonal allergies requiring treatment during time on AU-007 treatment and is consistent with a history of being treated for seasonal allergies. The rise in eosinophils was attributed to the allergy reaction. All patients given AU-007 with Proleukin® showed stable or a decrease in circulating eosinophils. This is consistent with the mechanism of action of AU-007 preventing IL-2 from interacting with the IL-2 trimeric receptor on eosinophils. The values represent the change from baseline of the absolute (abs) cell counts. Data collection is ongoing.

FIGS. 33A-33C show the CD8:Treg ratios over time (days) over an extended time period in the periphery, following administration of AU-007. FIG. 33A shows all available data per individual patient. (For key, see FIG. 33C) FIG. 33B presents the data per dosage group. FIG. 33C shows data per individual patient also receiving Proleukin® (aldesleukin). Consistent with the observations seen in the changes in Tregs and CD8+ T cells, there is an observed trend to an increase in the CD8+/Treg ratio with monotherapy. In the presence of Proleukin®, an increase in the CD8+/Treg ratio was observed, particularly at higher doses of Proleukin®. Consistent with the mechanism of action, higher doses (of low dose IL-2), and longer exposure trend to higher CD8+/Treg ratios with no observed drug-related toxicity. It is anticipated that increasing doses of Proleukin® will further enhance the peripheral response. The values represent the change from baseline of the absolute (abs) cell counts. Data collection is ongoing.

FIGS. 34A and 34B present the fold change in the expression of IFN-γ in patients dosed with AU-007+/−Proleukin®. A heat map of the change from baseline in the circulating levels of interferon gamma (IFN-γ). The small-dashed boxes represent a 20%-2-fold change, the large-dashed box a 2-5-fold change and the solid box >5-fold change. These preliminary results demonstrate that the longer a patient is on monotherapy (FIG. 34A), the more likely the patients is to have increases in circulating IFN-γ. This is consistent with the observations in circulating cell populations, particularly Treg and NK cells. The addition of low dose IL-2 in the presence of AU-007 (FIG. 34B) consistently increases IFN-γ in the peripheral circulation.

FIG. 35 presents the Phase 1/2 clinical study with a focus now on the Phase 1 Dose Escalation portion (1C).

FIG. 36 presents an updated AU-007+Aldesleukin Waterfall Plot: Best % Change vs. Baseline.

FIG. 37 presents an updated AU-007+Aldesleukin Spider Plot: % Change vs. Baseline Over Time, pointing out the greater than 30% cancer reduction in a patient with nasopharyngeal cancer. The data shown includes all response evaluable patients who received AU-007+aldesleukin (IL-2).

FIG. 38 presents an updated AU-007+Aldesleukin Waterfall Plot: Best % Change in Immune Sensitive Tumors. The data shown includes all response evaluable patients with non-G.I. cancer who received AU-007+aldesleukin (IL-2).

FIG. 39 presents computed tomography scans showing 40% shrinkage in the target lesions of a melanoma patient whose tumors progressed through prior anti-PD-1+CTLA4 therapy.

FIG. 40 presents computed tomography scans showing 20% shrinkage in first 8 weeks in the target lesions of a RCC patient whose tumors progressed through prior anti-PD-1 therapy.

FIGS. 41A and 41B present evidence of response of melanoma patients to administration of AU-007+IL-2 loading dose without addition of a checkpoint inhibitor (CPI); RP2D. FIG. 41A shows best response in the target lesions of melanoma patients who progressed on prior CPI therapy treated with imneskibart (AU-007)+low dose SQ IL-2 (n=14). FIG. 41B shows the percentage change over time (months) vs. baseline in the target lesions of the same melanoma patient population (n=14). The arrows indicate administration of a low-dose SQ IL-2 boost (135,000 IU/kg) for a patient. The solid circles indicate patients continuing in the clinical trial.

FIGS. 42A and 42B present early signs of anti-tumor activity in 5 melanoma patients who progressed on prior doublet* CPI therapy, and were then treated with the RP2D-1 (3 patients) or RP2D (2 patients): nivolumab+imneskibart+low dose SQ IL-2. Patients with BRAF/MEK mutations were eligible only if not treated with prior BRAF/MEK inhibitor. Patients had progressed on prior doublet checkpoint inhibitors: anti-CTLA-4+anti-PD-1 and/or anti-PD-1+anti-Lag-3. FIG. 42A shows best response in the target lesions of melanoma patients who had progressed on prior doublet CPI therapy (n=5; the patient with tumor reduction (−22%) and the patients with increases of +24% and +2% received RP2D-1 and the 2 patients with 0 change received RP2D). FIG. 42B shows percentage change over time vs. baseline in the target lesions of these melanoma patients who progressed on prior doublet CPI therapy. The arrows indicate administration of a low-dose SQ IL-2 boost (135,000 IU/kg) for a patient. The solid circles indicate patients continuing in the clinical trial.

FIGS. 43A and 43B show early evidence of response of NSCLC patients to administration of AU-007+IL-2 loading dose. FIG. 43A presents the best response (best change from baseline) in NSCLC patients treated with imneskibart (AU-007)+IL-2. FIG. 43B presents the percentage change over time versus baseline of target lesion sum of diameters in NSCLC patients treated with imneskibart (AU-007)+IL-2. Solid circle indicates patient is ongoing in the study.

FIGS. 44A and 44B present pharmacodynamic profiles of Tregs (FIG. 44A) and CD8/Treg Ratios (FIG. 44B) in Melanoma patients treated with the RP2D. FIG. 44A presents data showing the mean absolute Treg change in melanoma patients over time (days). FIG. 44B presents data showing the mean absolute CD8/Treg change over time (days). Day 1 through Day 3 are omitted from these graphs as all peripheral lymphocytes decrease over these days. This is considered an effect of IL-2 causing a brief redistribution of lymphocytes out of the peripheral blood. The filled circle is imneskibart+IL-2; Imneskibart 9 mg/kg, IL-2 135K IU/kg (n=14). The filled square is 9 mg/kg imneskibart+Nivo (480 mg nivolumab)+45K IL-2 (RP2D-1); n=3. FC is Fold Change.

FIGS. 45A-45C present data indicating that higher peripheral blood CD8/Treg ratio is associated with better efficacy vs low CD8/Treg ratio. FIG. 45A presents the time of patients on treatment; FIG. 45B presents the progression-free survival (PFS); and FIG. 45C presents the overall survival (OS). FC is Fold Change.

FIGS. 46A and 46B show the response of melanoma patients to administration of RP2D AU-007+IL-2 loading dose without nivolumab. FIG. 46A shows best response in the target lesions of melanoma patients who progressed on prior CPI therapy (anti-CTLA-4+anti-PD- and/or anti-PD-1+anti-LAG-3) treated with imneskibart (AU-007)+low dose SQ IL-2 (n=14). * (within each bar) marks patients that failed prior doublet CPI; solid circle marks patient continuing in the clinical trial. FIG. 46B shows the percentage change over time (months) vs. baseline in the target lesions of the same melanoma patient population (n=14). The arrows mark administration of a low-dose SQ IL-2 boost (135,000 IU/kg); [symbol] marks patient continuing in the clinical trial.

FIGS. 47A and 47B show best response in the target lesions of melanoma patients who had progressed on prior doublet CPI therapy (n=12). In FIG. 47A patients that received RP2D-1 exhibited changes in target lesions of +24%, +2%, and −65% (partial response). Among patients treated at the RP2D, 3 obtained a partial response with 52%, 74%, and 77% decrease in target lesions. The solid circles mark patients continuing in the clinical trial. The patient exhibiting a decrease in target lesions of 52% is in follow-up but is not receiving further treatment. FIG. 47B shows percentage change over time vs. baseline in the target lesions of these melanoma patients who progressed on prior doublet CPI therapy. The arrows mark administration of a low-dose SQ IL-2 boost (135,000 IU/kg) for a patient; [symbol] marks patient continuing in the clinical trial. The lowest graph indicates the patient that is in follow-up but is not receiving any treatment.

FIGS. 48A-48D show computed tomography scans from a 28-year old melanoma patient, where the cancer did not respond to prior checkpoint inhibitors anti-PD-1 and anti-CTLA-4 doublet therapy. FIG. 48A presents the baseline CT scan, wherein bilateral lung and lymph node metastases are shown (circled in black); FIG. 48B presents the 8-month CT scan of bilateral lung and lymph node metastases; FIG. 48C presents the baseline L lower lobe lung metastases circled in black; FIG. 48D presents the 8-month CT scan of L lower lobe lung metastases.

FIGS. 49A-49D show computed tomography scans from a 69-year old melanoma patient, where the cancer did not respond to checkpoint inhibitors anti-PD-1 and anti-LAG-3 doublet therapy. FIG. 49A presents the baseline CT scan, wherein L thorax subcutaneous metastases are shown (circled in black); FIG. 49B presents 4-month CT scan of the L thorax subcutaneous metastases; FIG. 49C presents the baseline L upper lobe lung metastasis; FIG. 49D presents the 4-month scan of the L upper lobe lung metastasis.

FIG. 50 shows RECIST evaluable melanoma patients in Phase 1 and 2 who received 9 mg/kg Q2W imneskibart combined with the single loading dose of 135,000 IU/kg aldesleukin. X-axis represents—Months on treatment. Left to the ‘0’ value on the X-axis is duration of Prior Doublet CPI therapy. Right to the ‘0’ value on the X-axis is the treatment duration of imneskibart+aldesleukin. Prior Doublet CPI therapy shown on the right for each patient (ipilimumab/nivolumab or nivolumab/relatlimab). The first Barfrom the top represents a patient that remains on study and not receiving treatment in follow-up; bars 3 and 6 from the top represent patients with ongoing treatment.

FIGS. 51A and 51B show intratumoral effector CD8 and NK cell increases after one 8-week treatment cycle with imneskibart+aldesleukin.

FIGS. 52A and 52B present Higher peripheral blood CD8/Treg Ratio Associated With Better Anti-Tumor Activity. FIG. 51A shows Progression-Free Survival over the time of the ongoing study. Results were calculated by deriving the median greatest Treg decrease across all patients (excluding the first 2 days on treatment) and dividing the patients who were ≤ the median or > than the median for CD8/Treg ratio fold change versus baseline. FIG. 52B shows the Progression Status at 16 Weeks. The Maximum CD8/Treg Fold Change vs. Baseline by Week 16 Progression Status. Patients were divided by progression status after 16 weeks (2 treatment Cycles) on treatment and the individual patient maximum CD8/Treg ratio fold increase over baseline was plotted for each patient. Boxplots summarize maximum CD8/Treg ratio fold increase by progression status group showing median (line in each box) with lower and upper box boundaries corresponding to the 25th and 75th percentile.

### DETAILED DESCRIPTION

In the following detailed description, numerous specific details are set forth in order to provide a thorough understanding of the antibodies disclosed herein. However, it will be understood by those skilled in the art that preparation and uses of antibodies disclosed herein may in certain cases be practiced without these specific details. In other instances, well-known methods, procedures, and components have not been described in detail so as not to obscure the disclosure presented herein.

Throughout this application, various references or publications are cited. Disclosures of these references or publications in their entireties are hereby incorporated by reference into this application in order to more fully describe the state of the art to which this invention pertains.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind an IL-2 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

As used herein, the term “antibody” encompasses an antibody fragment or fragments that retain binding specificity including, but not limited to, IgG, heavy chain variable region (VH), light chain variable region (VL), Fab fragments, F(ab′)2 fragments, scFv fragments, Fv fragments, a nanobody, minibodies, diabodies, triabodies, tetrabodies, and single domain antibodies (see, e.g., Hudson and Souriau, Nature Med. 9: 129-134 (2003)). Also encompassed are humanized, primatized, and chimeric antibodies as these terms are generally understood in the art.

A skilled artisan would appreciate that in certain embodiments, the term “anti-IL-2 antibody” as used herein is interchangeable with the term “anti-human-IL-2 antibody”, having all the same qualities and meanings. Similarly, as used throughout, in certain embodiments, the term “IL-2” is interchangeable with the term “human IL-2”, having all the same qualities and meanings.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen-binding site. In some embodiments, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

Wu and Kabat (Tai Te Wu, Elvin A. Kabat. An analysis of the sequences of the variable regions of bence jones proteins and myeloma light chains and their implications for antibody complementarity. Journal of Experimental Medicine, 132, 2, 8 (1970); Kabat E A, Wu T T, Bilofsky H, Reid-Miller M, Perry H. Sequence of proteins of immunological interest. Bethesda: National Institute of Health; 1983. 323 (1983)) pioneered the alignment of antibody peptide sequences, and their contributions in this regard were several-fold: Firstly, through study of sequence similarities between variable domains, they identified correspondent residues that to a greater or lesser extent were homologous across all antibodies in all vertebrate species, inasmuch as they adopted similar three-dimensional structure, played similar functional roles, interacted similarly with neighboring residues, and existed in similar chemical environments. Secondly, they devised a peptide sequence numbering system in which homologous immunoglobulin residues were assigned the same position number. One skilled in the art can unambiguously assign to any variable domain sequence what is now commonly called Kabat numbering without reliance on any experimental data beyond the sequence itself. Thirdly, Kabat and Wu calculated variability for each Kabat-numbered sequence position, by which is meant the finding of few or many possible amino acids when variable domain sequences are aligned. They identified three contiguous regions of high variability embedded within four less variable contiguous regions. Kabat and Wu formally demarcated residues constituting these variable tracts, and designated these “complementarity determining regions” (CDRs), referring to chemical complementarity between antibody and antigen. A role in three-dimensional folding of the variable domain, but not in antigen recognition, was ascribed to the remaining less-variable regions, which are now termed “framework regions”. Fourth, Kabat and Wu established a public database of antibody peptide and nucleic acid sequences, which continues to be maintained and is well known to those skilled in the art.

Chothia and coworkers (Cyrus Chothia, Arthur M. Lesk. Canonical structures for the hypervariable regions of immunoglobulins. Journal of Molecular Biology, 196, 4, 8 (1987)) found that certain sub portions within Kabat CDRs adopt nearly identical peptide backbone conformations, despite having great diversity at the level of amino acid sequence. These sub portions were designated as L1, L2 and L3 or H1, H2 and H3, where the “L” and the “H” designates the light chain and the heavy chains regions, respectively. These regions may be referred to as Chothia CDRs, which have boundaries that overlap with Kabat CDRs.

More recent studies have shown that virtually all antibody binding residues fall within regions of structural consensus. (Kunik, V. et al., PloS Computational Biology 8(2):e1002388 (February 2012)). In some embodiments, these regions are referred to as antibody binding regions. It was shown that these regions can be identified from the antibody sequence as well. “Paratome”, an implementation of a structural approach for the identification of structural consensus in antibodies, was used for this purpose. (Ofran, Y. et al., J. Immunol. 757:6230-6235 (2008)). While residues identified by Paratome cover virtually all the antibody binding sites, the CDRs (as identified by the commonly used CDR identification tools) miss significant portions of them. Antibody binding residues which were identified by Paratome but were not identified by any of the common CDR identification methods are referred to as Paratome-unique residues. Similarly, antibody binding residues that are identified by any of the common CDR identification methods but are not identified by Paratome are referred to as CDR-unique residues. Paratome-unique residues make crucial energetic contributions to antibody-antigen interactions, while CDRs-unique residues make a rather minor contribution. These results allow for better identification of antigen binding sites.

IMGT@ is the international ImMunoGeneTics information System®, (See, Nucleic Acids Res. 2015 January; 43 (Database issue):D413-22. doi: 10.1093/nar/gku1056. Epub 2014 Nov. 5 Free article. PMID: 25378316 LIGM:441 and Dev Comp Immunol. 2003 January; 27(1):55-77). IMGT is a unique numbering system for immunoglobulin and T cell receptor variable domains and Ig superfamily V-like domains (Lefranc et al., Dev Comp Immunol. 27: 55-77 (2003)). IMGT@presents a uniform numbering system for these IG and TcR variable domain sequences, based on aligning 5 or more IG and TcR variable region sequences, taking into account and combining the Kabat definition of FRs and CDRs, structural data, and Chothia's characterization of the hypervariable loops. IMGT is considered well known in the art as a universal numbering scheme for antibodies.

In some embodiments, identification of potential variant amino acid positions in the VH and VL domains uses the IMGT system of analysis. In some embodiments, identification of potential variant amino acid positions in the VH and VL domains uses the Paratome system of analysis. In some embodiments, identification of potential variant amino acid positions in the VH and VL domains uses the Kabat system of analysis. In some embodiments, identification of potential variant amino acid positions in the VH and VL domains uses the Clothia system of analysis.

In describing variant amino acid positions present in the VH and VL domains, in some embodiments the IMGT numbering is used. In describing variant amino acid positions present in the VH and VL domains, in some embodiments the Paratome numbering is used. In describing variant amino acid positions present in the VH and VL domains, in some embodiments the Kabat numbering is used. In describing variant amino acid positions present in the VH and VL domains, in some embodiments the Clothia numbering is used.

Antigen binding sequences are conventionally located within the heavy chain and light chain variable regions of an antibody. These heavy and light chain variable regions may, in certain instances, be manipulated to create new binding sites, for example to create antibodies or fragments thereof, that bind to a different antigen or to a different epitope of the same antigen. In some embodiments, as described herein, manipulating the sequences of a heavy chain variable region or the sequences of a light chain variable region, or both, would create a new binding site for a second antigen.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide, the linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)₂ fragments, scFv fragments, Fv fragments, minibodies, diabodies, triabodies, and tetrabodies.

### Methods of Use

In some embodiments, the present disclosure provides methods of treating a solid cancer in a subject comprising administering to said subject an anti-IL-2 antibody or a pharmaceutical composition thereof, a loading dose of IL-2 or a pharmaceutical composition thereof, and at least one booster dose of IL-2 or a pharmaceutical composition thereof. In some embodiments, the present disclosure provides methods of treating an unresectable locally advanced or metastatic solid cancer in a subject comprising administering to said subject an anti-IL-2 antibody or a pharmaceutical composition thereof, a loading dose of IL-2 or a pharmaceutical composition thereof, and at least one booster dose of IL-2 or a pharmaceutical composition thereof. In some embodiments, the present disclosure provides methods of treating an unresectable locally advanced or metastatic solid cancer comprising a cutaneous melanoma, a melanoma, a renal cell carcinoma (RCC), a non-small cell lung cancer (NSCLC), a squamous NSCLC, a non-squamous NSCLC, a head and neck carcinoma, a head and neck squamous cell carcinoma (HNSCC), a nasopharyngeal carcinoma, an esophageal carcinoma, a gastric carcinoma, a gastric or gastro-esophageal cancer, a gastroesophageal junction carcinoma, an esophageal squamous cell carcinoma, a cutaneous squamous cell carcinoma (cSCC), a hepato-cellular carcinoma (HCC), a colorectal cancer (CRC), a microsatellite instability high (MSHi) CRC, a microsatellite stable (MSHs) CRC, a cervical carcinoma, an endometrial carcinoma, a urothelial cancer, a bladder cancer, a ureteral cancer, a renal pelvis cancer, a malignant mesothelioma, a malignant pleural mesothelioma, a malignant peritoneal mesothelioma, a classical Hodgkin Lymphoma (cHL), a biliary tract carcinoma (BTC), a MSHi or mismatch repair deficient cancer, a Tumor Mutational Burden-High (TMB-H) cancer, Triple-negative breast cancer (TNBC), or a Merkel cell carcinoma (MCC), or a combination thereof, in a subject comprising administering to said subject an anti-IL-2 antibody or a pharmaceutical composition thereof, a loading dose of low dose IL-2 or a pharmaceutical composition thereof, and at least one booster dose of low dose IL-2 or a pharmaceutical composition thereof. In some embodiments, multiple doses of anti-IL-2 antibody are administered. In some embodiments, the pharmaceutical composition further comprises a pharmaceutically acceptable carrier.

In some embodiments, the present disclosure provides a method of treating a solid cancer in a subject comprising administering to said subject an anti-IL-2 antibody or a pharmaceutical composition thereof and nivolumab or a pharmaceutical composition thereof. In some embodiments, the present disclosure provides a method of treating an unresectable locally advanced or metastatic solid cancer in a subject comprising administering to said subject an anti-IL-2 antibody or a pharmaceutical composition thereof and nivolumab or a pharmaceutical composition thereof. In some embodiments, the present disclosure provides a method of treating an unresectable locally advanced or metastatic solid cancer comprising a cutaneous melanoma, a melanoma, a renal cell carcinoma (RCC), a non-small cell lung cancer (NSCLC), a squamous NSCLC, a non-squamous NSCLC, a head and neck carcinoma, a head and neck squamous cell carcinoma (HNSCC), a nasopharyngeal carcinoma, an esophageal carcinoma, a gastric carcinoma, a gastric or gastro-esophageal cancer, a gastroesophageal junction carcinoma, an esophageal squamous cell carcinoma, a cutaneous squamous cell carcinoma (cSCC), a hepato-cellular carcinoma (HCC), a colorectal cancer (CRC), a microsatellite instability high (MSHi) CRC, a microsatellite stable (MSHs) CRC, a cervical carcinoma, an endometrial carcinoma, a urothelial cancer, a bladder cancer, a ureteral cancer, a renal pelvis cancer, a malignant mesothelioma, a malignant pleural mesothelioma, a malignant peritoneal mesothelioma, a classical Hodgkin Lymphoma (cHL), a biliary tract carcinoma (BTC), a MSHi or mismatch repair deficient cancer, a Tumor Mutational Burden-High (TMB-H) cancer, Triple-negative breast cancer (TNBC), or a Merkel cell carcinoma (MCC), or a combination thereof, in a subject comprising administering to said subject an anti-IL-2 antibody or a pharmaceutical composition thereof and nivolumab or a pharmaceutical composition thereof.

In some embodiments, the present disclosure provides a method of treating a solid cancer in a subject comprising administering to said subject an anti-IL-2 antibody or a pharmaceutical composition thereof, a loading dose of IL-2 or a pharmaceutical composition thereof, and nivolumab or a pharmaceutical composition thereof. In some embodiments, the present disclosure provides a method of treating an unresectable locally advanced or metastatic solid cancer in a subject comprising administering to said subject an anti-IL-2 antibody or a pharmaceutical composition thereof, a loading dose of IL-2 or a pharmaceutical composition thereof, and nivolumab or a pharmaceutical composition thereof. In some embodiments, the present disclosure provides a method of treating an unresectable locally advanced or metastatic solid cancer comprising a cutaneous melanoma, a melanoma, a renal cell carcinoma (RCC), a non-small cell lung cancer (NSCLC), a squamous NSCLC, a non-squamous NSCLC, a head and neck carcinoma, a head and neck squamous cell carcinoma (HNSCC), a nasopharyngeal carcinoma, an esophageal carcinoma, a gastric carcinoma, a gastric or gastro-esophageal cancer, a gastroesophageal junction carcinoma, an esophageal squamous cell carcinoma, a cutaneous squamous cell carcinoma (cSCC), a hepato-cellular carcinoma (HCC), a colorectal cancer (CRC), a microsatellite instability high (MSHi) CRC, a microsatellite stable (MSHs) CRC, a cervical carcinoma, an endometrial carcinoma, a urothelial cancer, a bladder cancer, a ureteral cancer, a renal pelvis cancer, a malignant mesothelioma, a malignant pleural mesothelioma, a malignant peritoneal mesothelioma, a classical Hodgkin Lymphoma (cHL), a biliary tract carcinoma (BTC), a MSHi or mismatch repair deficient cancer, a Tumor Mutational Burden-High (TMB-H) cancer, Triple-negative breast cancer (TNBC), or a Merkel cell carcinoma (MCC), or a combination thereof, in a subject comprising administering to said subject an anti-IL-2 antibody or a pharmaceutical composition thereof, a loading dose of IL-2 or a pharmaceutical composition thereof, and nivolumab or a pharmaceutical composition thereof.

In some embodiments, the present disclosure provides methods of treating an unresectable locally advanced or metastatic solid tumor. In some embodiments, an unresectable locally advanced or metastatic solid cancer comprises a cutaneous melanoma, a melanoma, a renal cell carcinoma (RCC), a non-small cell lung cancer (NSCLC), a squamous NSCLC, a non-squamous NSCLC, a head and neck carcinoma, a head and neck squamous cell carcinoma (HNSCC), a nasopharyngeal carcinoma, an esophageal carcinoma, a gastric carcinoma, a gastric or gastro-esophageal cancer, a gastroesophageal junction carcinoma, an esophageal squamous cell carcinoma, a cutaneous squamous cell carcinoma (cSCC), a hepato-cellular carcinoma (HCC), a colorectal cancer (CRC), a microsatellite instability high (MSHi) CRC, a microsatellite stable (MSHs) CRC, a cervical carcinoma, an endometrial carcinoma, a urothelial cancer, a bladder cancer, a ureteral cancer, a renal pelvis cancer, a malignant mesothelioma, a malignant pleural mesothelioma, a malignant peritoneal mesothelioma, a classical Hodgkin Lymphoma (cHL), a biliary tract carcinoma (BTC), a MSHi or mismatch repair deficient cancer, a Tumor Mutational Burden-High (TMB-H) cancer, Triple-negative breast cancer (TNBC), or a Merkel cell carcinoma (MCC).

In some embodiments, the present disclosure provides methods of treating cutaneous melanoma, a melanoma, a renal cell cancer, head and neck carcinoma, urothelial cancer, non-small cell lung cancer (NSCLC), hepatocellular carcinoma, esophageal carcinoma, gastric carcinoma, gastroesophageal junction carcinoma, malignant pleural and peritoneal mesothelioma, classical Hodgkin Lymphoma (cHL), colorectal carcinoma, cervical carcinoma, endometrial carcinoma, biliary tract carcinoma (BTC), Merkel cell carcinoma (MCC), cutaneous squamous cell carcinoma (cSCC), triple-negative breast cancer (TNBC), or a combination thereof. In some embodiments, the renal cell cancer comprises a renal cell carcinoma (RCC). In some embodiments, the head and neck carcinoma comprises head and neck squamous cell carcinoma (HNSCC) or nasopharyngeal carcinoma (NPC). In some embodiments, a urothelial cancer comprises bladder cancer, ureteral cancer, renal pelvis cancer, or a combination thereof. In some embodiments, a NSCLC comprises a squamous NSCLC and in other embodiments, a non-squamous NSCLC. In some embodiments, a colorectal carcinoma comprises microsatellite instability high (MSHi) colorectal carcinoma. In other embodiments, a colorectal carcinoma comprises a microsatellite stable (MSHs) colorectal carcinoma.

In other embodiments, the present disclosure provides methods of treating any MSHi or mismatch repair deficient cancer, any Tumor Mutational Burden-High (TMB-H) cancer, or a combination thereof.

In some embodiments, the present disclosure provides methods of treating a melanoma, a metastatic melanoma, a primary melanoma and metastatic melanoma, a cutaneous melanoma, a melanoma, a renal cell carcinoma (RCC), a non-small cell lung cancer (NSCLC), a squamous NSCLC, a non-squamous NSCLC, a nasopharyngeal carcinoma, a urothelial cancer, an adrenal cortical carcinoma, a clear cell renal cell carcinoma (ccRCC), a triple-negative breast cancer (TNBC), a head and neck cancer, a head and neck squamous cell carcinoma (HNSCC), an esophageal carcinoma, a gastric carcinoma, a gastric or gastro-esophageal cancer, a gastroesophageal junction carcinoma, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), a microsatellite instability high (MSHi) CRC, a microsatellite stable (MSHs) CRC, an epithelial ovarian cancer, a cervical cancer, a cervical carcinoma, an endometrial cancer, an endometrial carcinoma, a thyroid cancer (follicular or papillary histology), a lung cancer, a bladder cancer, a uterine cancer, a ureteral cancer, a renal pelvis cancer, a gallbladder cancer, a malignant mesothelioma, a malignant pleural mesothelioma, a malignant peritoneal mesothelioma, a classical Hodgkin Lymphoma (cHL), a biliary tract carcinoma (BTC), or a Merkel cell carcinoma (MCC). In other embodiments, the present disclosure provides methods of treating or preventing a condition caused by IL-2 binding to endothelial CD25 expressing cells, e.g., pulmonary edema, or IL-2-induced vascular leakage.

In other embodiments, the present disclosure provides methods of treating a solid tumor. In some embodiments, a solid tumor comprises a cutaneous melanoma, a melanoma, a head and neck cancer, a head and neck squamous cell carcinoma (HNSCC), a pancreatic cancer, a lung cancer, a thyroid cancer, a non-small cell lung cancer (NSCLC), a nasopharyngeal carcinoma, a melanoma, an acral melanoma, a uveal melanoma, a colorectal cancer (CRC), a bladder cancer, cholangiocarcinoma (bile duct cancer), a uterine cancer, a cervical cancer, a gallbladder cancer, a cutaneious squamous carcinoma, or a renal cell carcinoma (RCC). In some embodiments, a solid tumor comprises a head and neck cancer, a pancreatic cancer, or a non-small cell lung cancer. In some embodiments, a solid tumor comprises a non-small cell lung cancer (NSCLC), a melanoma, a metastatic melanoma, a primary melanoma and a metastatic melanoma, or a renal cell carcinoma (RCC). In some embodiments, a solid tumor comprises a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma.

In some embodiments, a solid tumor comprises a head and neck cancer. In some embodiments, a solid tumor comprises a pancreatic cancer. In some embodiments, a solid tumor comprises a lung cancer. In some embodiments, a solid tumor comprises a thyroid cancer. In some embodiments, a solid tumor comprises a nasopharyngeal carcinoma. In some embodiments, a solid tumor comprises a melanoma. In some embodiments, a melanoma comprises an acral melanoma or a uveal melanoma. In some embodiments, a solid tumor comprises a colorectal cancer (CRC). In some embodiments, a solid tumor comprises a bladder cancer. In some embodiments, a solid tumor comprises cholangiocarcinoma (bile duct cancer). In some embodiments, a solid tumor comprises a uterine cancer. In some embodiments, a solid tumor comprises a cervical cancer. In some embodiments, a solid tumor comprises a gallbladder cancer. In some embodiments, a solid tumor comprises a renal cell carcinoma (RCC). In some embodiments, a head and neck cancer is a head and neck squamous carcinoma (HNSCC1). In some embodiments, a CRC has high MSI. In some embodiments, a melanoma has a wild-type BRAF gene. In some embodiments, a melanoma has a mutant BRAF gene. In some embodiments, a pancreatic cancer is an adenocarcinoma. In some embodiments, a non-small cell lung cancer (NSCLC) is a squamous cancer. In some embodiments, a NSCLC has a mutated epidermal growth factor receptor (EGFRm). In some embodiments, a solid tumor comprises a cutaneous squamous carcinoma.

In some embodiments, the present disclosure provides methods of treating a melanoma in a subject. In some embodiments, the present disclosure provides methods of treating a cutaneous melanoma in a subject. In some embodiments, the present disclosure provides methods of treating a locally advanced/metastatic cutaneous melanoma.

In some embodiments, the method described herein comprises treating the primary cancer and secondary metastasis of the cancer. In some embodiments, the method described herein comprises treating the secondary metastasis of the cancer. In some embodiments, the method described herein is a first line treatment of the cancer. In some embodiments, the method described herein is a second line treatment of the cancer. In some embodiments, the method described herein is a third line treatment of the cancer. In some embodiments, the method described herein is a fourth line treatment of the cancer. In some embodiments, the method described herein is a fifth line treatment of the cancer. In some embodiments, the method described herein is a sixth line treatment of the cancer. In some embodiments, the method described herein is a seventh line treatment of the cancer. In some embodiments, the method described herein is an eighth line treatment of the cancer. In some embodiments, the method described herein is a ninth line treatment of the cancer. In some embodiments, the method described herein is first-, second-, or third-line treatments of the cancer. In some embodiments, the method described herein is second and third line treatments of the cancer.

In some embodiments, the cancer comprises an unresectable locally advanced or metastatic cancer. In other embodiments, the cancer comprises an unresectable locally advanced or metastatic solid cancer. In some embodiments, the unresectable locally advanced or metastatic cancer comprises a melanoma, a renal cell carcinoma (RCC), a non-small cell lung cancer (NSCLC), a head and neck squamous cell carcinoma (HNSCC), gastric or gastro-esophageal cancer, esophageal squamous cell carcinoma, cutaneous squamous cell carcinoma (cSCC), pancreatic adenocarcinoma, cholangiocarcinoma (bile duct cancer), hepato-cellular carcinoma (HCC), colorectal cancer (CRC), epithelial ovarian cancer, cervical cancer, endometrial cancer, thyroid cancer having follicular or papillary histology, urothelial cancer, bladder cancer, uterine cancer, gallbladder cancer, or Merkel cell carcinoma.

In some embodiments, treating an unresectable locally advanced or metastatic cancer comprises treating the primary cancer and secondary metastasis of the cancer. In some embodiments, treating an unresectable locally advanced or metastatic cancer comprises treating the secondary metastasis of the cancer. In some embodiments, treating an unresectable locally advanced or metastatic cancer comprises a first line treatment of the cancer. In some embodiments, treating an unresectable locally advanced or metastatic cancer comprises a second line treatment of the cancer. In some embodiments, treating an unresectable locally advanced or metastatic cancer comprises a third line treatment of the cancer. In some embodiments, treating an unresectable locally advanced or metastatic cancer comprises a fourth line treatment of the cancer. In some embodiments, treating an unresectable locally advanced or metastatic cancer comprises a fifth line treatment of the cancer. In some embodiments, treating an unresectable locally advanced or metastatic cancer comprises a sixth line treatment of the cancer. In some embodiments, treating an unresectable locally advanced or metastatic cancer comprises a seventh line treatment of the cancer. In some embodiments, treating an unresectable locally advanced or metastatic cancer comprises an eighth line treatment of the cancer. In some embodiments, treating an unresectable locally advanced or metastatic cancer comprises a ninth line treatment of the cancer. In some embodiments, treating an unresectable locally advanced or metastatic cancer comprises second and third line treatments of the cancer.

In some embodiments, treating an unresectable locally advanced or metastatic cutaneous melanoma comprises treating the primary cutaneous melanoma and secondary metastasis of the cutaneous melanoma. In some embodiments, treating an unresectable locally advanced or metastatic cutaneous melanoma comprises treating the secondary metastasis of the cutaneous melanoma. In some embodiments, treating an unresectable locally advanced or metastatic cutaneous melanoma comprises a first line treatment of the cutaneous melanoma. In some embodiments, treating an unresectable locally advanced or metastatic cutaneous melanoma comprises a second line treatment of the cutaneous melanoma. In some embodiments, treating an unresectable locally advanced or metastatic cutaneous melanoma comprises a third line treatment of the cutaneous melanoma. In some embodiments, treating an unresectable locally advanced or metastatic cutaneous melanoma comprises a fourth line treatment of the cutaneous melanoma. In some embodiments, treating an unresectable locally advanced or metastatic cutaneous melanoma comprises a fifth line treatment of the cutaneous melanoma. In some embodiments, treating an unresectable locally advanced or metastatic cutaneous melanoma comprises a sixth line treatment of the cutaneous melanoma. In some embodiments, treating an unresectable locally advanced or metastatic cutaneous melanoma comprises a seventh line treatment of the cutaneous melanoma. In some embodiments, treating an unresectable locally advanced or metastatic cutaneous melanoma comprises an eighth line treatment of the cutaneous melanoma. In some embodiments, treating an unresectable locally advanced or metastatic cutaneous melanoma comprises a ninth line treatment of the cutaneous melanoma. In some embodiments, treating an unresectable locally advanced or metastatic cutaneous melanoma comprises second and third line treatments of the cutaneous melanoma.

In some embodiments, treating an unresectable locally advanced or metastatic melanoma comprises treating the primary cutaneous melanoma and secondary metastasis of the melanoma. In some embodiments, treating an unresectable locally advanced or metastatic melanoma comprises treating the secondary metastasis of the melanoma. In some embodiments, treating an unresectable locally advanced or metastatic melanoma comprises a first line treatment of the melanoma. In some embodiments, treating an unresectable locally advanced or metastatic melanoma comprises a second line treatment of the melanoma. In some embodiments, treating an unresectable locally advanced or metastatic melanoma comprises a third line treatment of the melanoma. In some embodiments, treating an unresectable locally advanced or metastatic melanoma comprises a fourth line treatment of the melanoma. In some embodiments, treating an unresectable locally advanced or metastatic melanoma comprises a fifth line treatment of the melanoma. In some embodiments, treating an unresectable locally advanced or metastatic melanoma comprises a sixth line treatment of the melanoma. In some embodiments, treating an unresectable locally advanced or metastatic melanoma comprises a seventh line treatment of the melanoma. In some embodiments, treating an unresectable locally advanced or metastatic melanoma comprises an eighth line treatment of the melanoma. In some embodiments, treating an unresectable locally advanced or metastatic melanoma comprises a ninth line treatment of the melanoma. In some embodiments, treating an unresectable locally advanced or metastatic melanoma comprises second and third line treatments of the melanoma.

In some embodiments, a “first line” treatment is the initial and preferred therapeutic option for a given medical condition or disease, chosen because it is considered the most effective and least risky based on evidence and clinical guidelines. In some embodiments, a “second line” treatment is a treatment that is given to a patient when the initial treatment (first-line therapy) is not effective or stops working. In some embodiments, a “third line” treatment is a treatment that is given to a patient when both initial treatment (first-line therapy) and subsequent treatment (second-line therapy) are not effective or stop working. Fourth line, fifth line, etc. treatments are similarly treatment that is given to a patient when prior treatments failed.

In some embodiments, treating cancer comprises treating subjects that failed to beneficially respond to prior checkpoint inhibitor (CPI) therapy. In some embodiments, treating cancer comprises treating subjects that failed to beneficially respond to prior doublet checkpoint inhibitor (CPI) therapy. In some embodiments of the present methods, a patient had at least 1, 2, 3, 4, 5, 6, 7, 8, 9, or 10 prior therapies. In some embodiments, doublet treatment is ipilimumab+nivolumab treatment. In some embodiments, doublet treatment is nivolumab+relatlimab treatment.

In some embodiments of the methods of treatment herein, the inclusion criteria for subjects is to have an ECOG Performance Status of 0 or 1. In some embodiments, the ECOG Performance Status refers to a standardized clinical scale developed by the Eastern Cooperative Oncology Group for assessing a subject's level of functioning, including the ability to perform daily activities, self care, and physical tasks. The ECOG Performance Status provides a numerical score that reflects the extent to which a disease affects the subject's daily living abilities and overall functional capacity. The ECOG Performance Status is typically measured on a discrete scale from 0 to 5, wherein lower scores indicate higher levels of functioning and higher scores indicate greater functional impairment. The ECOG Performance Status is widely used in clinical practice and clinical trials to characterize patient populations, monitor changes in functional status over time, and assess suitability for therapeutic interventions. Performance Status is defined as follows: 0—Fully active; able to carry on all pre-disease activities without restriction; 1—Restricted in physically strenuous activity but ambulatory and able to carry out light or sedentary work; 2—Ambulatory and capable of all self care but unable to carry out work activities; up and about more than 50% of waking hours; 3—Capable of only limited self care; confined to bed or chair more than 50% of waking hours; 4—Completely disabled; unable to carry out any self care; totally confined to bed or chair; 5—Dead. In some embodiments of the methods of treatment herein, the inclusion criteria for subjects is to have an ECOG Performance Status of 0. In some embodiments of the methods of treatment herein, the inclusion criteria for subjects is to have an ECOG Performance Status of 1.

In some embodiments of methods of treating an unresectable locally advanced or metastatic solid cancer in a subject comprising administering to the subject an anti-IL-2 antibody or pharmaceutical composition thereof, a loading dose of IL-2 at a low dose or a pharmaceutical composition thereof, and nivolumab or a pharmaceutical composition thereof, the unresectable locally advanced or metastatic solid cancer comprises a cutaneous melanoma, a melanoma, a renal cell carcinoma (RCC), a head and neck carcinoma, a head and neck squamous cell carcinoma (HNSCC), a nasopharyngeal carcinoma, an esophageal carcinoma, a gastric carcinoma, a gastric or gastro-esophageal cancer, a gastroesophageal junction carcinoma, an esophageal squamous cell carcinoma, a cutaneous squamous cell carcinoma (cSCC), a hepato-cellular carcinoma (HCC), a colorectal cancer (CRC), a microsatellite instability high (MSHi) CRC, a microsatellite stable (MSHs) CRC, a cervical carcinoma, an endometrial carcinoma, a urothelial cancer, a bladder cancer, a ureteral cancer, a renal pelvis cancer, a malignant mesothelioma, a malignant pleural mesothelioma, a malignant peritoneal mesothelioma, a classical Hodgkin Lymphoma (cHL), a biliary tract carcinoma (BTC), a MSHi or mismatch repair deficient cancer, a Tumor Mutational Burden-High (TMB-H) cancer, Triple-negative breast cancer (TNBC), or a Merkel cell carcinoma (MCC), and the treatment comprises a first-, second-, or third-line treatment.

In some embodiments of methods of treating an unresectable locally advanced or metastatic solid cancer in a subject comprising administering to the subject an anti-IL-2 antibody or pharmaceutical composition thereof, a loading dose of IL-2 at a low dose or a pharmaceutical composition thereof, and nivolumab or a pharmaceutical composition thereof, the unresectable locally advanced or metastatic solid cancer comprises NSCLC, a squamous NSCLC, or a non-squamous NSCLC, and the treatment consists of a first-line treatment.

In certain embodiments of methods disclosed herein, the method of treatment comprises (i) reducing the size of the tumor, (ii) inhibiting or reducing growth of the tumor, (iii) inhibiting or reducing metastases of said tumor, (iv) inhibiting the appearance of new lesions, (v) decreasing or shrinking target lesions, (vi) eliminating target lesions, or (vii) any combination thereof.

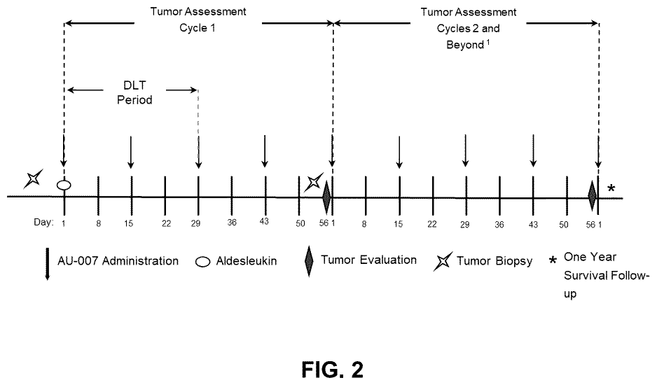

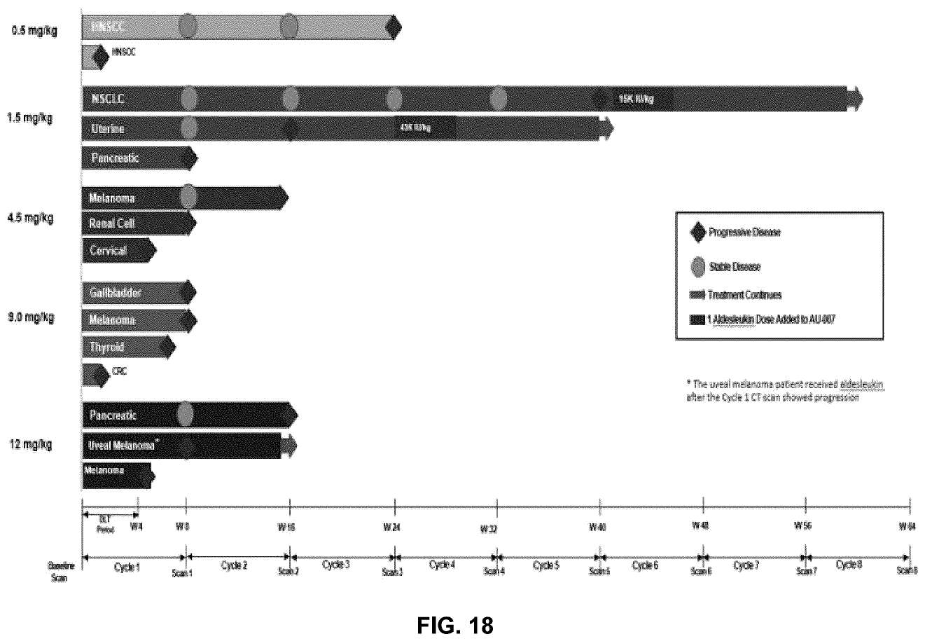

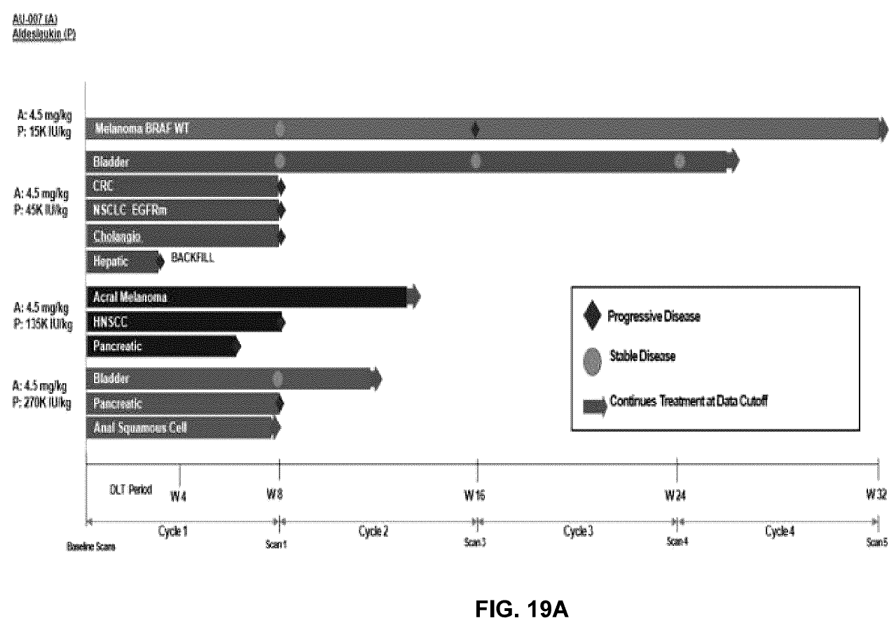

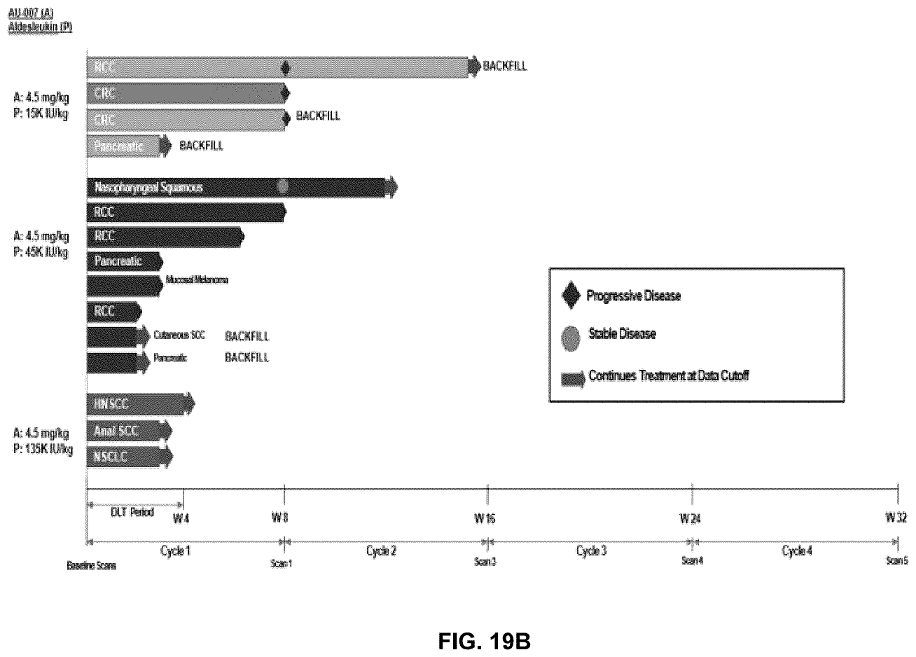

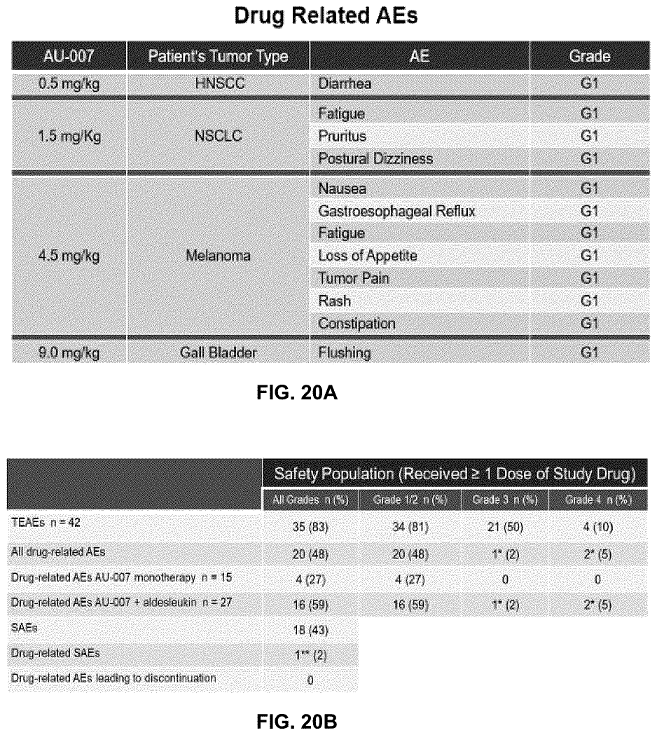

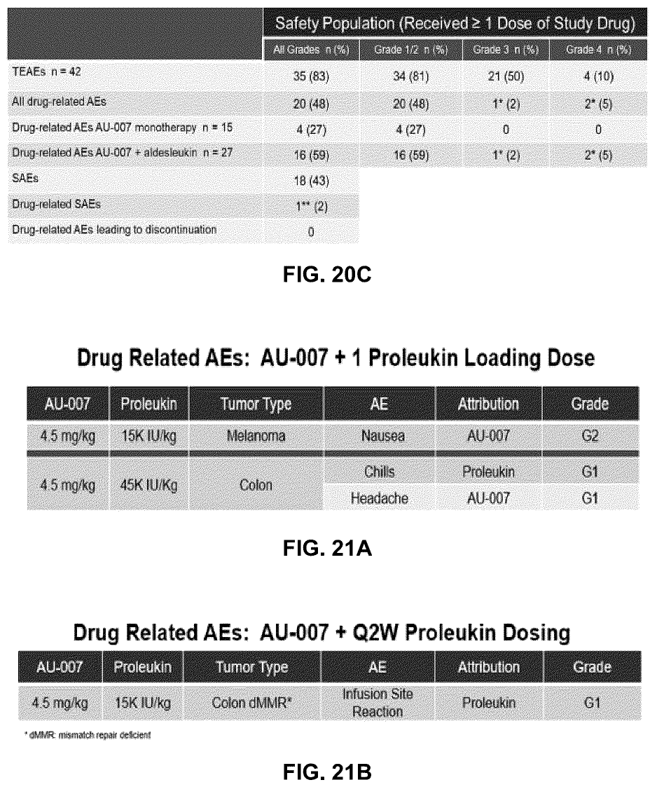

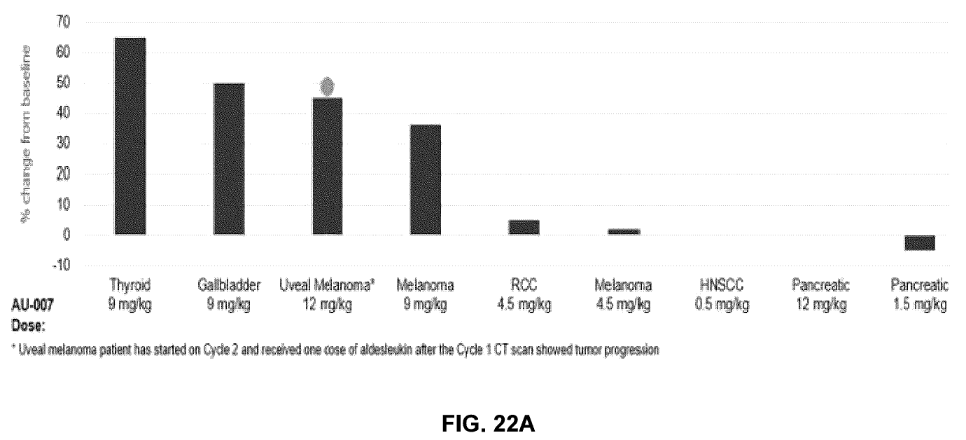

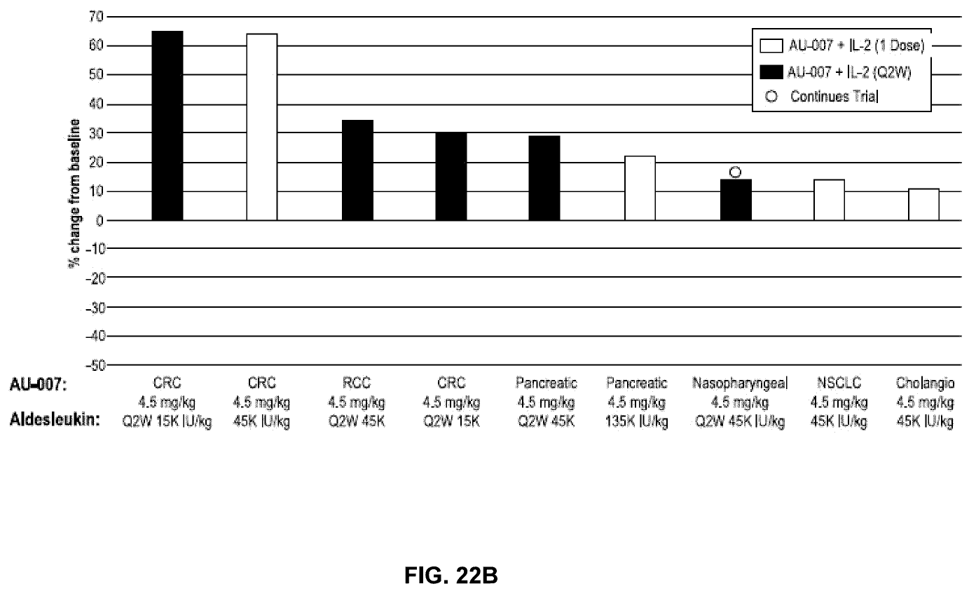

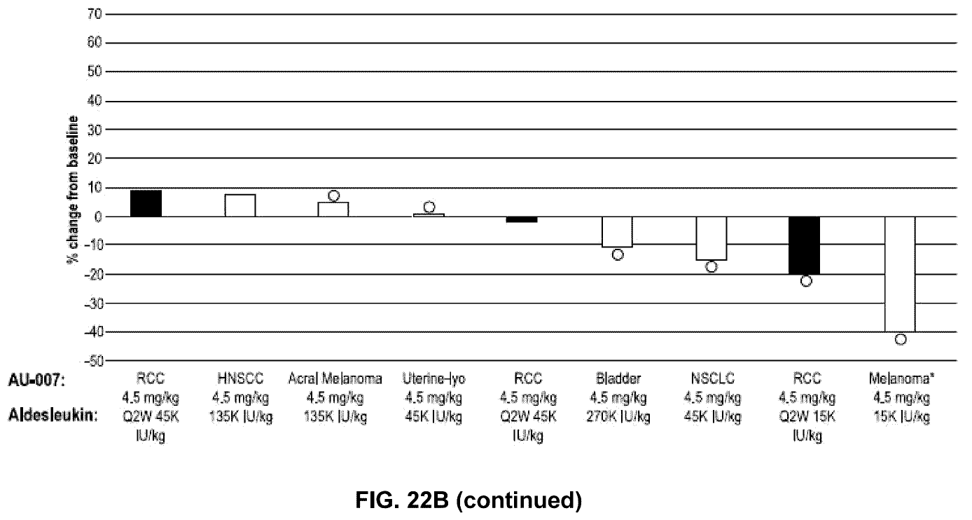

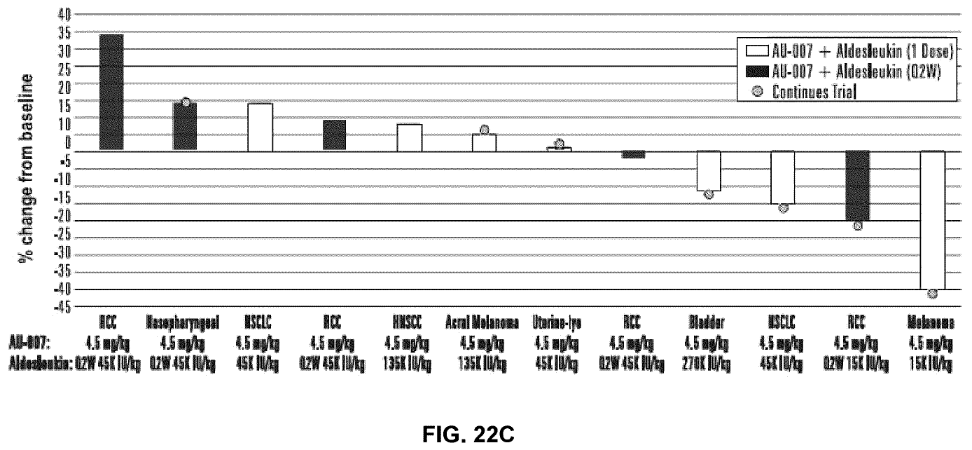

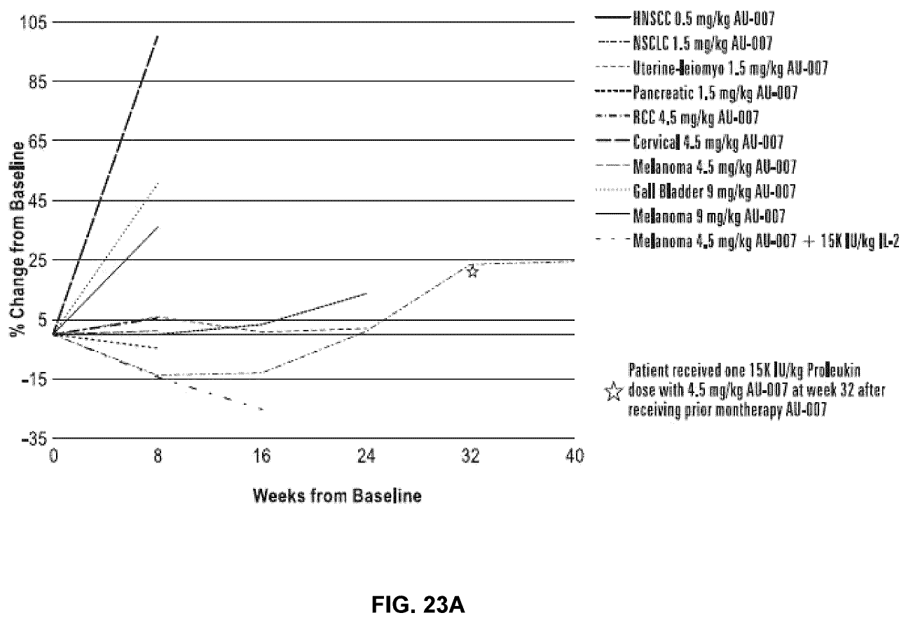

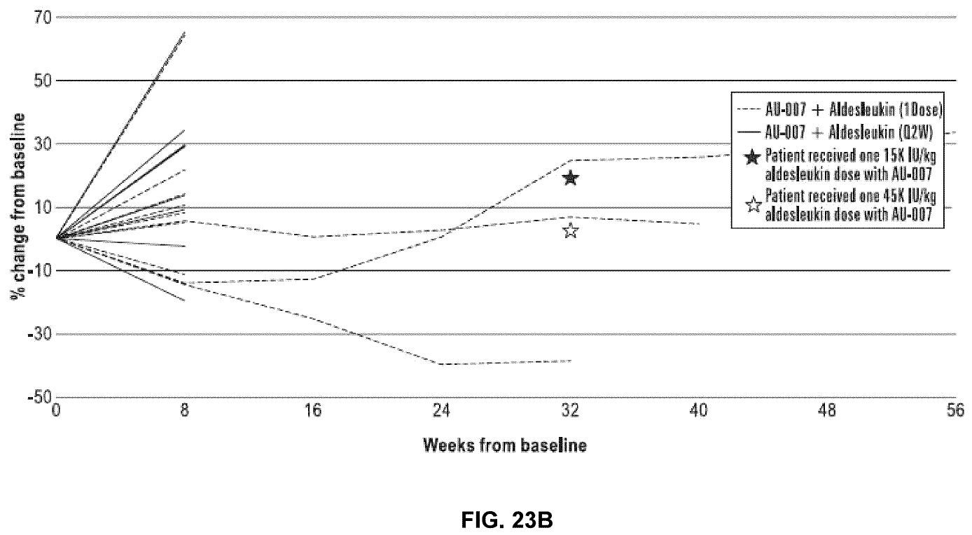

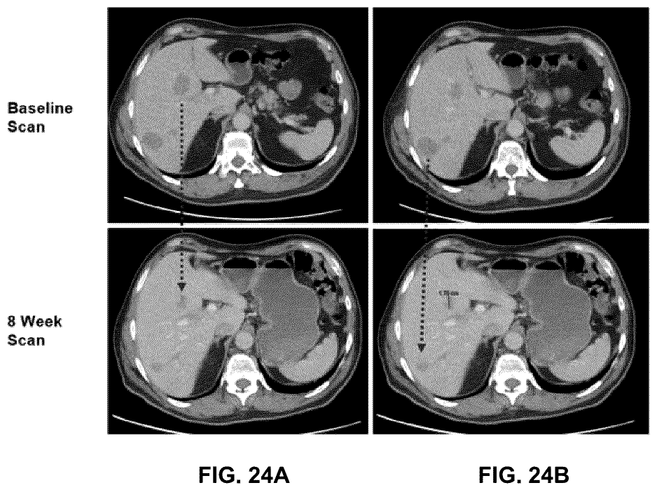

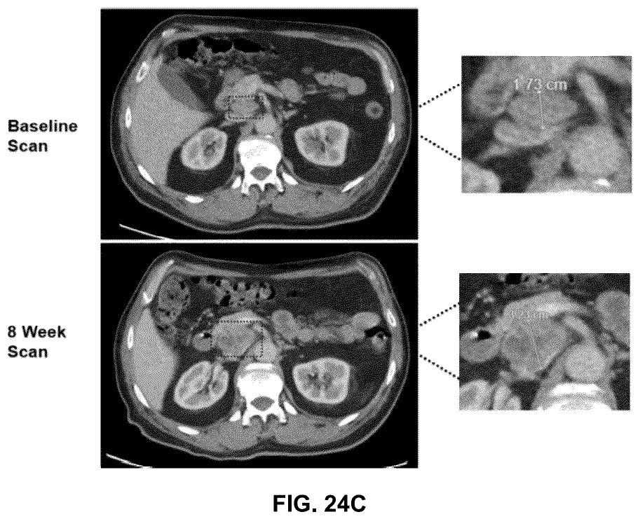

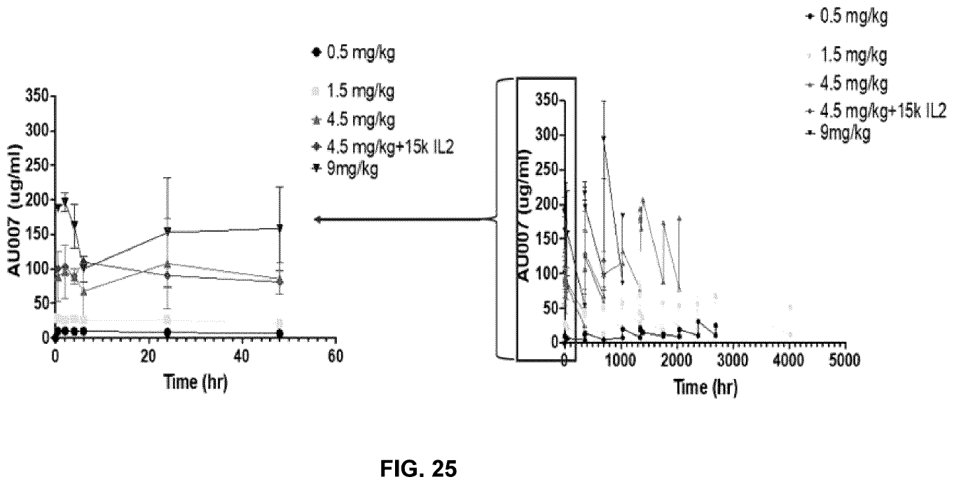

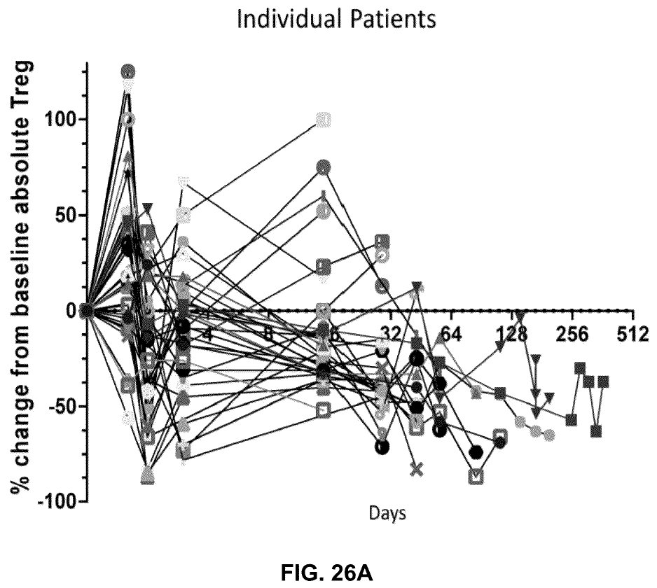

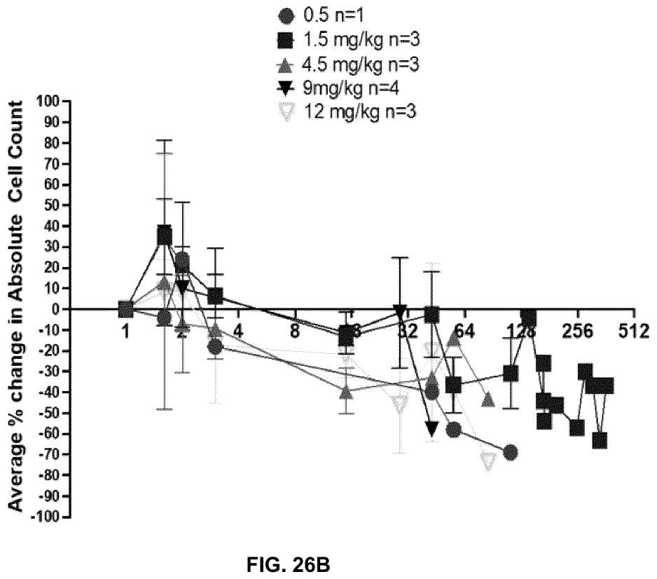

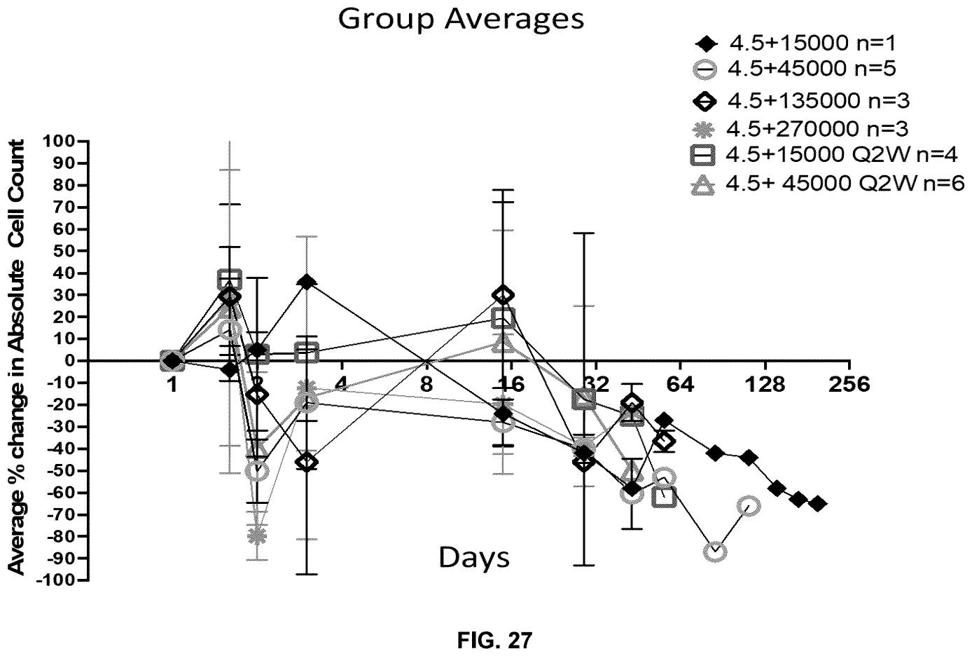

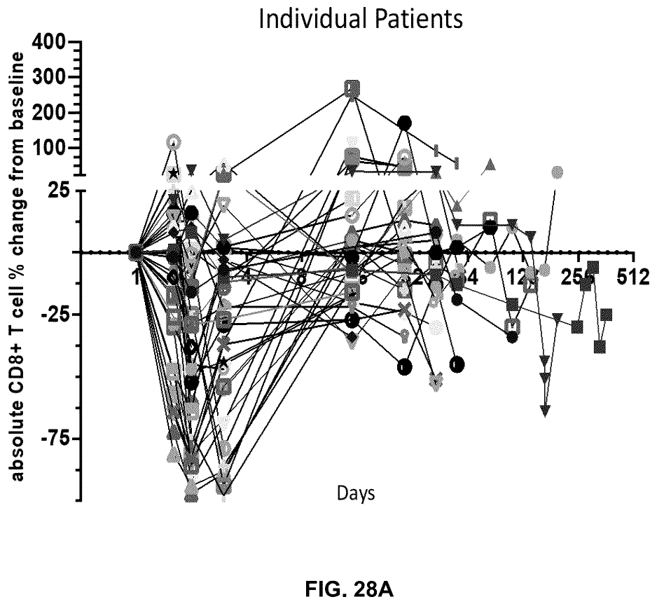

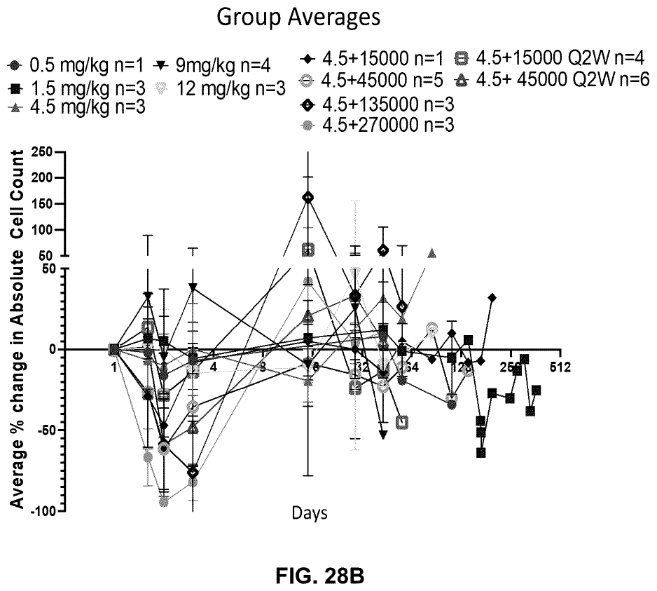

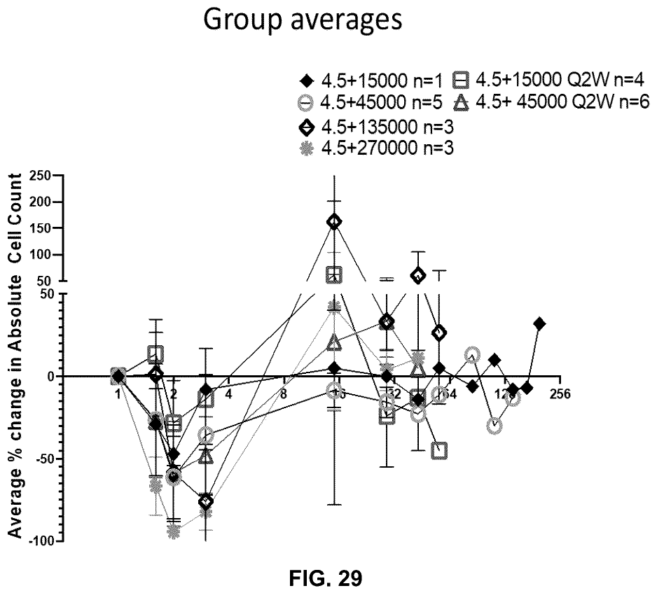

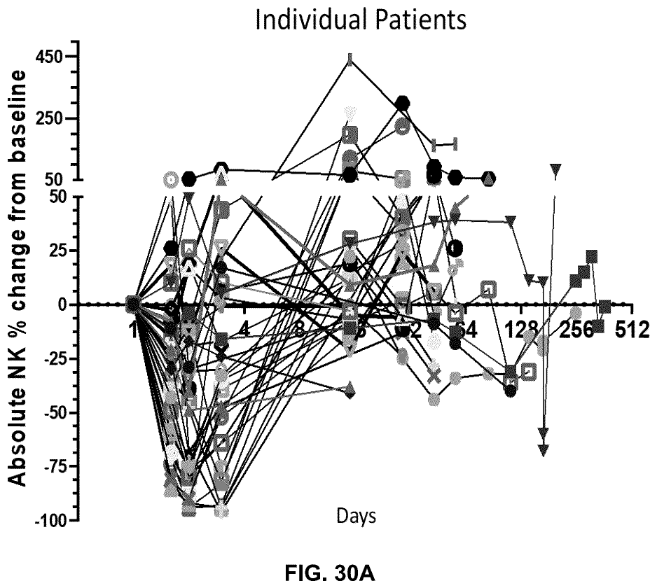

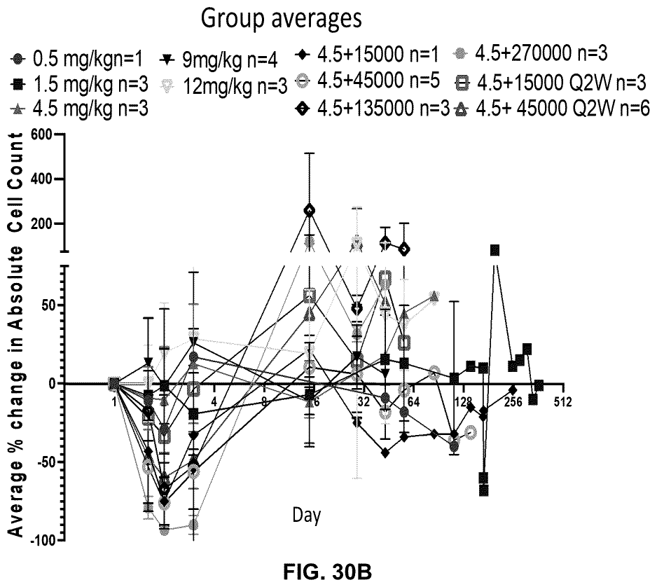

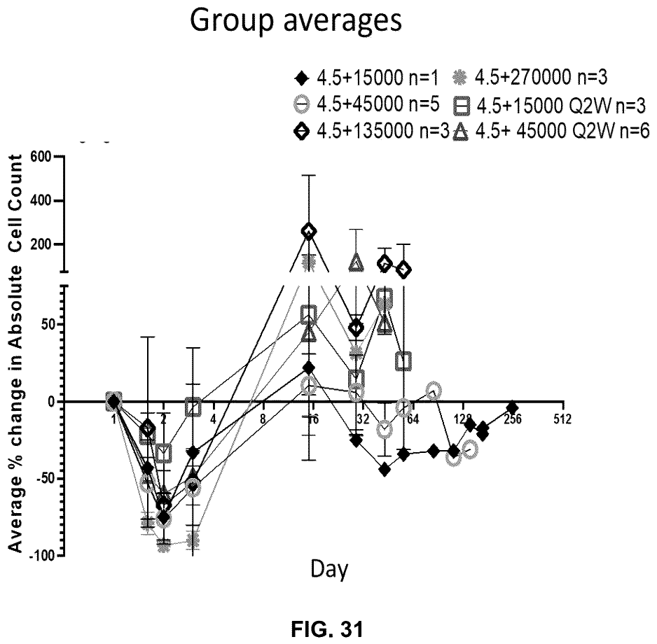

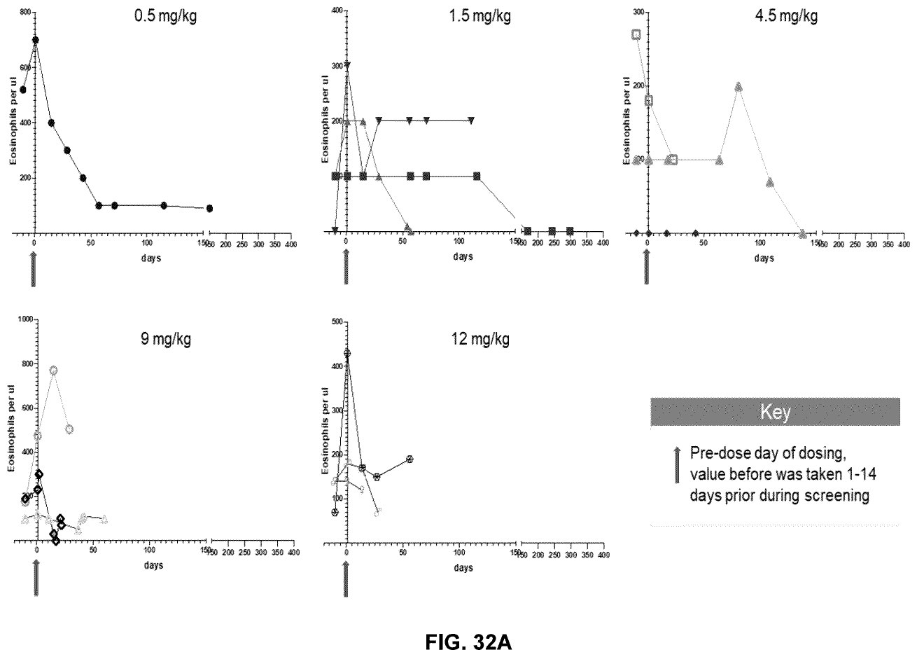

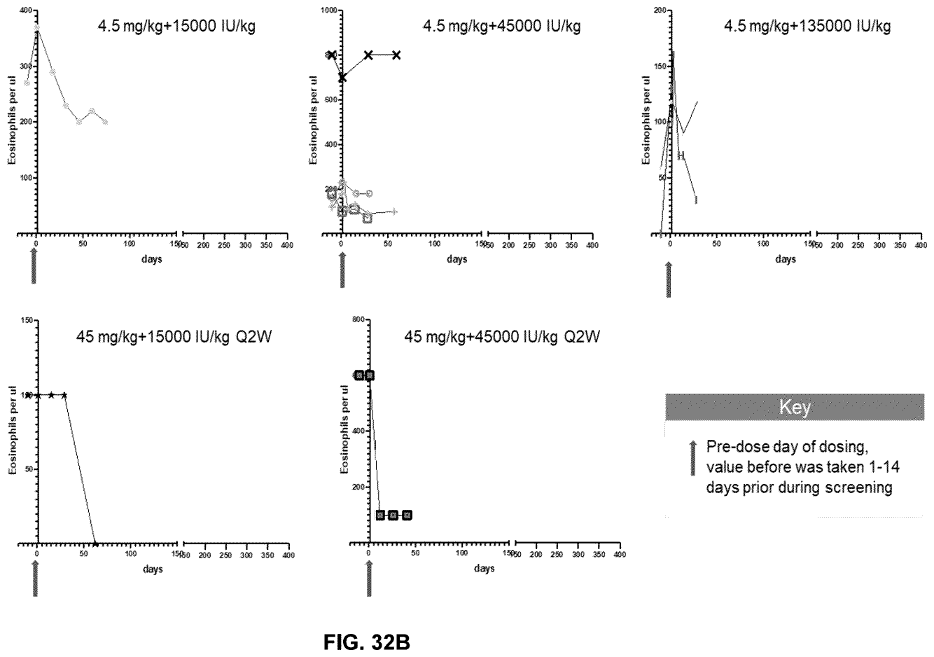

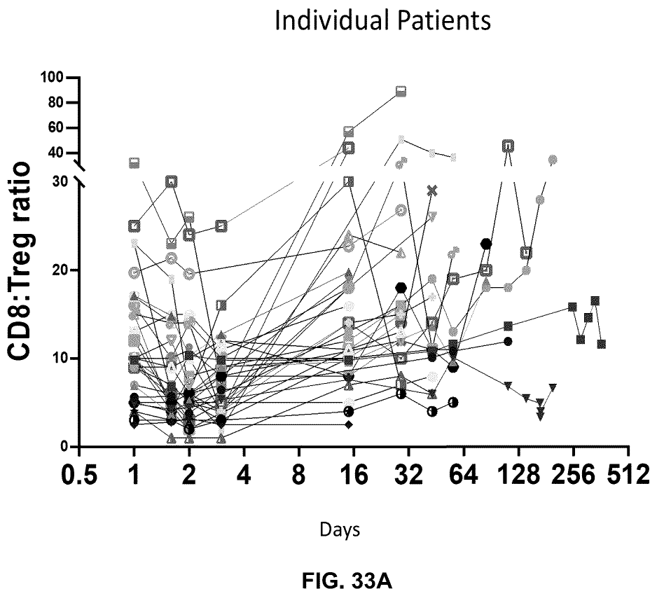

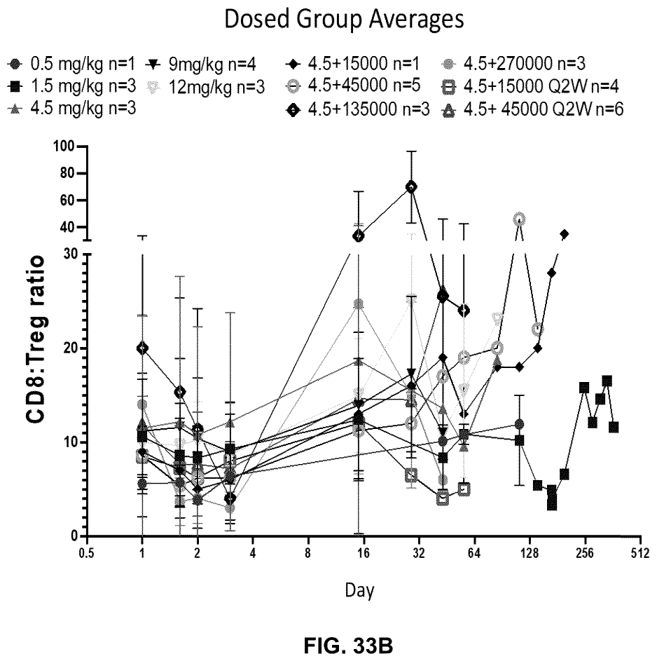

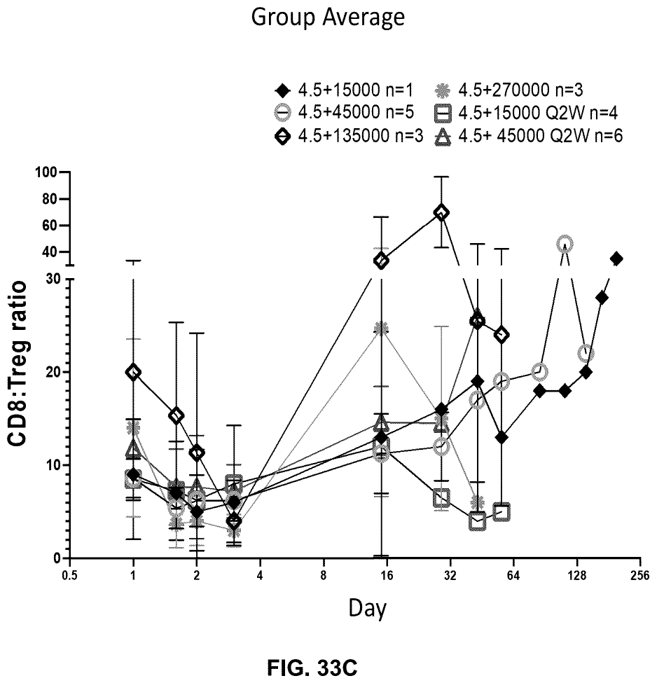

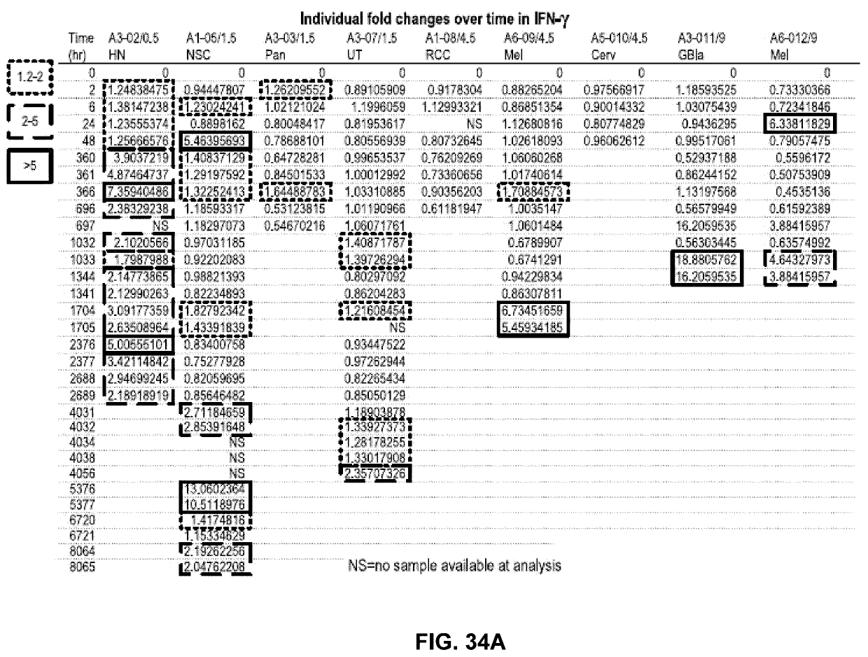

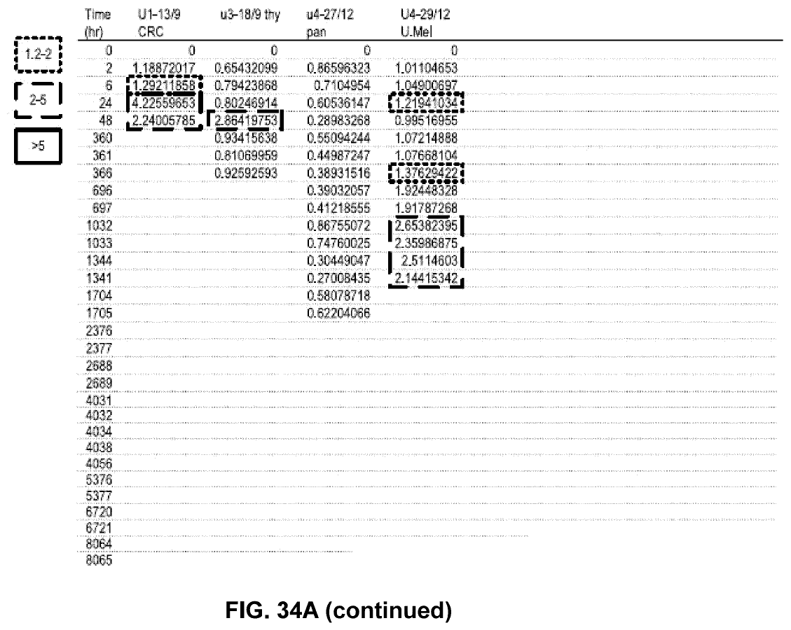

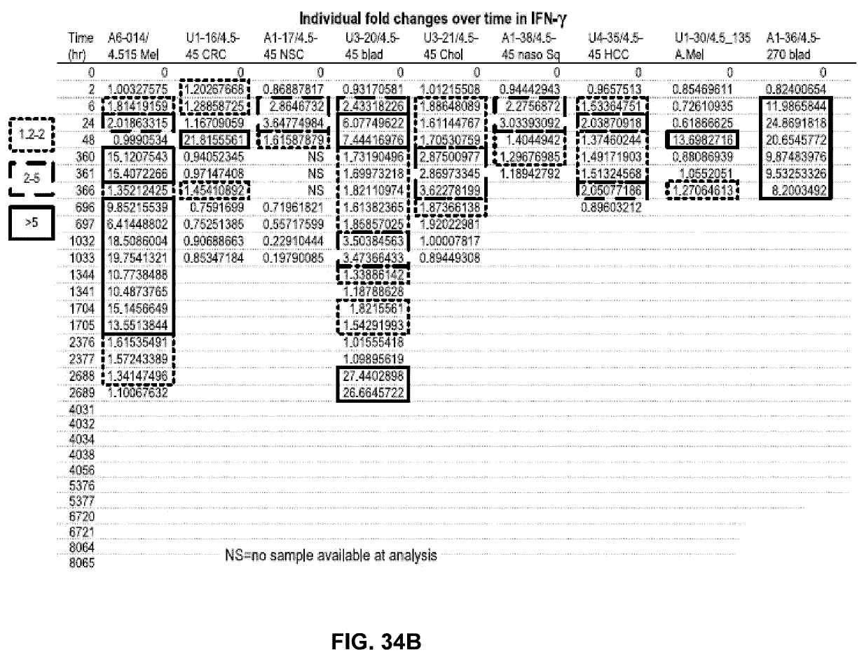

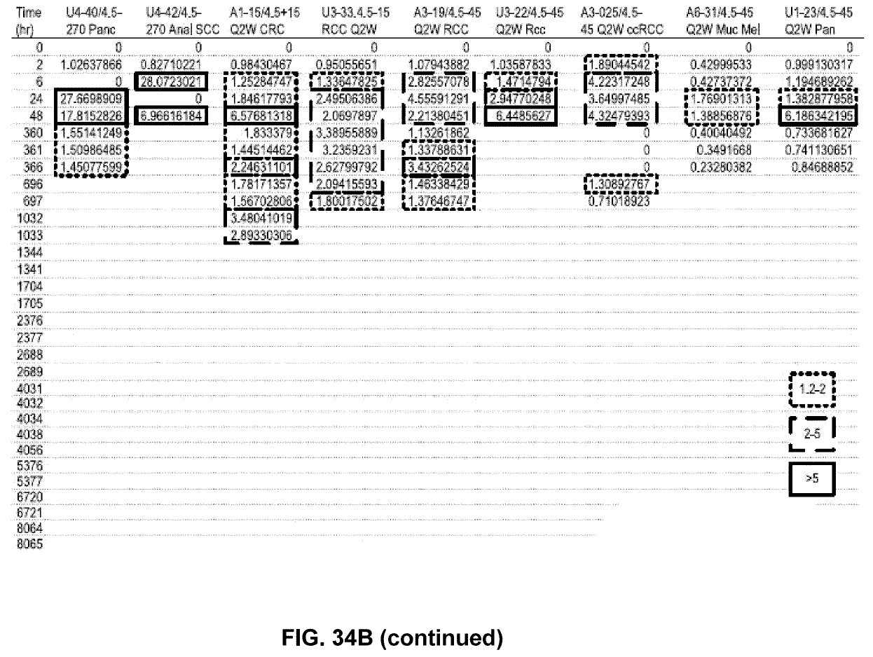

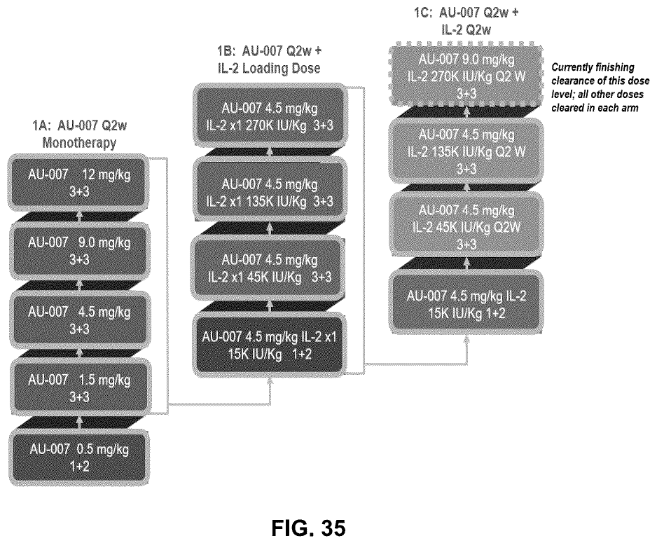

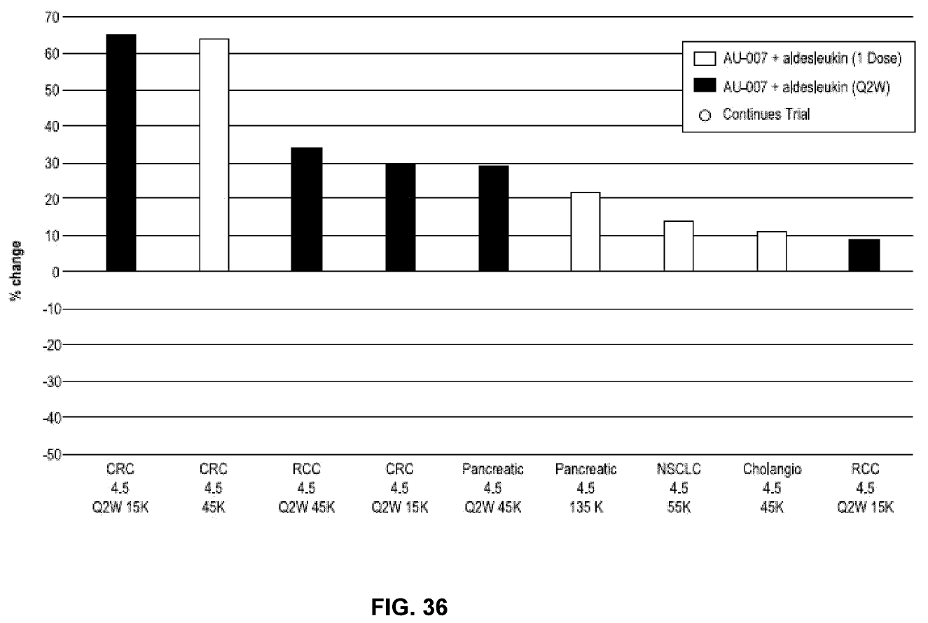

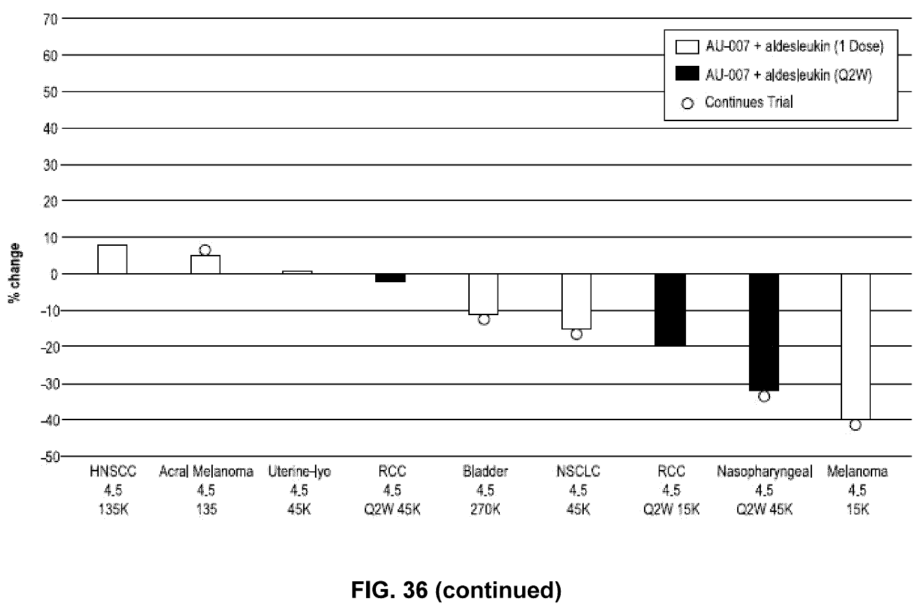

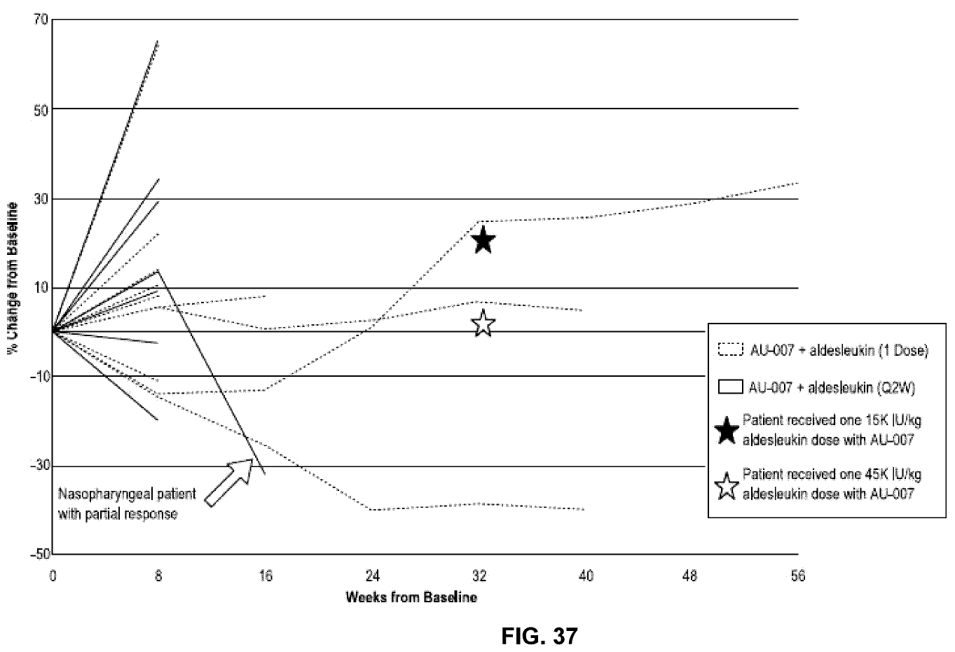

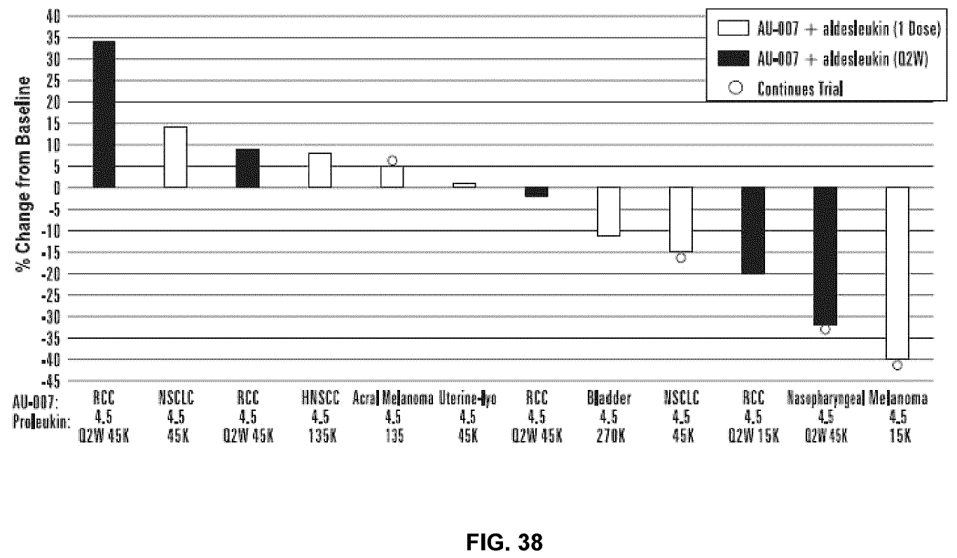

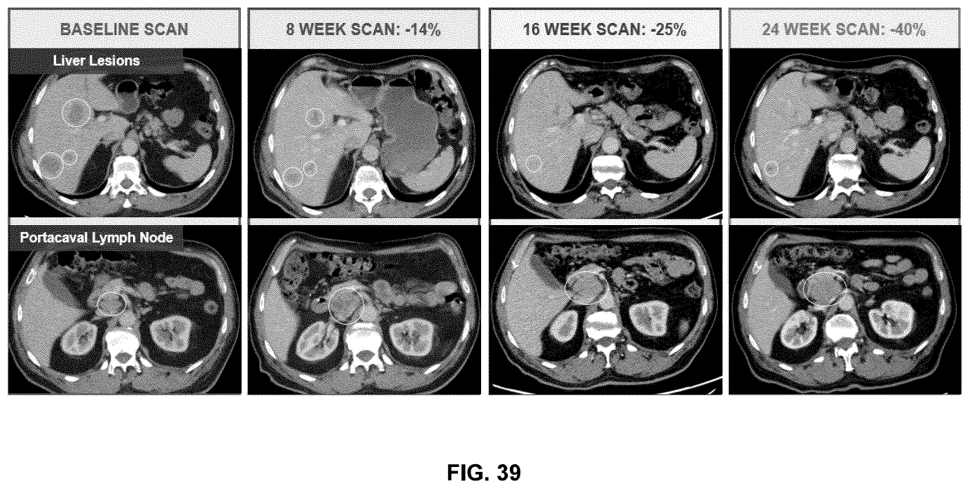

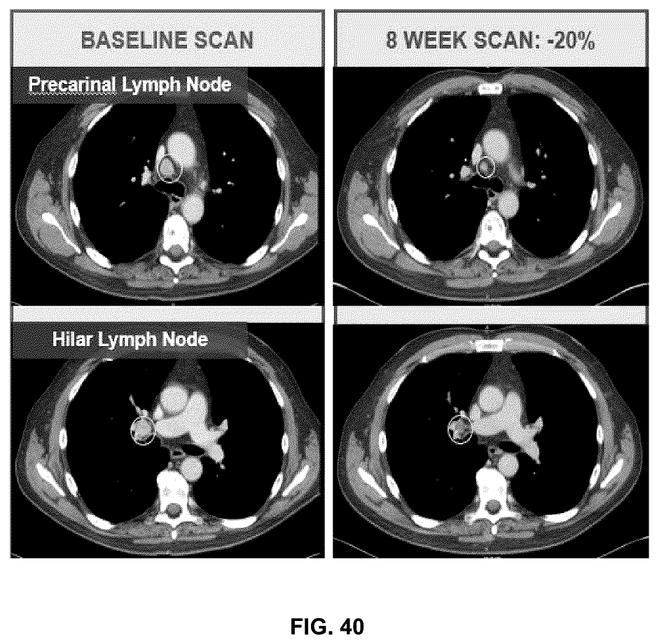

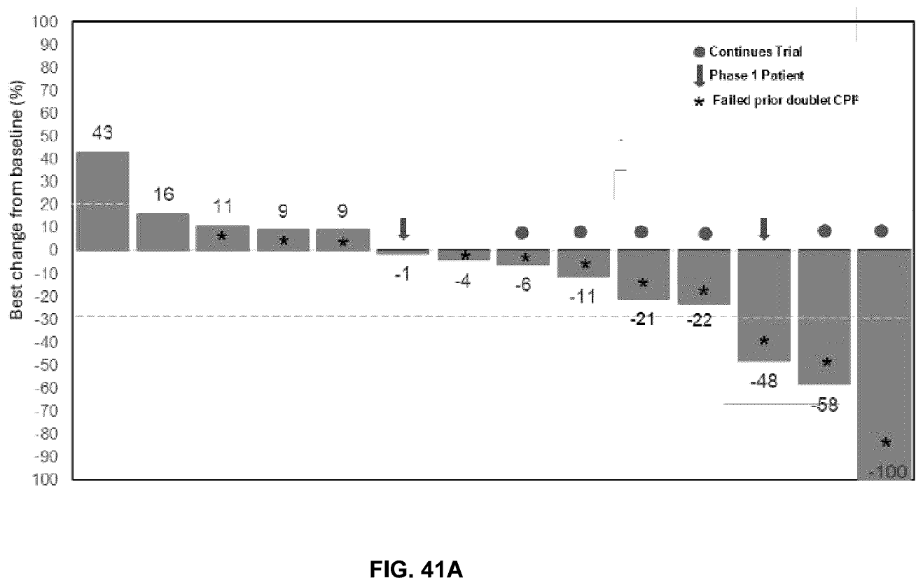

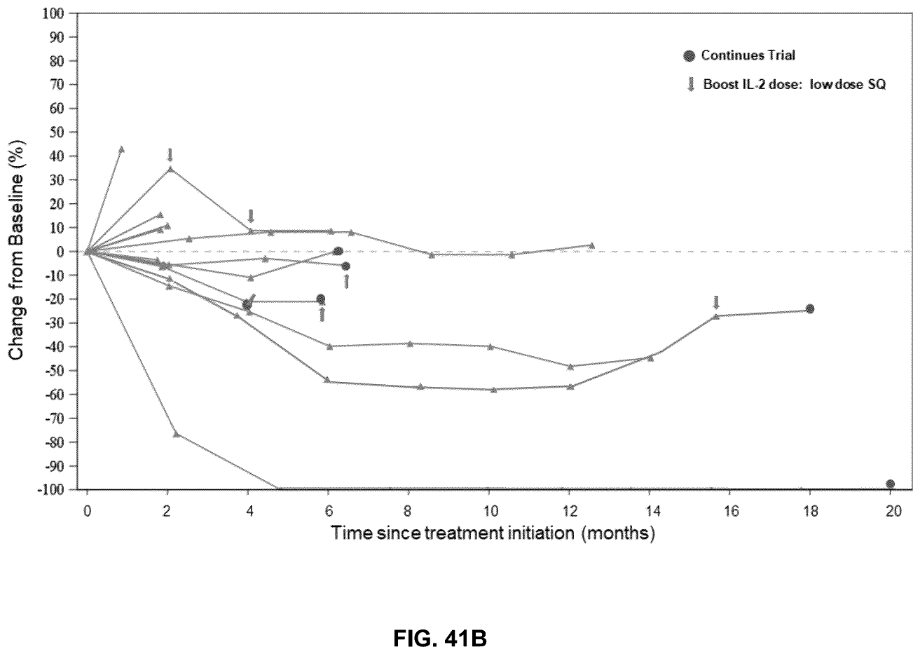

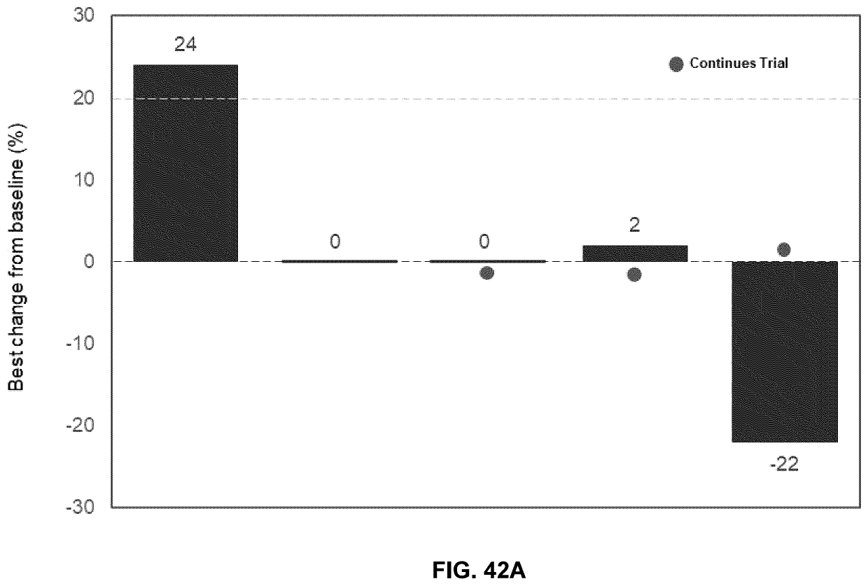

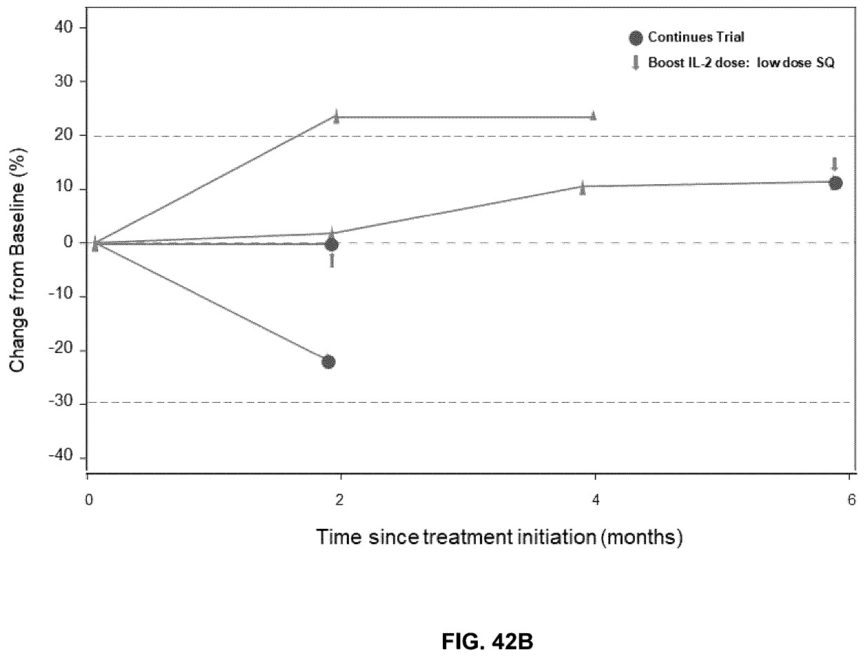

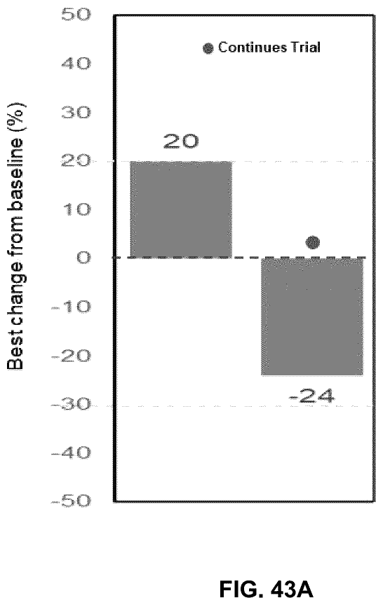

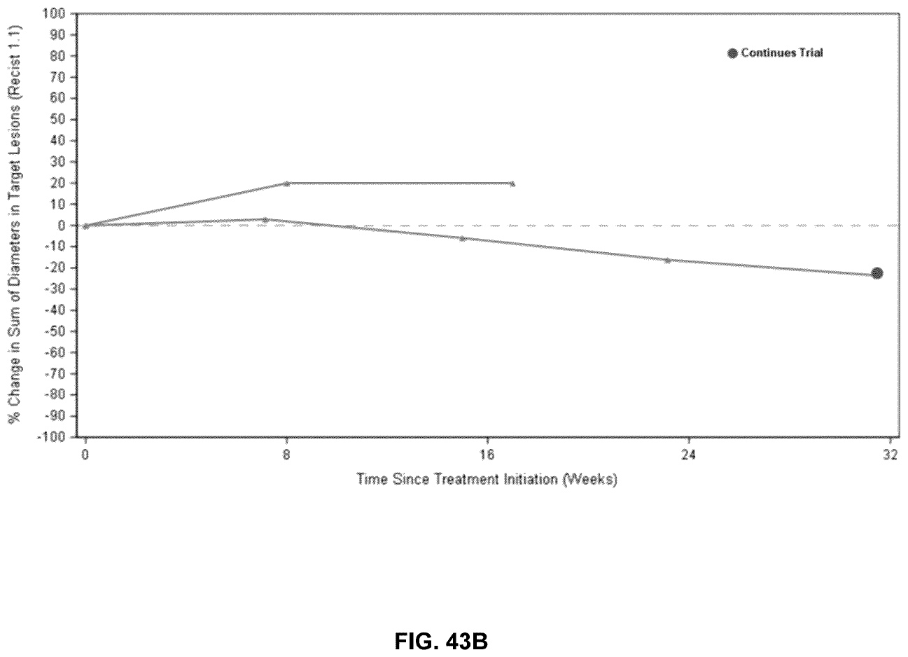

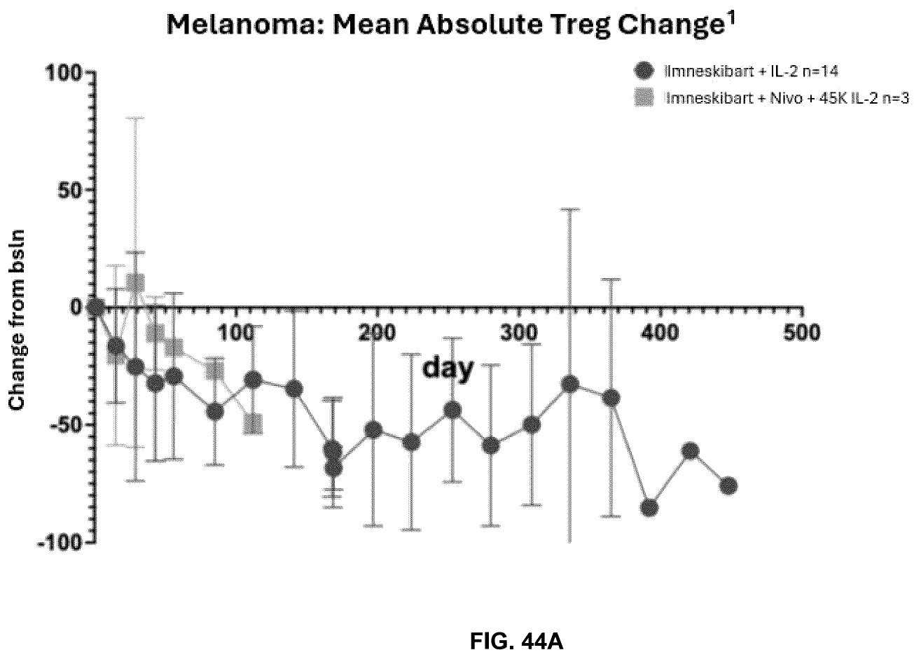

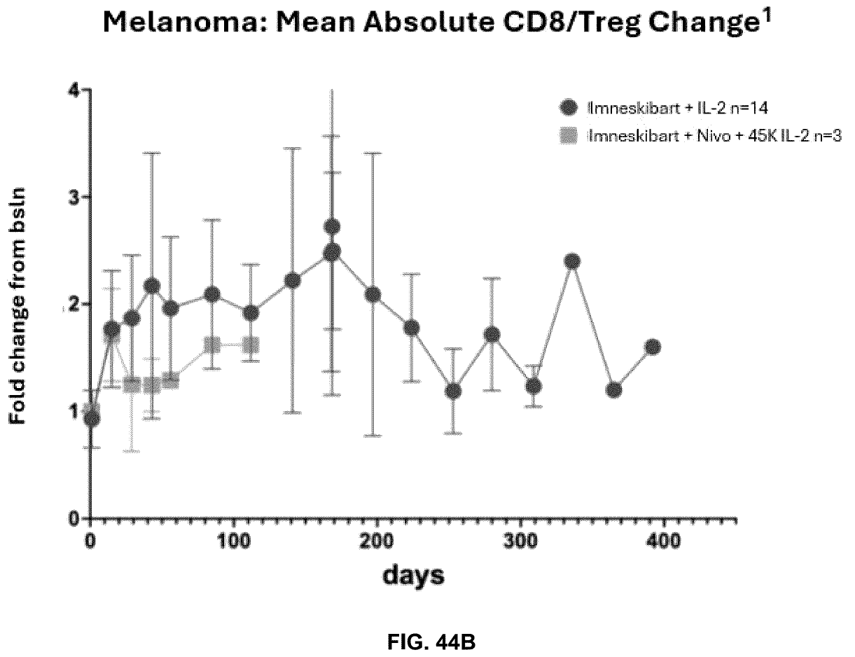

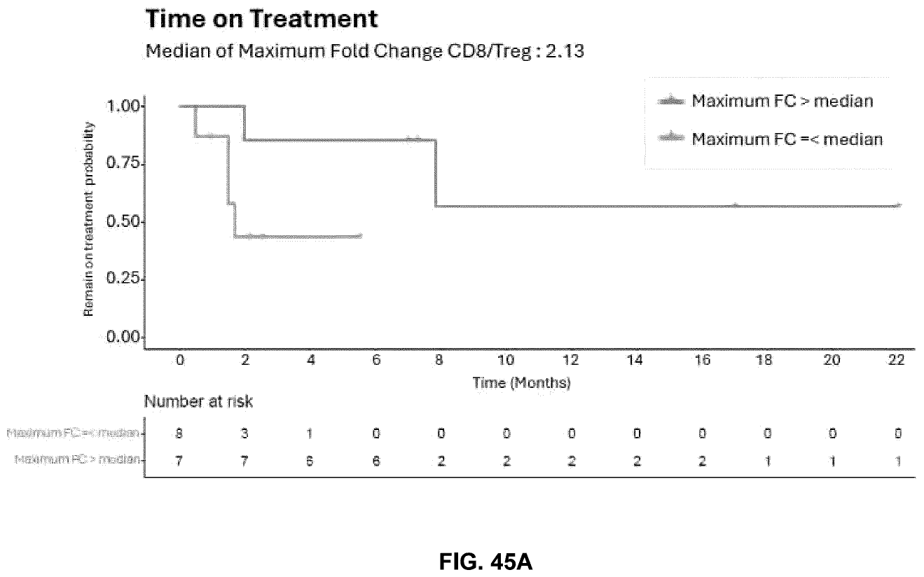

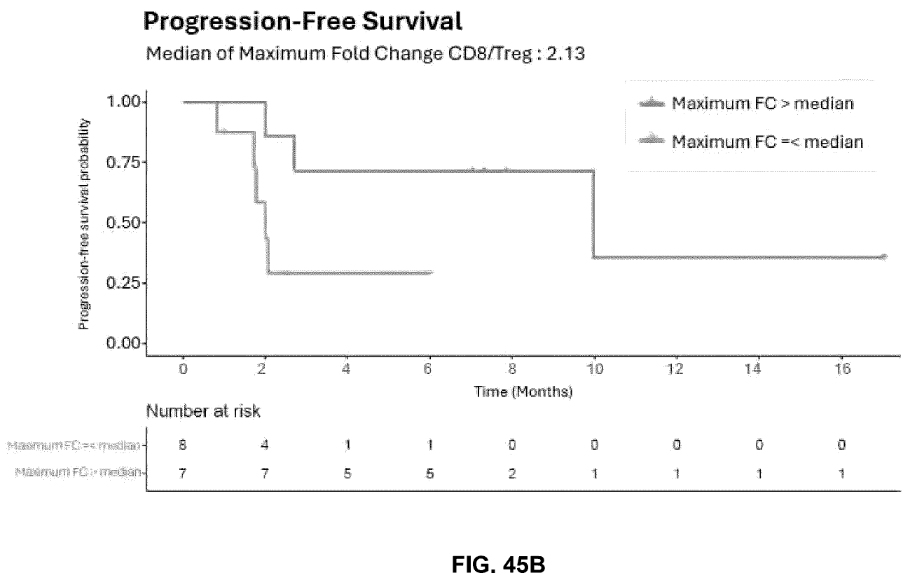

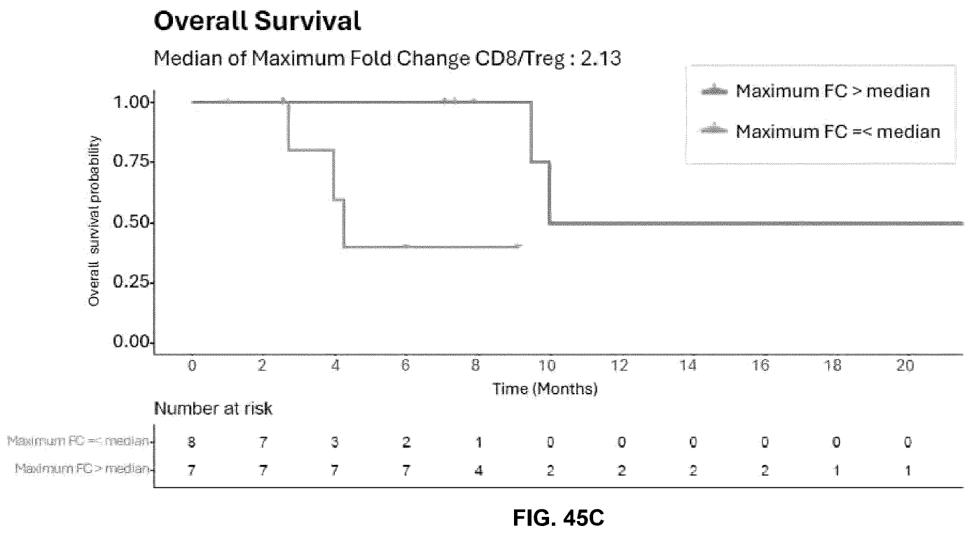

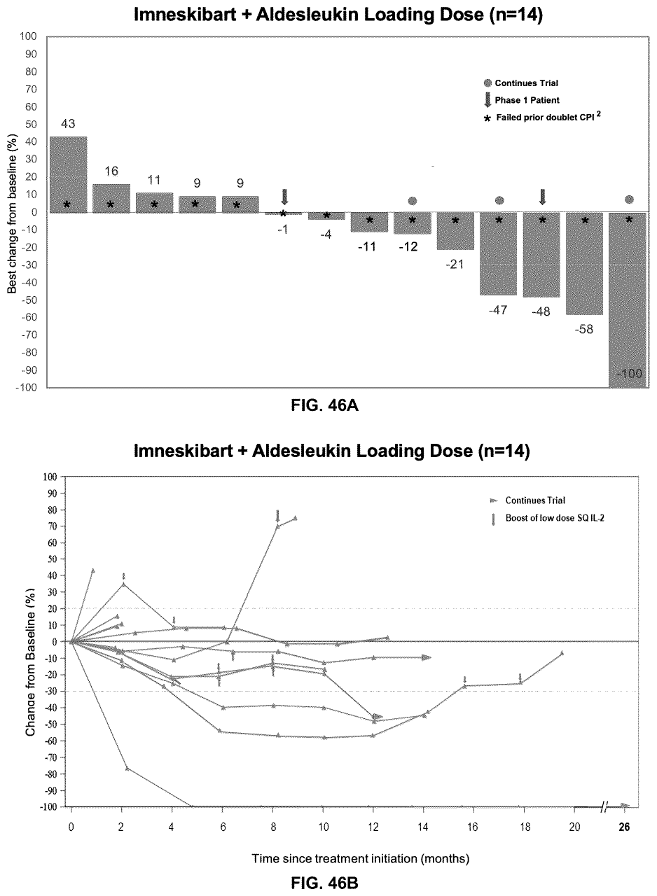

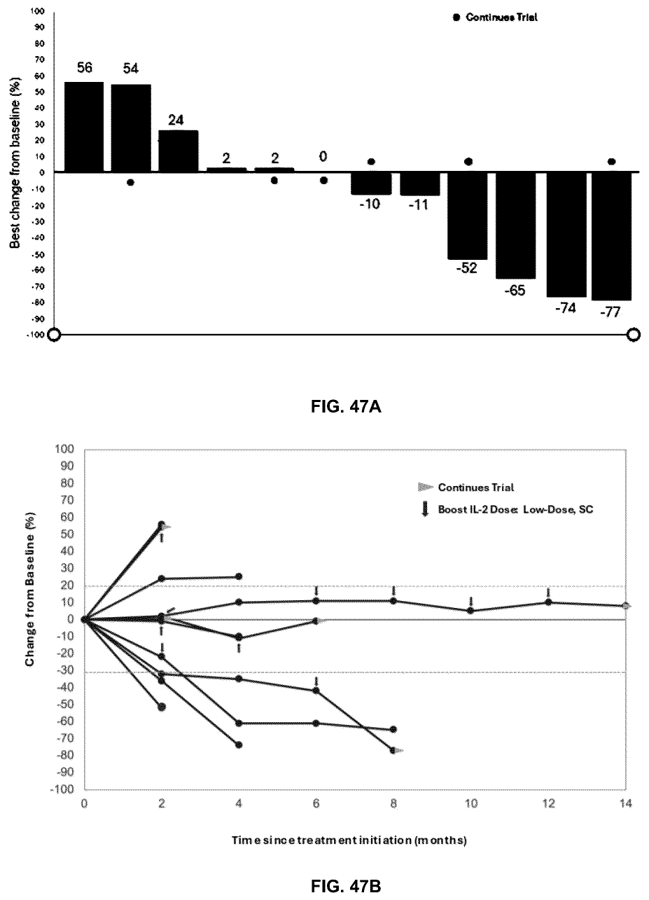

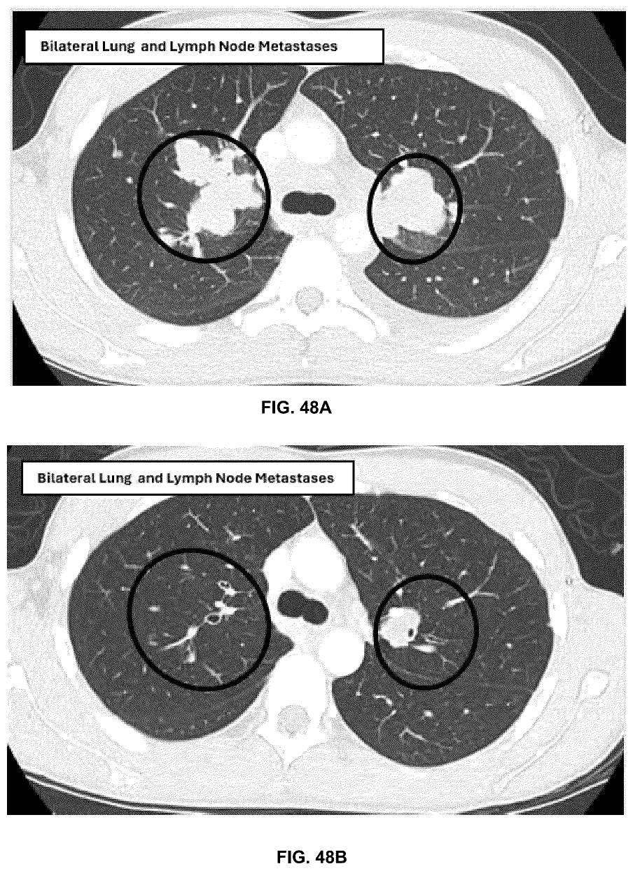

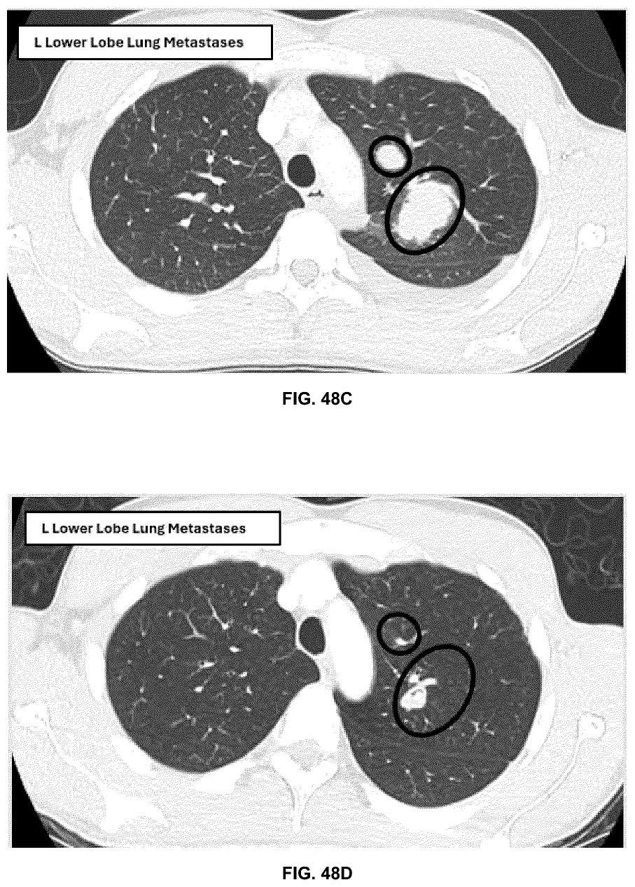

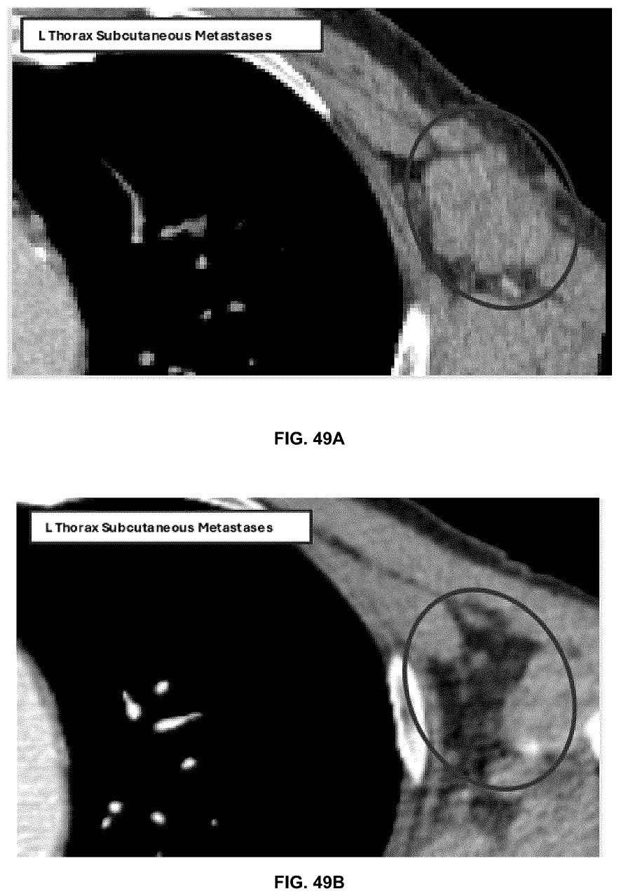

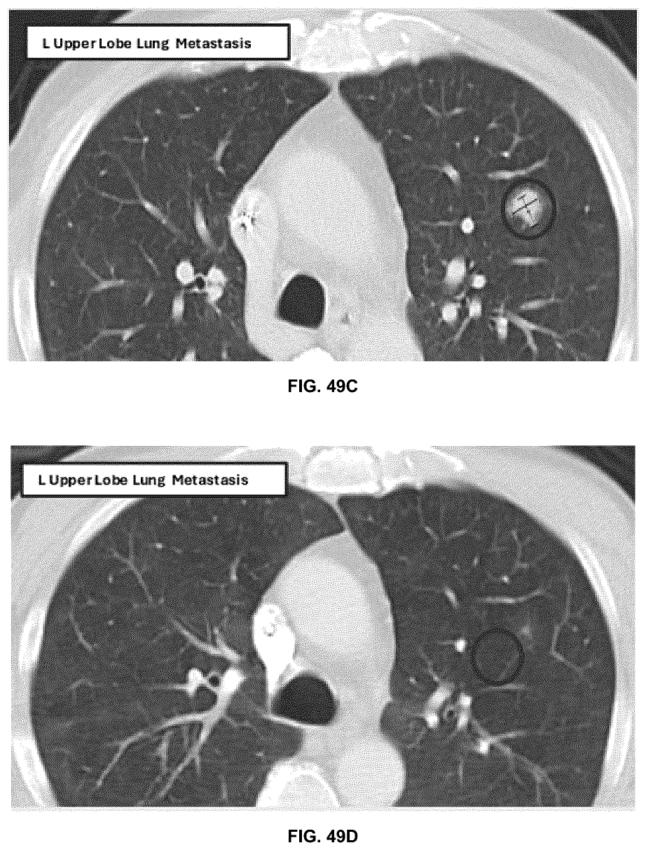

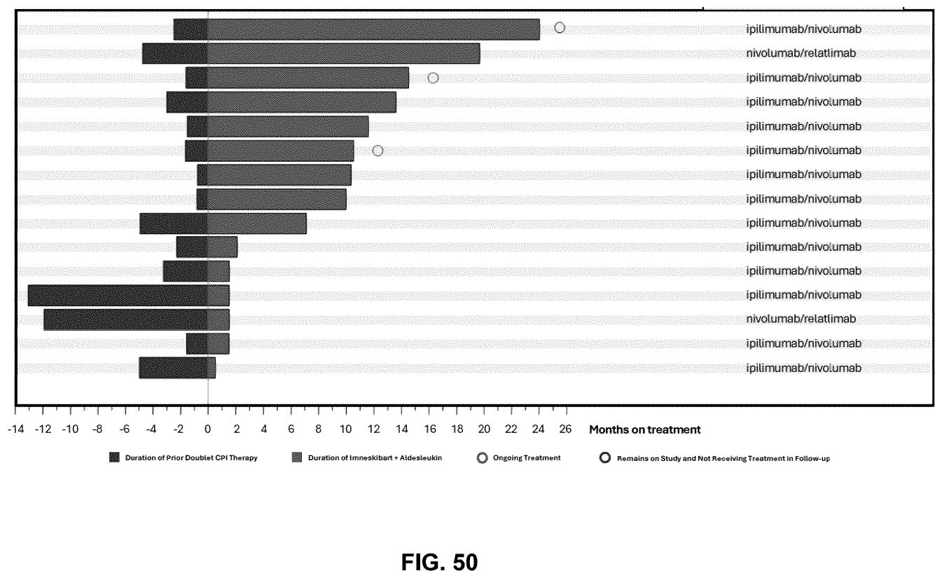

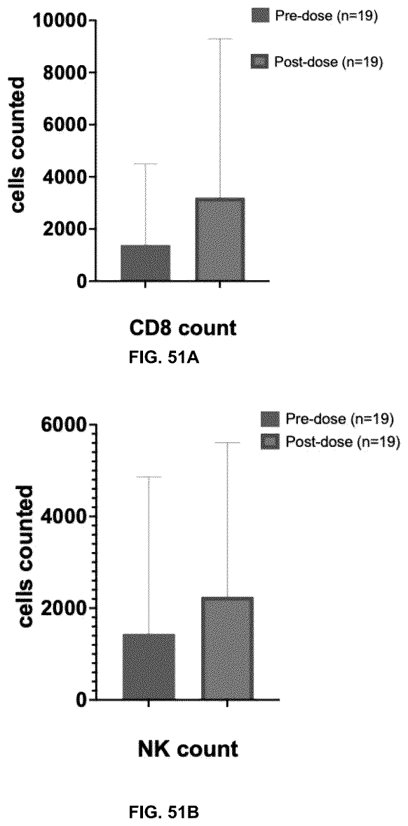

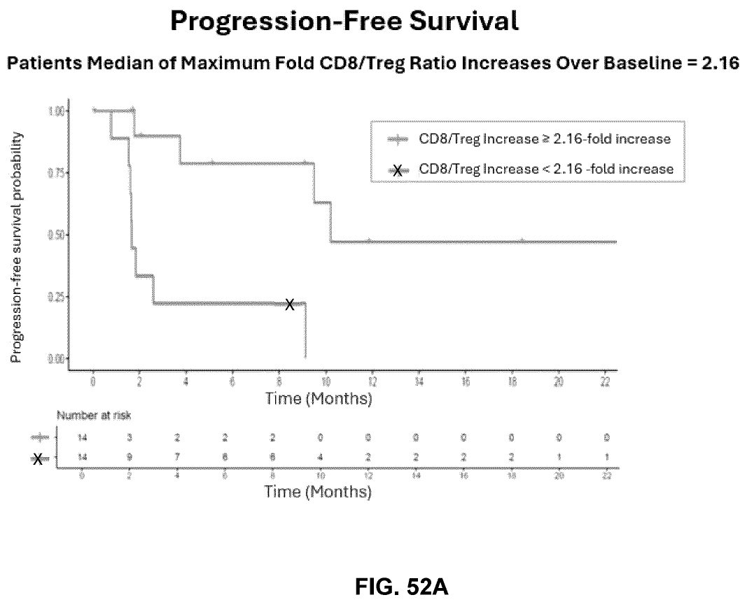

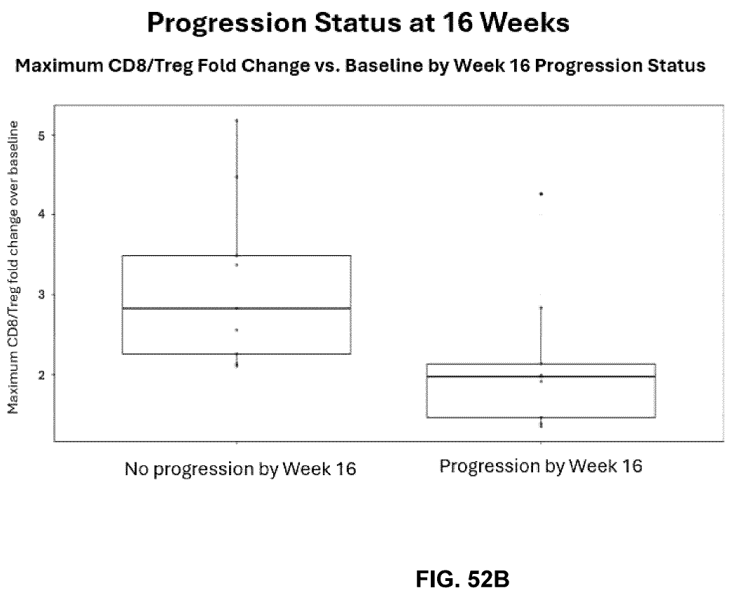

In some embodiments of methods disclosed herein, multiple doses of anti-IL-2 antibody are administered. In some embodiments, 2-50 doses of anti-IL-2 antibody are administered. In other embodiments, 20-50 doses of anti-IL-2 antibody are administered. In other embodiments, 10-40 doses of anti-IL-2 antibody are administered. In other embodiments, 20-30 doses of anti-IL-2 antibody are administered. In other embodiments, more than 20 doses of anti-IL-2 antibody are administered. In other embodiments, more than 30 doses of anti-IL-2 antibody are administered. In other embodiments, more than 40 doses of anti-IL-2 antibody are administered. In other embodiments, more than 50 doses of anti-IL-2 antibody are administered. In some embodiments, anti-IL-2 antibody doses are administered at regular intervals throughout the treatment. In some embodiments, the interval between anti-IL-2 antibody doses is once every 2 weeks, 4 weeks, 6 weeks, or 8 weeks. In some embodiments, the interval between anti-IL-2 antibody doses is 2 weeks (FIGS. 2 and 17).

In some embodiments, the IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5.

In other embodiments of methods discloses herein, a single dose of nivolumab is administered. In some embodiments, multiple doses of nivolumab are administered. In some embodiments, 2-50 doses of nivolumab are administered. In other embodiments, 20-50 doses of nivolumab are administered. In other embodiments, 10-40 doses of nivolumab are administered. In other embodiments, 20-30 doses of nivolumab are administered. In other embodiments, more than 20 doses of nivolumab are administered. In other embodiments, more than 30 doses of nivolumab are administered. In other embodiments, more than 40 doses of nivolumab are administered. In other embodiments, more than 50 doses of nivolumab are administered. In some embodiments, nivolumab doses are administered at regular intervals throughout the treatment. In some embodiments, the interval between nivolumab doses is once every 2 weeks, 4 weeks, 6 weeks, or 8 weeks. In some embodiments, the interval between nivolumab doses is 4 weeks (FIG. 17)

In some embodiments, a method as described herein comprises administering a loading dose of a low dose of IL-2 or a pharmaceutical composition thereof to the subject. In some embodiments, the method comprises further administering one or more booster doses of a low dose of IL-2 or a pharmaceutical composition thereof to the subject. In some embodiments, the method comprises further administering a single booster dose of a low dose of IL-2 or a pharmaceutical composition thereof to the subject. In other embodiments, the method comprises further administering two or more booster doses of a low dose of IL-2 or a pharmaceutical composition thereof to the subject. In other embodiments, the method comprises further administering three or more booster doses of a low dose of IL-2 or a pharmaceutical composition thereof to the subject. In other embodiments, the method comprises further administering four or more booster doses of a low dose of IL-2 or a pharmaceutical composition thereof to the subject. In other embodiments, the method comprises further administering five or more booster doses of a low dose of IL-2 or a pharmaceutical composition thereof to the subject. In other embodiments, the method comprises administering a loading dose of low dose IL-2 and two, three, four, five, or six booster doses of low dose IL-2. In other embodiments, the method comprises administering a loading dose of low dose IL-2 and 6-10 or 10 or more booster doses of low dose IL-2. In some embodiments, low dose IL-2 boosters are administered at regular intervals throughout the treatment. In some embodiments, the interval between low dose IL-2 boosters is about once every 2 weeks, 4 weeks, 6 weeks, or 8 weeks. In some embodiments, the interval between low dose IL-2 boosters is 2 weeks. In some embodiments, the interval between low dose IL-2 boosters is 4 weeks. In some embodiments, the interval between low dose IL-2 boosters is 6 weeks. In some embodiments, the interval between low dose IL-2 boosters is 8 weeks (FIG. 17).

In some embodiments of the methods described herein that comprise administering a booster dose of IL-2, the booster dose of IL-2 is administered to the subject with each treatment cycle, e.g., until objective tumor shrinkage is observed on radiologic imaging or physical exam. In some embodiments, objective tumor shrinkage is observed on radiologic imaging, physical exam, or a combination thereof. In other embodiments, the booster dose of IL-2 is administered to the subject if tumor volume is stable. In other embodiments, the booster dose of IL-2 is administered to the subject when the subject has one or more objective signs of worsening tumor growth kinetics. In some embodiments, the booster dose of IL-2 is administered to the subject if previously shrinking tumors become stable. In some embodiments, the booster dose of IL-2 is administered to the subject if there is an increase in one or more tumor markers. In some embodiments, the booster dose of IL-2 is administered to the subject if there is new tumor growth. In some embodiments, the booster dose of IL-2 is administered to the subject if one or more new tumors are detected. In other embodiments, the booster dose of IL-2 is administered to the subject if of new tumor growth appears in a tumor that was previously stable or had decreased in size. In other embodiments, the booster dose of IL-2 is administered to the subject if the tumor of the subject comprises any combination of the above. In some embodiments, a treatment cycle is 8 weeks. In other embodiments, a treatment cycle is 2, 4, 6, 10, or 12 weeks.

A skilled artisan would appreciate that a “target lesion” is a measurable (measurable defined per the RECIST (Response Evaluation Criteria in Solid Tumors) rules) tumor lesion (locally advanced or metastatic) that's measurement is added to the measurements of up to 4 other Target Lesions and the total combined diameters of the target lesions is used to follow if the tumor is increasing or decreasing in size after each cycle of treatment. In certain embodiments, methods described herein inhibit or reduce metastases. This may be observed by the absence of new lesions. In some embodiments, methods described herein result in decrease or shrinkage in a target lesion. This may be observed by measurements showing the reduction in size of a tumor. In some embodiments, methods described herein result in the disappearance of target lesions (a tumor). This too may be observed by measurements showing the reduction in size and disappearance of a tumor.

In some embodiments of the methods described herein that comprise administering a booster dose of IL-2, the booster dose of IL-2 is administered to the subject until at least Partial Response (PR) is achieved. In some embodiments of the methods described herein that comprise administering a booster dose of IL-2, the booster dose of IL-2 is administered to the subject with each treatment cycle, until at least Partial Response (PR) is achieved.

In some embodiments of the methods described herein that comprise administering a booster dose of IL-2, the booster dose of IL-2 is administered to the subject until at least complete response (CR) is achieved. In some embodiments of the methods described herein that comprise administering a booster dose of IL-2, the booster dose of IL-2 is administered to the subject with each treatment cycle, until at least complete response (CR) is achieved.

In some embodiments, Partial Response (PR) or complete response (CR) is evaluated according to RECIST.

In some embodiments of the methods described herein that comprise administering a booster dose of IL-2, the booster dose of IL-2 is administered to the subject until objective tumor shrinkage is observed. In some embodiments of the methods described herein that comprise administering a booster dose of IL-2, the booster dose of IL-2 is administered to the subject with each treatment cycle, until objective tumor shrinkage is observed.

In some embodiments, the booster dose of IL-2 is administered to the subject on day 1 of each cycle (Q8W). In some embodiments, the booster dose of IL-2 is administered to the subject until objective tumor shrinkage, or partial response, or complete response is observed on radiologic imaging or physical exam.

In some embodiments of the methods described herein that comprise administering a booster dose of IL-2, the booster dose of IL-2 is administered to the subject upon objective signs of worsening tumor growth kinetics: e.g., previously shrinking tumors becoming stable, or with new tumor growth.

In some embodiments, the administration of the one or more booster doses of IL-2 is prior to, concurrent with, or following the administration of said one or more additional doses of said anti-IL-2 antibody, one or more additional doses of said nivolumab, or both.

In certain embodiments, a booster IL-2 low dose is administered to a subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof.

In some embodiments, the anti-IL-2 antibody is administered at a dose of between about 0.5 mg/kg of a subject's body weight-12 mg/kg of said subject's body weight. In other embodiments, the anti-IL-2 antibody is administered at a dose of between about 4.5 mg/kg of a subject's body weight-12 mg/kg of said subject's body weight. In other embodiments, the anti-IL-2 antibody is administered at a dose of between about 9 mg/kg of a subject's body weight-12 mg/kg of said subject's body weight. In some embodiments, the anti-IL-2 antibody is administered at a dose of 9 mg/kg subject's body weight. In other embodiments, the anti-IL-2 antibody is administered at a dose of 12 mg/kg. In other embodiments, the anti-IL-2 antibody is administered at a dose of 6 mg/kg. In other embodiments, the anti-IL-2 antibody is administered at a dose of 3 mg/kg. In other embodiments, the anti-IL-2 antibody is administered at a dose of 10 mg/kg. In other embodiments, the anti-IL-2 antibody is administered at a dose of 15 mg/kg. In other embodiments, the anti-IL-2 antibody is administered at a dose of 20 mg/kg.

In some embodiments, the methods described herein further comprise the step of administering one or more additional doses of the anti-IL-2 antibody. In other embodiments, the methods described herein further comprise the step of administering one or more additional doses of a pharmaceutical composition comprising the anti-IL-2 antibody.

In some embodiments, the anti-IL-2 antibody is administered on a bi-weekly (once every two weeks) schedule. In other embodiments, the anti-IL-2 antibody is administered on a schedule as described hereinabove. In some embodiments, the anti-IL-2 antibody is administered weekly, bi-weekly, or once every three weeks.

In some embodiments, the schedule of administering the anti-IL-2 antibody may not be the same through the full course of the therapy, wherein administration may be bi-weekly for a time period and may then change to weekly, once every three weeks, once every four weeks, once every 5 weeks, or once every 6 weeks. Similarly, the schedule of administering the anti-IL-2 antibody may not be the same through the full course of the therapy, wherein administrations may be further apart, for example but not limited to once every 4-6 weeks and may then change to weekly, bi-weekly, or once every three weeks.

In some embodiment, the IL-2 is administered as a one-time loading dose and optionally as a booster dose as needed. In some embodiments, the loading dose of IL-2 is subcutaneously administered. In some embodiments, the booster dose of IL-2 is subcutaneously administered. As used herein, the terms subcutaneous, SC, and SQ, and the like, may be used interchangeably having all the same meanings and qualities.

In some embodiments, the loading dose of IL-2 is administered at a dose of between about 15,000 IU/kg of said subject's body weight-500,000 IU/kg of said subject's body weight. In other embodiments, the loading dose of IL-2 is administered at a dose of between about 45,000-270,000 IU/kg of the subject's body weight. In other embodiments, the loading dose of IL-2 is administered at a dose of between about 45,000 IU/kg-135,000 IU/kg of said subject's body weight.

In certain embodiments, disclosed herein is a method of treating an unresectable locally advanced or metastatic solid cancer in a subject, said method comprising administering to said subject multiple doses of an anti-IL-2 antibody or a pharmaceutical composition thereof, a loading dose of IL-2 or a pharmaceutical composition thereof, and at least one booster dose of IL-2 or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; wherein said loading dose and booster dose of IL-2 are subcutaneously administered at a dose of between about 15,000 IU/kg of said subject's body weight and 500,000 IU/kg of said subject's body weight; wherein said pharmaceutical composition(s) further comprises a pharmaceutically acceptable carrier, thereby treating said unresectable locally advanced or metastatic solid cancer in said subject.

In some embodiments, the IL-2 loading dose is administered at a dose of 135,000 IU/kg of said subject's body weight. In other embodiments, the IL-2 loading dose is administered at a dose of 270,000 IU/kg. In other embodiments, the IL-2 loading dose is administered at a dose of 500,000 IU/kg. In other embodiments, the IL-2 loading dose is administered at a dose of 100,000 IU/kg. In other embodiments, the IL-2 loading dose is administered at a dose of 75,000 IU/kg. In other embodiments, the IL-2 loading dose is administered at a dose of 50,000 IU/kg.

In some embodiments of methods disclosed herein, the booster dose of IL-2 is administered at a dose of between about 15,000 IU/kg of said subject's body weight-500,000 IU/kg of said subject's body weight. In other embodiments, the booster dose of IL-2 is administered at a dose of between about 45,000-270,000 IU/kg of the subject's body weight. In other embodiments, the booster dose of IL-2 is administered at a dose of between about 45,000 IU/kg-135,000 IU/kg of said subject's body weight.

In some embodiments, the IL-2 booster dose is administered at a dose of 135,000 IU/kg of said subject's body weight. In other embodiments, the IL-2 booster dose is administered at a dose of 270,000 IU/kg. In other embodiments, the IL-2 booster dose is administered at a dose of 500,000 IU/kg. In other embodiments, the IL-2 booster dose is administered at a dose of 100,000 IU/kg. In other embodiments, the IL-2 booster dose is administered at a dose of 75,000 IU/kg. In other embodiments, the IL-2 booster dose is administered at a dose of 50,000 IU/kg.

In some embodiments, the booster dose of IL-2 is administered once, twice, three times, four times, five times, or six times. In some embodiments, the booster dose of IL-2 is administered one week, two weeks, three weeks, four weeks, 5 weeks, 6 weeks, 7 weeks, or 8 weeks after the loading dose of IL-2. In some embodiments, the 2^{nd} booster dose of IL-2 is administered one week, two weeks, three weeks, four weeks, 5 weeks, 6 weeks, 7 weeks, or 8 weeks after the 1^{st} booster dose of IL-2. In some embodiments, a booster dose of IL-2 is administered one week, two weeks, three weeks, four weeks, 5 weeks, 6 weeks, 7 weeks, or 8 weeks after the prior dose of IL-2 to the subject.

In certain embodiments of methods disclosed herein, the anti-IL-2 antibody is administered at a dose of 9 mg/kg of said subject's body weight, the IL-2 loading dose is administered at a dose of 135,000 IU/kg of said subject's body weight, and the at least one booster dose of IL-2 is administered at a dose of 135,000 IU/kg of said subject's body weight.

In some embodiments, the anti-IL-2 antibody or composition thereof and the low dose IL-2 (loading or booster dose) or composition thereof are administered at a frequency or interval that is independent of one another.

In some embodiments, methods as described herein comprising the administration of an anti-IL-2 antibody or composition thereof and a low dose of IL-2 or composition thereof, the administration of the anti-IL-2 antibody or composition thereof and the administration of the low dose of IL-2 or composition thereof is concurrent. In other embodiments, the anti-IL-2 antibody or composition thereof is administered prior to the administration of the low dose of IL-2 or composition thereof. In other embodiments, the anti-IL-2 antibody or composition thereof is administered following the administration of the low dose of IL-2 or composition thereof.

In some embodiments, methods as described herein comprising the administration of an anti-IL-2 antibody or composition thereof and a loading dose of IL-2 or composition thereof, the administration of the anti-IL-2 antibody or composition thereof and the administration of the loading dose of IL-2 or composition thereof is concurrent. In other embodiments, the anti-IL-2 antibody or composition thereof is administered prior to the administration of the loading dose of IL-2 or composition thereof. In other embodiments, the anti-IL-2 antibody or composition thereof is administered following the administration of the loading dose of IL-2 or composition thereof.

In some embodiments, methods as described herein comprising the administration of an anti-IL-2 antibody or composition thereof and a booster dose of IL-2 or composition thereof, the administration of the anti-IL-2 antibody or composition thereof and the administration of the booster dose of IL-2 or composition thereof is concurrent. In other embodiments, the anti-IL-2 antibody or composition thereof is administered prior to the administration of the booster dose of IL-2 or composition thereof. In other embodiments, the anti-IL-2 antibody or composition thereof is administered following the administration of the booster dose of IL-2 or composition thereof.

In some embodiments, methods as described herein comprising the administration of an immune checkpoint inhibitor comprise the step of administering the immune checkpoint inhibitor or a pharmaceutical composition thereof prior to the administration of the anti-IL-2 antibody. In some embodiments, the immune checkpoint inhibitor is administered concurrent with the administration of the anti-IL-2 antibody. In some embodiments, the immune checkpoint inhibitor is administered following the administration of the anti-IL-2 antibody.

In certain embodiments of methods disclosed herein, the administration of said loading dose of IL-2 is prior to, concurrent with, or following the administration of said anti-IL-2 antibody. In certain embodiments of methods disclosed herein, the administration of said loading dose of IL-2 is prior to, concurrent with, or following the administration of said anti-IL-2 antibody and or said immune checkpoint inhibitor, i.e., nivolumab.

In some embodiments, the methods described herein further comprise the step of administering an immune checkpoint inhibitor. In other embodiments, the methods described herein further comprise the step of administering a pharmaceutical composition comprising an immune checkpoint inhibitor.

In certain embodiments of methods disclosed herein, a method comprises or further comprises administering a checkpoint inhibitor or a pharmaceutical composition thereof, said pharmaceutical composition further comprising a pharmaceutically acceptable carrier, wherein the checkpoint inhibitor comprises:
a PD-L1 checkpoint inhibitor, said PD-L1 checkpoint inhibitor optionally comprising atezolizumab, durvalumab, sugemalimab, envafolimab, or cosibelimab; or
a PD-1 checkpoint inhibitor, said PD-1 checkpoint inhibitor optionally comprising nivolumab, pembrolizumab, cemiplimab, camrelizumab, zimberelimab, tislelizumab, sintilimab, teriprizumab, prolgolimab, penpulimab, dostarlimab, genolimzumab, or retifanlimab
   and wherein said checkpoint inhibitor is administered prior to, concurrent with, or following the administration of said anti-IL-2 antibody, said loading dose or booster dose of IL-2, or both.

In some embodiments, the checkpoint inhibitor comprises a PD-1 checkpoint inhibitor. In some embodiments, the checkpoint inhibitor comprises a PD-L1 checkpoint inhibitor. In some embodiments, the checkpoint inhibitor comprises nivolumab.

In some embodiments, methods described herein comprise administering an immune checkpoint inhibitor to a subject at a dose of 240 mg. In some embodiments, methods described herein comprise administering an immune checkpoint inhibitor to a subject at a dose of 480 mg. In other embodiments, methods described herein comprise administering an immune checkpoint inhibitor to a subject at a dose of 120 mg. In other embodiments, methods described herein comprise administering an immune checkpoint inhibitor to a subject at a dose of 360 mg. In other embodiments, methods described herein comprise administering an immune checkpoint inhibitor to a subject at a dose of 600 mg. In other embodiments, methods described herein comprise administering an immune checkpoint inhibitor to a subject at a dose of between about 120-600 mg. In other embodiments, methods described herein comprise administering an immune checkpoint inhibitor to a subject at a dose of between about 240-480 mg.

In some embodiments, methods as described herein comprising the administration of an immune checkpoint inhibitor comprise the step of administering the immune checkpoint inhibitor or a pharmaceutical composition thereof prior to the administration of the low dose of IL-2. In some embodiments, the immune checkpoint inhibitor is administered concurrent with the administration of the low dose of IL-2. In some embodiments, the immune checkpoint inhibitor is administered following the administration of the low dose of IL-2. In some embodiments, the low dose of IL-2 is the loading dose of IL-2. In other embodiments, the low dose of IL-2 is the booster dose of IL-2.

In some embodiments, the methods described herein further comprise the step of administering nivolumab. In other embodiments, the methods described herein further comprise the step of administering a pharmaceutical composition comprising nivolumab.

In some embodiments, methods described herein comprise administering nivolumab to a subject at a dose of 240 mg. In some embodiments, methods described herein comprise administering nivolumab to a subject at a dose of 480 mg. In other embodiments, methods described herein comprise administering nivolumab to a subject at a dose of 120 mg. In other embodiments, methods described herein comprise administering nivolumab to a subject at a dose of 360 mg. In other embodiments, methods described herein comprise administering nivolumab to a subject at a dose of 480 mg. In other embodiments, methods described herein comprise administering nivolumab to a subject at a dose of 600 mg. In other embodiments, methods described herein comprise administering nivolumab to a subject at a dose of between about 120-600 mg. In other embodiments, methods described herein comprise administering nivolumab to a subject at a dose of between about 240-480 mg.

In some embodiments, therapeutic dosages of nivolumab are administered at repeated administrations, e.g., of the same dose, over a period of months. In some embodiments, therapeutic dosages of nivolumab are administered for up to 3 months. In some embodiments, therapeutic dosages of nivolumab are administered for at least 3 months. In some embodiments, therapeutic dosages of nivolumab are administered for up to 6 months. In some embodiments, therapeutic dosages of nivolumab are administered for at least 6 months. In some embodiments, therapeutic dosages of nivolumab are administered for up to 9 months. In some embodiments, therapeutic dosages of nivolumab are administered for at least 9 months. In some embodiments, therapeutic dosages of nivolumab are administered over a period of up to a year. In some embodiments, therapeutic dosages of nivolumab are administered over a period of at least a year. In some embodiments, therapeutic dosages of nivolumab are administered over a period of up to a year and a half. In some embodiments, therapeutic dosages of nivolumab are administered over a period of at least a year and a half. In some embodiments, therapeutic dosages of nivolumab are administered over a period of up to 2 years. In some embodiments, therapeutic dosages of nivolumab are administered over a period of at least 2 years.

In some embodiments, the present disclosure provides a method of treating an unresectable locally advanced or metastatic solid cancer in a subject, said method comprising administering to said subject an anti-IL-2 antibody at a dose of 9 mg/kg of said subject's body weight or a pharmaceutical composition thereof, a loading dose of IL-2 at a dose of 135,000 IU/kg of said subject's body weight or a pharmaceutical composition thereof, and nivolumab at a dose of 480 mg or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; and wherein said pharmaceutical composition further comprises a pharmaceutically acceptable carrier, thereby treating said unresectable locally advanced or metastatic solid cancer in said subject.

In some embodiments, the present disclosure provides a method of treating an unresectable locally advanced or metastatic solid cancer in a subject, said method comprising administering to said subject an anti-IL-2 antibody at a dose of 9 mg/kg of said subject's body weight or a pharmaceutical composition thereof, a loading dose of IL-2 at a dose of 135,000 IU/kg of said subject's body weight or a pharmaceutical composition thereof, and nivolumab at a dose of 240 mg or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; and wherein said pharmaceutical composition further comprises a pharmaceutically acceptable carrier, thereby treating said unresectable locally advanced or metastatic solid cancer in said subject.

In some embodiments, the present disclosure provides a method of treating an unresectable locally advanced or metastatic solid cancer in a subject, said method comprising administering to said subject an anti-IL-2 antibody at a dose of 9 mg/kg of said subject's body weight or a pharmaceutical composition thereof once every 2 weeks, a loading dose of IL-2 at a dose of 135,000 IU/kg of said subject's body weight or a pharmaceutical composition thereof, and nivolumab at a dose of 480 mg or a pharmaceutical composition thereof once every 4 weeks, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; and wherein said pharmaceutical composition further comprises a pharmaceutically acceptable carrier, thereby treating said unresectable locally advanced or metastatic cancer in said subject.

In some embodiments of methods disclosed herein, the anti-IL-2 antibody, the nivolumab, or both are administered intravenously (IV) and the IL-2 loading dose is administered subcutaneously.

In some embodiments, the present disclosure provides a method of treating an unresectable locally advanced or metastatic solid cancer in a subject, said method comprising administering to said subject an anti-IL-2 antibody at a dose of 9 mg/kg of said subject's body weight or a pharmaceutical composition thereof once every 2 weeks, a loading dose of IL-2 at a dose of 135,000 IU/kg of said subject's body weight or a pharmaceutical composition thereof, and nivolumab at a dose of 240 mg or a pharmaceutical composition thereof once every 4 weeks, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; and wherein said pharmaceutical composition further comprises a pharmaceutically acceptable carrier, thereby treating said unresectable locally advanced or metastatic cancer in said subject.

In some embodiments, methods as described herein comprising the administration of nivolumab comprise the step of administering nivolumab or a pharmaceutical composition thereof prior to the administration of the anti-IL-2 antibody. In some embodiments, nivolumab is administered concurrent with the administration of the anti-IL-2 antibody. In some embodiments, nivolumab is administered following the administration of the anti-IL-2 antibody.

In some embodiments, methods as described herein comprising the administration of nivolumab comprise the step of administering nivolumab or a pharmaceutical composition thereof prior to the administration of the low dose of IL-2. In some embodiments, nivolumab is administered concurrent with the administration of the low dose of IL-2. In some embodiments, nivolumab is administered following the administration of the low dose of IL-2.

In some embodiments, the administration of the loading dose of IL-2 is prior to, concurrent with, or following the administration of the anti-IL-2 antibody, the checkpoint inhibitor, or both. In some embodiments, the administration of the loading dose of IL-2 is prior to, concurrent with, or following the administration of the anti-IL-2 antibody, the nivolumab, or both.

In some embodiments, methods as described herein comprising the administration of a PD-L1 immune checkpoint inhibitor comprise the step of administering the PD-L1 immune checkpoint inhibitor or a pharmaceutical composition thereof prior to the administration of the anti-IL-2 antibody. In some embodiments, the PD-L1 immune checkpoint inhibitor is administered concurrent with the administration of the anti-IL-2 antibody. In some embodiments, the PD-L1 immune checkpoint inhibitor is administered following the administration of the anti-IL-2 antibody.

In some embodiments, methods as described herein comprising the administration of the PD-L1 immune checkpoint inhibitor comprise the step of administering the PD-L1 immune checkpoint inhibitor or a pharmaceutical composition thereof prior to the administration of the low dose of IL-2. In some embodiments, the PD-L1 immune checkpoint inhibitor is administered concurrent with the administration of the low dose of IL-2. In some embodiments, the PD-L1 immune checkpoint inhibitor is administered following the administration of the low dose of IL-2.

In some embodiments, methods as described herein comprising the administration of a PD-1 immune checkpoint inhibitor comprise the step of administering the PD-1 immune checkpoint inhibitor or a pharmaceutical composition thereof prior to the administration of the anti-IL-2 antibody. In some embodiments, the PD-1 immune checkpoint inhibitor is administered concurrent with the administration of the anti-IL-2 antibody. In some embodiments, the PD-1 immune checkpoint inhibitor is administered following the administration of the anti-IL-2 antibody.

In some embodiments, methods as described herein comprising the administration of the PD-1 immune checkpoint inhibitor comprise the step of administering the PD-1 immune checkpoint inhibitor or a pharmaceutical composition thereof prior to the administration of the low dose of IL-2. In some embodiments, the PD-1 immune checkpoint inhibitor is administered concurrent with the administration of the low dose of IL-2. In some embodiments, the PD-1 immune checkpoint inhibitor is administered following the administration of the low dose of IL-2.

In some embodiments, the methods described herein further comprise the step of administering one or more additional doses of nivolumab. In other embodiments, the methods described herein further comprise the step of administering one or more additional doses of a pharmaceutical composition comprising nivolumab.

In some embodiments, the nivolumab is administered at least once every week, bi-weekly (once every two weeks), once every three weeks, once every four weeks, once every five week, or at least once every 6 weeks. In some embodiments, the schedule of administering the nivolumab may not be the same through the full course of the therapy, wherein administration may be bi-weekly for a time period and may then change to weekly, once every three weeks, once every four weeks, once every 5 weeks, or once every 6 weeks. Similarly, the schedule of administering the nivolumab may not be the same through the full course of the therapy, wherein administrations may be further apart, for example but not limited to once every 4-6 weeks and may then change to weekly, bi-weekly, or once every three weeks.

In some embodiments, the anti-IL-2 antibodies described and exemplified herein, bind the portion of IL-2 that interacts with the alpha (CD25) receptor subunit that is a component of the IL-2 trimeric receptor (CD25/CD132/CD122, sometimes represented as α/β/γ) found on Treg cells, eosinophils, and pulmonary and vascular endothelial cells. In some embodiments, the anti-IL-2 antibodies disclosed herein prevent activation of the trimeric IL-2 receptor found on Tregs, eosinophils, and pulmonary and vascular endothelial cells. In some embodiments, the anti-IL-2 antibodies disclosed herein bound to IL-2, activate signaling through the IL-2 dimer receptor (CD132/CD122, sometimes represented as β/γ) found on naïve Teff cells, NK cells, and Natural killer T (NKT) cells. Thus, in some embodiments, the present disclosure provides a method of preventing activation of the trimeric IL-2 receptor found on Tregs, eosinophils, and pulmonary and vascular endothelial cells. In other embodiments, the present disclosure provides a method of activating signaling through the IL-2 dimer receptor (CD132/CD122, sometimes represented as β/γ) found on naïve Teff cells, NK cells, and Natural killer T (NKT) cells.

In some embodiments, administration of an anti-IL-2 antibody disclosed herein, for example but not limited to AU-007, provides a method of preventing activation of the trimeric IL-2 receptor found on Tregs, eosinophils, and pulmonary and vascular endothelial cells and provides a method of activating signaling through the IL-2 dimer receptor (CD132/CD122, sometimes represented as β/γ) found on naïve Teff cells, NK cells, and Natural Killer T (NKT) cells. A skilled artisan would appreciate that the anti-IL-2 antibody may function as an immunomodulator that either increases or decreases immune function. In some embodiments, an anti-IL-2 antibody described herein, for example AU-007, increases immune function. In other embodiments, an anti-IL-2 antibody described herein, for example AU-007, decreases immune functions. In some embodiments, an anti-IL-2 antibody, for example AU-007 may either increase or decrease immune function. In some embodiments, an anti-IL-2 antibody described herein, for example AU-007, is an IL-2 modulator.

A complex of IL-2 and anti-IL-2 antibodies induced proliferation of memory phenotype effector T cells (MP) CD8⁺ cells and NK cells, while there was a much smaller effect on CD4⁺ Tregs. Thus, the engineered anti-IL-2 antibodies disclosed herein would be useful in adjusting immune cell populations and inducing differential expansion of certain immune effector cells. In some embodiments, such differential expansion of immune effect cells would result in robust activation of the immune system and could be useful for treatment of tumors. Thus, in some embodiments, the present disclosure provides a method of inducing proliferation of memory phenotype effector T cells (MP) CD8⁺ cells and NK cells. In other embodiments, the present disclosure provides a method of activating the immune system of a subject having an unresectable locally advanced or metastatic solid cancer for example but not limited to a cutaneous melanoma.

In certain embodiments, the present disclosure provides a method of treating a cancer that progressed after prior checkpoint inhibitor therapy. In some embodiments, the present disclosure provides a method of treating a subject having cancer, wherein said subject was previously treated with an immune checkpoint inhibitor. In other embodiments, the subject being treated has cancer that is recalcitrant to treatment with one or more immune checkpoint inhibitors.

In certain embodiments, the present disclosure provides a method of treating a cancer that progressed after prior administration of nivolumab. In some embodiments, the present disclosure provides a method of treating a subject having cancer, wherein said subject was previously treated with nivolumab. In other embodiments, the subject being treated has a cancer that is recalcitrant to treatment with nivolumab.

In certain embodiments, the present disclosure provides a method of treating a cancer that progressed after prior PD-1 therapy. In some embodiments, the present disclosure provides a method of treating a subject having cancer, wherein said subject was previously treated with a PD-1 inhibitor. In other embodiments, the subject being treated has a cancer that is recalcitrant to treatment with one or more PD-1 inhibitors.

In certain embodiments, the present disclosure provides a method of treating a cancer that progressed after prior PD-L1 therapy. In some embodiments, the present disclosure provides a method of treating a subject having cancer, wherein said subject was previously treated with a PD-L1 inhibitor. In other embodiments, the subject being treated has a cancer that is recalcitrant to treatment with one or more PD-L1 inhibitors.

In some embodiments, a method as described herein further comprises administering to said subject an adjuvant treatment. In other embodiments, a method as described herein further comprises administering to said subject a neoadjuvant treatment. In other embodiments, a method as described herein further comprises administering to said subject an adjuvant treatment and a neoadjuvant treatment.

In some embodiments, an “adjuvant” treatment is administered after primary treatment, such as surgery, to eliminate residual cancer cells that may remain and reduce the risk of the cancer returning. In some embodiments, a “neoadjuvant” treatment is administered before the primary treatment, typically surgery, to shrink tumors, make surgery less invasive, treat micrometastases, and improve the overall success of cancer treatment.

In some embodiments, the adjuvant or neo-adjuvant treatment comprises chemotherapy, radiation therapy, hormone therapy, targeted therapy, immunotherapy, or any combination thereof.

In some embodiments, the present disclosure provides a method for reducing the size of an unresectable locally advanced or metastatic solid cancer. In other embodiments, the present disclosure provides a method for inhibiting the growth of an unresectable locally advanced or metastatic solid cancer. In other embodiments, the present disclosure provides a method for reducing the growth of an unresectable locally advanced or metastatic solid cancer. In other embodiments, the present disclosure provides a method for inhibiting the metastases of an unresectable locally advanced or metastatic solid cancer. In other embodiments, the present disclosure provides a method for reducing the metastases of an unresectable locally advanced or metastatic solid cancer. In some embodiments, an unresectable, locally advanced or metastatic solid cancer comprises any of the cancers disclosed herein.

In some embodiments, the present disclosure provides a method for reducing the size of an unresectable locally advanced or metastatic melanoma. In other embodiments, the present disclosure provides a method for inhibiting the growth of an unresectable locally advanced or metastatic melanoma. In other embodiments, the present disclosure provides a method for reducing the growth of an unresectable locally advanced or metastatic melanoma. In other embodiments, the present disclosure provides a method for inhibiting the metastases of an unresectable locally advanced or metastatic melanoma. In other embodiments, the present disclosure provides a method for reducing the metastases of a melanoma. In some embodiments, a melanoma comprises an unresectable locally advanced or metastatic cutaneous melanoma.

In some embodiments, the present disclosure provides a method for reducing the size of an unresectable locally advanced or metastatic cutaneous melanoma. In other embodiments, the present disclosure provides a method for inhibiting the growth of an unresectable locally advanced or metastatic cutaneous melanoma. In other embodiments, the present disclosure provides a method for reducing the growth of an unresectable locally advanced or metastatic cutaneous melanoma. In other embodiments, the present disclosure provides a method for inhibiting the metastases of an unresectable locally advanced or metastatic cutaneous melanoma. In other embodiments, the present disclosure provides a method for reducing the metastases of an unresectable locally advanced or metastatic cutaneous melanoma.

In some embodiments, the present disclosure provides a method for reducing the size of an unresectable, locally advanced or metastatic solid cancer. In other embodiments, the present disclosure provides a method for inhibiting the growth of an unresectable, locally advanced or metastatic solid cancer. In other embodiments, the present disclosure provides a method for reducing the growth of an unresectable, locally advanced or metastatic solid cancer. In other embodiments, the present disclosure provides a method for inhibiting the metastases of an unresectable, locally advanced or metastatic solid cancer. In other embodiments, the present disclosure provides a method for reducing the metastases of an unresectable, locally advanced or metastatic solid cancer. In some embodiments, an unresectable, locally advanced or metastatic solid cancer comprises any of the cancers disclosed herein.

In some embodiments, the present disclosure provides a method for reducing the size of an unresectable, locally advanced or metastatic melanoma In other embodiments, the present disclosure provides a method for inhibiting the growth of an unresectable, locally advanced or metastatic melanoma In other embodiments, the present disclosure provides a method for reducing the growth of an unresectable, locally advanced or metastatic melanoma In other embodiments, the present disclosure provides a method for inhibiting the metastases of an unresectable, locally advanced or metastatic melanoma In other embodiments, the present disclosure provides a method for reducing the metastases of an unresectable, locally advanced or metastatic melanoma In some embodiments, an unresectable, locally advanced or metastatic melanoma comprises a cutaneous melanoma.

In some embodiments, the present disclosure provides a method for reducing the size of the cutaneous melanoma. In other embodiments, the present disclosure provides a method for inhibiting the growth of the cutaneous melanoma. In other embodiments, the present disclosure provides a method for reducing the growth of the cutaneous melanoma.

In other embodiments, the present disclosure provides a method for inhibiting the metastases of the cutaneous melanoma. In other embodiments, the present disclosure provides a method for reducing the metastases of the cutaneous melanoma.

In other embodiments, the present disclosure provides a method for increasing the time before detection of a new lesion. In other embodiments, the present disclosure provides a method for decreasing a target lesion. In other embodiments, the present disclosure provides a method for shrinking a target lesion. In other embodiments, the present disclosure provides a method for obliterating or eradicating a target lesion. In other embodiments, the present disclosure provides a method for removing a target lesion. In other embodiments, the present disclosure provides a method for causing a target lesion to disappear.

In some embodiments, treatment of an unresectable locally advanced or metastatic solid cancer in a subject reduces the size of the solid cancer, inhibits or reduces growth of the solid cancer, or inhibits or reduces metastases of said solid cancer, or any combination thereof.

In some embodiments, treatment of an unresectable locally advanced or metastatic melanoma in a subject reduces the size of the melanoma, inhibits or reduces growth of the melanoma, or inhibits or reduces metastases of said melanoma, or any combination thereof.

In some embodiments, treatment of an unresectable locally advanced or metastatic cutaneous melanoma in a subject reduces the size of the cutaneous melanoma, inhibits or reduces growth of the cutaneous melanoma, or inhibits or reduces metastases of said cutaneous melanoma, or any combination thereof.

In some embodiments, the method comprises the step of administering an anti-IL-2 antibody. In other embodiments, the method comprises the step of administering an anti-IL-2 antibody and a low loading dose of IL-2. In other embodiments, the method comprises administering an anti-IL-2 antibody and one or more immune checkpoint inhibitors. In other embodiments, the method comprises administering an anti-IL-2 antibody and nivolumab. In other embodiments, the method comprises administering an anti-IL-2 antibody, a low loading dose of IL-2 and one or more immune checkpoint inhibitors. In other embodiments, the method comprises administering an anti-IL-2 antibody, a low loading dose of IL-2, and nivolumab. In other embodiments, the method comprises administering an anti-IL-2 antibody, a low loading dose of IL-2, one or more low booster doses of IL-2, and one or more immune checkpoint inhibitors. In other embodiments, the method comprises administering an anti-IL-2 antibody, a low loading dose of IL-2, one or more low booster doses of IL-2, and nivolumab.

As used throughout, the terms “cancer”, “tumor”, “solid tumor”, “solid cancer”, “unresectable locally advanced cancer” and “unresectable locally advanced solid cancer” may in some embodiments be used interchangeably having the same meanings and qualities.

In other embodiments, the present disclosure provides a method of treating an unresectable cutaneous melanoma. In other embodiments, the present disclosure provides a method of treating a locally advanced cutaneous melanoma. In other embodiments, the present disclosure provides a method of treating an unresectable locally advanced cutaneous melanoma. In some embodiments, the present disclosure provides a method of treating a metastatic cutaneous melanoma.

In other embodiments, a subject treated by a method disclosed herein has an unresectable cutaneous melanoma. In other embodiments, a subject treated by a method disclosed herein has a locally advanced cutaneous melanoma. In other embodiments, a subject treated by a method disclosed herein has an unresectable locally advanced cutaneous melanoma. In some embodiments, a subject treated by a method disclosed herein has a metastatic cutaneous melanoma.

In some embodiments, an unresectable tumor is a tumor that is not considered a candidate for surgical removal due to factors like advanced stage, metastasis, or being too large, too close to vital structures, or too difficult to access. In some embodiments, an unresectable tumor is an inoperable tumor having a technical or anatomical barrier to resection, such as complete encasement of a major blood vessel, that makes complete surgical removal uncertain or impossible.

In some embodiments, the cutaneous melanoma comprises an immune sensitive cutaneous melanoma.

In some embodiments, treatment of an unresectable locally advanced or metastatic cutaneous melanoma in a subject as described herein comprises maintenance treatments. In some embodiments, maintenance treatments are administered to maintain the absence of an unresectable locally advanced or metastatic cutaneous melanoma. In some embodiments, maintenance treatments are administered to maintain lack of metastasis of an unresectable locally advanced or metastatic cutaneous melanoma. In some embodiments, maintenance treatments are administered to inhibit metastasis of an unresectable locally advanced or metastatic cutaneous melanoma. In some embodiments, maintenance treatments are administered to maintain lack of growth of an unresectable locally advanced or metastatic cutaneous melanoma. In some embodiments, maintenance treatments are administered to inhibit growth of an unresectable locally advanced or metastatic cutaneous melanoma.

In some embodiments, treatment of cutaneous melanoma comprises prophylactic treatment of, for example, but not limited to, a subject harboring a genetic marker or markers with a high risk of developing cutaneous melanoma.

In some embodiments, the present disclosure provides a method of promoting differential growth of immune cells in a subject, comprising the step of preparing a composition comprising an anti-IL-2 antibody disclosed herein, and administering the composition to the subject, thereby promoting differential growth of immune cells in the subject. In some embodiments, the present disclosure provides a method of promoting differential growth of immune cells in a subject, comprising the step of preparing a composition comprising IL-2 and the anti-IL-2 antibody disclosed herein, and administering the composition to the subject, thereby promoting differential growth of immune cells in the subject. In some embodiments, the subject can be an animal or a human. In some embodiments, the immune cells can be CD8⁺ cells or NK cells.

In some embodiments, disclosed herein is a method of treating an unresectable locally advanced or metastatic cutaneous melanoma in a subject, comprising the step of administering to the subject a composition comprising an anti-IL-2 antibody as disclosed herein, wherein said antibody promotes differential growth of subsets of immune cells and decreases undesirable effects caused by IL-2, thereby treating unresectable locally advanced or metastatic cutaneous melanoma in said subject. In some embodiments, a method of treating a disease disclosed here comprises use of a composition comprising an anti-IL-2 antibody and IL-2. In some embodiments, a method of treating a disease comprises treating an unresectable locally advanced or metastatic cutaneous melanoma. In some embodiments, a method of treating a condition comprises treating a weak immune system and the treatment prophylactically boosts the immune system.

In some embodiments, methods of promoting differential growth of immune cells in a subject, comprise the step of preparing and administering a composition comprising an anti-IL-2 antibody disclosed herein. In some embodiments, methods of promoting differential growth of immune cells in a subject, comprise the step of preparing and administering a composition comprising IL-2. In some embodiments, methods of promoting differential growth of immune cells in a subject, comprise the step of preparing and administering a composition comprising an immune checkpoint inhibitor. In some embodiments, methods of promoting differential growth of immune cells in a subject, comprise the step of preparing and administering a composition comprising nivolumab. In some embodiments, methods of promoting differential growth of immune cells in a subject, comprise the step of preparing and administering an anti-IL-2 antibody and IL-2 or composition(s) thereof. In some embodiments, the IL-2 is a loading dose of IL-2. In some embodiments, the IL-2 is a booster dose of IL-2. In some embodiments, the loading dose or booster dose or both of IL-2 is low dose IL-2.

In some embodiments, methods of promoting differential growth of immune cells in a subject, comprise the step of preparing and administering an anti-IL-2 antibody and an immune checkpoint inhibitor or composition(s) thereof. In other embodiments, methods of promoting differential growth of immune cells in a subject, comprise the step of preparing and administering an anti-IL-2 antibody and a PD-L1 inhibitor or a PD-1 inhibitor or composition(s) thereof. In other embodiments, methods of promoting differential growth of immune cells in a subject, comprise the step of preparing and administering an anti-IL-2 antibody and nivolumab or composition(s) thereof. In other embodiments, the method comprises preparing and administering an anti-IL-2 antibody, an immune checkpoint inhibitor, and IL-2 or composition(s) thereof. In other embodiments, the method comprises preparing and administering an anti-IL-2 antibody, a PD-L1 inhibitor or a PD-1 inhibitor, and IL-2 or composition(s) thereof. In other embodiments, the method comprises preparing and administering an anti-IL-2 antibody, nivolumab, and IL-2 or composition(s) thereof. In some embodiments, the IL-2 is a loading dose of IL-2. In some embodiments, the IL-2 is a booster dose of IL-2. In some embodiments, the loading dose or booster dose or both of IL-2 is low dose IL-2.

In some embodiments, the methods described herein comprise the steps of:
(a) preparing a composition comprising an anti-IL-2 antibody as disclosed herein; and
(b) administering the composition from (a) to the subject.

In some embodiments, the methods described herein comprise the steps of:
(a) preparing a composition comprising an anti-IL-2 antibody as disclosed herein;
(b) preparing a composition comprising IL-2 as disclosed herein;
(c) preparing a composition comprising an immune checkpoint inhibitor as disclosed herein; and
(d) administering the composition from (a), (b), and (c) as disclosed herein, to the subject. In some embodiments, the immune checkpoint inhibitor comprises a PD-L1 checkpoint inhibitor. In other embodiments, the immune checkpoint inhibitor comprises a PD-1 checkpoint inhibitor. In some embodiments, the immune checkpoint inhibitor is nivolumab.

In some embodiments, the methods described herein comprise the steps of:
(a) preparing a composition comprising an anti-IL-2 antibody as disclosed herein;
(b) preparing a composition comprising a loading dose of IL-2 as disclosed herein;
(c) preparing a composition comprising an immune checkpoint inhibitor as disclosed herein; and
(d) administering the composition from (a), (b), and (c) as disclosed herein, to the subject, wherein additional doses of the anti-IL-2 antibody are administered for the duration of the treatment, and additional doses of the immune checkpoint inhibitor are administered for the duration of the treatment, and if analysis of objective signs warrants, an at least one booster IL-2 dose is administered. In some embodiments, the immune checkpoint inhibitor comprises a PD-L1 checkpoint inhibitor. In other embodiments, the immune checkpoint inhibitor comprises a PD-1 checkpoint inhibitor. In some embodiments, the immune checkpoint inhibitor is nivolumab.

In some embodiments, the methods described herein comprise the steps of:
(a) preparing a composition comprising an anti-IL-2 antibody as disclosed herein;
(b) preparing a composition comprising a loading dose of IL-2 as disclosed herein;
(c) preparing a composition comprising nivolumab as disclosed herein; and
(d) administering the composition from (a), (b), and (c) as disclosed herein, to the subject, wherein additional doses of the anti-IL-2 antibody are administered for the duration of the treatment, and additional doses of nivolumab are administered for the duration of the treatment, and if analysis of objective signs warrants, an at least one booster IL-2 dose is administered.

In certain embodiments of methods disclosed herein, the method further comprises the step of administering one or more (a) additional doses of said anti-IL-2 antibody or a pharmaceutical composition thereof, (b) additional doses of said nivolumab or a pharmaceutical composition thereof, or (c) booster doses of IL-2 or a pharmaceutical composition thereof, or (d) any combination thereof, to said subject, wherein said pharmaceutical composition(s) further comprise(s) a pharmaceutically acceptable carrier. In some embodiments of the methods, (a) said additional doses of said anti-IL-2 antibody or a pharmaceutical composition thereof are administered once every two weeks; or (b) said additional doses of said nivolumab or a pharmaceutical composition thereof are administered once every four weeks; or (c) any combination thereof. In some embodiments of the methods, (a) said additional doses of said anti-IL-2 antibody or a pharmaceutical composition thereof are administered once every two weeks; or (b) said additional doses of said nivolumab or a pharmaceutical composition thereof are administered once every two weeks; or (c) any combination thereof. In some embodiments, the booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof, and wherein said IL-2 booster dose is administered subcutaneously. In some embodiments of the methods, the administration of said one or more booster doses of IL-2 is prior to, concurrent with, or following the administration of said one or more additional doses of said anti-IL-2 antibody, one or more additional doses of said nivolumab, or both, optionally wherein the at least one IL-2 booster dose is administered at a dose of 135,000 IU/kg of said subject's body weight.

In some embodiments, the subject can be an animal or a human.

In some embodiments, treatment with the anti-IL-2 antibodies expands immune cells comprising one or more of naïve T cells, memory T cells, CD8⁺ T cells, NK cells, and Natural Killer T cells. In some embodiments, treatment with the anti-IL-2 antibodies decreases one or more undesirable effects caused by IL-2 such as activation of regulatory T cells, apoptosis of CD25⁺ T effector cells, pulmonary edema, pneumonia, and IL-2-induced vascular leakage.

In some embodiments, an anti-IL-2 antibody as described herein is engineered or modified. In some embodiments, the engineered or modified anti-IL-2 antibodies comprise a heavy chain variable region and a light chain variable region having the sequences of SEQ ID NO:6 and SEQ ID NO:7.

In another embodiment, the engineered or modified anti-IL-2 antibodies comprise a heavy chain variable region having complementarity determining region (CDR) 1, CDR2 and CDR3. In some embodiments, the heavy chain CDR1, CDR2 and CDR3 comprise amino acid sequences of SEQ ID NOs:1-3 respectively.

In other embodiments, the engineered or modified anti-IL-2 antibodies comprise a light chain variable region having complementarity determining region (CDR) 1, CDR2 and CDR3. In some embodiments, the light chain CDR1, CDR2 and CDR3 comprise amino acid sequences of SEQ ID NO:4, DAS, and SEQ ID NO: 5 respectively.

In other embodiments, the amino acid sequence of the full length heavy chain of the anti-IL-2 antibody is as set forth in SEQ ID NO: 8 and the amino acid sequence of the full length light chain of the anti-IL-2 antibody is as set forth in SEQ ID NO: 9. In some embodiments, the engineered anti-IL-2 antibody is an IgG. In other embodiments, the engineered anti-IL-2 antibody is an IgA. In other embodiments, the engineered anti-IL-2 antibody is an IgM.

In other embodiments, the engineered anti-IL-2 antibody is an IgE. In other embodiments, the engineered anti-IL-2 antibody is an IgD. In other embodiments, the engineered anti-IL-2 antibody is a Fv. In other embodiments, the engineered anti-IL-2 antibody is a scFv. In other embodiments, the engineered anti-IL-2 antibody is a Fab. In other embodiments, the engineered anti-IL-2 antibody is a F(ab′)₂. In some embodiments, the IgG is of the subclass of IgG1. In other embodiments, the IgG is of the subclass of IgG2. In other embodiments, the IgG is of the subclass of IgG3. In other embodiments, the IgG is of the subclass of IgG4. In some embodiments, the engineered antibody is part of a minibody. In other embodiments, the engineered antibody is part of a diabody. In other embodiments, the engineered antibody is part of a triabody antibody.

In some embodiments, the engineered anti-IL-2 antibody comprises a heavy chain comprising a mutation that reduces binding to a fragment crystallizable gamma receptor (FcγR; Fcγ receptor). In some embodiments, the mutation comprises L234A, L235A mutations.

In some embodiments, the polypeptides disclosed herein may be administered to a subject directly, or by administering to the subject a nucleic acid sequence encoding the polypeptides. In some embodiments, the nucleic acid sequence may be carried by a vector.

In some embodiments, a polynucleotide sequence encoding an engineered anti-IL-2 antibody is used in a method of treating a subject with a disease or condition as described herein, wherein the polynucleotide encodes an antibody comprising a heavy chain variable region having the amino acid sequence of SEQ ID NO:6 In some embodiments, a polynucleotide sequence encoding an engineered anti-IL-2 antibody is used in a method of treating a subject with a disease or condition as described herein, wherein the polynucleotide encodes an antibody comprising a light chain variable region having the amino acid sequence of SEQ ID NO:7. In some embodiments, a polynucleotide sequence encoding an engineered anti-IL-2 antibody is used in a method of treating a subject with a disease or condition as described herein, wherein the polynucleotide encodes an antibody comprising a heavy chain variable region and a light chain variable region having the amino acid sequences of one of SEQ ID NOs:6 and 7.

In some embodiments of a method of using a polynucleotide to treat a disease or condition as described above, the polynucleotide encodes an engineered anti-IL-2 antibody that can be an IgG, IgA, IgM, IgE, IgD, a Fv, a scFv, a Fab, or a F(ab′)₂. The IgG can be of the subclass of IgG1, IgG2, IgG3, or IgG4. In some embodiments, the polynucleotide encodes an engineered antibody which is part of a minibody, a diabody, or a triabody antibody.

In some embodiments a polynucleotide sequence encoding an engineered anti-IL-2 antibody is used in a method of treating a subject with a disease or condition as described herein, wherein the polynucleotide sequence comprises the sequence of SEQ ID NO: 10

SEQ ID NO: 10)
(caggtccaactggtgcagtccggtgccgaagttaaaaaacctgggtctt
ccgttaaagtttcttgcaaagcctctggctacagcatcaccgatgacctg
attcactgggtccgtcaggctccaggtcaaggtctggaatggatgggttg
gatcgatccagaagacggtgaaaccaactatgcccagaaattccagggtc
gtgtaaccctgaccgccgacacctccacctctaccgcctacatggagtta
agtagcctgcgttcagaggataccgcagtgtactactgcgctcgttcact
ggactccacctggatctacccattcgcatactggggtcagggcaccctgg
taaccgttagtagcggcggtggtggtagcggaggcggaggatcaggtgga
ggcggcagtgacatcgtgatgacccagtctcctgactccttggccgtctc
tctgggcgaacgtgcaactatcaactgcaaatccagccagagcttactgc
gtcgcggtaatcagaaaaaccaccttgcatggtatcagcagaaaccaggt
cagccaccaaaattactgatctatgacgcatctaccggtcaaagcggtgt
cccagatcgtttcagcggttccggctccggtactgacttcaccctgacca
tctcttcccttcaggccgaagatgtggccgtgtattactgcctgcagagc
tacatcaccccacctactttcggtgctggtactaaagttgaaatcaaa;.

In some embodiments of a method of treating a disease or condition as described herein, the immune effector cells that are activated by the treatment are CD8+ cells or NK cells. In some embodiments, the anti-IL-2 antibodies disclosed herein, or a complex of IL-2 and the anti-IL-2 antibodies disclosed herein, exhibits pronounced effect in inducing proliferation of MP CD8⁺ cells and NK cells, while there was much smaller effect on CD4⁺ Tregs. In certain embodiments, there is no effect on CD4⁺ Tregs.

In certain embodiments, methods of use of an anti-IL-2 antibody disclosed herein provide a pro-stimulatory effect. In some embodiments, said use comprises the anti-IL-2 antibody. In some embodiments, said use comprises the anti-IL-2 antibody and an IL-2 (loading or booster dose), a PD-1 inhibitor, a PD-L1 inhibitor, nivolumab, or a combination thereof.

Thus, the engineered anti-IL-2 antibodies disclosed herein are useful in adjusting immune cell populations and inducing differential expansion of certain immune effector cells in a method of treating a disease such as an unresectable locally advanced or metastatic cutaneous melanoma, or treating a condition such as IL-2 induced pulmonary edema, or IL-2-induced vascular leakage.

In some embodiments, a method disclosed herein for treating an unresectable locally advanced or metastatic solid cancer comprises administering an anti-IL-2 antibody or a pharmaceutical composition thereof, a loading dose of IL-2 or a pharmaceutical composition thereof, and nivolumab or a pharmaceutical composition thereof to a subject further comprises administering an additional one or more immune checkpoint inhibitors targeting one or more immune checkpoints to the subject. In some embodiments, a subject is treated with said immune checkpoint inhibitors concurrently, before, or after treatment with an anti-IL-2 antibody. In some embodiments, a subject is treated with said immune checkpoint inhibitors concurrently, before, or after treatment with low dose IL-2, which in some embodiments, is a loading does and in other embodiments, is a booster dose. In some embodiments, a subject is treated with said immune checkpoint inhibitors concurrently, before, or after treatment with nivolumab. In some embodiments of a method of treating disclosed herein, an immune checkpoint comprises a Programmed death-ligand 1 (PD-L1), a Programmed cell death protein 1 (PD-1), a Cytotoxic T-lymphocyte protein 4 (CTLA-4), a T-cell immunoreceptor with Ig and ITIM domains (TIGIT), a Metalloproteinase inhibitor 3 (TIM-3), B7 homolog 3 (B7-H3), a cluster of differentiation 73 (CD73), a Lymphocyte-activation gene 3 (LAG3), a cluster of differentiation 27 (CD27), a cluster of differentiation 70 (CD70), a Tumor necrosis factor ligand superfamily member 9 (4-1 BB), a Glucocorticoid-Induced TNFR-Related (GITR), a Tumor necrosis factor receptor superfamily member 4 (OX40), a cluster of differentiation 47 (SIRP-alpha (CD47)), a cluster of differentiation 39 (CD39), an Immunoglobulin Like Domain Containing Receptor 2 (ILDR2), a V-Domain Ig Suppressor Of T Cell Activation (VISTA), a B and T lymphocyte attenuator (BTLA), or a V-set domain containing T cell activation inhibitor 1 (VTCN-1), or a combination thereof. In some embodiments, the one or more immune checkpoint inhibitors comprises avelumab, atezolizumab, durvalumab, sugemalimab, cosibelimab, envafolimab, nivolumab, pembrolizumab, cemiplimab, camrelizumab, zimberelimab, tislelizumab, sintilimab, teriprizumab, prolgolimab, penpulimab, dostarlimab, genolimzumab, retifanlimab, or a combination thereof.

In some embodiments, the PD-L1 immune checkpoint inhibitor comprises avelumab, atezolizumab, durvalumab, sugemalimab, cosibelimab or envafolimab. In some embodiments, the PD-1 immune checkpoint inhibitor comprises nivolumab, pembrolizumab, cemiplimab, camrelizumab, zimberelimab, tislelizumab, sintilimab, teriprizumab, prolgolimab, penpulimab, dostarlimab, genolimzumab, or retifanlimab.

In some embodiments, the checkpoint inhibitor comprises avelumab. In other embodiments, the checkpoint inhibitor comprises atezolizumab. In other embodiments, the checkpoint inhibitor comprises durvalumab. In other embodiments, the checkpoint inhibitor comprises sugemalimab. In other embodiments, the checkpoint inhibitor comprises or envafolimab. In some embodiments, the checkpoint inhibitor comprises cosibelimab. In other embodiments, the checkpoint inhibitor comprises pembrolizumab. In other embodiments, the checkpoint inhibitor comprises cemiplimab. In other embodiments, the checkpoint inhibitor comprises camrelizumab. In other embodiments, the checkpoint inhibitor comprises zimberelimab. In other embodiments, the checkpoint inhibitor comprises tislelizumab. In other embodiments, the checkpoint inhibitor comprises sintilimab. In other embodiments, the checkpoint inhibitor comprises teriprizumab. In other embodiments, the checkpoint inhibitor comprises prolgolimab. In other embodiments, the checkpoint inhibitor comprises penpulimab. In other embodiments, the checkpoint inhibitor comprises dostarlimab. In other embodiments, the checkpoint inhibitor comprises genolimzumab. In other embodiments, the checkpoint inhibitor comprises retifanlimab.

In some embodiments, the anti-IL-2 antibody, the low dose IL-2 (loading or booster dose) and the immune checkpoint inhibitor are comprised in different pharmaceutical compositions.

In some embodiments, the immune checkpoint inhibitor is administered prior to the anti-IL-2 antibody administration. In other embodiments, the immune checkpoint inhibitor is administered concurrent with the anti-IL-2 antibody administration. In other embodiments, the immune checkpoint inhibitor is administered following the administration of the anti-IL-2 antibody.

In some embodiments, the immune checkpoint inhibitor is administered prior to the low dose IL-2 administration. In other embodiments, the immune checkpoint inhibitor is administered concurrently with the low dose IL-2 administration. In other embodiments, the immune checkpoint inhibitor is administered following the administration of the low dose IL-2.

As discussed above, in some embodiments, a therapeutic method of treatment as disclosed herein, further comprises administering an additional active agent comprising a checkpoint inhibitor. One skilled in the art would appreciate that a combination therapy comprising an anti-IL-2 antibody therapy in the presence or absence of IL-2, and additionally comprising a checkpoint inhibitor may utilize any of the therapeutic compositions or formulations comprising an anti-IL-2 antibody+/−IL-2, and a checkpoint inhibitor as provided herein. In some embodiments, at least two checkpoint inhibitors are used in a combination therapy.

In some embodiments of a method of use of a combination as described herein, a subject comprises a mammalian subject. In some embodiments, a subject comprises a human subject. In some embodiments, a subject suffers from immune deficiency. Treatment of an immune deficient subject would, in some embodiments, comprise a prophylactic treatment.

### Combination Therapies

In some embodiments, an anti-IL-2 antibody or composition thereof as disclosed herein, is used as part of a combination therapy. In some embodiments, an anti-IL-2 antibody or composition thereof as disclosed herein, is used in combination with IL-2 or composition thereof as disclosed herein. In some embodiments, an anti-IL-2 antibody or composition thereof as disclosed herein, is used in combination with IL-2 or composition thereof as disclosed herein and with an immune checkpoint inhibitor or composition thereof as disclosed herein. In some embodiments, an anti-IL-2 antibody or composition thereof as disclosed herein, is used in combination with an immune checkpoint inhibitor or composition thereof as disclosed herein. In some embodiments, the IL-2 is low-dose IL-2. In some embodiments, the IL-2 is a loading dose of IL-2. In some embodiments, the IL-2 is a booster dose of IL-2. In some embodiments, IL-2 is administered as a loading dose and as a booster dose at a later time period depending on the need of the patient.

In some embodiments of the methods described herein that comprise administering a booster dose of IL-2, the booster dose of IL-2 is administered to the subject until at least Partial Response (PR) is achieved. In some embodiments of the methods described herein that comprise administering a booster dose of IL-2, the booster dose of IL-2 is administered to the subject with each treatment cycle, until at least Partial Response (PR) is achieved.

In some embodiments of the methods described herein that comprise administering a booster dose of IL-2, the booster dose of IL-2 is administered to the subject until at least complete response (CR) is achieved. In some embodiments of the methods described herein that comprise administering a booster dose of IL-2, the booster dose of IL-2 is administered to the subject with each treatment cycle, until at least complete response (CR) is achieved.

In some embodiments, Partial Response (PR) or complete response (CR) is evaluated according to RECIST.

In some embodiments of the methods described herein that comprise administering a booster dose of IL-2, the booster dose of IL-2 is administered to the subject until objective tumor shrinkage is observed. In some embodiments of the methods described herein that comprise administering a booster dose of IL-2, the booster dose of IL-2 is administered to the subject with each treatment cycle, until objective tumor shrinkage is observed.

In some embodiments, the booster dose of IL-2 is administered to the subject on day 1 of each cycle (Q8W). In some embodiments, the booster dose of IL-2 is administered to the subject until objective tumor shrinkage, or partial response, or complete response is observed on radiologic imaging or physical exam.

In some embodiments, the terms “combination” and “combination therapy” may be used interchangeably having all the same meanings and qualities.

In some embodiments, an anti-IL-2 antibody or a composition thereof, is used in combination with an immune checkpoint inhibitor. In some embodiments, the term “immune checkpoint inhibitor” may encompass any compound or molecule capable of inhibiting the function of a checkpoint protein. In some embodiments, the term “immune checkpoint inhibitor” may encompass any compound or molecule which targets immune checkpoint proteins. An artisan would appreciate that “immune checkpoints” are key regulators of the immune system that when stimulated can dampen the immune response to an immunologic stimulus. Checkpoint inhibitors can block inhibitory checkpoints, and thereby restore immune system function. In some embodiments, the one or more checkpoint inhibitors comprise immune checkpoint inhibitors.

A skilled artisan would appreciate that the terms “immune checkpoint inhibitors” (ICIs), “checkpoint inhibitors,” and the like may be used interchangeably herein having all the same qualities and meanings, wherein an immune checkpoint inhibitor encompasses compounds that inhibit the activity or control mechanism(s) of the immune system. Immune system checkpoints, or immune checkpoints, are inhibitory pathways in the immune system that generally act to maintain self-tolerance or modulate the duration and amplitude of physiological immune responses to minimize collateral tissue damage. Checkpoint inhibitors can inhibit an immune system checkpoint by inhibiting the activity of a protein in the pathway.

Immune checkpoint inhibitor targets include, but are not limited to PD-1, PD-L1, CTLA-4, TIGIT, TIM-3, B7-H3, CD73, LAG3, CD27, CD70, 4-1 BB, GITR, OX40, SIRP-alpha (CD47), CD39, ILDR2, VISTA, BTLA, and VTCN-1. In some embodiments, an anti-IL-2 antibody therapy is used in combination with an immune checkpoint inhibitor, wherein the target of the immune checkpoint inhibitor comprises PD-1, PD-L1, CTLA-4, TIGIT, TIM-3, B7-H3, CD73, LAG3, CD27, CD70, 4-1 BB, GITR, OX40, SIRP-alpha (CD47), CD39, ILDR2, VISTA, BTLA, or VTCN-1, or any combination thereof.

Checkpoint inhibitors may include antibodies, or antigen binding fragments thereof, other binding proteins, biologic therapeutics, or small molecules, that bind to and block or inhibit the activity of one or more of PD-1, PD-L1, CTLA-4, TIGIT, TIM-3, B7-H3, CD73, LAG3, CD27, CD70, 4-1 BB, GITR, OX40, SIRP-alpha (CD47), CD39, ILDR2, VISTA, BTLA, or VTCN-1. Illustrative checkpoint inhibitors include but are not limited to those listed in Table 1 below.

TABLE 1
Non-Limiting Examples of Checkpoint Inhibitors
and the Immune Checkpoint Inhibitor Target.

| Drug name | ICI Target |
|---|---|
| Avelumab (Bavencio ®) | anti-PD-L1 |
| Atezolizumab (Tecentriq ®) | anti-PD-L1 |
| Durvalumab (Imfinzi ®) | anti-PD-L1 |
| Sugemalimab (Cejemly ®) | anti-PD-L1 |
| Envafolimab | anti-PD-L1 |
| Cosibelimab (Unloxcyt ®) | anti-PD-L1 |
| Nivolumab (Opdivo)- ®)-BMS | anti-PD-1 |
| Pembrolizumab (Keytruda) ®) | anti-PD-1 |
| Cemiplimab (Libtayo ®) | anti-PD-1 |
| Camrelizumab | anti-PD-1 |
| Zimberelimab (Glimta ®) | anti-PD-1 |
| Tislelizumab (Tevimbra ®) | anti-PD-1 |
| Sintilimab (Tyvyt ®) | anti-PD-1 |
| Teriprizumab | anti-PD-1 |
| Prolgolimab | anti-PD-1 |
| Penpulimab | anti-PD-1 |
| Dostarlimab (Jemperli ®) | anti-PD-1 |
| Genolimzumab | anti-PD-1 |
| Retifanlimab (Zynyz ®) | anti-PD-1 |
| Ipilimumab (Yervoy) ®) | anti-CTLA4 |
| Tiragolumab | anti-TIGIT |
| Domvanalimab | anti-TIGIT |
| Vibostolimab | anti-TIGIT |
| BMS-986207 | anti-TIGIT |
| EOS-448 (belrestotug ®) | anti-TIGIT |
| COM-902 | anti-TIGIT |
| Sabatolimab | anti-TIM3 |
| Cobolimab | anti-TIM3 |
| BMS-986258 | anti-TIM3 |
| INCAGN-02390 | anti-TIM3 |
| S-95018 | anti-TIM3 |
| Omburtamab | anti-B7-H3 |
| MGC-018 | anti-B7-H3 |
| Enoblituzumab | anti-B7-H3 |
| Oleclumab | anti-CD73 |
| BMS-986179 | anti-CD73 |
| NZV-930 | anti-CD73 |
| CPX-006 | anti-CD73 |
| MK-4280 (favezelimab ®) | anti-LAG3 |
| Sym-022 | anti-LAG3 |
| Ieramilimab | anti-LAG3 |
| BI-754111 (miptenalimab ®) | anti-LAG3 |
| MK-5890 (boserolimab ®) | anti-CD27 |
| Varlilumab | anti-CD27 |
| Cusatuzumab | anti-CD70 |
| Vorsetuzumab | anti-CD70 |
| Urelumab | anti-4-1BB (agonist) |
| Utomilumab | anti-4-1BB (agonist) |
| ATOR-1017 (evunzekibart ®) | anti-4-1BB (agonist) |
| RO-7122290 | anti-4-1BB (agonist) |
| INCAGN-01876 (ragifilimab ®) | anti-GITR (agonist) |
| BMS-986156 | anti-GITR (agonist) |
| TRX-518 | anti-GITR (agonist) |
| GWN-323 | anti-GITR (agonist) |
| BMS-986178 | anti-OX40 (agonist) |
| INCAGN-1949 | anti-OX40 (agonist) |
| GSK-3174998 | anti-OX40 (agonist) |
| BGB-A-445 | anti-OX40 (agonist) |
| BI-765063 | anti- SIRP-alpha (CD47) |
| ALX-148 (evorpacept ®) | SIRP-alpha (CD47) |
| IPH-52 | anti-CD39 |
| TTX-030 | anti-CD39 |
| BAY-1905254 (bapotulimab ®) | anti-ILDR2 |
| Onvatilimab | anti-VISTA |
| K01401-020 | anti-VISTA |
| JS-004 | anti-BTLA |
| FPA-150 | anti-VTCN1 |
| Relatlimab | Anti-LAG3 |
| Relatlimab + Nivolumab (Opdualag ™) | Anti-LAG3 + anti-PD1 |

In some embodiments, the checkpoint inhibitor comprises a PD-1 inhibitor. In some embodiments, the checkpoint inhibitor comprises a PD-L1 inhibitor. In some embodiments, the checkpoint inhibitor comprises a CTLA-4 inhibitor. In some embodiments, the checkpoint inhibitor comprises a TIGIT inhibitor. In some embodiments, the checkpoint inhibitor comprises a TIM-3 inhibitor. In some embodiments, the checkpoint inhibitor comprises a B37-H3 inhibitor. In some embodiments, the checkpoint inhibitor comprises a CD73 inhibitor. In some embodiments, the checkpoint inhibitor comprises a LAG3 inhibitor. In some embodiments, the checkpoint inhibitor comprises a CD27 inhibitor. In some embodiments, the checkpoint inhibitor comprises a CD70 inhibitor. In some embodiments, the checkpoint inhibitor comprises a 4-1BB agonist binder. In some embodiments, the checkpoint inhibitor comprises a GITR agonist binder. In some embodiments, the checkpoint inhibitor comprises a OX40 agonist binder. In some embodiments, the checkpoint inhibitor comprises a SIRP-alpha (CD47) inhibitor. In some embodiments, the checkpoint inhibitor comprises a CD39 inhibitor. In some embodiments, the checkpoint inhibitor comprises a ILDR2 inhibitor. In some embodiments, the checkpoint inhibitor comprises a VISTA inhibitor. In some embodiments, the checkpoint inhibitor comprises a BTLA inhibitor. In some embodiments, the checkpoint inhibitor comprises a VTCN-1 inhibitor.

In some embodiments, the checkpoint inhibitor comprises a combination of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1 BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1 BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor.

In some embodiments, a pharmaceutical composition for use in a combination therapy, as described herein, comprises an effective amount of a checkpoint inhibitor, as described herein, and a pharmaceutically acceptable carrier.

In some embodiments, a composition disclosed herein comprises a checkpoint inhibitor and a pharmaceutically acceptable carrier. In some embodiments, a composition disclosed herein comprises a combination of checkpoint inhibitors, and a pharmaceutically acceptable carrier. In some embodiments, a composition comprises a checkpoint inhibitor comprising a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1 BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1 BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, or a VTCN-1 inhibitor, and a pharmaceutically acceptable carrier. In some embodiments, a composition comprises at least two checkpoint inhibitors selected from a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1 BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1 BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor, and a pharmaceutically acceptable carrier.

In certain embodiments, when more than one checkpoint inhibitor is used in a therapeutic method described herein, each checkpoint inhibitor is comprised within a separate composition. In certain embodiments, when more than one checkpoint inhibitor is used in a therapeutic method described herein, checkpoint inhibitor may be comprised within the same composition.

In some embodiments, a combination therapy comprises use of an anti-IL-2 antibody or composition thereof as described herein, and a checkpoint inhibitor or a composition thereof. In some embodiments, a combination therapy comprises use of an anti-IL-2 antibody or composition thereof, IL-2 or composition thereof as described herein, and a checkpoint inhibitor or a composition thereof.

As used herein, in some embodiments the terms “combination” and “combination therapy” may be used interchangeably having all the same meanings and qualities.

In some embodiments, a combination of the present disclosure comprises an anti-IL-2 antibody or composition thereof and low dose of IL-2 or composition thereof as described herein, and a checkpoint inhibitor or a composition thereof. In some embodiments, a combination of the present disclosure comprises an anti-IL-2 antibody or composition thereof and low dose loading dose (low loading dose) of IL-2 or composition thereof as described herein, and a checkpoint inhibitor or a composition thereof. In some embodiments, a combination of the present disclosure comprises an anti-IL-2 antibody or composition thereof and a low dose booster dose (low booster dose) of IL-2 or composition thereof as described herein, and a checkpoint inhibitor or a composition thereof.

In some embodiments, a combination as described herein comprises an anti-IL-2 antibody formulated administration at a dose of 9 mg/kg. In other embodiments, the anti-IL-2 antibody is formulated for administration at a dose of between about 0.5 mg/kg of a subject's body weight-12 mg/kg of said subject's body weight. In other embodiments, the anti-IL-2 antibody is formulated for administration at a dose of between about 4.5 mg/kg of a subject's body weight-12 mg/kg of said subject's body weight. In other embodiments, the anti-IL-2 antibody is formulated for administration at a dose of between about 9 mg/kg of a subject's body weight 12 mg/kg of said subject's body weight. In some embodiments, a combination as described herein comprises an anti-IL-2 antibody formulated for intravenous (IV) administration.

In some embodiments, a combination as described herein comprises an anti-IL-2 antibody formulated for IV infusion and administration at a dose of 9 mg/kg. In other embodiments, the anti-IL-2 antibody is formulated for IV infusion and administration at a dose of between about 0.5 mg/kg of a subject's body weight-12 mg/kg of said subject's body weight. In other embodiments, the anti-IL-2 antibody is formulated for IV infusion and administration at a dose of between about 4.5 mg/kg of a subject's body weight-12 mg/kg of said subject's body weight. In other embodiments, the anti-IL-2 antibody is formulated for IV infusion and administration at a dose of between about 9 mg/kg of a subject's body weight 12 mg/kg of said subject's body weight. In some embodiments, a combination as described herein comprises an anti-IL-2 antibody formulated for intravenous (IV) administration.

In some embodiments, a combination as described herein comprises a loading low dose of IL-2 or a composition thereof at a dose of 135,000 IU/kg. In some embodiments, a combination as described herein comprises a booster dose of IL-2 at a dose of 135,000 IU/kg. In other embodiments, the loading or booster dose is formulated for subcutaneous injection and administration at a dose of between about 45,000 IU/kg of a subject's body weight-270,000 IU/kg of said subject's body weight. In other embodiments, the loading or booster dose is formulated for subcutaneous injection and administration at a dose of between about 45,000 IU/kg of a subject's body weight-135,000 IU/kg of said subject's body weight. In other embodiments, the loading or booster dose is formulated for subcutaneous injection and administration at a dose of between 15,000 IU/kg of said subject's body weight-500,000 IU/kg of said subject's body weight. In some embodiments, a combination as described herein comprises a low dose of IL-2 formulated for subcutaneous injection and administration.

In some embodiments, a combination as described herein comprises the immune checkpoint inhibitor formulated for administration at a dose of 480 mg. In some embodiments, a combination as described herein comprises nivolumab formulated for administration at a dose of 480 mg. In some embodiments, a combination as described herein comprises an immune checkpoint inhibitor formulated for administration. In some embodiments, a combination as described herein comprises nivolumab formulated for administration. In some embodiments, the immune checkpoint inhibitor comprises nivolumab. In certain embodiments, a checkpoint inhibitor is formulated for IV administration.

In some embodiments, a combination as described herein comprises the immune checkpoint inhibitor formulated for IV infusion and administration at a dose of 480 mg. In some embodiments, a combination as described herein comprises nivolumab formulated for IV infusion and administration at a dose of 480 mg. In some embodiments, a combination as described herein comprises an immune checkpoint inhibitor formulated for IV infusion and administration. In some embodiments, a combination as described herein comprises nivolumab formulated IV infusion and for administration. In some embodiments, the immune checkpoint inhibitor comprises nivolumab.

In some embodiments, a combination as described herein comprises nivolumab formulated for administration at a dose of 240 mg. In some embodiments, a combination as described herein comprises nivolumab formulated for administration at a dose of 480 mg. In some embodiments, a combination as described herein comprises nivolumab formulated for administration at a dose of 120 mg. In some embodiments, a combination as described herein comprises nivolumab formulated for administration at a dose of 360 mg. In some embodiments, a combination as described herein comprises nivolumab formulated for administration at a dose of 600 mg. In some embodiments, a combination as described herein comprises nivolumab formulated for administration at a dose of 120-600 mg. In some embodiments, a combination as described herein comprises nivolumab formulated for administration at a dose of 240-480 mg.

In some embodiments, a combination as described herein comprises nivolumab formulated for IV infusion and administration at a dose of 240 mg. In some embodiments, a combination as described herein comprises nivolumab formulated for IV infusion and administration at a dose of 480 mg. In some embodiments, a combination as described herein comprises nivolumab formulated for IV infusion and administration at a dose of 120 mg. In some embodiments, a combination as described herein comprises nivolumab formulated for IV infusion and administration at a dose of 360 mg. In some embodiments, a combination as described herein comprises nivolumab formulated for IV infusion and administration at a dose of 600 mg. In some embodiments, a combination as described herein comprises nivolumab formulated for IV infusion and administration at a dose of 120-600 mg. In some embodiments, a combination as described herein comprises nivolumab formulated for IV infusion and administration at a dose of 240-480 mg.

In certain embodiments, a combination therapy disclosed herein comprises an anti-IL-2 antibody or a pharmaceutical composition thereof, a loading dose of IL-2 or a pharmaceutical composition thereof, and nivolumab or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5; wherein said anti-IL-2 antibody is formulated for administration at a dose of 9 mg/kg of a subject's body weight, the loading dose of IL-2 is formulated for subcutaneous administration at a dose of 135,000 IU/kg of a subject's body weight, and said nivolumab is formulated for administration at a dose of 480 mg; and wherein said pharmaceutical composition(s) further comprises a pharmaceutically acceptable carrier.

In some embodiments, a combination therapy may include an immune checkpoint inhibitor targeting PD-1 or PD-L1. In some embodiments the immune checkpoint inhibitor targeting PD-1 comprises any of nivolumab, pembrolizumab, cemiplimab, camrelizumab, zimberelimab, tislelizumab, sintilimab, teriprizumab, prolgolimab, penpulimab, dostarlimab, genolimzumab, or retifanlimab. In some embodiments the immune checkpoint inhibitor targeting PD-L1 comprises any of avelumab, atezolizumab, durvalumab, sugemalimab, envafolimab, or cosibelimab.

In some embodiments, a combination as described herein comprises pembrolizumab formulated for IV infusion and administration at a dose of 300 mg. In some embodiments, a combination as described herein comprises pembrolizumab formulated for IV infusion and administration at a dose of 600 mg. In some embodiments, a combination as described herein comprises pembrolizumab formulated for IV infusion and administration at a dose of 2 mg/kg of the subject's body weight. In some embodiments, a combination as described herein comprises pembrolizumab formulated for IV infusion and administration. In some embodiments, the immune checkpoint inhibitor comprises pembrolizumab.

In some embodiments, a combination as described herein comprises pembrolizumab formulated for IV infusion and administration at a dose of 150 mg. In some embodiments, a combination as described herein comprises pembrolizumab formulated for IV infusion and administration at a dose of 75 mg. In some embodiments, a combination as described herein comprises pembrolizumab formulated for IV infusion and administration at a dose of 450 mg. In some embodiments, a combination as described herein comprises pembrolizumab formulated for IV infusion and administration at a dose of 750 mg. In some embodiments, a combination as described herein comprises pembrolizumab formulated for IV infusion and administration at a dose of 150-750 mg. In some embodiments, a combination as described herein comprises pembrolizumab formulated for IV infusion and administration at a dose of 300-600 mg.

In some embodiments, a combination as described herein comprises cemiplimab formulated for IV infusion and administration at a dose of 350 mg. In some embodiments, a combination as described herein comprises cemiplimab formulated for IV administration. In some embodiments, the immune checkpoint inhibitor comprises cemiplimab.

In some embodiments, a combination as described herein comprises cemiplimab formulated for IV infusion and administration at a dose of 150 mg. In some embodiments, a combination as described herein comprises cemiplimab formulated for IV infusion and administration at a dose of 225 mg. In some embodiments, a combination as described herein comprises cemiplimab formulated for IV infusion and administration at a dose of 475 mg. In some embodiments, a combination as described herein comprises cemiplimab formulated for IV infusion and administration at a dose of 600 mg. In some embodiments, a combination as described herein comprises cemiplimab formulated for IV infusion and administration at a dose of 150-600 mg. In some embodiments, a combination as described herein comprises cemiplimab formulated for IV infusion and administration at a dose of 350-400 mg.

In some embodiments, a combination as described herein comprises camrelizumab formulated for IV infusion and administration at a dose of 200 mg. In some embodiments, a combination as described herein comprises camrelizumab formulated for IV administration. In some embodiments, the immune checkpoint inhibitor comprises camrelizumab.

In some embodiments, a combination as described herein comprises camrelizumab formulated for IV infusion and administration at a dose of 100 mg. In some embodiments, a combination as described herein comprises camrelizumab formulated for IV infusion and administration at a dose of 150 mg. In some embodiments, a combination as described herein comprises camrelizumab formulated for IV infusion and administration at a dose of 300 mg. In some embodiments, a combination as described herein comprises camrelizumab formulated for IV infusion and administration at a dose of 400 mg. In some embodiments, a combination as described herein comprises camrelizumab formulated for IV infusion and administration at a dose of 100-400 mg. In some embodiments, a combination as described herein comprises camrelizumab formulated for IV infusion and administration at a dose of 200-300 mg.

In some embodiments, a combination as described herein comprises zimberelimab formulated for IV infusion and administration at a dose of 240 mg. In some embodiments, a combination as described herein comprises zimberelimab formulated for IV administration. In some embodiments, the immune checkpoint inhibitor comprises zimberelimab.

In some embodiments, a combination as described herein comprises zimberelimab formulated for IV infusion and administration at a dose of 100 mg. In some embodiments, a combination as described herein comprises zimberelimab formulated for IV infusion and administration at a dose of 175 mg. In some embodiments, a combination as described herein comprises zimberelimab formulated for IV infusion and administration at a dose of 350 mg. In some embodiments, a combination as described herein comprises zimberelimab formulated for IV infusion and administration at a dose of 500 mg. In some embodiments, a combination as described herein comprises zimberelimab formulated for IV infusion and administration at a dose of 100-500 mg. In some embodiments, a combination as described herein comprises zimberelimab formulated for IV infusion and administration at a dose of 240-300 mg.

In some embodiments, a combination as described herein comprises tislelizumab formulated for IV infusion and administration at a dose of 150 mg. In some embodiments, a combination as described herein comprises tislelizumab formulated for IV infusion and administration at a dose of 200 mg. In some embodiments, a combination as described herein comprises tislelizumab formulated for IV infusion and administration at a dose of 300 mg. In some embodiments, a combination as described herein comprises tislelizumab formulated for IV administration. In some embodiments, the immune checkpoint inhibitor comprises tislelizumab.

In some embodiments, a combination as described herein comprises tislelizumab formulated for IV infusion and administration at a dose of 100 mg. In some embodiments, a combination as described herein comprises tislelizumab formulated for IV infusion and administration at a dose of 400 mg. In some embodiments, a combination as described herein comprises tislelizumab formulated for IV infusion and administration at a dose of 100-400 mg. In some embodiments, a combination as described herein comprises tislelizumab formulated for IV infusion and administration at a dose of 150-300 mg.

In some embodiments, a combination as described herein comprises sintilimab formulated for IV infusion and administration at a dose of 200 mg. In some embodiments, a combination as described herein comprises sintilimab formulated for IV administration. In some embodiments, the immune checkpoint inhibitor comprises sintilimab.

In some embodiments, a combination as described herein comprises sintilimab formulated for IV infusion and administration at a dose of 100 mg. In some embodiments, a combination as described herein comprises sintilimab formulated for IV infusion and administration at a dose of 150 mg. In some embodiments, a combination as described herein comprises sintilimab formulated for IV infusion and administration at a dose of 300 mg. In some embodiments, a combination as described herein comprises sintilimab formulated for IV infusion and administration at a dose of 400 mg. In some embodiments, a combination as described herein comprises sintilimab formulated for IV infusion and administration at a dose of 100-400 mg. In some embodiments, a combination as described herein comprises sintilimab formulated for IV infusion and administration at a dose of 200-300 mg.

In some embodiments, a combination as described herein comprises teriprizumab formulated for IV infusion and administration at a dose of 200 mg. In some embodiments, a combination as described herein comprises teriprizumab formulated for IV administration. In some embodiments, the immune checkpoint inhibitor comprises teriprizumab.

In some embodiments, a combination as described herein comprises teriprizumab formulated for IV infusion and administration at a dose of 100 mg. In some embodiments, a combination as described herein comprises teriprizumab formulated for IV infusion and administration at a dose of 150 mg. In some embodiments, a combination as described herein comprises teriprizumab formulated for IV infusion and administration at a dose of 300 mg. In some embodiments, a combination as described herein comprises teriprizumab formulated for IV infusion and administration at a dose of 400 mg. In some embodiments, a combination as described herein comprises teriprizumab formulated for IV infusion and administration at a dose of 100-400 mg. In some embodiments, a combination as described herein comprises teriprizumab formulated for IV infusion and administration at a dose of 200-300 mg.

In some embodiments, a combination as described herein comprises prolgolimab formulated for IV infusion and administration at a dose of 250 mg. In some embodiments, a combination as described herein comprises prolgolimab formulated for IV administration. In some embodiments, the immune checkpoint inhibitor comprises prolgolimab.

In some embodiments, a combination as described herein comprises prolgolimab formulated for IV infusion and administration at a dose of 100 mg. In some embodiments, a combination as described herein comprises prolgolimab formulated for IV infusion and administration at a dose of 150 mg. In some embodiments, a combination as described herein comprises prolgolimab formulated for IV infusion and administration at a dose of 350 mg. In some embodiments, a combination as described herein comprises prolgolimab formulated for IV infusion and administration at a dose of 500 mg. In some embodiments, a combination as described herein comprises prolgolimab formulated for IV infusion and administration at a dose of 100-500 mg. In some embodiments, a combination as described herein comprises prolgolimab formulated for IV infusion and administration at a dose of 250-300 mg.

In some embodiments, a combination as described herein comprises penpulimab formulated for IV infusion and administration at a dose of 200 mg. In some embodiments, a combination as described herein comprises penpulimab formulated for IV administration. In some embodiments, the immune checkpoint inhibitor comprises penpulimab.

In some embodiments, a combination as described herein comprises penpulimab formulated for IV infusion and administration at a dose of 100 mg. In some embodiments, a combination as described herein comprises penpulimab formulated for IV infusion and administration at a dose of 150 mg. In some embodiments, a combination as described herein comprises penpulimab formulated for IV infusion and administration at a dose of 300 mg. In some embodiments, a combination as described herein comprises penpulimab formulated for IV infusion and administration at a dose of 400 mg. In some embodiments, a combination as described herein comprises penpulimab formulated for IV infusion and administration at a dose of 100-400 mg. In some embodiments, a combination as described herein comprises penpulimab formulated for IV infusion and administration at a dose of 200-300 mg.

In some embodiments, a combination as described herein comprises dostarlimab formulated for IV infusion and administration at a dose of 500 mg. In some embodiments, a combination as described herein comprises dostarlimab formulated for IV infusion and administration at a dose of 1000 mg. In some embodiments, a combination as described herein comprises dostarlimab formulated for IV administration. In some embodiments, the immune checkpoint inhibitor comprises dostarlimab.

In some embodiments, a combination as described herein comprises dostarlimab formulated for IV infusion and administration at a dose of 125 mg. In some embodiments, a combination as described herein comprises dostarlimab formulated for IV infusion and administration at a dose of 250 mg. In some embodiments, a combination as described herein comprises dostarlimab formulated for IV infusion and administration at a dose of 750 mg. In some embodiments, a combination as described herein comprises dostarlimab formulated for IV infusion and administration at a dose of 1500 mg. In some embodiments, a combination as described herein comprises dostarlimab formulated for IV infusion and administration at a dose of 125-1500 mg. In some embodiments, a combination as described herein comprises dostarlimab formulated for IV infusion and administration at a dose of 500-10000 mg.

In some embodiments, a combination as described herein comprises genolimzumab formulated for IV infusion and administration at a dose of 150 mg. In some embodiments, a combination as described herein comprises genolimzumab formulated for IV infusion and administration at a dose of 300 mg. In some embodiments, a combination as described herein comprises genolimzumab formulated for IV administration. In some embodiments, the immune checkpoint inhibitor comprises genolimzumab.

In some embodiments, a combination as described herein comprises genolimzumab formulated for IV infusion and administration at a dose of 50 mg. In some embodiments, a combination as described herein comprises genolimzumab formulated for IV infusion and administration at a dose of 100 mg. In some embodiments, a combination as described herein comprises genolimzumab formulated for IV infusion and administration at a dose of 400 mg. In some embodiments, a combination as described herein comprises genolimzumab formulated for IV infusion and administration at a dose of 500 mg. In some embodiments, a combination as described herein comprises genolimzumab formulated for IV infusion and administration at a dose of 150-500 mg. In some embodiments, a combination as described herein comprises genolimzumab formulated for IV infusion and administration at a dose of 200-300 mg.

In some embodiments, a combination as described herein comprises retifanlimab formulated for IV infusion and administration at a dose of 500 mg. In some embodiments, a combination as described herein comprises retifanlimab formulated for IV administration. In some embodiments, the immune checkpoint inhibitor comprises retifanlimab.

In some embodiments, a combination as described herein comprises retifanlimab formulated for IV infusion and administration at a dose of 250 mg. In some embodiments, a combination as described herein comprises retifanlimab formulated for IV infusion and administration at a dose of 100 mg. In some embodiments, a combination as described herein comprises retifanlimab formulated for IV infusion and administration at a dose of 750 mg. In some embodiments, a combination as described herein comprises retifanlimab formulated for IV infusion and administration at a dose of 1000 mg. In some embodiments, a combination as described herein comprises retifanlimab formulated for IV infusion and administration at a dose of 100-1000 mg. In some embodiments, a combination as described herein comprises retifanlimab formulated for IV infusion and administration at a dose of 250-500 mg.

In some embodiments, a combination as described herein comprises avelumab formulated for IV infusion and administration at a dose of 800 mg. In some embodiments, a combination as described herein comprises avelumab formulated for IV administration. In some embodiments, the immune checkpoint inhibitor comprises avelumab.

In some embodiments, a combination as described herein comprises avelumab formulated for IV infusion and administration at a dose of 400 mg. In some embodiments, a combination as described herein comprises avelumab formulated for IV infusion and administration at a dose of 200 mg. In some embodiments, a combination as described herein comprises avelumab formulated for IV infusion and administration at a dose of 1200 mg. In some embodiments, a combination as described herein comprises avelumab formulated for IV infusion and administration at a dose of 1600 mg. In some embodiments, a combination as described herein comprises avelumab formulated for IV infusion and administration at a dose of 200-1600 mg. In some embodiments, a combination as described herein comprises avelumab formulated for IV infusion and administration at a dose of 400-800 mg.

In some embodiments, a combination as described herein comprises atezolizumab formulated for IV infusion and administration at a dose of 840 mg. In some embodiments, a combination as described herein comprises atezolizumab formulated for IV infusion and administration at a dose of 1200 mg. In some embodiments, a combination as described herein comprises atezolizumab formulated for IV infusion and administration at a dose of 1580 mg. In some embodiments, a combination as described herein comprises atezolizumab formulated for IV administration. In some embodiments, the immune checkpoint inhibitor comprises atezolizumab.

In some embodiments, a combination as described herein comprises atezolizumab formulated for IV infusion and administration at a dose of 400 mg. In some embodiments, a combination as described herein comprises atezolizumab formulated for IV infusion and administration at a dose of 1000 mg. In some embodiments, a combination as described herein comprises atezolizumab formulated for IV infusion and administration at a dose of 1400 mg. In some embodiments, a combination as described herein comprises atezolizumab formulated for IV infusion and administration at a dose of 1800 mg. In some embodiments, a combination as described herein comprises atezolizumab formulated for IV infusion and administration at a dose of 400-1400 mg. In some embodiments, a combination as described herein comprises atezolizumab formulated for IV infusion and administration at a dose of 840-1680 mg.

In some embodiments, a combination as described herein comprises durvalumab formulated for IV infusion and administration at a dose of 1500 mg. In some embodiments, a combination as described herein comprises durvalumab formulated for IV infusion and administration at a dose of 1,120 mg. In some embodiments, a combination as described herein comprises durvalumab formulated for IV infusion and administration at a dose of 20 mg/kg weight of the subject. In some embodiments, a combination as described herein comprises durvalumab formulated for IV infusion and administration at a dose of 10 mg/kg weight of the subject. In some embodiments, a combination as described herein comprises durvalumab formulated for IV administration. In some embodiments, the immune checkpoint inhibitor comprises durvalumab.

In some embodiments, a combination as described herein comprises durvalumab formulated for IV infusion and administration at a dose of 1000 mg. In some embodiments, a combination as described herein comprises durvalumab formulated for IV infusion and administration at a dose of 1,300 mg. In some embodiments, a combination as described herein comprises durvalumab formulated for IV infusion and administration at a dose of 1750 mg. In some embodiments, a combination as described herein comprises durvalumab formulated for IV infusion and administration at a dose of 2000 mg. In some embodiments, a combination as described herein comprises durvalumab formulated for IV infusion and administration at a dose of 1000-2000 mg. In some embodiments, a combination as described herein comprises durvalumab formulated for IV infusion and administration at a dose of 10 mg/kg-20 mg/kg weight of the subject. In some embodiments, a combination as described herein comprises durvalumab formulated for IV infusion and administration at a dose of 1,120-1,500 mg.

In some embodiments, a combination as described herein comprises sugemalimab formulated for IV infusion and administration at a dose of 1200 mg. In some embodiments, a combination as described herein comprises sugemalimab formulated for IV administration. In some embodiments, the immune checkpoint inhibitor comprises sugemalimab.

In some embodiments, a combination as described herein comprises sugemalimab formulated for IV infusion and administration at a dose of 600 mg. In some embodiments, a combination as described herein comprises sugemalimab formulated for IV infusion and administration at a dose of 300 mg. In some embodiments, a combination as described herein comprises sugemalimab formulated for IV infusion and administration at a dose of 1500 mg. In some embodiments, a combination as described herein comprises sugemalimab formulated for IV infusion and administration at a dose of 1750 mg. In some embodiments, a combination as described herein comprises sugemalimab formulated for IV infusion and administration at a dose of 300 mg-1750 mg. In some embodiments, a combination as described herein comprises sugemalimab formulated for IV infusion and administration at a dose of 1,200-1,500 mg.

In some embodiments, a combination as described herein comprises envafolimab formulated for IV infusion and administration at a dose of 400 mg. In some embodiments, a combination as described herein comprises envafolimab formulated for IV administration. In some embodiments, the immune checkpoint inhibitor comprises envafolimab.

In some embodiments, a combination as described herein comprises envafolimab formulated for IV infusion and administration at a dose of 200 mg. In some embodiments, a combination as described herein comprises envafolimab formulated for IV infusion and administration at a dose of 100 mg. In some embodiments, a combination as described herein comprises envafolimab formulated for IV infusion and administration at a dose of 600 mg. In some embodiments, a combination as described herein comprises envafolimab formulated for IV infusion and administration at a dose of 800 mg. In some embodiments, a combination as described herein comprises envafolimab formulated for IV infusion and administration at a dose of 100-800 mg. In some embodiments, a combination as described herein comprises envafolimab formulated for IV infusion and administration at a dose of 400-600 mg.

In some embodiments, a combination as described herein comprises cosibelimab formulated for IV infusion and administration at a dose of 1200 mg. In some embodiments, a combination as described herein comprises cosibelimab formulated for IV administration. In some embodiments, the immune checkpoint inhibitor comprises cosibelimab.

In some embodiments, a combination as described herein comprises cosibelimab formulated for IV infusion and administration at a dose of 600 mg. In some embodiments, a combination as described herein comprises cosibelimab formulated for IV infusion and administration at a dose of 300 mg. In some embodiments, a combination as described herein comprises cosibelimab formulated for IV infusion and administration at a dose of 1500 mg. In some embodiments, a combination as described herein comprises cosibelimab formulated for IV infusion and administration at a dose of 1,800 mg. In some embodiments, a combination as described herein comprises cosibelimab formulated for IV infusion and administration at a dose of 300-1,800 mg. In some embodiments, a combination as described herein comprises cosibelimab formulated for IV infusion and administration at a dose of 1,200-1,500 mg.

In some embodiments, a combination as described herein comprises an anti-IL-2 antibody comprising a heavy chain variable region (VH) comprising heavy chain complementarity determining regions (HCDRs) HCDR1, HCDR2 and HCDR3 wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3. In some embodiments, a combination as described herein comprises an anti-IL-2 antibody comprising a VH amino acid sequence as set forth in SEQ ID NO: 6. In some embodiments, a combination as described herein comprises an anti-IL-2 antibody comprising a full length heavy chain having an amino acid sequence as set forth in SEQ ID NO:8.

In some embodiments, a combination as described herein comprises an anti-IL-2 antibody comprising a light chain variable region (VL) comprising light chain complementarity determining regions (LCDRs) LCDR1, LCDR2 and LCDR3, said LCDR1 comprises the amino acid sequence of SEQ ID NO:4, said LCDR2 comprises the amino acid sequence of DAS, and said LCDR3 comprises the amino acid sequence of SEQ ID NO:5. In some embodiments, a combination as described herein comprises an anti-IL-2 antibody comprising a VL amino acid sequence as set forth in SEQ ID NO: 7. In some embodiments, a combination as described herein comprises an anti-IL-2 antibody comprising a full length light chain having an amino acid sequence as set forth in SEQ ID NO:9.

In some embodiments, a combination as described herein comprises an anti-IL-2 antibody comprising a heavy chain variable region (VH) comprising heavy chain complementarity determining regions (HCDRs) HCDR1, HCDR2 and HCDR3 wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, and a light chain variable region (VL) comprising light chain complementarity determining regions (LCDRs) LCDR1, LCDR2 and LCDR3, said LCDR1 comprises the amino acid sequence of SEQ ID NO:4, said LCDR2 comprises the amino acid sequence of DAS, and said LCDR3 comprises the amino acid sequence of SEQ ID NO:5. In some embodiments, a combination as described herein comprises an anti-IL-2 antibody comprising a VH amino acid sequence as set forth in SEQ ID NO: 6 and a VL amino acid sequence as set forth in SEQ ID NO: 7. In some embodiments, a combination as described herein comprises an anti-IL-2 antibody comprising a full length heavy chain having an amino acid sequence as set forth in SEQ ID NO:8 and a full length light chain having an amino acid sequence as set forth in SEQ ID NO:9.

In some embodiments, a combination as described herein comprises an anti-IL-2 antibody comprising an IgG, IgA, IgM, IgE, IgD, a Fv, a scFv, a Fab, a F(ab′)₂, a minibody, a diabody, or a triabody. In some embodiments, a combination as described herein comprises an anti-IL-2 antibody comprising a heavy chain comprising a mutation that reduces binding to an Fcγ receptor. In some embodiments, the mutation comprises a L234A mutation. In other embodiments, the mutation comprises a L235A mutation. In other embodiments, the mutation comprises both L234A and L235A mutations. In some embodiments, a combination as described herein comprises an anti-IL-2 antibody that is an human IgG1 antibody with a LALA Fc mutation.

In some embodiments, a combination therapy comprises use of an anti-IL-2 antibody or composition thereof as described herein, and at least two checkpoint inhibitors or a composition thereof. In some embodiments, a combination therapy comprises use of an anti-IL-2 antibody or composition thereof and IL-2 as described herein, and at least two checkpoint inhibitors or a composition thereof.

In some embodiments, a combination therapy comprises a second composition comprising one or more checkpoint inhibitors, as described herein.

In some embodiments, a combination therapy comprises use of anti-IL-2 antibody BDG17.069 or composition thereof; and IL-2 as described herein; and a checkpoint inhibitor or a composition thereof as described herein. In some embodiments, a combination therapy comprises use of BDG17.069 or composition thereof; and a low dose of IL-2 as described herein; and a checkpoint inhibitor or a composition thereof as described herein. In some embodiments, a combination therapy comprises use of BDG17.069 or composition thereof; and a low dose of IL-2 as described herein; and a checkpoint inhibitor or a composition thereof, wherein said checkpoint inhibitor is selected from a PD-1 inhibitor, a PD-L1, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1 BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor.

In some embodiments, a combination therapy comprises use of BDG17.069 or composition thereof; and a low dose of IL-2 as described herein; and a checkpoint inhibitor or a composition thereof, wherein said checkpoint comprises PD-L1. In some embodiments, a combination therapy comprises use of BDG17.069 or composition thereof; and a low dose of IL-2 (aldesleukin); and nivolumab or a composition thereof. In certain embodiments of a combination therapy comprising BDG17.069 or composition thereof; and a low dose of IL-2 (aldesleukin); and nivolumab or a composition thereof, the IL-2 is administered by subcutaneous injection. In some embodiments, the low dose of IL-2 is a loading dose. In other embodiments, the low dose of IL-2 is a booster dose.

Thus, in some embodiments, a combination therapy comprises the use of BDG17.069 or composition thereof; and a loading dose of IL-2 as described herein; and a checkpoint inhibitor or a composition thereof, wherein said checkpoint comprises PD-L1. In some embodiments, a combination therapy comprises use of BDG17.069 or composition thereof; and a loading dose of IL-2 (aldesleukin); and nivolumab or a composition thereof. In certain embodiments of a combination therapy comprising BDG17.069 or composition thereof; and a loading dose of IL-2 (aldesleukin); and nivolumab or a composition thereof, the IL-2 is administered by subcutaneous injection. In some embodiments, the loading dose of IL-2 is a loading dose. In other embodiments, the loading dose of IL-2 is a booster dose.

In some embodiments, a combination therapy comprises use of BDG17.069 or composition thereof; and a booster dose of IL-2 as described herein; and a checkpoint inhibitor or a composition thereof, wherein said checkpoint comprises PD-L1. In some embodiments, a combination therapy comprises use of BDG17.069 or composition thereof; and a booster dose of IL-2 (aldesleukin); and nivolumab or a composition thereof. In certain embodiments of a combination therapy comprising BDG17.069 or composition thereof; and a booster dose of IL-2 (aldesleukin); and nivolumab or a composition thereof, the IL-2 is administered by subcutaneous injection.

In some embodiments of a combination therapy, the IL-2 administered comprises a low dose of IL-2. In some embodiments of a combination therapy, the IL-2 administered is by subcutaneous administration. In some embodiments of a combination therapy, the IL-2 administered comprises a low dose of IL-2 administered by subcutaneous administration.

In some embodiments of a combination therapy, an anti-IL-2 antibody and IL-2 are comprised in the same composition as a checkpoint inhibitor. In some embodiments, an anti-IL-2 antibody and IL-2 are comprised in different compositions from each other and from a checkpoint inhibitor. In some embodiments, an anti-IL-2 antibody, IL-2, and a checkpoint inhibitor are comprised in the same composition. In some embodiments, an anti-IL-2 antibody and IL-2 are comprised in a composition, and a checkpoint inhibitor is comprised in a different composition. In some embodiments, an anti-IL-2 antibody and a checkpoint inhibitor are comprised in a composition, and IL-2 is comprised in a different composition.

In some embodiments of a combination therapy, BDG17.069 and aldesleukin are comprised in the same composition as a PD-L1 checkpoint inhibitor. In some embodiments of a combination therapy, BDG17.069 and aldesleukin are comprised in the same composition as nivolumab. In some embodiments, BDG17.069 and aldesleukin are comprised in different compositions from each other and from nivolumab. In some embodiments, BDG17.069 and aldesleukin and nivolumab are comprised in the same composition. In some embodiments, BDG17.069 and aldesleukin are comprised in a composition, and nivolumab is comprised in a different composition. In some embodiments, BDG17.069 and nivolumab are comprised in a composition, and aldesleukin is comprised in a different composition. In some embodiments of a combination therapy, the order of administration of an anti-IL-2 antibody or a composition thereof and a checkpoint inhibitor or a composition thereof, may be in any order. In some embodiments of a combination therapy, the order of administration of BDG17.069 or a composition thereof or a composition thereof and nivolumab or a composition thereof, may be in any order. In some embodiments of a combination therapy, the order of administration of an anti-IL-2 antibody or a composition thereof, IL-2 or a composition thereof, and a checkpoint inhibitor or a composition thereof, may be in any order. In some embodiments of a combination therapy, the order of administration of BDG17.069 or a composition thereof, aldesleukin or a composition thereof, and nivolumab or a composition thereof, may be in any order. For example, but not limited to the anti-IL-2 antibody may be administered prior to, concurrent with, or following administration of the checkpoint inhibitor. Similarly, a combination of an anti-IL-2 antibody and IL-2 may be administered prior to, concurrent with, or following administration of the checkpoint inhibitor. For example, but not limited to the BDG17.069 may be administered prior to, concurrent with, or following administration of the nivolumab. Similarly, a combination of BDG17.069 and aldesleukin may be administered prior to, concurrent with, or following administration of the nivolumab. In some embodiments, the anti-IL-2 antibody may be administered prior to, concurrent with, or following administration of the at least two checkpoint inhibitors. Similarly, a combination of an anti-IL-2 antibody and IL-2 may be administered prior to, concurrent with, or following administration of the at least two checkpoint inhibitors.

In some embodiments, administration of a combination therapy with a checkpoint inhibitor comprises concurrent administration of an anti-IL-2 antibody or a composition thereof and the checkpoint inhibitor. In some embodiments, administration of a combination therapy with a checkpoint inhibitor comprises concurrent administration of BDG17.069 or a composition thereof and nivolumab. In some embodiments, administration of a combination therapy with a checkpoint inhibitor comprises concurrent administration of an anti-IL-2 antibody and IL-2, or composition(s) thereof and the checkpoint inhibitor. In some embodiments, administration of a combination therapy with a checkpoint inhibitor comprises concurrent administration of BDG17.069 and aldesleukin, or composition(s) thereof and nivolumab. In some embodiments, administration of a combination therapy with a checkpoint inhibitor comprises prior administration of an anti-IL-2 antibody or a composition thereof before the checkpoint inhibitor. In some embodiments, administration of a combination therapy with a checkpoint inhibitor comprises prior administration of BDG17.069 or a composition thereof before the nivolumab. In some embodiments, administration of a combination therapy with a checkpoint inhibitor comprises prior administration of an anti-IL-2 antibody and IL-2, or composition(s) thereof before the checkpoint inhibitor. In some embodiments, administration of a combination therapy with a checkpoint inhibitor comprises prior administration of BDG17.069 and aldesleukin, or composition(s) thereof before the nivolumab. In some embodiments, administration of a combination therapy with a checkpoint inhibitor comprises later administration of an anti-IL-2 antibody or a composition thereof following administration of the checkpoint inhibitor. In some embodiments, administration of a combination therapy with a checkpoint inhibitor comprises later administration of BDG17.069 or a composition thereof following administration of the nivolumab. In some embodiments, administration of a combination therapy with a checkpoint inhibitor comprises later administration of an anti-IL-2 antibody and IL-2, or composition(s) thereof following the administration of the checkpoint inhibitor. In some embodiments, administration of a combination therapy with a checkpoint inhibitor comprises later administration of BDG17.069 and aldesleukin, or composition(s) thereof following the administration of the nivolumab.

In some embodiments, a combination therapy comprises use of a checkpoint inhibitor and an anti-IL-2 antibody comprising a heavy chain variable region having the sequence of SEQ ID NO:6. In some embodiments, a combination therapy comprises use of a checkpoint inhibitor and an anti-IL-2 antibody comprising a light chain variable region having the sequence of SEQ ID NO:7. In some embodiments, a combination therapy comprises use of a checkpoint inhibitor and an anti-IL-2 antibody comprising a heavy chain variable region and a light chain variable region having the sequences of SEQ ID NOs: 6 and 7. In some embodiments, a combination therapy comprises use of a checkpoint inhibitor and an anti-IL-2 antibody comprising a heavy chain variable domain comprising CDR1, CDR2 and CDR3 regions comprising amino acid sequences of SEQ ID NOs:1-3 respectively. In some embodiments, a combination therapy comprises use of a checkpoint inhibitor and an anti-IL-2 antibody comprising a light chain variable domain comprising CDR1, CDR2 and CDR3 regions comprising amino acid sequences of SEQ ID NO: 4, DAS, and SEQ ID NO: 5, respectively. In some embodiments, a combination therapy comprises use of a checkpoint inhibitor and an anti-IL-2 antibody comprising a heavy chain variable domain comprising CDR1, CDR2 and CDR3 regions comprising amino acid sequences of SEQ ID NOs:1-3, respectively, and a light chain variable domain comprising CDR1, CDR2, and CDR3 regions comprising amino acid sequences of SEQ ID NO: 4, DAS, and SEQ ID NO: 5, respectively.

In some embodiments, a combination therapy comprises use of a checkpoint inhibitor and an anti-IL-2 antibody comprising anti-IL-2 clone BDG 17.069.

In some embodiments, a combination therapy comprises use of a checkpoint inhibitor; and an anti-IL-2 antibody comprising clone BDG 17.069; and IL-2.

In certain embodiments, use of a combination therapy is for treating an unresectable locally advanced or metastatic cutaneous melanoma. In certain embodiments, use of a combination therapy is for treating an unresectable locally advanced or metastatic cutaneous melanoma that progressed after prior checkpoint inhibitor therapy.

### Engineered Anti-IL-2 Antibodies

The present disclosure provides engineered anti-human IL-2 antibodies that bind human IL-2 with high affinity (e.g., 12.7 pM to 48 pM) to a pre-defined binding epitope. The antibodies bind to IL-2 in a manner that completely prevents CD25 binding, yet spares the binding of IL-2 to CD122, thereby modulating immune responses towards immune stimulation by directly activating and expanding effector cells without interacting with CD25-expressing cells (e.g., regulatory T-cells, short lived cytotoxic T-cells, pulmonary endothelial cells and vascular endothelial cells). Thus, the antibody/IL-2 complex would drive a robust immune response to clear a tumor by expanding and activating effector cells such as NK cells, central memory T cells and tumor-specific T-cells while inhibiting IL-2 activation induced cell death of the short lived CD25+cytotoxic T-cells that are important for tumor clearance. The antibody/IL-2 complex would also decrease immunosuppression caused by the regulatory arm of the immune system. Moreover, the antibody/IL-2 complex prevent undesired interactions of IL-2 with vascular and pulmonary CD25-expressing cells, thereby preventing severe syndromes of IL-2 induced vascular leakage and IL-2 induced pulmonary edema. In some embodiments, the activity of the engineered anti-IL-2 antibodies described herein is dependent on the pre-defined epitope to which they are designed to bind.

In some embodiments, the IL-2 antibodies disclosed herein, block IL-2 binding to CD25. In some embodiments, the IL-2 antibodies disclosed herein binds IL-2 and prevent newly secreted endogenous IL-2 from binding to Tregs, effectively blocking the negative feedback loop of IL-2 to Tregs. In some embodiments, the IL-2 antibodies disclosed herein prevent Treg expansion. In some embodiments, the IL-2 antibodies disclosed herein block IL-2 binding to vascular endothelium. In some embodiments, the IL-2 antibodies disclosed herein block IL-2 binding to pulmonary endothelium. In some embodiments, the IL-2 antibodies disclosed herein block IL-2 binding to vascular and pulmonary endothelium.

In some embodiments, an anti-human anti-IL-2 antibody described herein inhibits binding of IL-2 with an IL-2 receptor alpha (IL-2 Ra, i.e., CD25) subunit and therefore inhibits binding to the trimer IL-2 Rαβγ receptor. In certain embodiments, anti-IL-2 antibodies that inhibit binding of IL-2 with a trimer IL-2 receptor (IL-2 Rαβγ) do not inhibit binding of IL-2 with the dimer IL-2 receptor (IL-2 Rβγ).

Targeting IL-2 to different cell populations can be used to either modulate the immune response toward immunosuppression or towards immune activation. The anti-human IL-2 antibodies disclosed herein are designed to bind with high affinity to an IL-2 epitope that blocks IL-2 binding to CD25. As a result, IL-2 is prevented from binding to short-lived CD8+ cytotoxic T cells or regulatory T cells that express high level of CD25 but is redirected to preferentially bind to effector T cells to stimulate enhanced immune response. Moreover, since IL-2 binding to CD25-expressing endothelial cells is also blocked, IL-2 induced pulmonary edema and vascular leaking would also be prevented.

In some embodiments, the present disclosure provides an anti-IL-2 antibody designed to enhance T cell immune response and to prevent severe edema symptoms of acute pneumonia induced by IL-2. The anti-IL-2 antibody binds specifically to human IL-2 with high affinity at a pre-defined epitope that blocks IL-2 binding to the alpha chain of the IL-2 receptors (CD25) while sparing binding to the main signaling beta chain and gamma chain complex of the receptor (CD122/CD132). Consequently, in the presence of such antibody, IL-2 would be directed to immune cells responsible for tumor clearance and away from cells that slow the immune response or cause the edema. The formation of this IL-2/antibody immunocomplex will direct IL-2 to bind and activate exclusively naive and memory T lymphocytes, NK cells, and Natural Killer T lymphocytes while preventing activation of regulatory T cells and apoptosis of short-lived CD25+ cytotoxic T effector cells. Altogether, the end result is an effective immune response, for example, tumor clearance. In addition, this treatment will prevent the toxicity caused by IL-2 binding to endothelial CD25 expressing cells. In other embodiments, treatment with the anti-IL-2 antibodies disclosed herein would be effective in preventing the toxicity caused by IL-2 binding to endothelial CD25 expressing cells, e.g., pulmonary edema, or IL-2-induced vascular leakage.

The cytokine IL-2 is critical for the expansion of T cells. However, in addition to its pro-stimulatory role IL-2 also induces some adverse side effects like lung edema and vascular leak syndrome through its binding to endothelium expressing the CD25 receptor.

In some embodiments, the present disclosure provides engineered anti-IL-2 antibodies resulting from introducing amino acid variations to a parent anti-IL-2 antibody. In some embodiments, the one or more of the amino acid variations are introduced in a CDR region. In other embodiments, the one or more amino acid variations are introduced within a framework (FR) region. In yet another embodiment, the amino acid variations are introduced to both the CDR region and the framework (FR) region. One of ordinary skill in the art would readily employ various standard techniques known in the art to introduce amino acid variations into an anti-IL-2 antibody and then test the resulting modified antibodies for any changes of binding to IL-2. While standard techniques may be used, the resultant binding pattern of the newly created antibodies is not predictable and must be analyzed to determine functionality.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In other embodiments, the VH and VL domains may be combined in a suitable buffer containing an enzyme and/or a cofactor which can promote dimerization of the VH and VL domains. In other embodiments, the VH and VL domains may be combined in a suitable vehicle that allows them to react with each other in the presence of a suitable reagent and/or catalyst.

In certain embodiments, the VH and VL domains may be contained within longer polypeptide sequences, which may include for example but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g., CH1, CH2, CH3, CH4, Ck, Cl). In some embodiments, the longer polypeptides may comprise multiple copies of one or both of the VH and VL domains generated according to the method disclosed herein; for example, when the polypeptides generated herein are used to form a diabody or a triabody.

In some embodiments, the Fc region comprises at least one mutation that reduces Fc-gamma binding, i.e., binding to a Fcγ receptor (FcγRs). In some embodiments, reduced binding is abolished binding, which binding to the Fcγ receptor is not detectable. In some embodiments, reduced binding reduces the binding affinity to a Fcγ receptor. In some embodiments, reduced binding reduces the on rate for binding to a Fcγ receptor. In some embodiments, reduced binding reduces the off rate of binding to a Fcγ receptor. In some embodiments, a mutation that reduces the Fc-gamma binding comprises L234A, L235A mutations, also known as LALA mutations. In some embodiments, a mutation that reduces the Fc-gamma binding comprises a P329G mutation in addition to the L234A, L235A mutations. In some embodiments, an antibody described herein comprises a heavy chain comprising a mutation that reduces binding to Fcγ receptor.

In some embodiments, the present disclosure provides an engineered (or modified) anti-IL-2 antibody, wherein the antibody comprises a heavy chain variable region having the sequence of SEQ ID NO: 6. In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure provides an engineered (or modified) anti-IL-2 antibody, wherein the antibody comprises a light chain variable region having the sequence of SEQ ID NO: 7. In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure provides an engineered (or modified) anti-IL-2 antibody, wherein the antibody comprises a heavy chain variable region and a light chain variable region having the sequences of on SEQ ID NOs:6 and 7. In some embodiments, the engineered anti-IL-2 antibody comprises the sequences of SEQ ID NOs:6 and 7.

In some embodiments, an isolated anti-IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein the HCDR1 comprises the amino acid sequence of SEQ ID NO:1, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of DAS, the LCDR3 comprises the amino acid sequence of SEQ ID NO:5.

In some embodiments, the VH and VL have the amino acid sequences wherein the VH comprises the amino acid sequence of SEQ ID NO:6, the VL comprises the amino acid sequence of SEQ ID NO:7.

In some embodiments, an antibody comprises a heavy chain sequence and a light chain sequence, said heavy chain sequence set forth in SEQ ID NO: 8 and said light chain sequence set forth in SEQ ID NO: 9. In some embodiments, an antibody comprising a heavy chain sequence and a light chain sequence, said heavy chain sequence set forth in SEQ ID NO: 8 and said light chain sequence set forth in SEQ ID NO: 9.

In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure also provides isolated polynucleotide sequence encoding a heavy chain variable region of an anti-IL-2 antibody, wherein the heavy chain variable region comprises the amino acid sequence of SEQ ID NO: 6. In other embodiments, the present disclosure also provides a vector comprising the above-mentioned polynucleotide sequences. In view of the amino acid sequences disclosed herein, one of ordinary skill in the art would readily construct a vector or plasmid to encode for the amino acid sequences. In other embodiments, the present disclosure also provides a host cell comprising the vector provided herein. Depending on the uses and experimental conditions, one of skill in the art would readily employ a suitable host cell to carry and/or express the above-mentioned polynucleotide sequences.

In some embodiments, the present disclosure also provides isolated polynucleotide sequence encoding a light chain variable region of an anti-IL-2 antibody, wherein the light chain variable region comprises the amino acid sequence of SEQ ID NO: 7. In other embodiments, the present disclosure also provides a vector comprising the above-mentioned polynucleotide sequences. In view of the amino acid sequences disclosed herein, one of ordinary skill in the art would readily construct a vector or plasmid to encode for the amino acid sequences. In other embodiments, the present disclosure also provides a host cell comprising the vector provided herein. Depending on the uses and experimental conditions, one of skill in the art would readily employ a suitable host cell to carry and/or express the above-mentioned polynucleotide sequences.

In view of the sequences for the heavy chain variable regions and light chain variable regions disclosed herein, one of ordinary skill in the art would readily employ standard techniques known in the art to construct an anti-IL-2 scFv. In some embodiments, polynucleotide sequences encoding for such anti-IL-2 scFv could have the sequence of SEQ ID NO: 10.

In certain embodiments, an isolated polynucleotide sequence disclosed herein, encoding the heavy chain variable region of an anti-IL-2 antibody, comprises a VH amino acid sequence set forth in the amino acid sequence of SEQ ID NO: 6. In some embodiments, a vector comprises the polynucleotide sequence of SEQ ID NO: 6. In some embodiments, a host cell comprising the vector comprising the polynucleotide sequence of SEQ ID NO: 6.

In certain embodiments, an isolated polynucleotide sequence disclosed herein, encoding the light chain variable region of an anti-IL-2 antibody, comprises a VL amino acid sequence as set forth in the amino acid sequence of SEQ ID NO: 7. In some embodiments, a vector comprises the polynucleotide sequence comprising the amino acid sequence of SEQ ID NO: 7. In some embodiments, a host cell comprises a vector comprising the polynucleotide sequence encoding the amino acid sequence of SEQ ID NO: 7. In some embodiments, an isolated polynucleotide sequence encodes an anti-IL-2 scFv, wherein the polynucleotide sequence encodes SEQ ID NO: 10. In some embodiments, a vector comprises an isolated polynucleotide sequence encodes an anti-IL-2 scFv, wherein the polynucleotide sequence encodes SEQ ID NO: 10. In some embodiments, a host cell comprises a vector comprising an isolated polynucleotide sequence encoding an anti-IL-2 scFv, wherein the polynucleotide sequence encodes SEQ ID NO: 10.

In other embodiments, the present disclosure also provides an isolated anti-IL-2 antibody, wherein the antibody comprises a heavy chain variable region having complementarity determining region (CDR) 1, CDR2 and CDR3. In some embodiments, the CDR1, CDR2 and CDR3 comprise the amino acid sequences of SEQ ID NOs:1-3, respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In other embodiments, the present disclosure also provides an isolated anti-IL-2 antibody, wherein the antibody comprises a light chain variable region having complementarity determining region (CDR) 1, CDR2 and CDR3. In some embodiments, the CDR1, CDR2 and CDR3 comprise amino acid sequences of SEQ ID NO: 4, DAS, and SEQ ID NO: 5, respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In other embodiments, the present disclosure also provides an isolated anti-IL-2 antibody, wherein the antibody comprises a heavy chain variable region having complementarity determining region (CDR) 1, CDR2 and CDR3, and a light chain variable region having CDR1, CDR2 and CDR3. In some embodiments, the heavy chain CDR1, CDR2 and CDR3 comprise the amino acid sequences of SEQ ID NOs:1-3, respectively. In some embodiments, the light chain CDR1, CDR2 and CDR3 comprise the amino acid sequences of SEQ ID NO: 4, DAS, and SEQ ID NO: 5, respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In some embodiments, an anti-IL-2 antibody as described herein is referred to as “BDG17.069” or “AU-007”, which may be used interchangeably with the term “anti-IL-2 antibody”, having all the same qualities and meanings. In addition, the term “imneskibart” may be used interchangeably with the terms “BDG17.069” or “AU-007” having all the same qualities and meanings, wherein imneskibart is the human monoclonal antibody also known as BDG17.069 or AU-007 with drug-like properties that provides advantages over non-natural biologics.

### Pharmaceutical Compositions

In some embodiments, disclosed herein are compositions for therapeutic use. In some embodiments, a composition described herein comprises an anti-IL-2 antibody as disclosed herein and a pharmaceutically acceptable carrier.

As used herein, the terms “composition” and pharmaceutical composition” may in some embodiments, be used interchangeably having all the same qualities and meanings. In some embodiments, disclosed herein is a pharmaceutical composition for the treatment of a condition or disease as described herein.

In some embodiments, disclosed herein are pharmaceutical compositions for use in a combination therapy.

In other embodiments, disclosed herein are compositions for use in treating an unresectable locally advanced or metastatic cutaneous melanoma.

In some embodiments, compositions comprise an anti-IL-2 antibody and a pharmaceutically acceptable carrier. In some embodiments, compositions comprise an anti-IL-2 antibody and IL-2, and a pharmaceutically acceptable carrier. In some embodiments, compositions comprise an anti-IL-2 antibody, IL-2, and an immune checkpoint inhibitor, and a pharmaceutically acceptable carrier. In some embodiments, compositions comprise an anti-IL-2 antibody, IL-2, and nivolumab, and a pharmaceutically acceptable carrier.

In some embodiments, compositions comprise an anti-IL-2 antibody and a pharmaceutically acceptable carrier. In some embodiments, compositions comprise IL-2, and a pharmaceutically acceptable carrier. In some embodiments, compositions comprise an immune checkpoint inhibitor and a pharmaceutically acceptable carrier. In some embodiments, compositions comprise nivolumab and a pharmaceutically acceptable carrier.

In some embodiments, an anti-IL-2 antibody and IL-2 are comprised in the same composition. In some embodiments, an anti-IL-2 antibody and IL-2 are comprised in different compositions. In some embodiments, an anti-IL-2 antibody and an immune checkpoint inhibitor are comprised in the same composition. In some embodiments, an anti-IL-2 antibody and an immune checkpoint inhibitor are comprised in different compositions. In some embodiments, an anti-IL-2 antibody and nivolumab are comprised in the same composition. In some embodiments, an anti-IL-2 antibody and nivolumab are comprised in different compositions. In some embodiments, an anti-IL-2 antibody, IL-2, and an immune checkpoint inhibitor are comprised in the same composition. In some embodiments, an anti-IL-2 antibody, IL-2, and an immune checkpoint inhibitor are comprised in different compositions. In some embodiments, an anti-IL-2 antibody, IL-2, and nivolumab are comprised in the same composition. In some embodiments, an anti-IL-2 antibody, IL-2 and nivolumab are comprised in different compositions.

In some embodiments, administration of a combination of an anti-IL-2 antibody and IL-2, or composition(s) thereof are concurrent. In some embodiments, administration of a combination of an anti-IL-2 antibody and IL-2, or composition(s) thereof comprises administration of an anti-IL-2 antibody or a composition thereof, prior to the IL-2 or a composition thereof. In some embodiments, administration of a combination of an anti-IL-2 antibody and IL-2, or composition(s) thereof comprises administration of an anti-IL-2 antibody or a composition thereof, following administration of the IL-2 or a composition thereof.

In some embodiments, administration of a combination of an anti-IL-2 antibody, IL-2, and an immune checkpoint inhibitor or composition(s) thereof are concurrent. In some embodiments, administration of a combination of an anti-IL-2 antibody, IL-2, and an immune checkpoint inhibitor or composition(s) thereof comprises administration of an anti-IL-2 antibody or a composition thereof, prior to the IL-2 and or the immune checkpoint inhibitor, or compositions thereof. In some embodiments, administration of a combination of an anti-IL-2 antibody, IL-2, and an immune checkpoint inhibitor or composition(s) thereof comprises administration of an anti-IL-2 antibody or a composition thereof, following administration of the IL-2 and or an immune checkpoint inhibitor, or a composition thereof.

In some embodiments, administration of a combination of an anti-IL-2 antibody, IL-2, and nivolumab or composition(s) thereof are concurrent. In some embodiments, administration of a combination of an anti-IL-2 antibody, IL-2, and nivolumab or composition(s) thereof comprises administration of an anti-IL-2 antibody or a composition thereof, prior to the IL-2 and or nivolumab, or compositions thereof. In some embodiments, administration of a combination of an anti-IL-2 antibody, IL-2, and nivolumab or composition(s) thereof comprises administration of an anti-IL-2 antibody or a composition thereof, following administration of the IL-2 and or nivolumab, or a composition thereof.

In some embodiments, administering a loading dose of IL-2 or a composition thereof, comprises administering prior to, concurrent with, or following administration of the anti-IL-2 antibody, nivolumab, or both. In certain embodiments of administering an anti-IL-2 antibody, IL-2, and or an immune checkpoint inhibitor, for example nivolumab comprises administering a pharmaceutical composition comprising the anti-IL-2 antibody, the IL-2, and or the immune checkpoint inhibitor (e.g., nivolumab), wherein the composition further comprises a pharmaceutically acceptable carrier. In some embodiments, each of the anti-IL-2 antibody, the IL-2, and the immune checkpoint inhibitor (e.g., nivolumab) are comprised in separate compositions that further comprise a pharmaceutically acceptable carrier.

A skilled artisan would appreciate that a “pharmaceutical composition” may encompass a preparation of one or more of the active ingredients described herein with other chemical components such as physiologically suitable carriers and excipients. The purpose of a pharmaceutical composition is to facilitate administration of an active agent, for example but not limited to an antibody or a compound, to an organism.

In some embodiments, disclosed herein is a pharmaceutical composition for a therapy use treating a subject with a weakened immune system. In some embodiments, disclosed herein is a pharmaceutical composition for a therapy use treating a subject suffering from an unresectable locally advanced or metastatic cutaneous melanoma. In some embodiments, disclosed herein is a pharmaceutical composition for use as part of a combination therapy for treating a subject with a weakened immune system. In some embodiments, disclosed herein is a pharmaceutical composition for use as part of a combination therapy for use treating a subject suffering from an unresectable locally advanced or metastatic cutaneous melanoma.

A skilled artisan would appreciate that the phrases “physiologically acceptable carrier”, “pharmaceutically acceptable carrier”, “physiologically acceptable excipient”, and “pharmaceutically acceptable excipient”, may be used interchangeably may encompass a carrier, excipient, or a diluent that does not cause significant irritation to an organism and does not abrogate the biological activity and properties of the administered active ingredient.

A skilled artisan would appreciate that an “excipient” may encompass an inert substance added to a pharmaceutical composition to further facilitate administration of an active ingredient. In some embodiments, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences”, Mack Publishing Co., Easton, PA, latest edition, which is incorporated herein by reference.

In some embodiments, the composition as disclosed herein comprises a therapeutic composition. In some embodiments, the composition as disclosed herein comprises a therapeutic efficacy.

### Administration

In some embodiments, methods disclosed herein comprise administering compositions comprising an anti-IL-2 antibody as disclosed herein, in conjunction with a loading dose of IL-2. The method further comprises administering a booster dose of IL-2. In other embodiments, methods disclosed herein administer a combination therapy comprising compositions comprising an anti-IL-2 antibody as disclosed herein and a loading dose of IL-2. The method further comprises administering a booster dose of IL-2.

In some embodiments, methods disclosed herein comprise administering compositions comprising an anti-IL-2 antibody as disclosed herein and an immune checkpoint inhibitor as disclosed herein, in conjunction with a loading dose of IL-2. In some embodiments, the method further comprises administering a booster dose of IL-2. In other embodiments, methods disclosed herein administer a combination therapy comprising compositions comprising an anti-IL-2 antibody as disclosed herein and an immune checkpoint inhibitor as disclosed herein, and a loading dose of IL-2. In some embodiments, the method further comprises administering a booster dose of IL-2.

In some embodiments of the methods described herein that comprise administering a booster dose of IL-2, the booster dose of IL-2 is administered to the subject until at least Partial Response (PR) is achieved. In some embodiments of the methods described herein that comprise administering a booster dose of IL-2, the booster dose of IL-2 is administered to the subject with each treatment cycle, until at least Partial Response (PR) is achieved.

In some embodiments of the methods described herein that comprise administering a booster dose of IL-2, the booster dose of IL-2 is administered to the subject until at least complete response (CR) is achieved. In some embodiments of the methods described herein that comprise administering a booster dose of IL-2, the booster dose of IL-2 is administered to the subject with each treatment cycle, until at least complete response (CR) is achieved.

In some embodiments, Partial Response (PR) or complete response (CR) is evaluated according to RECIST.

In some embodiments of the methods described herein that comprise administering a booster dose of IL-2, the booster dose of IL-2 is administered to the subject until objective tumor shrinkage is observed. In some embodiments of the methods described herein that comprise administering a booster dose of IL-2, the booster dose of IL-2 is administered to the subject with each treatment cycle, until objective tumor shrinkage is observed.

In some embodiments, the booster dose of IL-2 is administered to the subject on day 1 of each cycle (Q8W). In some embodiments, the booster dose of IL-2 is administered to the subject until objective tumor shrinkage, or partial response, or complete response is observed on radiologic imaging or physical exam.

The anti-IL-2 antibody disclosed herein can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The above pharmaceutical compositions comprising the polypeptides disclosed herein can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Delivery can also be directed to any area of the respiratory system (e.g., lungs) via intubation. Alternatively, administration can be intratumoral, e.g., local or intravenous injection.

In certain embodiments, an anti-IL-2 antibody or composition thereof as described herein is administered intravenously (IV). In some embodiments, nivolumab or composition thereof is administered intravenously (IV). In some embodiments, an immune checkpoint inhibitor or composition thereof is administered intravenously (IV). In some embodiments, low dose IL-2 or composition thereof is administered subcutaneously. In some embodiments, a loading dose of IL-2 or composition thereof is administered subcutaneously. In some embodiments, a booster dose of IL-2 or composition thereof is administered subcutaneously.

In some embodiments, IL-2 subcutaneous administration is at much lower doses and much less frequently than the approved regimen of intravenously administered aldesleukin. In some embodiments, wherein a subject receives both an anti-IL-2 antibody and IL-2, the route of administration of the anti-IL-2 antibody is by intravenous injection and the route of administration of the IL-2 is by subcutaneous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

In some embodiments, where an anti-IL-2 antibody and IL-2 are administered, they may be administered in the same composition. In some embodiments, where an anti-IL-2 antibody and IL-2 are administered, they may be administered in separate compositions. In some embodiments, where an anti-IL-2 antibody and an immune checkpoint inhibitor, for example nivolumab are administered, they may be administered in the same composition. In some embodiments, where an anti-IL-2 antibody and an immune checkpoint inhibitor, for example nivolumab are administered, they may be administered in separate compositions.

In some embodiments, IL-2 may be administered prior to, concurrent with, or following the step of administering the anti-IL-2 antibody. In some embodiments, IL-2 administration is prior to administering the anti-IL-2 antibody. In some embodiments, IL-2 administration is concurrent with administering the anti-IL-2 antibody. In some embodiments, IL-2 administration follows the step of administering the anti-IL-2 antibody.

In some embodiments, the immune checkpoint inhibitor or nivolumab may be administered prior to, concurrent with, or following the step of administering the anti-IL-2 antibody. In some embodiments, the immune checkpoint inhibitor or nivolumab administration is prior to administering the anti-IL-2 antibody. In some embodiments, the immune checkpoint inhibitor or nivolumab administration is concurrent with administering the anti-IL-2 antibody. In some embodiments, the immune checkpoint inhibitor or nivolumab administration follows the step of administering the anti-IL-2 antibody.

In some embodiments, IL-2 may be administered prior to, concurrent with, or following the step of administering the immune checkpoint inhibitor or nivolumab. In some embodiments, IL-2 administration is prior to administering the immune checkpoint inhibitor or nivolumab. In some embodiments, IL-2 administration is concurrent with administering the immune checkpoint inhibitor or nivolumab. In some embodiments, IL-2 administration follows the step of administering the immune checkpoint inhibitor or nivolumab.

In some embodiments, administration of an anti-IL-2 antibody comprises including a loading dose of IL-2 with the anti-IL-2 antibody. In some embodiments, administration of an anti-IL-2 antibody comprises a combination therapy, wherein anti-IL-2 antibody and an IL-2 loading dose is administered to a subject at regular intervals, while an IL-2 booster dose is administered to a subject when there is one or more objective signs of worsening tumor growth kinetics, which in some embodiments comprise (a) previously shrinking tumors becoming stable, (b) an increase in tumor markers, (c) an increase in tumor growth, (d) an increase in appearance of new tumor growth in a tumor that had previously been stable, (e) an increase in appearance of new tumor growth in a tumor that had previously decreased in size, or (f) any combination thereof.

In some embodiments, an IL-2 booster dose is administered to a subject if tumor volume is stable, if tumor volume is unchanged, if one or more previously shrinking tumors becomes stable, if there is an increase in one or more tumor markers, if one or more new tumors are detected, if tumor growth is detected in one or more tumors that had previously been stable or had decreased in size, or any combination thereof

In some embodiments, an anti-IL-2 antibody is administered weekly, bi-weekly, or once every three weeks. In some embodiments, IL-2 is administered as a one-time dose. In some embodiments, the anti-IL-2 antibody and the IL-2 are administered independent of one another.

A skilled artisan would appreciate that duration of a treatment therapy, for example but not limited to for treating an unresectable locally advanced or metastatic solid cancer, may be based on the response or lack of response by the cancer to the treatment. In some embodiments, duration of a method of treating an unresectable locally advanced or metastatic solid cancer comprises about 2 years for patients who are receiving benefit from the treatment therapy. Benefit may in some embodiments be measured by tumor stabilization, tumor shrinkage, inhibiting or reducing growth of the tumor, inhibiting or reducing metastases of the tumor, inhibiting the production of new lesions, elimination of lesions, or any combination thereof. In some embodiments, duration of a method of treating an unresectable locally advanced or metastatic solid cancer comprises less than 2 years for patients who are receiving benefit from the treatment therapy. In some embodiments, duration of a method of treating an unresectable locally advanced or metastatic solid cancer comprises 1 year for patients who are receiving benefit from the treatment therapy. In some embodiments, duration of a method of treating an unresectable locally advanced or metastatic solid cancer comprises less than 1 years for patients who are receiving benefit from the treatment therapy.

In some embodiments of a method of treatment disclosed herein using a combination therapy, the administration of each component may be on a different treatment schedule for different durations. For example but not limited to administration of IL-2 booster doses, wherein the number of booster doses administered may vary widely between patients. In some embodiments, a patient receives at least one additional IL-2 dose (a “booster” dose). In some embodiments, a patient receives at least 1, 2, 3, 4, 5, 6, 7, 8, 9, 10, 11, or 12 additional IL-2 doses (“booster” doses) over a 2 year period. In some embodiments, a patient receives less than 12 booster IL-2 doses over a 2 year period. In some embodiments, a patient receives 6 booster IL-2 doses over a 1 year period. In some embodiments, a patient receives less than 6 booster IL-2 doses over a 1 year period. In some embodiments, a patient receives 1, 2, 3, 4, 5, or 6 booster IL-2 doses over a 1 year period. In some embodiments, a patient receives 3 booster IL-2 doses over a 6 month period. In some embodiments, a patient receives less than 3 booster IL-2 doses over a 6 month period. In some embodiments, a patient receives 1, 2, or 3 booster IL-2 doses over a 6 month period. In some embodiments, the duration of administration of booster doses of IL-2 as part of a combination therapy varies from patient to patient. In some embodiments, a patient receives a single booster dose over a 1 year period. In some embodiments, a patient receives a single booster dose over a 2 year period. In some embodiments, a patient receives up to 6 booster doses over a 1 year period. In some embodiments, a patient receives up to 12 booster doses over a 2 year period. In some embodiments, a patient receives 1, 2, 3, 4, 5, or 6 booster doses over a 1 year period. In some embodiments, a patient receives 1, 2, 3, 4, 5, 6, 7, 8, 9, 10, 11, or 12 booster doses over a 2 year period. In some embodiments, a patient receives booster doses intermittently over a 6 month-two year period. In some embodiments, a patient receives booster doses intermittently over a 6-month-2 year period based on a clinician's review of one or more objective signs of worsening tumor growth kinetics.

In some embodiments, therapeutic dosages of anti-IL-2 are administered at repeated administrations, e.g., of the same dose, over a period of months. In some embodiments, therapeutic dosages of anti-IL-2 are administered for up to 3 months. In some embodiments, therapeutic dosages of anti-IL-2 are administered for at least 3 months. In some embodiments, therapeutic dosages of anti-IL-2 are administered for up to 6 months. In some embodiments, therapeutic dosages of anti-IL-2 are administered for at least 6 months. In some embodiments, therapeutic dosages of anti-IL-2 are administered for up to 9 months. In some embodiments, therapeutic dosages of anti-IL-2 are administered for at least 9 months. In some embodiments, therapeutic dosages of anti-IL-2 are administered over a period of up to a year. In some embodiments, therapeutic dosages of anti-IL-2 are administered over a period of at least a year. In some embodiments, therapeutic dosages of anti-IL-2 are administered over a period of up to a year and a half. In some embodiments, therapeutic dosages of anti-IL-2 are administered over a period of at least a year and a half. In some embodiments, therapeutic dosages of anti-IL-2 are administered over a period of up to 2 years. In some embodiments, therapeutic dosages of anti-IL-2 are administered over a period of at least 2 years.

In some embodiments, therapeutic dosages of nivolumab are administered at repeated administrations, e.g., of the same dose, over a period of months. In some embodiments, therapeutic dosages of nivolumab are administered for up to 3 months. In some embodiments, therapeutic dosages of nivolumab are administered for at least 3 months. In some embodiments, therapeutic dosages of nivolumab are administered for up to 6 months. In some embodiments, therapeutic dosages of nivolumab are administered for at least 6 months. In some embodiments, therapeutic dosages of nivolumab are administered for up to 9 months. In some embodiments, therapeutic dosages of nivolumab are administered for at least 9 months. In some embodiments, therapeutic dosages of nivolumab are administered over a period of up to a year. In some embodiments, therapeutic dosages of nivolumab are administered over a period of at least a year. In some embodiments, therapeutic dosages of nivolumab are administered over a period of up to a year and a half. In some embodiments, therapeutic dosages of nivolumab are administered over a period of at least a year and a half. In some embodiments, therapeutic dosages of nivolumab are administered over a period of up to 2 years. In some embodiments, therapeutic dosages of nivolumab are administered over a period of at least 2 years.

In some embodiments, therapeutic dosages of the immune checkpoint inhibitor are administered at repeated administrations, e.g., of the same dose, over a period of months. In some embodiments, therapeutic dosages of the immune checkpoint inhibitor are administered for up to 3 months. In some embodiments, therapeutic dosages of the immune checkpoint inhibitor are administered for at least 3 months. In some embodiments, therapeutic dosages of the immune checkpoint inhibitor are administered for up to 6 months. In some embodiments, therapeutic dosages of the immune checkpoint inhibitor are administered for at least 6 months. In some embodiments, therapeutic dosages of the immune checkpoint inhibitor are administered for up to 9 months. In some embodiments, therapeutic dosages of the immune checkpoint inhibitor are administered for at least 9 months. In some embodiments, therapeutic dosages of the immune checkpoint inhibitor are administered over a period of up to a year. In some embodiments, therapeutic dosages of the immune checkpoint inhibitor are administered over a period of at least a year. In some embodiments, therapeutic dosages of the immune checkpoint inhibitor are administered over a period of up to a year and a half. In some embodiments, therapeutic dosages of the immune checkpoint inhibitor are administered over a period of at least a year and a half. In some embodiments, therapeutic dosages of the immune checkpoint inhibitor are administered over a period of up to 2 years. In some embodiments, therapeutic dosages of the immune checkpoint inhibitor are administered over a period of at least 2 years. In some embodiments, the immune checkpoint inhibitor is a PD-L1 immune checkpoint inhibitor. In other embodiments, the immune checkpoint inhibitor is a PD-1 immune checkpoint inhibitor.

In some embodiments, the dose of an anti-IL-2 antibody disclosed herein is between about 0.5 mg/kg and 12 mg/kg. In some embodiments, the dose of an anti-IL-2 antibody disclosed herein is about 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 m g/kg, 8.5 mg/kg, 9.0 mg/kg, 9.5 mg/kg, 10.0 mg/kg, 10.5 mg/kg, 11.0 mg/kg, 11.5 mg/kg, and 12 mg/kg.

In some embodiments, the dose of an anti-IL-2 antibody disclosed herein is 0.5 mg/kg. In some embodiments, the dose of an anti-IL-2 antibody disclosed herein is 1.0 mg/kg. In some embodiments, the dose of an anti-IL-2 antibody disclosed herein is 1.5 mg/kg. In some embodiments, the dose of an anti-IL-2 antibody disclosed herein is 2.0 mg/kg. In some embodiments, the dose of an anti-IL-2 antibody disclosed herein is 2.5 mg/kg. In some embodiments, the dose of an anti-IL-2 antibody disclosed herein is 3.0 mg/kg. In some embodiments, the dose of an anti-IL-2 antibody disclosed herein is 3.5 mg/kg. In some embodiments, the dose of an anti-IL-2 antibody disclosed herein is 4.0 mg/kg. In some embodiments, the dose of an anti-IL-2 antibody disclosed herein is 4.5 mg/kg. In some embodiments, the dose of an anti-IL-2 antibody disclosed herein is 5.0 mg/kg. In some embodiments, the dose of an anti-IL-2 antibody disclosed herein is 5.5 mg/kg. In some embodiments, the dose of an anti-IL-2 antibody disclosed herein is 6.0 mg/kg. In some embodiments, the dose of an anti-IL-2 antibody disclosed herein is 6.5 mg/kg. In some embodiments, the dose of an anti-IL-2 antibody disclosed herein is 7.0 mg/kg. In some embodiments, the dose of an anti-IL-2 antibody disclosed herein is 7.5 mg/kg. In some embodiments, the dose of an anti-IL-2 antibody disclosed herein is 8.0 mg/kg. In some embodiments, the dose of an anti-IL-2 antibody disclosed herein is 8.5 mg/kg. In some embodiments, the dose of an anti-IL-2 antibody disclosed herein is 9.0 mg/kg. In some embodiments, the dose of an anti-IL-2 antibody disclosed herein is 9.5 mg/kg. In some embodiments, the dose of an anti-IL-2 antibody disclosed herein is 10.0 mg/kg. In some embodiments, the dose of an anti-IL-2 antibody disclosed herein is 10.5 mg/kg. In some embodiments, the dose of an anti-IL-2 antibody disclosed herein is 11.0 mg/kg. In some embodiments, the dose of an anti-IL-2 antibody disclosed herein is 11.5 mg/kg. In some embodiments, the dose of an anti-IL-2 antibody disclosed herein is 12.0 mg/kg. In some embodiments, the dose of an anti-IL-2 antibody disclosed herein is 12.5 mg/kg. In some embodiments, the dose of an anti-IL-2 antibody disclosed herein is 4.5-12 mg/kg. In some embodiments, the dose of an anti-IL-2 antibody disclosed herein is 9-12 mg/kg.

In some embodiments, the dose of IL-2 comprises a low dose. One skilled in the art would appreciate that a low dose of IL-2 may encompass dose level below those provided in studies currently known in the art. In certain embodiments, an IL-2 dose between about 10×10³ IU/kg-300×10³ IU/kg encompasses a low dose of IL-2. In certain embodiments, an IL-2 dose between about 10×10³ IU/kg-500×10³ IU/kg encompasses a low dose of IL-2. In certain embodiments, an IL-2 dose between about 15×10³ IU/kg-500×10³ IU/kg encompasses a low dose of IL-2. In certain embodiments, an IL-2 dose between about 45×10³ IU/kg-270×10³ IU/kg encompasses a low dose of IL-2. In certain embodiments, an IL-2 dose between about 45×10³ IU/kg-135×10³ IU/kg encompasses a low dose of IL-2. In certain embodiments, an IL-2 dose is 135×10³ IU/kg encompasses a low dose of IL-2.

In some embodiments, the dose of IL-2 is between about 10×10³ IU/kg-500×10³ IU/kg. In some embodiments, the dose of IL-2 is between about 10×10³ IU/kg-300×10³ IU/kg. In some embodiments, the dose of IL-2 is between about 15×10³ IU/kg-270×10³ IU/kg. In some embodiments, the dose of IL-2 is about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500×10³ IU/kg. In some embodiments, the dose of IL-2 is about 15×10³ IU/kg. In some embodiments, the dose of IL-2 is about 45×10³ IU/kg. In some embodiments, the dose of IL-2 is about 135×10³ IU/kg. In some embodiments, the dose of IL-2 is about 270×10³ IU/kg. In some embodiments, the dose of IL-2 is about 300×10³ IU/kg. In some embodiments, the dose of IL-2 is about 400×10³ IU/kg. In some embodiments, the dose of IL-2 is about 500×10³ IU/kg. In some embodiments, the dose of IL-2 is 15×10³ IU/kg. In some embodiments, the dose of IL-2 is 45×10³ IU/kg. In some embodiments, the dose of IL-2 is 135×10³ IU/kg. In some embodiments, the dose of IL-2 is 270×10³ IU/kg. In some embodiments, the dose of IL-2 is 300×10³ IU/kg. In some embodiments, the dose of IL-2 is 400×10³ IU/kg. In some embodiments, the dose of IL-2 is 500×10³ IU/kg.

In some embodiments, methods described herein comprise administering a PD-1 checkpoint inhibitor to a subject at a dose of 240 mg. In other embodiments, methods described herein comprise administering a PD-1 checkpoint inhibitor to a subject at a dose of 120 mg. In other embodiments, methods described herein comprise administering a PD-1 checkpoint inhibitor to a subject at a dose of 360 mg. In other embodiments, methods described herein comprise administering a PD-1 checkpoint inhibitor to a subject at a dose of 600 mg. In other embodiments, methods described herein comprise administering a PD-1 checkpoint inhibitor to a subject at a dose of between about 120-600 mg. In other embodiments, methods described herein comprise administering a PD-1 checkpoint inhibitor to a subject at a dose of between about 240-480 mg. In some embodiments, methods described herein comprise administering a PD-1 checkpoint inhibitor to a subject at a dose of 240 mg once every 2 weeks. In other embodiments, methods described herein comprise administering a PD-1 checkpoint inhibitor to a subject at a dose of 480 mg once every 4 weeks. In other embodiments, methods described herein comprise administering a PD-1 checkpoint inhibitor to a subject at a dose of 240 mg once every 2 weeks.

In some embodiments, methods described herein comprise administering nivolumab to a subject at a dose of 240 mg. In other embodiments, methods described herein comprise administering nivolumab to a subject at a dose of 120 mg. In other embodiments, methods described herein comprise administering nivolumab to a subject at a dose of 360 mg. In other embodiments, methods described herein comprise administering nivolumab to a subject at a dose of 600 mg. In other embodiments, methods described herein comprise administering nivolumab to a subject at a dose of between about 120-600 mg. In other embodiments, methods described herein comprise administering nivolumab to a subject at a dose of between about 240-480 mg. In some embodiments, methods described herein comprise administering nivolumab to a subject at a dose of 240 mg once every 2 weeks. In other embodiments, methods described herein comprise administering nivolumab to a subject at a dose of 480 mg once every 4 weeks. In other embodiments, methods described herein comprise administering a PD-1 checkpoint inhibitor to a subject at a dose of 240 mg once every 2 weeks.

In some embodiments, methods described herein comprise administering an anti-IL-2 antibody to a subject at a dose of 9 mg/kg of the subject's body weight, IL-2 at a loading dose of 135,000 IU/kg of the subject's body weight, and IL-2 at a booster dose of 135,000 IU/kg of the subject's body weight. In some embodiments, the methods further comprise the step of administering nivolumab at a dose of 480 mg to the subject.

In some embodiments, methods described herein comprise administering an anti-IL-2 antibody to a subject at a dose of 7 mg/kg of the subject's body weight-11 mg/kg of the subject's body weight, IL-2 at a loading dose of 100,000 IU/kg of the subject's body weight-270,000 IU/kg of the subject's body weight, and IL-2 at a booster dose of 100,000 IU/kg of the subject's body weight-270,000 IU/kg of the subject's body weight. In some embodiments, the methods further comprise the step of administering nivolumab at a dose of 240 mg per subject-600 mg per subject to the subject.

In other embodiments, methods described herein comprise administering an anti-IL-2 antibody to a subject at a dose of 9 mg/kg of the subject's body weight, IL-2 at a loading dose of 135,000 IU/kg of the subject's body weight, and nivolumab at a dose of 480 mg per subject. In some embodiments, the methods further comprise the step of administering IL-2 at a booster dose of 135,000 IU/kg of the subject's body weight to the subject.

In other embodiments, methods described herein comprise administering an anti-IL-2 antibody to a subject at a dose of 7 mg/kg of the subject's body weight-11 mg/kg of the subject's body weight, IL-2 at a loading dose of 100,000 IU/kg of the subject's body weight-270,000 IU/kg of the subject's body weight, and nivolumab at a dose of 240 mg per subject-600 mg per subject. In some embodiments, the methods further comprise the step of administering IL-2 at a booster dose of 100,000 IU/kg of the subject's body weight-270,000 IU/kg of the subject's body weight to the subject.

In some embodiments when administering an anti-IL-2 antibody disclosed herein and a single loading dose of IL-2, the dose of anti-IL-2 antibody is 4.5 mg/kg and the dose of IL-2 is 15×10³ IU/kg. In some embodiments when administering an anti-IL-2 antibody disclosed herein and a single loading dose of IL-2, the dose of anti-IL-2 antibody is 4.5 mg/kg and the dose of IL-2 is 45×10³ IU/kg. In some embodiments when administering an anti-IL-2 antibody disclosed herein and a single loading dose of IL-2, the dose of anti-IL-2 antibody is 4.5 mg/kg and the dose of IL-2 is 135×10³ IU/kg. In some embodiments when administering an anti-IL-2 antibody disclosed herein and a single loading dose of IL-2, the dose of anti-IL-2 antibody is 4.5 mg/kg and the dose of IL-2 is 270×10³ IU/kg. In some embodiments when administering an anti-IL-2 antibody disclosed herein and a single loading dose of IL-2, the dose of anti-IL-2 antibody is 4.5 mg/kg and the dose of IL-2 is 300×10³ IU/kg. In some embodiments when administering an anti-IL-2 antibody disclosed herein and a single loading dose of IL-2, the dose of anti-IL-2 antibody is 4.5 mg/kg and the dose of IL-2 is 400×10³ IU/kg. In some embodiments when administering an anti-IL-2 antibody disclosed herein and a single loading dose of IL-2, the dose of anti-IL-2 antibody is 4.5 mg/kg and the dose of IL-2 is 500×10³ IU/kg.

In some embodiments when administering an anti-IL-2 antibody disclosed herein and a single loading dose of IL-2, the dose of anti-IL-2 antibody is 9.0 mg/kg and the dose of IL-2 is 15×10³ IU/kg. In some embodiments when administering an anti-IL-2 antibody disclosed herein and a single loading dose of IL-2, the dose of anti-IL-2 antibody is 9.0 mg/kg and the dose of IL-2 is 45×10³ IU/kg. In some embodiments when administering an anti-IL-2 antibody disclosed herein and a single loading dose of IL-2, the dose of anti-IL-2 antibody is 9.0 mg/kg and the dose of IL-2 is 135×10³ IU/kg. In some embodiments when administering an anti-IL-2 antibody disclosed herein and a single loading dose of IL-2, the dose of anti-IL-2 antibody is 9.0 mg/kg and the dose of IL-2 is 270×10³ IU/kg. In some embodiments when administering an anti-IL-2 antibody disclosed herein and a single loading dose of IL-2, the dose of anti-IL-2 antibody is 9.0 mg/kg and the dose of IL-2 is 300×10³ IU/kg. In some embodiments when administering an anti-IL-2 antibody disclosed herein and a single loading dose of IL-2, the dose of anti-IL-2 antibody is 9.0 mg/kg and the dose of IL-2 is 400×10³ IU/kg. In some embodiments when administering an anti-IL-2 antibody disclosed herein and a single loading dose of IL-2, the dose of anti-IL-2 antibody is 9.0 mg/kg and the dose of IL-2 is 500×10³ IU/kg.

In some embodiments when administering an anti-IL-2 antibody disclosed herein and a single loading dose of IL-2, the dose of anti-IL-2 antibody is 12 mg/kg and the dose of IL-2 is 15×10³ IU/kg. In some embodiments when administering an anti-IL-2 antibody disclosed herein and a single loading dose of IL-2, the dose of anti-IL-2 antibody is 12 mg/kg and the dose of IL-2 is 45×10³ IU/kg. In some embodiments when administering an anti-IL-2 antibody disclosed herein and a single loading dose of IL-2, the dose of anti-IL-2 antibody is 12 mg/kg and the dose of IL-2 is 135×10³ IU/kg. In some embodiments when administering an anti-IL-2 antibody disclosed herein and a single loading dose of IL-2, the dose of anti-IL-2 antibody is 12 mg/kg and the dose of IL-2 is 270×10³ IU/kg. In some embodiments when administering an anti-IL-2 antibody disclosed herein and a single loading dose of IL-2, the dose of anti-IL-2 antibody is 12 mg/kg and the dose of IL-2 is 300×10³ IU/kg. In some embodiments when administering an anti-IL-2 antibody disclosed herein and a single loading dose of IL-2, the dose of anti-IL-2 antibody is 12 mg/kg and the dose of IL-2 is 400×10³ IU/kg. In some embodiments when administering an anti-IL-2 antibody disclosed herein and a single loading dose of IL-2, the dose of anti-IL-2 antibody is 12 mg/kg and the dose of IL-2 is 250×10³ IU/kg.

In some embodiments when administering a combination therapy comprising an anti-IL-2 antibody disclosed herein and IL-2, wherein the dose of anti-IL-2 antibody is between about 0.1 mg/kg and 12 mg/kg anti-IL-2 antibody and the dose of IL-2 is between about 10×10³ IU/kg-300×10³ IU/kg. In some embodiments when administering a combination therapy comprising an anti-IL-2 antibody disclosed herein and IL-2, wherein the dose of anti-IL-2 antibody is between about 0.1 mg/kg and 12 mg/kg anti-IL-2 antibody and the dose of IL-2 is between about 10×10³ IU/kg-500×10³ IU/kg. In some embodiments when administering a combination therapy comprising an anti-IL-2 antibody disclosed herein and IL-2, wherein the dose of anti-IL-2 antibody is between about 0.5 mg/kg and 12 mg/kg anti-IL-2 antibody and the dose of IL-2 is between about 10×10³ IU/kg-300×10³ IU/kg. In some embodiments when administering a combination therapy comprising an anti-IL-2 antibody disclosed herein and IL-2, wherein the dose of anti-IL-2 antibody is between about 0.5 mg/kg and 12 mg/kg anti-IL-2 antibody and the dose of IL-2 is between about 10×10³ IU/kg-500×10³ IU/kg.

In some embodiments when administering a combination therapy comprising an anti-IL-2 antibody disclosed herein and IL-2, the dose of anti-IL-2 antibody is 0.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 500×10³ IU/kg.

In some embodiments when administering a combination therapy comprising an anti-IL-2 antibody disclosed herein and IL-2, the dose of anti-IL-2 antibody is 1.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 500×10³ IU/kg.

In some embodiments when administering a combination therapy comprising an anti-IL-2 antibody disclosed herein and IL-2, the dose of anti-IL-2 antibody is 4.5 mg/kg and the dose of IL-2 is about 10×10³ IU/kg-300 IU/kg. In some embodiments when administering a combination therapy comprising an anti-IL-2 antibody disclosed herein and IL-2, the dose of anti-IL-2 antibody is 4.5 mg/kg and the dose of IL-2 is about 10×10³ IU/kg-500 IU/kg.

In some embodiments when administering a combination therapy comprising an anti-IL-2 antibody disclosed herein and IL-2, the dose of anti-IL-2 antibody is 4.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 500×10³ IU/kg. In some embodiments when administering a combination therapy comprising an anti-IL-2 antibody disclosed herein and IL-2, the dose of anti-IL-2 antibody is 9.0 mg/kg and the dose of IL-2 is about 10×10³ IU/kg-300 IU/kg. In some embodiments when administering a combination therapy comprising an anti-IL-2 antibody disclosed herein and IL-2, the dose of anti-IL-2 antibody is 9.0 mg/kg and the dose of IL-2 is about 10×10³ IU/kg-500 IU/kg.

In some embodiments when administering a combination therapy comprising an anti-IL-2 antibody disclosed herein and IL-2, the dose of anti-IL-2 antibody is 9.0 mg/kg and the dose of IL-2 is 15×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 45×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 135×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 270×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 300×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 400×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 500×10³ IU/kg.

In some embodiments when administering a combination therapy comprising an anti-IL-2 antibody disclosed herein and IL-2, the dose of anti-IL-2 antibody is 12 mg/kg and the dose of IL-2 is 15×10³ IU/kg. In some embodiments when administering a combination therapy comprising an anti-IL-2 antibody disclosed herein and IL-2, the dose of anti-IL-2 antibody is 12 mg/kg and the dose of IL-2 is 45×10³ IU/kg. In some embodiments when administering a combination therapy comprising an anti-IL-2 antibody disclosed herein and IL-2, the dose of anti-IL-2 antibody is 12 mg/kg and the dose of IL-2 is 135×10³ IU/kg. In some embodiments when administering a combination therapy comprising an anti-IL-2 antibody disclosed herein and IL-2, the dose of anti-IL-2 antibody is 12 mg/kg and the dose of IL-2 is 270×10³ IU/kg. In some embodiments when administering a combination therapy comprising an anti-IL-2 antibody disclosed herein and IL-2, the dose of anti-IL-2 antibody is 12 mg/kg and the dose of IL-2 is 300×10³ IU/kg. In some embodiments when administering a combination therapy comprising an anti-IL-2 antibody disclosed herein and IL-2, the dose of anti-IL-2 antibody is 12 mg/kg and the dose of IL-2 is 400×10³ IU/kg. In some embodiments when administering a combination therapy comprising an anti-IL-2 antibody disclosed herein and IL-2, the dose of anti-IL-2 antibody is 12 mg/kg and the dose of IL-2 is 500×10³ IU/kg.

In some embodiments, the dose of IL-2 is considered low when compared with other therapies. In some embodiments, a low dose of IL-2 is between about 10×10³ IU/kg-500×10³ IU/kg. In some embodiments, a low dose of IL-2 is between about 10×10³ IU/kg-500×10³ IU/kg. In some embodiments, a low dose of IL-2 is between about 15×10³ IU/kg-500×10³ IU/kg. In some embodiments, a low dose of IL-2 is between about 45×10³ IU/kg-500×10³ IU/kg. In some embodiments, a low dose of IL-2 is equal to or less than about 500×10³ IU/kg. In some embodiments, a composition comprises an anti-IL-2 antibody comprising anti-IL-2 clone BDG 17.069.

In some embodiments, methods described herein comprise administering an anti-IL-2 antibody to a subject once every 2 weeks, a loading IL-2 dose administered to the subject once, and a booster IL-2 dose administered to the subject as needed. In some embodiments, the booster IL-2 dose is administered to the subject once every 8 weeks. In some embodiments, the methods further comprise the step of administering nivolumab to the subject once every 2 weeks.

In some embodiments, methods described herein comprise administering an anti-IL-2 antibody administered to a subject once every 1-4 weeks, a loading IL-2 dose administered to the subject once, and a booster IL-2 dose administered to the subject as needed. In some embodiments, the booster IL-2 dose is administered to the subject once every 4-16 weeks. In some embodiments, the methods further comprise the step of administering nivolumab to the subject once every 1-4 weeks.

In other embodiments, methods described herein comprise administering an anti-IL-2 antibody to a subject once every 2 weeks, a loading IL-2 dose administered to the subject once, and nivolumab to the subject once every 2 weeks. In some embodiments, the methods further comprise the step of administering IL-2 to the subject as needed. In some embodiments, the booster IL-2 dose is administered to the subject once every 8 weeks.

In other embodiments, methods described herein comprise administering an anti-IL-2 antibody to a subject once every 1-4 weeks, a loading IL-2 dose administered to the subject once, and an immune checkpoint inhibitor, for example nivolumab to the subject once every 1-4 weeks. In some embodiments, the methods further comprise the step of administering IL-2 to the subject as needed in a booster dose. In some embodiments, the booster IL-2 dose is administered to the subject once every 4-16 weeks.

In some embodiments of a method of use of a combination therapy described herein, the anti-IL-2 antibody is administered once every two weeks, the nivolumab is administered once every four weeks, and the IL-2 booster is administered once every 8 weeks if analysis of one or more objective signs of worsening tumor growth kinetics indicates the booster is warranted. In some embodiments, one or more objective signs comprises previously shrinking tumors becoming stable, increase in tumor markers, tumor growth, or appearance of new tumor growth in a tumor that had previously been stable or had decreased in size, or any combination thereof. In some embodiments, with each cycle of treatment an analysis of one or more objective signs is performed, wherein a skilled clinician determines if an IL-2 booster dose should be administered as part of the method.

As used herein, the terms “booster” or “booster dose” may encompass an additional dose of a therapeutic agent for example IL-2, to enhance or prolong its effect. AU-007 is an anti-IL-2 antibody that binds to IL-2 with pM affinity on its CD25 binding epitope and completely inhibits its binding to CD25, without hindering its binding to CD132/CD122). The presence of AU-007 therefore modifies the function of IL-2 such that the cells expressing the high affinity trimeric IL-2R complex can no longer use IL-2 while the cells expressing the moderate affinity dimeric receptor can still use IL-2, thus favoring the expansion and activation of that population.

In some embodiments, administration of a “boost” (booster dose) of IL-2 may be necessary in situations where there are very low levels of IL-2 available for AU-007 to capture. Patients who are tolerating treatment and are clinically stable can receive an additional dose on Day 1 of each therapeutic cycle until objective tumor shrinkage is observed on radiologic imaging or physical exam. A skilled investigator would understand when to administer an additional dose of aldesleukin (IL-2) with objective signs of worsening tumor growth kinetics: for example but not limited to previously shrinking tumors becoming stable or with new tumor growth.

A skilled artisan would appreciate that a booster dose is a supplementary dose of a therapeutic compound, such as IL-2, given after a loading dose. In some embodiments, the booster dose is administered to provide additional IL-2 to the subject after blood levels of IL-2 are depleted over time. In some embodiments, the anti-IL-2 antibody binds to IL-2 and thereby blocks IL-2 from binding to Treg cells, eosinophils, and pulmonary and vascular endothelial cells expressing the high affinity trimeric receptor (CD25/CD132/CD122). Excess IL-2 that is not complexed to the anti-IL-2 antibody binds to the moderate affinity dimeric receptor (CD132/CD122), which favors the expansion and activation of Teff cells, NK cells, and Natural killer T (NKT) cells.

### Formulations

Pharmaceutical compositions disclosed herein comprising anti-IL-2 antibodies, or a combination of anti-IL-2 antibodies and IL-2, or checkpoint inhibitors, can be conveniently provided as sterile liquid preparations, e.g., isotonic aqueous solutions, suspensions, emulsions, dispersions, or viscous compositions, which may be buffered to a selected pH, Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the anti-IL-2 antibodies, or a combination of anti-IL-2 antibodies and IL-2, or checkpoint inhibitors, described herein and utilized in practicing the methods disclosed herein, in the required amount of the appropriate solvent with various amounts of the other ingredients, as desired. In other embodiments, each component is formulated separately. For example, a sterile injectable solution is prepared for the anti-IL-2 antibody, a sterile injectable solution is prepared for the IL-2, and a sterile injectable solution is prepared for the checkpoint inhibitor, for example but not limited to nivolumab. Such formulations may be in admixture with a suitable carrier, diluent, or excipient such as sterile water, physiological saline, glucose, dextrose, or the like. The formulations can also be lyophilized. The formulations can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the formulations, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

In certain embodiments, the terms “pharmaceutical composition”, “composition”, and “formulation” may be used interchangeably having the same meanings and qualities.

The compositions or formulations described herein can be isotonic, i.e., they can have the same osmotic pressure as blood and lacrimal fluid. The desired isotonicity of the compositions as disclosed herein may be accomplished using sodium chloride, or other pharmaceutically acceptable agents such as dextrose, boric acid, sodium tartrate, propylene glycol or other inorganic or organic solutes. Sodium chloride may be preferred particularly for buffers containing sodium ions.

Viscosity of the compositions, if desired, can be maintained at the selected level using a pharmaceutically acceptable thickening agent. Methylcellulose may be preferred because it is readily and economically available and is easy to work with.

Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

In some embodiments, a composition is formulated to be at a pH between about pH 5.0-6.0. In some embodiments, a composition is formulated to be at a pH between about pH 5.0-7.0. In some embodiments, a composition is formulated to be at a pH between about pH 5.0-6.5. In some embodiments, a composition is formulated to be at a pH between about pH 5.0-5.5. In some embodiments, a composition is formulated to be at a pH between about pH 5.5-6.0. In some embodiments, a composition is formulated to be at a pH between about pH 5.5-6.5. In some embodiments, a composition is formulated to be at a pH between about pH 5.0. In some embodiments, a composition is formulated to be at a pH between about pH 5.5. In some embodiments, a composition is formulated to be at a pH between about pH 6.0. In some embodiments, a composition is formulated to be at a pH between about pH 6.5.

In some embodiments, a composition is formulated to be at a pH between about pH 5.0-6.0 and comprises a buffer. In some embodiments, the buffer comprises a pharmaceutically acceptable buffer. In some embodiments, the buffer comprises a histidine buffer or a citrate buffer. In some embodiments, the buffer comprises a histidine buffer. In some embodiments, the buffer comprises a citrate buffer.

In some embodiments, a composition is formulated to be at a pH between about pH 5.0-6.0 and comprises a buffer selected from a histidine buffer and a citrate buffer. In some embodiments, a composition is formulated to be at a pH between about pH 5.0-6.0 and comprises a histidine buffer. In some embodiments, a composition is formulated to be at a pH between about pH 5.0-6.0 and comprises a citrate buffer.

In some embodiments, a composition further comprises at least one of sucrose, methionine, or PS80, or any combination thereof. In some embodiments, a composition further comprises sucrose. In some embodiments, a composition further comprises methionine. In some embodiments, a composition further comprises PS80.

In some embodiments, a composition comprises an anti-IL-2 antibody as disclosed herein and is formulated to be at a pH between about pH 5.0-6.0 and comprises a buffer selected from a histidine buffer and a citrate buffer. In some embodiments, the composition further comprises IL-2.

Those skilled in the art will recognize that the components of the compositions or formulations should be selected to be chemically inert and will not affect the viability or efficacy of the early apoptotic cell populations as described herein, for use in the methods disclosed herein. This will present no problem to those skilled in chemical and pharmaceutical principles, or problems can be readily avoided by reference to standard texts or by simple experiments (not involving undue experimentation), from this disclosure and the documents cited herein.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

A skilled artisan would appreciate that the term “about”, may encompass a deviance of between 0.0001-5% from the indicated number or range of numbers. In some instances, the term “about”, may encompass a deviance of between 1-10% from the indicated number or range of numbers. In some instances, the term “about”, encompasses a deviance of up 10 to 25% from the indicated number or range of numbers.

As used herein, the term “dose” may encompass, in some embodiments, a measured quantity of a therapeutic agent to be taken at one time. A skilled artisan would appreciate that administration of a dose encompasses providing the given measured quantity of the therapeutic agent to a subject.

### EXAMPLES

### Example 1

Phase 1/2 Clinical Trial for AU-007 with Recombinant Human IL-2 (Aldesleukin)

Engineered anti-IL-2 antibodies designed to redirect IL-2 activity toward effector T cells and NK cells while avoiding stimulation of regulatory T cells (Tregs) and CD25+ endothelial cells were generated and characterized as described in US-2024/0287169, which is incorporated herein by reference in its entirety.

AU-007 is a human mAb that binds IL-2 on its CD25 binding epitope. AU-007 bound IL-2 cannot bind trimeric (CD25, CD122, CD132) IL-2 receptors (IL-2R) on regulatory T cells (Tregs), vascular endothelium, or eosinophils, but IL-2's binding to dimeric IL-2R (CD122, CD132) on T effector (T eff) and NK cells is unhindered. AU-007 thus redirects IL-2 towards T eff and NK cell activation, while diminishing Treg activation and vascular leak. AU-007 uniquely redirects IL-2 generated from T eff cell expansion, converting a Treg-mediated autoinhibitory loop into an immune stimulating loop (as described in US-2024/0287169, which is incorporated herein by reference in its entirety). CDR sequences are defined according to IMGT numbering.

The heavy chain variable region and light chain variable region amino acid sequences of Clone 17.069 are presented in Table 2. Clone 17.069 comprises the LALA mutation (L234A, L235A mutations).

TABLE 2
VH and VL Amino Acid Sequences of
Anti-IL-2 Clones.

| | Heavy Chain | Light Chain |
|---|---|---|
| Clone | Variable Region | Variable Region |
| 17.069 | QVQLVQSGAEVKKPGSSVKV | DIVMTQSPDSLAVSLGERAT |
| | SCKASGYSITDDLIHWVRQA | INCKSSQSLLRRGNQKNHLA |
| | PGQGLEWMGWIDPEDGETNY | WYQQKPGQPPKLLIYDASTG |
| | AQKFQGRVTLTADTSTSTAY | QSGVPDRFSGSGSGTDFTLT |
| | MELSSLRSEDTAVYYCARSL | ISSLQAEDVAVYYCLQSYIT |
| | DSTWIYPFAYWGQGTLVTVS | PPTFGAGTKVEIK |
| | S | (SEQ ID NO: 7) |
| | (SEQ ID NO: 6) | |

The CDR sequences for Clone 17.069 is provided in Table 3.

TABLE 3
CDR Amino Acid Sequences of Anti-IL-2 Clones.

| | Heavy Chain | | | Light Chain | | |
|---|---|---|---|---|---|---|
| Clone | CDR1 | CDR2 | CDR3 | CDR1 | CDR2 | CDR3 |
| 17.069 | GYSI | IDPE | ARSLD | QSLL | DAS | LQSY |
| | TDDL | DGET | STWIY | RRGN | | ITPP |
| | (SEQ | (SEQ | PFAY | QKNH | | T |
| | ID | ID | (SEQ | (SEQ | | (SEQ |
| | NO: | NO: | ID | ID | | ID |
| | 1) | 2) | 3) | NO: | | NO: |
| | | | | 4) | | 5) |

The full length amino acid sequences for Clone 17.069 is provided in Table 4 below.

TABLE 4
Full-length Amino Acid Sequences
of Anti-IL-2 Clones.

| Clone | Heavy Chain (LALA) | Light Chain |
|---|---|---|
| BDG | QVQLVQSGAEVKKPGSSVKV | DIVMTQSPDSLAVSLGERAT |
| 17.069 | SCKASGYSITDDLIHWVRQA | INCKSSQSLLRRGNQKNHLA |
| | PGQGLEWMGWIDPEDGETNY | WYQQKPGQPPKLLIYDASTG |
| | AQKFQGRVTLTADTSTSTAY | QSGVPDRFSGSGSGTDFTLT |
| | MELSSLRSEDTAVYYCARSL | ISSLQAEDVAVYYCLQSYIT |
| | DSTWIYPFAYWGQGTLVTVS | PPTFGAGTKVEIKRTVAAPS |
| | SASTKGPSVFPLAPSSKSTS | VFIFPPSDEQLKSGTASVVC |
| | GGTAALGCLVKDYFPEPVTV | LLNNFYPREAKVQWKVDNAL |
| | SWNSGALTSGVHTFPAVLQS | QSGNSQESVTEQDSKDSTYS |
| | SGLYSLSSVVTVPSSSLGTQ | LSSTLTLSKADYEKHKVYAC |
| | TYICNVNHKPSNTKVDKKVE | EVTHQGLSSPVTKSFNRGEC |
| | PKSCDKTHTCPPCPAPEAAG | (SEQ ID NO: 9) |
| | GPSVFLFPPKPKDTLMISRT | |
| | PEVTCWWVDVSHEDPEVKFN | |
| | WYVDGVEVHNAKTKPREEQY | |
| | NSTYRVVSVLTVLHQDWLNG | |
| | KEYKCKVSNKALPAPIEKTI | |
| | SKAKGQPREPQVYTLPPSRD | |
| | ELTKNQVSLTCLVKGFYPSD | |
| | IAVEWESNGQPENNYKTTPP | |
| | VLDSDGSFFLYSKLTVDKSR | |
| | WQQGNVFSCSVMHEALHNHY | |
| | TQKSLSLSPGK | |
| | (SEQ ID NO: 8) | |

AU-007 may be formulated in 20 mM Histidine, 8% sucrose, 10 mM Methionine, 0.04% PS80, pH5.5 (F4).

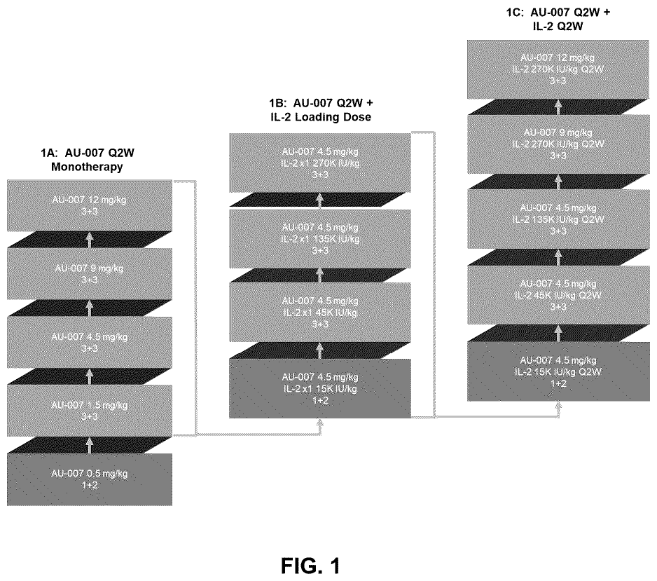

A clinical study was designed to test the efficacy of AU-007 with recombinant IL-2 (aldesleukin) in patients with cancer. In the study, 3 dose escalation arms were followed by cohort expansion (FIG. 1). Arm 1A evaluated escalating doses (0.5-12 mg/kg of said subject's body weight) of AU-007 (IV once every 2 weeks [Q2W]). Arm 1B evaluated AU-007 Q2W+ a single escalating loading low doses (15K-270K IU/kg of said subject's body weight) of aldesleukin administered subcutaneously (SC). Arm 1C evaluated AU-007+ escalating low doses of SC aldesleukin, both Q2W. There were at least twenty solid tumor types allowed in the dose escalation arms. Cohort expansion Arm 2B evaluated 9 mg/kg of said subject's body weight AU-007+one loading dose of aldesleukin at a dose of 135K IU/kg of the subject's body weight. Tumor assessments were conducted at the end of each 8-week cycle.

In the B and C Arms (or B and C regimens) of the study, recombinant human IL-2 (aldesleukin) was administered subcutaneously, at much lower doses (15K IU/kg, 45K IU/kg, 135K IU/kg, or 270K IU/kg of said subject's body weight) and much less frequently than the approved regimen of intravenously administered aldesleukin, which is 600,000 IU/kg of said subject's body weight every 8 hours for up to 14 administrations.

In FIG. 1, the terms “3+3” and “1+2” refer to the size of the cohort at a given dose level. For 3+3, the first 3 enrolled patients were administered AU-007 at the dose level that is noted. Since no dose-limiting toxicities (DLTs) were observed, administration was escalated to the next highest dose level. However, if any of the first 3 patients had experienced a dose-limiting toxicity that is drug-related, then an additional three more patients would have received treatment at that same dose level to see if they have DLTs before escalating. 1+2 follows the same principle—start with one patient, if DLTs had been observed, two more patients would have been added. No DLTs were observed in the first single patient, and dosages were escalated.

The 20 solid tumor histologies of patients enrolled in the trial are:
1. Urothelial cancer arising in the bladder, renal pelvis, ureter, or urethra that has progressed during or following an anti-PDx therapy and, if eligible, a platinum-containing regimen.
2. Adrenocortical carcinoma that is unresectable, locally advanced, or metastatic.
3. Clear cell renal cell carcinoma (ccRCC) progressing during or following at least 2 approved therapeutic regimens (e.g., small molecule inhibitors, anti-PDx therapy).
4. Melanoma that is either locally unresectable or metastatic:
   a. BRAF wt: progressed after receiving anti-PD-1 containing therapy with or without an anti-CTLA-4; or
   b. BRAF mut: progressed after a BRAF+MEK inhibitor.
5. Triple-negative breast cancer that is unresectable locally advanced or metastatic and that is refractory to standard 1^{st} line therapy, which may include cytotoxic chemotherapy alone and/or poly ADP ribose polymerase (PARP) inhibitors for breast cancer gene (BRCA), 1 or 2 mutations, and/or anti-PDx therapy in MSI-H/dMMR positive tumors.
6. Head and neck squamous cell carcinoma (HNSCC) that has progressed during or following treatment with an anti-PDx (unless ineligible, e.g., patients failing chemotherapy and PD-L1 combined positive score (CPS)<1) and platinum-based chemotherapy (unless ineligible for platinum chemotherapy) for metastatic or recurrent disease.
7. Gastric or gastro-esophageal cancer progressing during or after cytotoxic chemotherapy (e.g., paclitaxel, fluoropyrimidine, platinum agents) with or without trastuzumab (for HER2 overexpressing adenocarcinoma) and with or without anti PD-1 inhibitor therapy. Patients with a CPS ≥1 should have received an anti PD-1 containing regimen (unless intolerant or therapy unavailable).
8. Esophageal squamous cell carcinoma progressing during or after cytotoxic chemotherapy (e.g., paclitaxel, fluoropyrimidine, platinum agents) with an anti PD-1 therapy. Patients with a CPS ≥10 should have received an anti PD-1 containing regimen (unless intolerant or therapy unavailable).
9. Cutaneous squamous cell carcinoma (cSCC): recurrent or metastatic cSCC that is not curable by surgery or radiation.
10. Pancreatic adenocarcinoma that is unresectable locally advanced or metastatic and received at least one line of chemotherapy (e.g., FOLFIRINOX) (unless ineligible or not feasible).
11. Cholangiocarcinoma that is unresectable locally advanced or metastatic in patients who have had 1 line of systemic chemotherapy, unless the patient is ineligible for chemotherapy.
12. Hepato-cellular carcinoma (HCC) progressing during or following an approved therapeutic regimen (unless ineligible).
13. Colorectal cancer (CRC):
   a. K-Ras wild type: Patients who have progressed during or after, or are ineligible for, both irinotecan-based and oxaliplatin-based chemotherapy and who are relapsed or refractory to at least 1 prior systemic therapy that included an anti-epidermal growth factor receptor (EGFR) antibody, such as cetuximab or panitumumab; or
   b. K-Ras mutant: Patients who have progressed during or after, or are ineligible for, both irinotecan and oxaliplatin based chemotherapy (±bevacizumab).
14. Epithelial ovarian cancer progressing during or following at least one prior cytotoxic chemotherapeutic regimen (unless ineligible), and subsequent poly ADP ribose polymerase (PARP) inhibitor therapy in BRCA mutation positive patients (unless ineligible).
15. Cervical cancer progressing during or following first-line cytotoxic chemotherapy and second-line cytotoxic chemotherapy or anti-PDx therapy in PD-L1 positive (CPS ≥1) or MSI-H/dMMR positive tumors (unless ineligible).
16. Endometrial cancer in patients progressing on or following either cytotoxic chemotherapy (±trastuzumab) or hormone therapy, and an anti-PDx therapy in MSI-H/dMMR positive tumors.
17. Thyroid cancer (follicular or papillary histology) that is iodine refractory.
18. Non-small cell lung cancer (NSCLC) that has progressed during or following treatment with platinum-based chemotherapy and an anti-PDx therapy for unresectable locally advanced or metastatic disease. NSCLC harboring an activating EGFR mutation (excluding Exon 20 insertion mutations) or anaplastic lymphoma kinase (ALK) rearrangement must have progressed following available EGFR or ALK-targeted therapy in addition to treatment with platinum-based chemotherapy (unless ineligible for platinum therapy).
19. Merkel Cell Carcinoma: metastatic Merkel cell carcinoma that is not curable by surgery or radiation.
20. Nasopharyngeal cancer.

In addition to the 20 solid tumor histologies listed above, patients entered into the trial may be suffering any of the solid tumors including a melanoma, a metastatic melanoma, a primary melanoma and metastatic melanoma, a renal cell carcinoma (RCC), a non-small cell lung cancer (NSCLC), a bladder cancer, a head and neck cancer, a head and neck squamous cell carcinoma (HNSCC), a nasopharyngeal carcinoma, a urothelial cancer, an adrenal cortical carcinoma, a clear cell renal cell carcinoma (ccRCC), a triple-negative breast cancer, a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a uterine cancer, a gallbladder cancer, or a Merkel cell carcinoma, or any tumors that are microsatellite instabilities (MSI)-high tumors. In some cases, the cancer may be unresectable locally advanced or metastatic cancer.

Adverse events were graded by the Common Terminology Criteria for Adverse Events (CTCAE). Efficacy evaluation was based on PD (Pharmacodynamic) markers of immune stimulation, total IL-2 (bound to AU-007+free IL-2) and objective responses.

Evaluation of efficacy is based on PD markers of immune stimulation, total IL-2 (bound to AU-007+free IL-2), and objective response.

Peripheral blood samples were taken prior to dosing on Day 1 and following dosing at 4 hours, and on Days 2, 3, 15, 29, and 43 of cycle 1 and pre-dose/Day 29 on all other cycles. Dosing with AU-007 was done via intravenous dosing by weight-based dosing (mg/kg). Proleukin® (aldesleukin) was dosed subcutaneously by weight-based dose (IU/kg). Whole blood was stained for CD4+ T cells, CD8+ T cells, CD4+ Tregs, NK cells, B cells, and monocytes. Samples were analyzed by flow cytometry using TruCount™ for absolute cell counts. Changes from baseline values were determined off absolute counts. In addition, differential hematology counts were taken on Days 1, 15, and 29 of all cycles (or as needed per treating physician's decision) for safety evaluation and to examine eosinophil levels (eosinophils express the IL-2 trimeric receptor). Serum was taken prior to dosing on Day 1 and at 2 hours, 6 hours, and Days 2, 3, 15 pre/post, and 6 hours, 29 pre/post, 43 pre/post, and subsequent Day 1 of each cycle at pre/post. Samples were analyzed for INF-γ (LOQ 31 fg/ml), IL-2 (LOQ 31 fg/ml), and sCD25 (10 fg/ml) using the ECL Mesoscale Dynamics platform.

### Example 2

### Clinical Trial Methodology

### Assessments of Subjects During the Screening Period (Pre-Treatment), During the Treatment Period, and Post-Treatment

The Screening Period was conducted within 28 days of Cycle 1 Day 1 and included the following procedures:
Physical examination including height and weight
Vital signs
Performance status
Medical history
Cancer history
Routine blood tests: comprehensive chemistry panel, special chemistry, hematology, coagulation, endocrine test
Tumor markers appropriate for tumor type (e.g., CA-125, CEA, CA19-9, LDH)
Routine urine test
Electrocardiogram
Urine or blood pregnancy test (women of childbearing potential)
CT or MRI scan or ultrasound or positron emission tomography (PET) scan if appropriate
Brain CT or MRI scan if appropriate
PD-L1 testing (if not previously tested)
Biopsy

The Treatment Period (Day 1 to Day 56 of each cycle) includes the following procedures:
Physical examination
Interim medical history
Study drug administration
Post-treatment observation
Vital signs
Performance status
Routine blood tests: comprehensive chemistry panel, special chemistry, hematology, coagulation, endocrine test
Tumor markers
Research blood tests: pharmacokinetics, cytokines and soluble biomarkers, flow cytometry, anti-drug antibodies
Routine urine test
Electrocardiogram
Urine or blood pregnancy test (women of childbearing potential)
CT or MRI scan or ultrasound or positron emission tomography (PET) scan if appropriate
Brain CT or MRI scan if appropriate
Biopsy Cycle 2 Day 1 (optional in Dose Escalation)

### Post-Treatment Assessments

Following the last dose of study drug, all patients are followed by electronic (e.g., email) or telephone communication for safety and survival at Day 28 and Day 90, and then every 2 months for survival during a one-year Survival Follow-up Period.

### Safety

Dose-limiting toxicity criteria are defined as follows:

### Hematologic Dose-Limiting Toxicity

Grade 4 neutropenia lasting >7 days.
Grade 3 neutropenic fever.
Grade 3 thrombocytopenia with clinically significant bleeding.

### Hepatic Non-hematologic Dose Limiting Toxicity

Grade 3 aspartate aminotransferase (AST) or alanine aminotransferase (ALT) increase >10× the upper limit of normal (ULN).
Grade 3 AST or ALT increase >5.0 to 10.0×ULN and not resolving to Grade 2 (i.e., >3.0 to 5.0×ULN) within 7 days and Grade 1 (i.e., >ULN to 3.0×ULN) within 14 days (unless patient enrolled with elevated transaminases secondary to metastases, then must resolve to baseline). Steroids must be tapered to ≤10 mg of prednisone or equivalent per day by Day 14.
Grade 3 bilirubin increase >5×ULN.
Grade 3 bilirubin increase >3.0 to 5.0×ULN and not resolving to Grade 2 (i.e., >1.5 to 3.0×ULN) within 7 days and Grade 1 (i.e., >ULN to 1.5×ULN) within 14 days. Steroids must be tapered to ≤10 mg of prednisone or equivalent per day by Day 14.
Any event meeting the Hy's law criteria (all 3 features must apply):
   AST and/or ALT >3×ULN.
   Concurrent elevation of total bilirubin >2×ULN.
   No alternative etiology can be identified for combination increases of AST/ALT and total bilirubin.

### Non-Hematologic Dose Limiting Toxicity

Non-Hematologic DLTs are Grade >3 Non-Hematologic AEs with the Following Exceptions:
Grade 3 electrolyte abnormality lasting <72 hours without associated clinical complications and responds to therapy.
Grade 3 fever lasting <72 hours and not associated with hemodynamic instability.
Grade 3 nausea or vomiting resolving to ≤Grade 1 within 72 hours with or without medical intervention.
Grade 3 amylase and/or lipase elevations not associated with either clinical or radiographic evidence of pancreatitis requiring hospitalization.
Grade 3 diarrhea, constipation, abdominal pain, cramping, dyspepsia, or dysphagia resolving to Grade ≤1 within 72 hours with or without medical therapy.
Grade 3 fatigue resolving to Grade 1 within <7 days.
Grade 3 CLS.
Grade 3 IRR including cytokine release syndrome lasting ≤12 hours and responding to medical intervention. Grading is based on overall IRR event, and not grade of individual signs or symptoms including:
   Fever, chills, nausea or vomiting, diarrhea, hypotension, hypertension, or tachycardia.
Grade 3 endocrinopathy controlled with hormone supplementation.
Grade 3 skin toxicity resolving to Grade ≤2 within 7 days with oral corticosteroids.
Grade 3 inflammatory reaction secondary to anti-tumor response (e.g., metastatic sites, lymph nodes) resolving to Grade ≤1 within 7 days.

### Grade 2 Non-Hematologic AEs May be Considered DLTs

Grade 2 AEs that are prolonged inordinately, based upon the medical judgment of the Investigator, and/or lead to permanent discontinuation of AU-007 due to patient intolerance.

Discontinuation criteria include:
Patient meets criteria for objective progression by modified Response Evaluation Criteria In Solid Tumors (RECIST) v1.1, i.e., progression that is confirmed by a second scan after receiving one more cycle of AU-007.
Occurrence of a DLT
Sponsor, Investigator, or regulatory agency terminates the study.
Withdrawal of patient due to an adverse event or serious adverse event.
Patient experiences a medical condition that requires treatment with prohibited concomitant medication.
Withdrawal of patient consent.
Completion of protocol-defined therapy.
Pregnancy.

Patients who remain clinically stable (per RECIST definition below) and do not experience dose limiting toxicities (DLTs) or unacceptable toxicity during Cycle 1 may continue to receive additional 8-week treatment cycles until discontinuation criteria are met.

### Efficacy

Tumor assessment (or evaluation) (CT scan, MRI, PET scan, or ultrasound) is carried out at the end of each cycle, on Study Day 56. Tumor evaluation refers to imaging (CT scan, MRI, PET scan, or ultrasound) to measure tumors and assess for any new tumors—see RECIST definition below.

Response is based on RECIST v1.1:
Complete Response: Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on the study (this includes the baseline sum if that is the smallest on study). In addition to the relative increase of 20%, the sum must also demonstrate an absolute increase of at least 5 mm. The appearance of one or more new lesions is also considered progression.
Stable Disease (SD): Neither sufficient shrinkage to qualify for PR nor sufficient increase to qualify for PD, taking as reference the smallest sum diameters while on study.

Exploratory biopsies are optionally conducted to evaluate the effect of the treatment on immune cell infiltration within the tumor. Biopsies are not conducted as part of determining a patient's response to treatment in conjunction with CT scans.

Patient blood samples for cytokines and soluble biomarkers were obtained per the following schedule:

### Cycle 1:

Day 1 pre-dose (prior to first study drug administration on a given day) and 2 and 6 hours post-dose
Day 2
Day 3
Day 15 pre-dose, end of infusion, and 6 hours post-dose
Day 29 pre-dose and end of infusion
Day 43 pre-dose and end of infusion

### Cycle 2:

Day 1 pre-dose and end of infusion
Day 15 pre-dose and end of infusion
Day 43 pre-dose and end of infusion

### Cycles 3 and Beyond:

Day 1 pre-dose and end of infusion

### Example 3

### Dose Selection for Phase 2 Trials (Recommended Phase 2 Dose; RP2D)

### Methods

The assay below examines safety assessments conducted under the clinical protocol. Pharmacokinetics (PK) assays, and pharmacodynamic (PD) assays were carried out for the B regimen (Arm B) and C regimen (Arm C) to identify the doses to be used in Phase 2 Expansion Cohorts.

Pharmacokinetic data were derived using standard antibody PK techniques using a capture electrochemiluminescent (ECL) assay by coating a mouse anti-AU-007 antibody on an ECL plate followed by incubating with serum. Detection was done using sulfa-Tagged anti-AU-007 (different from capture).

Pharmacodynamic measures included changes in circulating cell populations and peripheral interferon gamma (IFN-γ) expression.

Circulating cells populations (CD8+ T and NK effector cells; and Treg Cells) were determined by flow cytometry and peripheral blood cytokines (IL-2, and IFN-γ), and sCD25 were determined using electrochemiluminescence (SPLEX and VPlex kits; https://www.mesoscale.com/en/products_and_services/assay_kits).

Data from the assays were collated and compared across dosing regimens and levels to determine the dose to be used in the expansion.

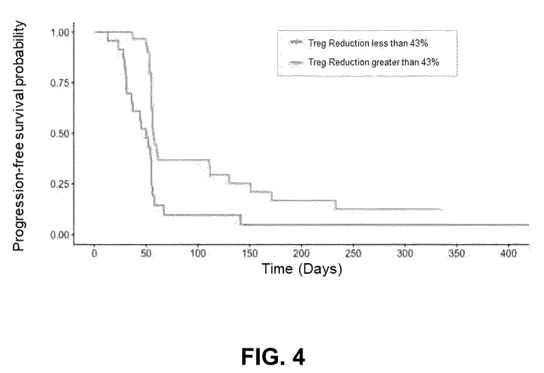

To assess the level of correlation between progression free survival (PFS) and Treg reduction, the largest reduction achieved after Day 3 of treatment was calculated for each participant. Patients were then grouped by those who achieved less than the median Treg reduction and those who achieved more than the median Treg reduction, and PFS was plotted on a Kaplan-Meier curve (FIG. 4). This allows determination of whether a larger Treg reduction is linked to better clinical outcomes, providing evidence for the immunological effects of treatment and their relationship to disease progression.

Progression-free survival (PFS) is defined as the number of days between the first dose of study drug and the first occurrence of objective progressive disease or death, whichever occurs first. Patients without progression or death are censored at the time of the last tumor assessment. Censoring is an approach to account for incomplete data at the time of the analysis. For example, there may be patients who are in between visits for determining their tumor measurements, or their vital status (alive or dead), in which case the data point used for the PFS analysis would be the last time they had a tumor assessment. The assessments as provided herein are based on the last known data point.

A Cox proportional hazards model of PFS was utilized to evaluate the risk of progression by dose. The model includes effects for AU-007 dose, aldesleukin dose, aldesleukin regimen (B or C Regimen), disease type (RCC, melanoma, or other), and other biomarkers.

The plots show the estimated relative risk (aka hazard ratio) of progression or death for each combination of aldesleukin dose, AU-007 dose, and disease (holding other variables constant). The reference group is a single aldesleukin dose of 135,000 IU/kg and AU-007 dose of 4.5 mg/kg in the “other” disease type, so the plotted values are indexed against that group.

### Results

### Pharmacokinetics Results

Table 5 presents PK data showing the pharmacokinetic data of patients based on AU-007 dosage and administration regime (Arm A, B, or C).

TABLE 5
AU-007 Pharmacokinetic Profile.

| | AU-007 Dose | | Cmax | AUClast |
|---|---|---|---|---|
| Arm | (mg/kg) | N | (mg/mL) | (d*mg/mL) |
| 1A* | 0.5 | 2 | 10.8 (16) | 53.7 (105) |
| 1A | 1.5 | 3 | 29.6 (13) | 231 (12) |
| 1A | 4.5 | 3 | 110 (15) | 828 (42) |
| 1A | 9 | 4 | 255 (21) | 1,700 (22) |
| 1A | 12 | 3 | 282 (9.1) | 1,910 (39) |
| 1B** | 4.5 | 12 | 132 (47) | 639 (39) |
| 1C*** | 4.5 | 21 | 155 (42) | 773 (46) |
| 1C | 9 | 4 | 223 (12) | 1,350 (25) |

*Arm 1A − AU-007 Q2W − monotherapy
**Arm 1B − AU-007 Q2W + IL-2 loading dose − 15K; 45K; 135K; 270K IU/kg
***Arm 1C − AU-007 Q2W + IL-2 Q2W − 15K; 45K; 135K; 270K IU/kg

The AUC and Cmax were both approximately dose-proportional over the dose range tested (Table 5). The characteristics were typical of an IgG1-LALA monoclonal antibody, with an approximate 15-day half-life in humans. In general, comparable AU-007 PK exposure (AUC) occurred when dosed alone (Arm 1A) or in combination with aldesleukin (Arms 1B and 1C).

There was no evidence of neutralizing anti-drug antibodies. These data demonstrate that AU-007 with or without IL-2 combination demonstrates low potential for immunogenicity.

### Peripheral Cell Populations

The mean fold change in peripheral cell populations on Day 43 following treatment initiation, regardless of AU-007 dose (by evaluating Arm B patient samples only—AU-007 4.5 mg/kg) was evaluated at each aldesleukin dose level (6).

Day 43 was the end of Cycle 1 lab collection timepoint and was an adequate timeframe to determine changes.

TABLE 6
Mean Fold Change* in Effector cells (CD8
and NK cells) and Tregs at Day 43

| | | CD8 | NK | Treg | CD8/Treg |
|---|---|---|---|---|---|
| IL-2 Dose Level | N | Cells | Cells | Cells | Ratio |
| 15K IU/kg | 1 | 0.86 | 0.56 | 0.42 | 2.05 |
| 45K IU/kg | 4 | 0.76 | 0.82 | 0.45 | 1.45 |
| 135K IU/kg | 2 | 1.61 | 2.11 | 0.81 | 1.97 |
| 270K IU/kg | 2 | 0.99 | 1.31 | 0.56 | 1.95 |

*Fold change was determined relative to the Day 1 pre-dose sample.

The largest fold change in CD8, NK, and Treg cells on day 43 was seen at the 135,000 IU/kg dose level, with the highest expansion of CD8+ T and NK effector cells and a CD8/Treg ratio of 1.97-fold increase (Table 6). These results suggest that concentrations greater than 135K IU/kg gain no further peripheral blood pharmacodynamic advantage.

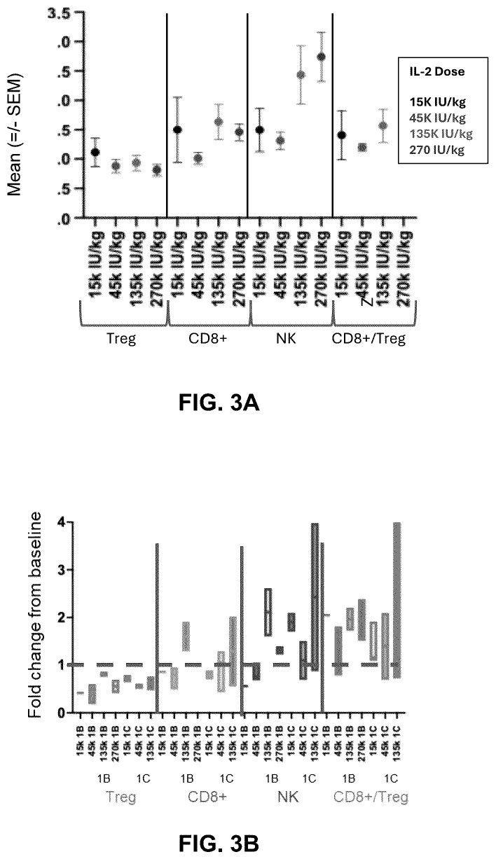

The fold change in peripheral cell population (Treg, NK, CD8+ cell populations, and the CD8/Treg ratio) was evaluated on Day 15 after a first dose of 4.5 mg/kg AU-007 and escalating doses of aldesleukin (FIG. 3A). The Treg population did not change or was decreased relative to baseline (relative to the Day 1 pre-dose sample) when measured on Day 15. Ascending doses of aldesleukin tended to increase CD8+ T cells and NK cells and favored a higher CD8/Treg ratio, with maximum effect seen at 135,000 IU/kg aldesleukin (FIG. 3A).

Day 43 levels of Treg, NK, CD8+ cell populations, and the CD8/Treg ratio in the aldesleukin Dose Escalation cohorts treated with 4.5 mg/kg AU-007 and 15,000 IU/kg, 45,000 IU/kg, 135,000 IU/kg (Arm 1B and 1C), or 270,000 IU/kg (Arm 1B) aldesleukin were also assessed. For all doses, the Treg population was decreased relative to baseline (relative to the Day 1 pre-dose sample). Ascending doses of subcutaneous aldesleukin tended to increase CD8+ T cells and NK cells and favored a higher CD8/Treg ratio (FIG. 3B).

The significance of decreased Treg levels on the duration of progression-free survival (PFS) was examined. The median Treg reduction across Phase 1 and 2 patients was 43% (analysis included patients from Phase 1 and 2 with available pre and post samples at the time of analysis (not including Arm A). A comparison of PFS between patients with less than 43% reduction in Tregs vs. greater than 43% reduction in Tregs showed that patients with a Treg cell reduction of greater than the median of 43% is associated with longer PFS across Phase 1 and 2 than those with lower Treg reduction (FIG. 4).

### Circulating Interferon Gamma (IFN-γ)

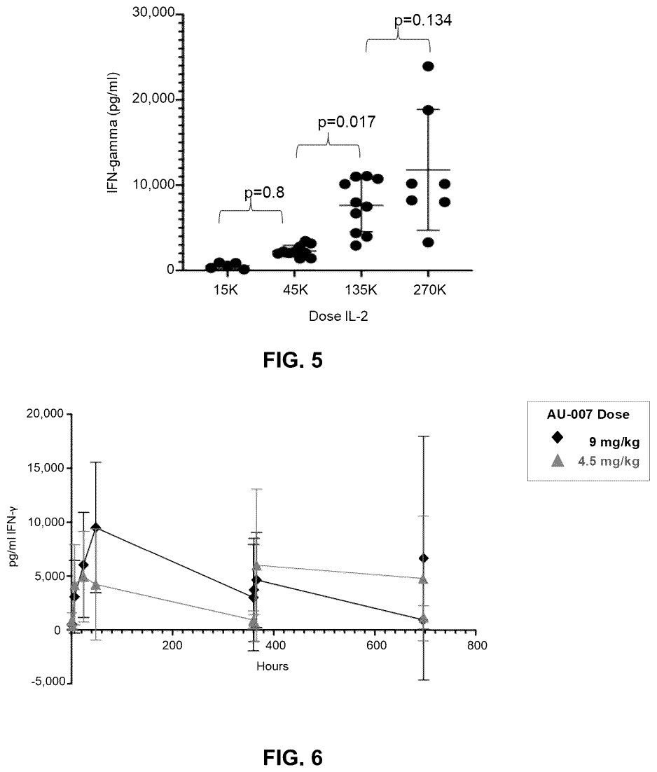

The effect of subcutaneous aldesleukin dose escalation (FIG. 5) or AU-007 dose (FIG. 6) on the concentration of circulating IFN-γ was examined using the ECL Mesoscale Dynamics platform. Circulating IFN-γ from serum samples generally rose with increasing doses of aldesleukin; however, no significant differences were observed between 135,000 IU/kg and 270,000 IU/kg aldesleukin. Circulating IFN-γ was measured at a pre-dose time point and then 2 hours, 6 hours, 24 hours, and 48 hours post aldesleukin dose. Additionally, IFN-γ was measured on day 15 pre-dose and 6 hours post dose administration of aldesleukin, days 29 and 43 on pre-dose and end of infusion, Cycle 2 day 1, 15 and 43 pre dose and end of infusion (EOI). All other cycles are pre-dose and EOI day 1

Changes in circulating IFN-γ levels were also observed across both AU-007 dose levels (4.5 mg/kg and 9.0 mg/kg). IFN-γ levels were higher in the first two weeks after dosing with 9 mg/kg AU-007 plus 135,000 IU/kg aldesleukin compared to dosing with 4.5 mg/kg AU-007 plus 135,000 IU/kg aldesleukin (FIG. 6).

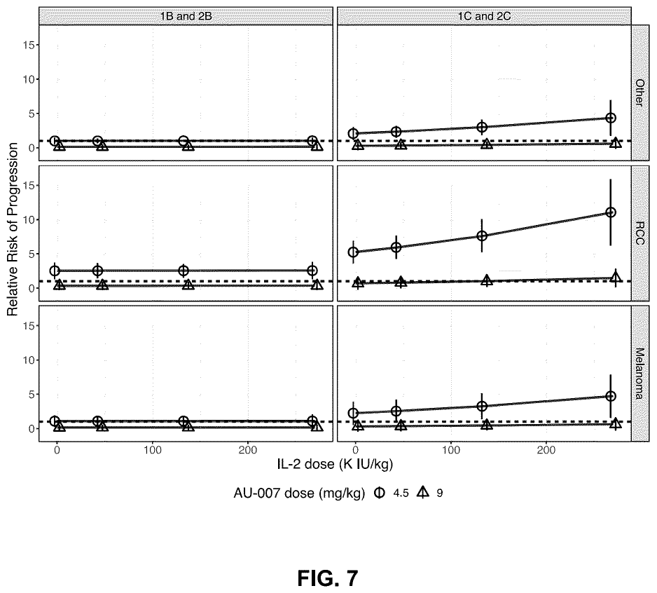

A Cox proportional hazards model of PFS was utilized to evaluate the risk of progression by dose. The model includes effects for AU-007 dose, aldesleukin dose, aldesleukin regimen (B or C), disease type (RCC, melanoma, or other), and other biomarkers (FIG. 7).

The plots show the estimated relative risk (aka hazard ratio) of progression or death for each combination of aldesleukin dose, AU-007 dose, and disease (holding other variables constant). The reference group is a single aldesleukin dose of 135,000 IU/kg and AU-007 dose of 4.5 mg/kg in the “other” disease type, so the plotted values are indexed against that group.

Based on modeling of data from the study, dosing with 9 mg/kg AU-007 was associated with a lower risk of disease progression compared to 4.5 mg/kg AU-007 (7). The differences between the risk of progression as modeled for each dose level was more distinct in the Arm C cohorts.

The IFN-γ results show that a combination of 9 mg/kg AU-007 plus 135,000 IU/kg aldesleukin is optimal at enhancing IFN-γ production (FIG. 6) with a lower relative risk of progression (FIG. 7) as compared to a combination of 4.5 mg/kg AU-007 plus 135,000 IU/kg aldesleukin.

### Additional Data Supporting AU-007 Dose Selection

There was no substantial safety difference observed across all the AU-007 doses evaluated. All doses demonstrated mostly mild events manageable with over-the-counter medications (data not shown).

There were also no definitive differences in efficacy signals between the 4.5, 9 and 12 mg/kg dose levels.

Preclinical cynomolgus monkey TK predicted that even the second dose level evaluated in escalation (1.5 mg/kg AU-007) would bind >1000-fold the projected IL-2 serum concentrations achieved with the 270K IU/kg aldesleukin

As there were no safety or efficacy differences between the doses, the AU-007 RP2D was selected based on AU-007 coverage of IL-2 molecules (8). AU-007 achieves its maximal efficacy with the lowest amounts of unbound IL-2 (endogenous and exogenously administered) in a patient's body. Unbound IL-2 preferentially binds to the trimeric IL-2R found on Tregs (100-fold higher affinity of the trimeric IL-2R for IL-2 vs the dimeric IL-2R) and thus would increase the Treg population.

Therefore, we targeted a 10,000-15,000 overage of AU-007 to IL-2 (135,000 IU/Kg) on a molecule-to-molecule ratio, and the 9 mg/kg AU-007 dose was calculated to provide a ~13,000-fold overage of AU-007 to IL-2 administered at 135,000 IU/Kg.

In view of the above results, 9 mg/kg AU-007 was selected for the RP2D.

### Conclusions

The AU-007 PK data demonstrated dose-proportionality over the dose range tested, showed no signs of neutralizing ADA activity, and have an estimated initial T_{1/2} of approximately 15 days in humans (US-2024-0287169, incorporated herein by reference).

The data on escalating doses of aldesleukin demonstrate that higher aldesleukin levels increased peripheral IFN-γ levels and circulating effector cell populations, with the greatest benefit achieved at 135,000 IU/kg of aldesleukin.

AU-007 at all dose levels, regardless of aldesleukin dosing regimen decreased Treg cells in the periphery. Greater reductions in Tregs were associated with longer PFS.

Taken together, the data showed that AU-007 can control and redirect the endogenously produced IL-2 and the exogenously administered IL-2 to reduce the Treg population and increase peripheral IFN-γ levels and circulating CD8+ T and NK effector cell populations.

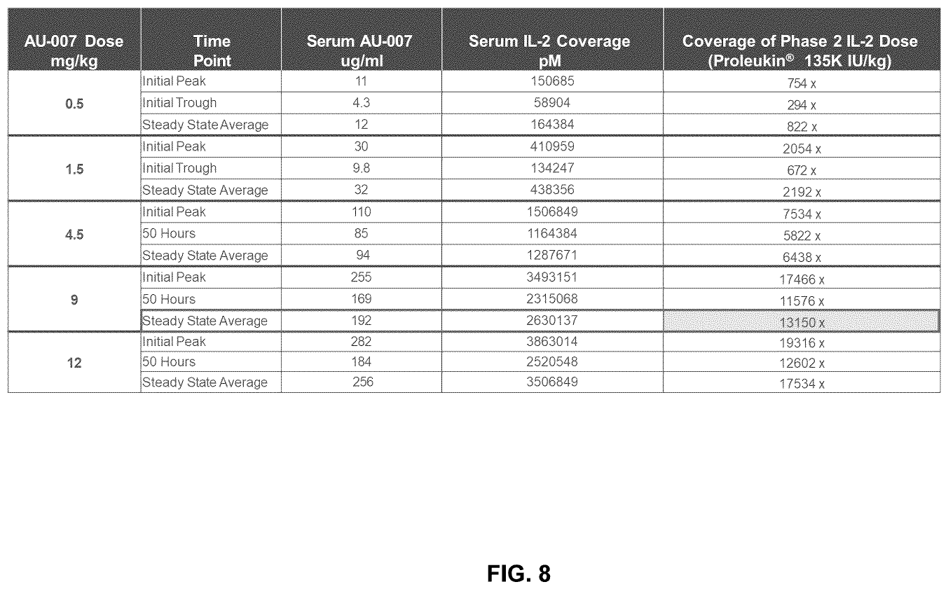

Dosing with 9 mg/kg of AU-007 was optimal at enhancing IFN-γ production (FIG. 6). In addition, the 9 mg/kg AU-007 dose was calculated to provide a ~13,000-fold coverage of AU-007 to IL-2 administered at 135,000 IU/Kg (FIG. 8).

Based on these results, 9 mg/kg AU-007 Q2W and 135,000 IU/kg subcutaneous aldesleukin with both a single loading aldesleukin dose (2B) and Q2W aldesleukin (2C) schedules were evaluated in the Phase 2 expansion cohorts.

### Example 4

### Regimen Selection for Aldesleukin Administration

### Methods

Data from patients in Arm B and Arm C regimens of Phase 1 or Phase 2 clinical trial were compared to determine the best regimen for administration of AU-007 plus aldesleukin:
Arm B regimen: AU-007 Q2W plus a single loading dose of aldesleukin; and
Arm C regimen: AU-007 Q2W plus aldesleukin Q2W.

Melanoma and renal cell carcinoma (RCC) patients were initially selected for Phase 2 Cohort Expansion.

Table 7 summarizes the number of melanoma and renal cell carcinoma (RCC) patients who received the Arm B regimen or the Arm C regimen.

TABLE 7
The number of melanoma and renal cell carcinoma (RCC) patients
who received the Arm B regimen or the Arm C regimen.

| | Arm B Regimen | Arm C Regimen |
|---|---|---|
| | AU-007 Q2W + Single | AU-007 Q2W + |
| Diagnosis | Dose Aldesleukin (n) | Aldesleukin Q2W (n) |
| Melanoma | 7 | 6 |
| Renal Cell Carcinoma | 5 | 8 |

### Pharmacodynamic Methods

Patient blood samples for cytokines and soluble biomarkers were obtained per the schedule described in Example 2 above. Circulating cells were determined by flow cytometry, and peripheral blood cytokines were determined using electrochemiluminescence. To assess the level of correlation between PFS and Treg reduction, the largest reduction achieved after Day 3 was calculated for each participant.

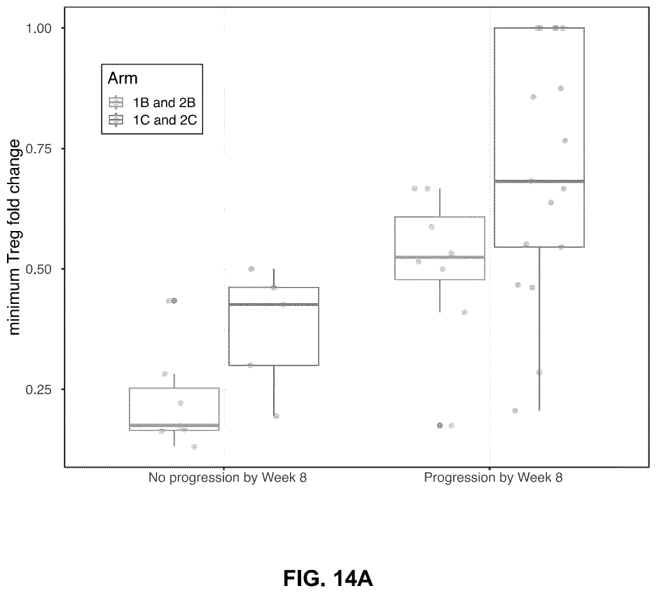

Patients were then grouped by those who achieved less than the median Treg reduction and those who achieved more than the median Treg reduction, and PFS was plotted on a Kaplan-Meier curve (FIG. 13).

### Comparison of Efficacy Results

### Melanoma Patients

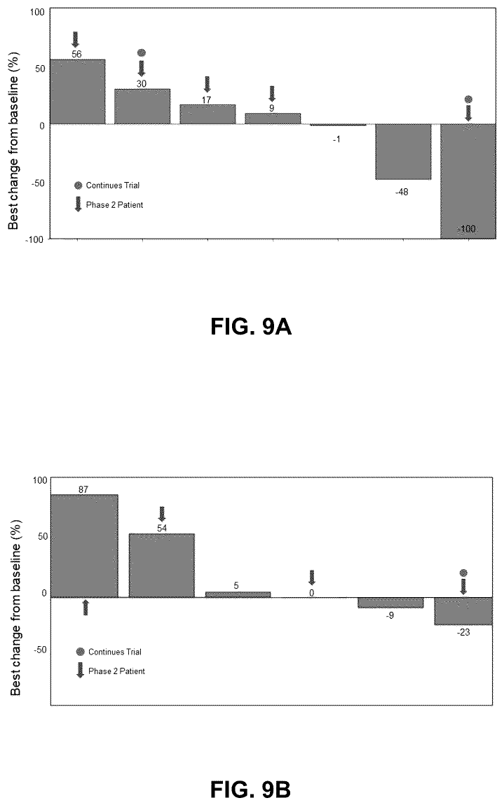

Greater reductions in target tumor size and longer durations of response were seen in melanoma patients receiving the Arm B regimen (AU-007 Q2W plus a single aldesleukin dose), with two patients achieving a 48% and 100% reduction (FIG. 9A) as compared to the Arm C regimen (AU-007 Q2W+SC Aldesleukin Q2W) with two patients achieving a 9% and 23% reduction (FIG. 9B).

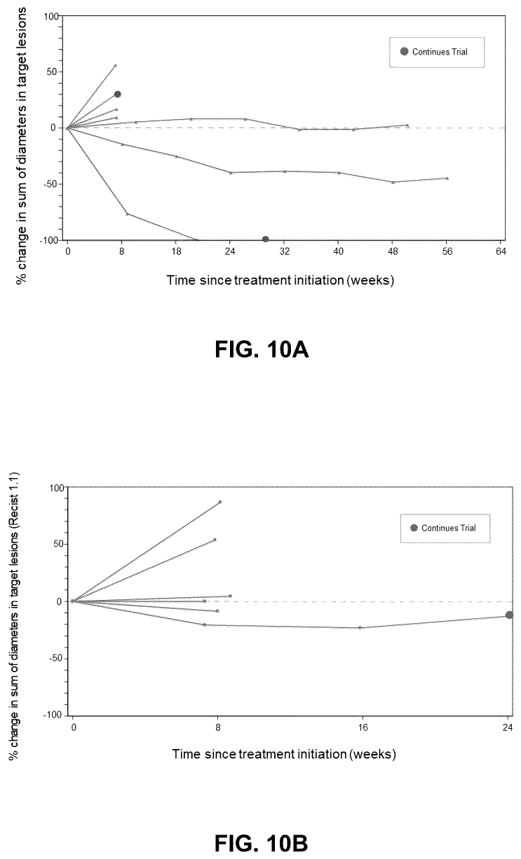

The percentage change in the sum of diameters in target lesions over time (vs. baseline) in Melanoma patients in Phase 1 and Phase 2 patients receiving AU-007 Q2W+single SC aldesleukin loading dose (Arm B Dosing Regimen; FIG. 10A) and in patients receiving AU-007 Q2W+SC aldesleukin Q2W (Arm C Dosing Regimen; FIG. 10B) were evaluated.

Among melanoma patients receiving AU-007 Q2W plus a single aldesleukin dose, response was maintained to 50 and 56 weeks, with one patient continuing on treatment at approximately Week 30 (FIG. 10A). Among melanoma patients receiving AU-007 plus aldesleukin Q2W, most patients discontinued at Week 8 (end of Cycle 1); one patient continues on treatment at Week 24 (FIG. 10B).

### Renal Cell Carcinoma (RCC) Patients

Target tumor size changes were similar for RCC patients in the Arm B and Arm C regimens, with longer duration of response in one Arm B patient (32 weeks at data cut-off) compared to the Arm C patients (data not shown; one discontinued at Week 8 (−22% change) and one was ongoing at Week 16 [−7% change]).

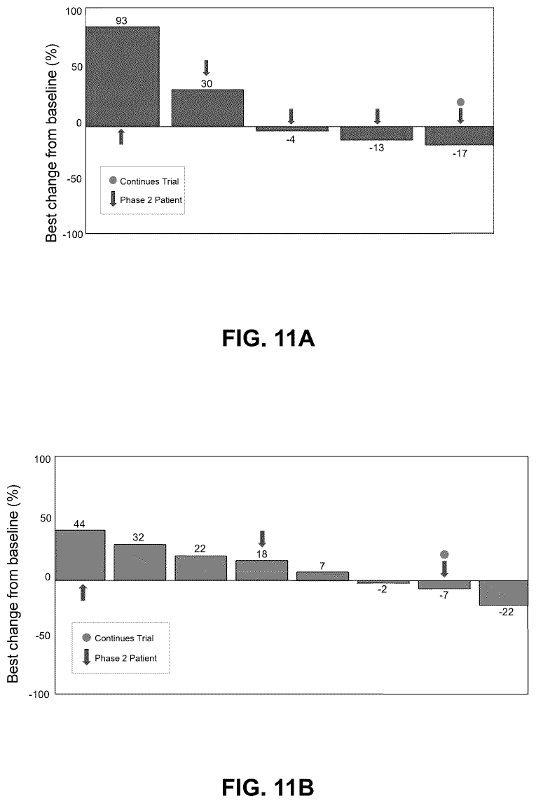

FIG. 11A and FIG. 11B show best response in RCC patients in Phase 1 and Phase 2 receiving AU-007 Q2W+single SC aldesleukin Loading Dose (Arm B Dosing Regimen) and AU-007 Q2W+SC aldesleukin Q2W (Arm C Dosing Regimen), respectively.

Target tumor reductions of 4%, 13%, and 17% were documented in RCC patients receiving AU-007 plus a single aldesleukin dose (FIG. 11A) as compared to target tumor reductions of 2%, 7%, and 22% documented in RCC patients receiving AU-007 plus aldesleukin Q2W (FIG. 11B).

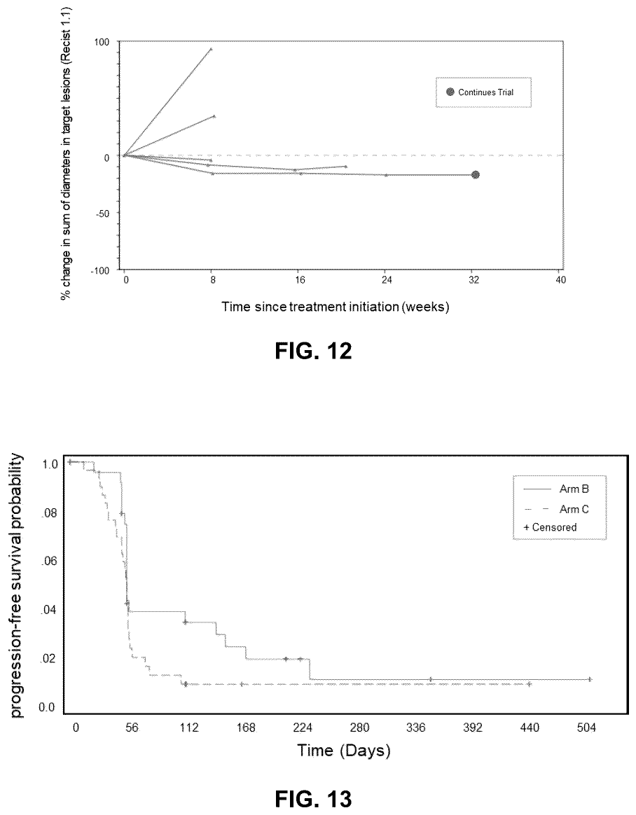

The percentage change in the sum of diameters in target lesions over time (vs. baseline) for RCC patients receiving AU-007 Q2W+single SC aldesleukin loading dose (Arm B Dosing Regimen; FIG. 12). One patient receiving AU-007 Q2W plus a single aldesleukin dose continues on treatment at Week 32.

Progression-free survival (PFS) was evaluated for all patients (all tumor types) receiving Arm B (n=23) vs. Arm C (n=39) regimen in Phase 1 and 2 (FIG. 13). A Kaplan-Meier plot comparing preliminary PFS data from the Arm B vs. Arm C dosing regimens, across all patients in Phase 1 and Phase 2, demonstrated a trend to longer PFS for patients receiving the Arm B dosing regimen (FIG. 13). Most patients with progression or death at 56 days (end of Cycle 1) were from the Phase 1 dose escalation portion of the study consisting mostly of heavily pre-treated gastrointestinal cancers or cancers not typically associated with response to immunotherapies (e.g., uterine, thyroid cancers).

### Pharmacodynamic Results

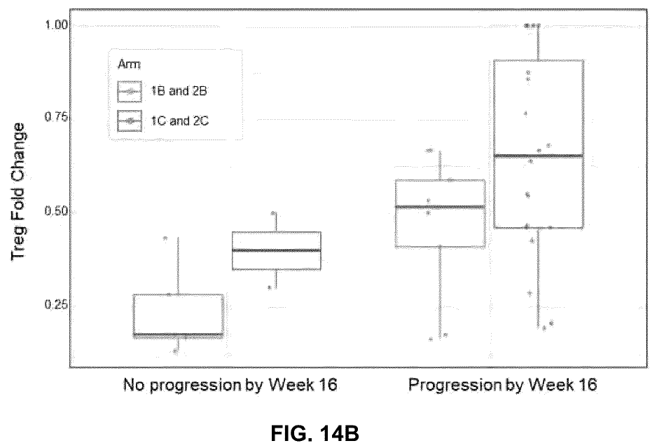

As described above, FIG. 4 demonstrates that Treg cell reduction is associated with longer PFS. The association between Treg cell reduction and longer PFS per dose regimen was evaluated at Week 8 (end of Cycle 1; FIG. 14A) and Week 16 (end of Cycle 2 FIG. 14B). The decrease in Tregs (excluding the first 2 days on treatment) was expressed as a fold change vs. baseline (e.g., 0.25 is a 75% decrease). The results demonstrated that patients with greater Treg decreases were less likely to have a progressed status in Cycles 1 and 2 for both Arm B and Arm C regimens (FIGS. 14A-14B).

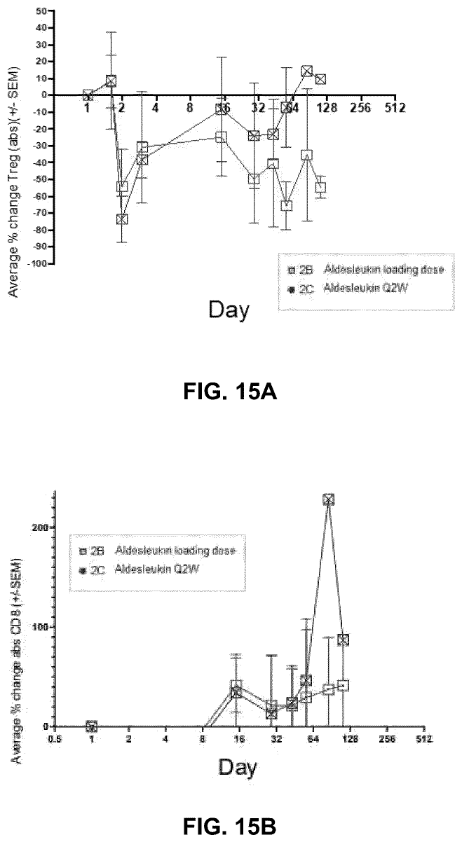

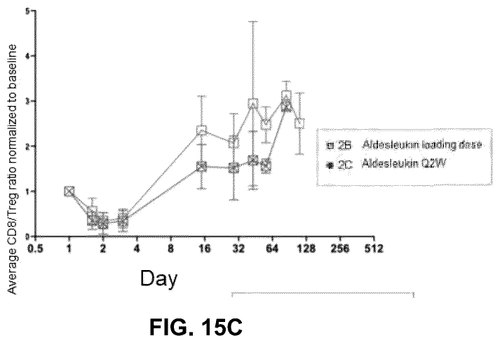

The percent changes in Tregs (FIG. 15A) and CD8 cells (FIG. 15B), and the CD8/Treg ratio (FIG. 15C) in Arm 2B compared to Arm 2C regimens were evaluated. Peripheral Tregs decreased with both Arm 2B and 2C dosing regimens (FIG. 15A), with Arm 2B dosing trending toward greater and more durable decreases of Tregs. Peripheral CD8 cells (FIG. 15B) demonstrated similar increases for Arm B and C dosing regimens, ranging from 30-50% higher than baseline.

The CD8/Treg ratio (FIG. 15C) trended higher for the Arm 2B dosing regimen since the B and C regimens increased CD8 cells to the same extent while the Treg decrease was greater on the B dosing regimen.

### Conclusions

Decrease in Tregs appears to be a critical determinant of observed efficacy, with greater decreases in patients receiving a SC aldesleukin loading dose compared to SC aldesleukin Q2W. Therefore, the single SC aldesleukin loading dose regimen (Arm B dosing) was chosen as the regimen for further clinical development based on the following factors:
The additional IL-2 administration with the Arm C regimen did not improve efficacy vs. the Arm B regimen's single SC aldesleukin loading dose (FIGS. 9A-9B and 11A-11B), and patients on the Arm B regimen trend toward having deeper and more durable tumor shrinkage with prolonged PFS (FIGS. 10A-10B).
The Arm B regimen had a strong trend to deeper and more durable Treg decreases (FIGS. 14A-14B and 15A) that are associated with longer PFS in early data (FIG. 13), leading to a greater CD8/Treg ratio.
More prolonged IL-2 exposure on the Arm C dosing regimen may be driving the Effector T (Teff) cells to exhaustion.
The Arm C dosing regimen caused greater and more prolonged increases of interferon-gamma (IFN-γ) vs. the Arm B regimen (data not shown).
Prolonged exposure to IFN-γ may be immune suppressive.

### Example 5

AU-007 in Combination with Single-Dose Aldesleukin and Optional Aldesleukin Boost Administration (Phase 2B)

### Treatment Methods

Patients with unresectable locally advanced or metastatic malignant neoplasms including clear cell renal cell carcinoma (ccRCC), cutaneous melanoma that has progressed during or following treatment with either PD-1 or PD-L1, or unresectable locally advanced or metastatic PD-L1-positive non-small cell lung cancer (NSCLC) were enrolled in Arm 2B of the Cohort Expansion phase of the study.

Unresectable locally advanced tumors cannot be resected and have spread locally (in contrast to metastatic tumors that spread from the original location to another part of the body).

Inclusion criteria for ccRCC patients included patient's cancer progressing during or following at least 2 approved therapeutic regimens (for example but not limited to small molecule inhibitors, anti-PD-1 or anti-PD-L1 therapy).

Inclusion criteria for cutaneous melanoma patients:
Patients with BRAF mutations must either be ineligible for or have refused a BRAF+MEK inhibitor.
Must have objective progression after receiving at least two cycles of prior doublet therapy (anti-PD-1/anti-CTLA-4 or anti-PD-1/anti-LAG-3).
“Objective progression” is considered measurable/quantifiable, i.e., Response Evaluation Criteria in Solid Tumors (RECIST v1.1 definition of progression—see definition below).
Progressive Disease is defined as having at least a 20% increase in the sum of diameters of target lesions, taking as reference the smallest sum on study (this includes the baseline sum if that is the smallest on study). Progression is a >/=20% increase in the sum of the tumors compared to the smallest sum from any prior study scan. In addition to the relative increase of 20%, the sum must also demonstrate an absolute increase of at least 5 mm. The appearance of one or more new lesions is also considered progression.
Radiographic progression 4 weeks prior to the first dose of study drug to rule out late response to most recent therapy. The requirement for documented radiologic progression may be waived after review by Medical Monitor (e.g., in the case of progression beyond 12 weeks after starting a doublet). This study's baseline scan can serve to document confirmed progression to most recent therapy. The most recent therapy is the last therapy the patient received before starting this study.
Lactate dehydrogenase (LDH) ≤2.5× upper limit of normal.

Inclusion criteria for NSCLC patients:
Patients with unresectable locally advanced or metastatic, PD-L1-positive (tumor proportion score [TPS]1%), non-small cell lung cancer (NSCLC) not harboring an activating EGFR mutation or ALK rearrangement and has progressed during or following treatment with an anti-PDx (either PD-1 or PD-L1) with or without platinum-based chemotherapy are eligible for enrollment in Part 3 of the study.

Patients received the recommended Phase 2 dose (RP2D) of 9.0 mg/kg AU-007 (administered IV Q2W) plus a single loading dose of 135,000 IU/kg aldesleukin (administered SC) as described in FIG. 2. Patients who remained clinically stable (per RECIST definition as previously elaborated) and do not experience DLTs or unacceptable toxicity during Cycle 1 continue to receive additional 8-week treatment cycles with AU-007 until discontinuation criteria are met. Tumor assessments were conducted on Study Day 56 of each cycle. Response was based on RECIST v1.1 as previously defined.

An additional dose or doses of 135,000 IU/kg aldesleukin were administered at Investigator and Sponsor discretion based on objective tumor findings and pharmacodynamic data in patients who were tolerating treatment and were clinically stable as follows:
1. Aldesleukin administered with each cycle (Q8W, Day 1 of cycle) until objective tumor shrinkage is observed on radiologic imaging or physical exam;
2. Aldesleukin administered with objective signs of worsening tumor growth kinetics: e.g., previously shrinking tumors becoming stable, or with new tumor growth.

Patients who are tolerating treatment and are clinically stable can receive an additional dose of IL-2 (Aldesleukin) on Day 1 of each cycle until objective tumor shrinkage is observed on radiologic imaging or physical exam. The investigator can also administer an additional dose of aldesleukin with objective signs of worsening tumor growth kinetics: e.g., previously shrinking tumors becoming stable, or with new tumor growth.

### Results

### Patients Receiving the RP2D Dose Plus an Additional Dose or Doses of 135,000 IU/Kg Aldesleukin (‘Boost IL-2 Dosing’)

### Patient AU08-0066

Patient AU08-0066 received an IL-2 boost of 135,000 IU/kg on Cycle 4 Day 1 (C4D1; 24 weeks since treatment initiation).

Patient AU08-0066 is a 56-year-old female with metastatic clear cell renal cell carcinoma, diagnosed 11 Apr. 2019 upon right radical nephrectomy, who enrolled in Arm 2B Expansion Cohort (9.0 mg/kg AU-007+135,000 IU/kg aldesleukin loading dose). Prior systemic therapies were adjuvant sunitinib May-November 2019 with progressive disease and 48 cycles of nivolumab 10 Dec. 2019-20 Nov. 2023 with progressive disease.

At screening for study CP-AU-007-01, three baseline target lesions (lung right lower lobe, and two bone lesions) were identified totaling 76 mm, and non-target lesions included axilla, mediastinal, and abdomen lymph nodes and a pancreas mass.

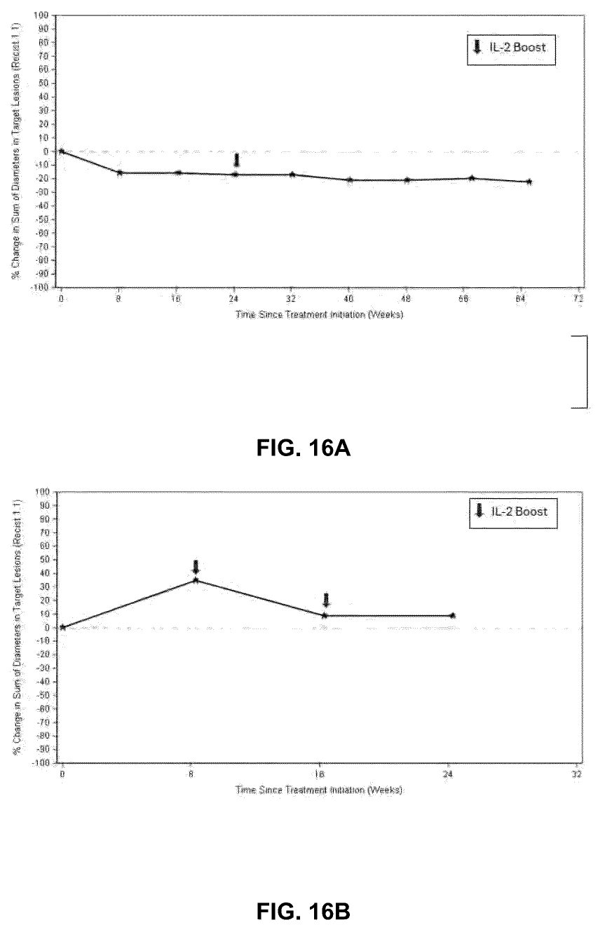

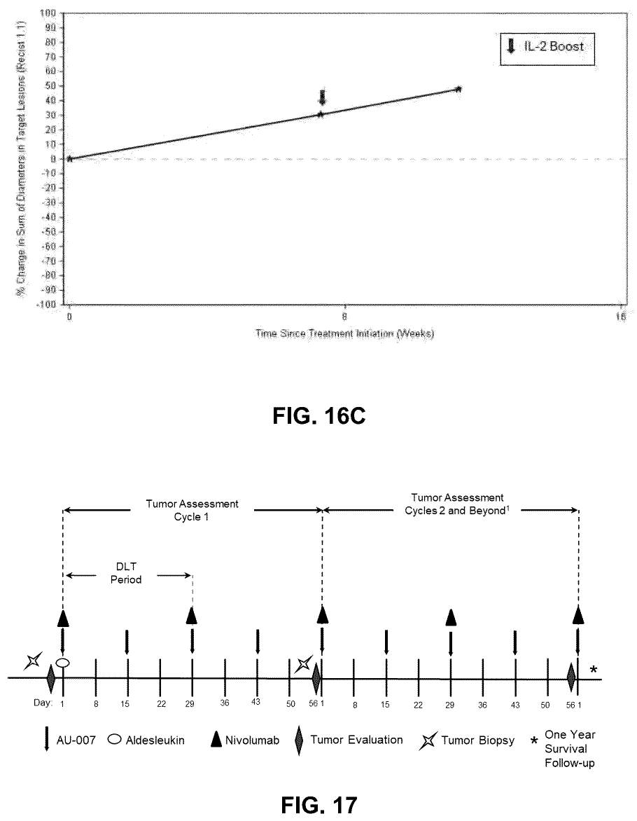

Cycle 1 Day 1 of AU-007+aldesleukin was administered 5 Mar. 2024 (FIG. 16A, Time 0). The patient had stable disease per RECIST at the end of Cycle 1 and Cycle 2 with a 16% decrease in target lesion volume (Table 8). At the end of Cycle 3, the target lesion volume had decreased 17% from baseline and the patient received an additional dose of 135,000 IU/kg aldesleukin on 20 Aug. 2024 (Cycle 4 Day 1; C4D1, arrow in FIG. 16A) to improve upon the response. Target lesions remained stable through Cycle 4 and further decreased by 21% at the end of Cycle 5 (26 Nov. 2024) and Cycle 6 (21 Jan. 2025); decreased by 20% at the end of Cycle 7 (25 Mar. 2025), 22% at the end of Cycle 8 (20 May 2025) (Table 8), and 24% at the end of Cycle 9 (15 Jul. 2025; data not presented in the FIG.). The patient continues on study with stable disease in Cycle 10.

TABLE 8
Target Tumor Volume (mm) at Baseline and End of Each 8-Week Cycle (% change from baseline)

| | | End of | End of | End of | End of | End of | End of | End of | End of |
|---|---|---|---|---|---|---|---|---|---|
| | Baseline* | Cycle 1 | Cycle 2 | Cycle 3** | Cycle 4 | Cycle 5 | Cycle 6 | Cycle 7 | Cycle 8 |
| Target tumor | 76 | 64 | 64 | 63 | 63 | 60 | 60 | 61 | 59 |
| volume (mm) | | | | | | | | | |
| % change | | (−16) | (−16) | (−17) | (−17) | (−21) | (−21) | (−20) | (−22) |
| from | | | | | | | | | |
| baseline | | | | | | | | | |

*1^{st} AU-007 + loading dose of aldesleukin administered
**Aldesleukin booster

### Patient US01-0098

Patient US01-0098 received IL-2 boosts of 135,000 IU/kg on Cycle 2 Day 1 (C2D1; 8 weeks since treatment initiation) and Cycle 3 Day 1 (C3D1; 16 weeks since treatment initiation).

Patient US01-0098 was a 46-year-old female with metastatic melanoma diagnosed 9 Sep. 2008 who enrolled in Arm 2B Expansion Cohort (9.0 mg/kg AU-007+135,000 IU/kg aldesleukin loading dose). Prior systemic therapy was 4 cycles of ipilimumab/nivolumab 6 Jun. 2024-21 Oct. 2024 with progressive or recurring disease.

At screening for study CP-AU-007-01, the patient had one target lesion (right inguinal lymph node) measuring 23 mm, and non-target lesions of the lung and left para-aortic, left common iliac, and left inguinal lymph nodes. Cycle 1 Day 1 of AU-007+aldesleukin was administered 25 Nov. 2024 (FIG. 16B, Time 0). The target tumor progressed (35% increase) at the end of Cycle 1 after which the patient received a first booster dose of 135,000 IU/kg aldesleukin. The target tumor was stable compared to baseline (9% increase) at the end of Cycle 2 (after which the patient received a 2^{nd} booster dose of 135,000 IU/kg aldesleukin) and Cycle 3 (Table 9; FIG. 16B). The patient continued to Cycle 4, receiving the final AU-007 dose on Cycle 4 Day 29, after which she discontinued treatment and was lost to follow-up for further evaluation.

TABLE 9
Target Tumor Volume (mm) at Baseline and End of
Each 8-Week Cycle (% change from baseline).

| | | End of | End of | End of |
|---|---|---|---|---|
| | Baseline* | Cycle 1** | Cycle 2** | Cycle 3 |
| Target tumor | 23 | 31 | 25 | 25 |
| volume (mm) | | | | |
| % change from | | (+35) | (+9) | (+9) |
| baseline | | | | |

*1^{st} AU-007 + loading dose of aldesleukin administered
**Aldesleukin booster administered

### Patient AU03-0084

Patient AU03-0084 received an IL-2 boost of 135,000 IU/kg on Cycle 2 Day 1 (C2D1; 8 weeks since treatment initiation).

Patient AU03-0084 was a 77-year-old female with metastatic melanoma, initially diagnosed with locally resectable disease in 1983 with first evidence of metastasis in December 2021, who enrolled in Arm 2B Expansion Cohort (9.0 mg/kg AU-007+135,000 IU/kg aldesleukin loading dose). Prior systemic therapies were neoadjuvant ipilimumab/nivolumab December 2021-May 2022 with a complete response, and lifirafenib/mirdametinib 2023-2024 with progressive or recurring disease.

At screening for study CP-AU-007-01, three target tumor lesions were identified (right upper breast, abdominal wall, mesenteric lymph node nodule) totaling 46 mm, and non-target lesions of right groin and presacral lymph nodes. Cycle 1 Day 1 of AU-007+aldesleukin was administered 30 Jul. 2024. The patient had progressive disease at the end of Cycle 1, with a 30% increase in target tumors (Table 10). The patient continued to Cycle 2 and received an aldesleukin boost of 135,000 IU/kg on Cycle 2 Day 1 (C2D1) (FIG. 16C). The patient discontinued with progressive disease on Cycle 2 Day 56.

TABLE 10
Target Tumor Volume (mm) at Baseline and End of Cycle 1
and Cycle 2 Day 15 (% change from baseline)*, **.
Target tumor volume (mm) at baseline, end of Cycle 1,
and Cycle 2 Day 15 (% change from baseline)

| | | |
|---|---|---|
| 46 | 60 | 68 |
| | (+30) | (+48) |

*1^{st} AU-007 + loading dose of aldesleukin administered
** Aldesleukin booster administered C2D1.

Data on additional patients that received an aldesleukin booster is presented in Table 11 and in more detail hereinbelow.

TABLE 11
Data on Additional Patients that Received an Aldesleukin Booster.

| | | AU-007 | IL-2 | Patient | Aldesleukin |
|---|---|---|---|---|---|
| | | dose and | dose and | Identifier | booster timing |
| Arm/Cohort | Tumor | schedule | schedule | Code | (cycle and day) |
| Arm 1B Cohort 2 | Bladder | 4.5 mg/kg | 45,000 | US03-0020 | C12D15 |
| Patient started at 4.5 | | Q2W | IU/kg | | 135,000 |
| mg/kg AU-007 and | | | | | IU/kg |
| increased to 9 mg/kg on | | | | | |
| Cycle 5 day 1. | | | | | |
| Arm 2C: switched to Arm | RCC | 9 mg/kg | 135,000 | AU03-0081 | C4D29, |
| 2B C3D1 and | | Q2W | Q2W | | C5D15 |
| discontinued Q2W IL-2. | | | | | |
| Arm 2C; switched to Arm | Melanoma | 9 mg/kg | 135,000 | US03-0073 | C4D1 |
| 2B C3D43 and | | Q2W | Q2W | | |
| discontinued Q2W IL-2. | | | | | |

### Patient US03-0020—Additional Data

Patient started at 4.5 mg/kg AU-007 and increased to 9 mg/kg on Cycle 5 day 1 and received a loading dose of 45K IU/kg; and a boost of 135K IU/kg in cycle 12, day 15. This patient had non-target disease only, meaning the tumor was not measurable per RECIST so the lesions were only noted and tracked as “present” or “absent” (Table 12).

TABLE 12
Tumor Data for Patient US03-0020

| Non-target | Urinary | Left Iliac |
|---|---|---|
| Lesions | Bladder | Lymph Nodes |
| Baseline | Present | Present |
| Cycle 1 | Present | Present |
| Day 56 | | |
| Cycle 2 | Present | Present |
| Day 56 | | |
| Cycle 3 | Present | Present |
| Day 56 | | |
| Cycle 4 | Present | Present |
| Day 56 | | |
| Cycle 5 | Present | Present |
| Day 56 | | |
| Cycle 6 | Present | Present |
| Day 56 | | |
| Cycle 7 | Present | Absent |
| Day 56 | | |
| Cycle 8 | Present | Absent |
| Day 56 | | |
| Cycle 9 | Present | Absent |
| Day 56 | | |
| Cycle 10 | Present | Absent |
| Day 56 | | |
| Cycle 11 | Present | Absent |
| Day 56 | | |
| Cycle 12 | Present | Absent |
| Day 56 | | |
| Cycle 13 | Present | Absent |
| Day 56 | | |
| Cycle 14 | Present | Absent |
| Day 56 | | |
| Cycle 15 | Data not | Data not |
| Day 56 | entered | entered |

At the end of Cycle 11, there was some concern for a slight increase in tumor burden per PET scan, and the ctDNA level was slightly increased. Boost administered 28 Jan. 2025 (Cycle 12 Day 15). The patient remains in stable disease, currently in Cycle 16.

ctDNA or circulating tumor DNA, are small fragments of DNA shed by tumors into the bloodstream. An increasing level of ctDNA during cancer treatment often predicts disease progression, treatment failure, and poor survival outcomes. Increased ctDNA may correlate with tumor burden, disease progression, and metastasis. High ctDNA levels can indicate residual disease or recurrence after treatment, and rising ctDNA can signal tumor growth or resistance to therapy. Monitoring ctDNA levels can provide valuable information on tumor burden and response to therapy, sometimes detecting progression earlier than traditional methods like imaging.

cTDNA Measurements:
Cycle 2 Day 1 (20 Jun. 2023): 0.00
Cycle 2 Day 29 (18 Jul. 2023): 0.05
Cycle 6 Day 1 (24 Jan. 2024): 0.08
Cycle 12 Day 1 (7 Jan. 2025): 0.12
Cycle 12 Day 43 (25 Feb. 2025): 0.68
Cycle 16 Day 15 (3 Sep. 2025): “negative” per investigator.

### Patient US03-0073—Additional Data

Patient US03-0073 switched to Arm 2B and discontinued Q2W IL-2 on Cycle 3 Day 43 (9 Oct. 2024), received an IL-2 boost on Cycle 4 Day 1 (23 Oct. 2024), and showed an increase in target tumor size and a new tumor at Cycle 4 Day 56 (17 Dec. 2024) (Table 13). The patient was discontinued with progressive disease at the end of Cycle 4.

TABLE 13
Tumor Data for Patient US03-0073

| | Total Target | Non-target |
|---|---|---|
| | Lesion Size (mm) | lesions (1) |
| Baseline | 39 | Present |
| Cycle 1 | 31 | Present |
| Day 56 | | |
| Cycle 2 | 30 | Present |
| Day 56 | | |
| Cycle 3 | 34 | Present |
| Day 56 | | |
| Cycle 4 | 38 (presence | Present |
| Day 56 | of new lesion | |
| | therefore | |
| | progressive | |
| | disease | |

### Patient AU03-0081—Additional Data

Patient AU03-0081 switched to Arm 2B and discontinued Q2W IL-2 on Cycle 3 Day 1 (21 Oct. 2024). The patient received IL-2 boosts on Cycle 4 Day 29 (13 Jan. 2025) and Cycle 5 Day 15 (26 Feb. 2025) with initial increasing tumor size, then showed a decrease in target tumor size on the Cycle 7 Day 56 scan (25 Jul. 2025) (Table 14); therefore, the boost administration may be considered beneficial. The patient remains on treatment in Cycle 8.

TABLE 14
Tumor Data for Patient AU03-0081

| | Total Target | Non-target |
|---|---|---|
| | Lesion Size (mm) | lesions (2) |
| Baseline | 69 | Present |
| Cycle 1 | 64 | Present |
| Day 56 | | |
| Cycle 2 | 70 | Present |
| Day 56 | | |
| Cycle 3 | 75 | Present |
| Day 56 | | |
| Cycle 4 | — | — |
| Day 56 | | |
| Cycle 5 | 79 | Present |
| Day 56 | | |
| Cycle 6 | 78 | Present |
| Day 56 | | |
| Cycle 7 | 66 | Present |
| Day 56 | | |
| Cycle 8 | Pending | |
| Day 56 | | |

These optional Aldesleukin boost administration (Phase 2B) studies remain ongoing.

### Example 6

AU-007 in Combination with Single-Dose Aldesleukin and Nivolumab

The assay below examined safety assessments conducted under the clinical protocol. Pharmacokinetics (PK) assays, and pharmacodynamic (PD) assays were carried out for the B regimen and C regimen to identify the doses to be used in Phase 2 Expansion Cohorts.

Patients with cutaneous melanoma with the following features are eligible for enrollment in Part 4 of the study, in which AU-007 is administered in combination with single-dose Aldesleukin and Nivolumab:
Unresectable locally advanced or metastatic cutaneous melanoma that has progressed during or following treatment with either PD-1 or PD-1. Unresectable locally advanced tumors indicate tumors that cannot be resected, and have spread locally (compared to metastatic tumors that has spread from the original location to another part of the body).
Patients with BRAF mutations must either be ineligible for or have refused a BRAF+MEK inhibitor.
Must have objective progression after receiving at least two cycles of prior doublet therapy (anti-PD-1/anti-CTLA-4 or anti-PD-1/anti-LAG-3). “Objective progression” is considered measurable/quantifiable, i.e., Response Evaluation Criteria in Solid Tumors (RECIST v1.1 definition of progression—see definition below).
Progressive Disease is defined as having at least a 20% increase in the sum of diameters of target lesions, taking as reference the smallest sum on study (this includes the baseline sum if that is the smallest on study). Progression is a >/=20% increase in the sum of the tumors compared to the smallest sum from any prior study scan. In addition to the relative increase of 20%, the sum must also demonstrate an absolute increase of at least 5 mm. The appearance of one or more new lesions is also considered progression.
Radiographic progression ≥4 weeks prior to the first dose of study drug to rule out late response to most recent therapy. The requirement for documented radiologic progression may be waived after review by Medical Monitor (e.g., in the case of progression beyond 12 weeks after starting a doublet). This study's baseline scan can serve to document confirmed progression to most recent therapy. The most recent therapy is the last therapy the patient received before starting this study.
Lactate dehydrogenase (LDH) ≤2.5× upper limit of normal

FIG. 17 provides a timeline of the treatment and tumor assessment cycles for Study 4, which examines the effect of combination treatment of AU-007, aldesleukin, and nivolumab. Cycles are 8 weeks (56 days). AU-007 was administered 4 times in a cycle (Day 1, 15, 29, 43—see black arrow); Aldesleukin was administered as a loading dose once in Cycle 1 (Day 1—see ellipse); and Nivolumab was administered twice in a cycle (Day 1 and 29—see black arrowhead).

For patients who continued to subsequent cycles, they began with AU-007 and Nivolumab on Cycle 2 Day 1, then AU-007 administered on Days 15, 29 (with Nivolumab), and 43 of Cycle 2; and so on.

Tumor evaluation (black diamond) is carried out at the end of each cycle. Tumor evaluation refers to imaging (CT scan, MRI, PET scan, or ultrasound) to measure tumors and assess for any new tumors—see RECIST definition herein).

Biopsy (FIG. 17, 4-point star) tissue was evaluated for the immune-modulating effect of the treatment within tumor by evaluating immune cell infiltration. Biopsy tissue was not used in determining a patient's response to treatment in conjunction with CT scans.

The Dose-Limiting Toxicity (DLT) period lasted 4 weeks and informed dose escalation decisions. All patients treated in the safety run-in were followed for 4 weeks and their safety assessed before moving to the next dose level.

### Safety Run-In Cohorts

For the combination therapy of: (i) AU-007; (ii) aldesleukin; and (iii) Nivolumab, an initial safety run-in of the combination therapy was conducted as a 3+3 dose escalation cohort (Table 15) in the Dose Limiting Toxicity (DLT) Evaluation Period (FIG. 17), in which the RP2D-1 doses were used (Cohort 1 in Table 15). In RP2D-1, 9 mg/kg AU-007 was administered IV Q2W, 45,000 IU/kg aldesleukin loading dose was administered SC, and 480 mg Nivolumab was administered IV.

TABLE 15
Part 4 Safety-run in Dose Levels.

Dose Evaluated

| | Aldesleukin | AU-007 | Nivolumab |
|---|---|---|---|
| Cohort | Loading Dose | Q2W | Q4W |
| (n) | (IU/kg) | (mg/kg) | (mg) |
| 1 | 45,000 | 9 | 480 |
| (3 + 3) | | | |
| RP2D − 1 + | | | |
| Nivolumab | | | |
| −1 | 15,000 | 9 | 480 |
| (3 + 3) | | | |
| RP2D − 2 + | | | |
| Nivolumab | | | |
| 2 | 135,000 | 9 | 480 |
| (3 + 3) | | | |
| RP2D + | | | |
| Nivolumab | | | |

When a DLT is observed during the ‘DLT evaluation period’ in any of the initial 3 patients enrolled, 3 patients were added. If two or more DLTs were observed in the initial 6 patients, then the RP2D-2 (Cohort −1) were evaluated in combination with Nivolumab-IL-2 loading dose is 15,000 IU/kg (RP2D-2, Table 15).

If no DLTs were observed at the RP2D-1 dose level, a cohort of 3 patients were evaluated at the RP2D+Nivolumab (Cohort 2) as described for the first cohort.

If no further DLTs were observed, then the Cohort Expansion proceeds with the AU-007+aldesleukin RP2D in combination with Nivolumab. The RP2D of AU-007 and aldesleukin was 9 mg/kg AU-007 (administered IV Q2W) and 135,000 IU/kg aldesleukin (administered SC). Nivolumab was administered IV at a dose of 480 mg at the time of the initial dose of AU-007 plus aldesleukin and then once every 4 weeks (Q4W).

Dose escalation decisions were driven by clinical safety and all available pharmacokinetic (PK) and pharmacodynamic (PD) data during the DLT Evaluation Period, defined as the time between the day of the initial dose up to the day of the 3^{rd} dose administration of AU-007 (planned Study Day 29 of Cycle 1; FIG. 17).

Enrollment of the Cohort Expansion was initiated at the dose determined from the safety run-in.

### Cohort Expansion

Patients enrolled in the Part 4 Cohort expansion received the dose determined from the safety run-in as described in FIG. 17.

Patients who remained clinically stable and did not experience DLTs or unacceptable toxicity during Cycle 1 continued to receive additional 8-week treatment cycles with AU-007 plus Nivolumab until discontinuation criteria as previously defined are met. Tumor assessments occurred on Study Day 56 of each cycle. Response was based on RECIST v1.1 as previously defined.

An additional dose or doses of 135,000 IU/kg aldesleukin was administered at Investigator and Sponsor discretion based on objective tumor findings and pharmacodynamic data in patients who were tolerating treatment and were clinically stable as follows (Protocol Section 3.4.1):
1. Aldesleukin administered with each cycle (Q8W, Day 1 of cycle) until objective tumor shrinkage is observed on radiologic imaging or physical exam,
2. Aldesleukin administered with objective signs of worsening tumor growth kinetics: e.g., previously shrinking tumors becoming stable, or with new tumor growth.
   Preliminary of Results of AU-007 in Combination with Single-Dose Aldesleukin and Nivolumab

Enrollment is currently ongoing in Cohort 1 (RP2D-1+Nivolumab)—3 patients are enrolled.
1 patient ongoing in Cycle 3 with a 2% increase in target lesions at the end of Cycle 1 and a 10% increase at the end of Cycle 2.
2 patients ongoing in Cycle 1 with end of Cycle tumor assessments set for 20 and 27 August.
   No patients enrolled in Part 4 of this study have received an aldeleukin booster to date.

### Example 7

Objective: Continued evaluation of anti-IL2 antibody clone BDG17.069 (AU-007) in subject with cancer. (Continued description and exemplification of the clinical trial presented in Example 1.)

### Study Design—Updates

This example provides updated data of studies described in Example 6 above and further describes studies (Clinical Phase 1/2 Trial) of the anti-IL-2 antibody in combination with IL-2 (single loading dose or co-administration). During the trial, pharmacodynamic markers in the periphery and tumor biopsies were/are being collected to investigate the activity of AU-007 or AU-007+IL-2. Provided here is data from the ongoing studies performed evaluating anti-IL-2 antibody clone BDG17.069 (AU-007) as (1A) a monotherapy, (1B) in combination with a single loading dose of aldesleukin (Aldesleukin is the generic name for the trade drug names Proleukin® (aldesleukin)®, and (1C) with both BDG17.069 and aldesleukin given once every 2 weeks (Q2W). AU-007 monotherapy evaluates doses sufficiently high to ensure enough AU-007 is available to bind all IL-2 molecules: both exogenously administered (aldesleukin) and endogenous IL-2. Aldesleukin is administered subcutaneously, at much lower doses and much less frequently than the approved IV regimen. Immunophenotyping of peripheral blood and the inflammatory cytokine interferon-gamma (IFN-γ) as well as peripheral eosinophils were examined, and preliminary results are updated here.

FIG. 1 presents an updated version of the Phase I Dose Escalation clinical trial scheme. Solid outline indicates studies in progress or completed. Dashed outline indicates studies just begun.) Recombinant human IL-2 (aldesleukin) was, is, and will be administered subcutaneously, at much lower doses and much less frequently than the approved regimen (600,000 IU/kg every 8 hours for 14 administrations) of intravenously administered aldesleukin.

Patients enrolled in this Phase 1/2 study include but are not limited to those with unresectable locally advanced or metastatic cancer. Patient inclusion criteria specifies adults 18 years old with any of 20 solid tumor histologies in dose escalation. In some cases the cancer has progressed. In some cases, the subjects are not eligible for treatment with standard/approved therapies. In some cases, use of known therapies with these patients has been unsuccessful in halting the cancer. The list of 20 solid tumor histologies is presented in Example 1.

As provided in Example 1, in addition to the 20 solid tumor histologies listed above, patient's entered into the trial may be suffering any of the solid tumors including a melanoma, a metastatic melanoma, a primary melanoma and metastatic melanoma, a renal cell carcinoma (RCC), a non-small cell lung cancer (NSCLC), a bladder cancer, a head and neck cancer, a head and neck squamous cell carcinoma (HNSCC), a nasopharyngeal carcinoma, a urothelial cancer, an adrenal cortical carcinoma, a clear cell renal cell carcinoma (ccRCC), a triple-negative breast cancer, a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a uterine cancer, a gallbladder cancer, or a Merkel cell carcinoma, or any tumors that are microsatellite instabilities (MSI)-high tumors. In some cases, the cancer may be unresectable locally advanced or metastatic cancer.

Adverse events were graded by the Common Terminology Criteria for Adverse Events (CTCAE). Efficacy evaluation was based on PD (Pharmacodynamic) markers of immune stimulation, total IL-2 (bound to AU-007+free IL-2) and objective responses.

Efficacy based on PD markers of immune stimulation, total IL-2 (bound to AU-007+free IL-2), and objective response.

Tumor assessments occur at the end of each 8-week cycle.

42 patients enrolled as of Oct. 13, 2023: Arm 1A: 15; Arm 1B: 12; Arm 1C: 15.

| Patient Demographics | | Tumor Histologies Evaluated in the Trial | |
|---|---|---|---|
| Patient Characteristics n = 42 | | Cancer Diagnosis (n, %) | |
| Mean age, years (range) | 63 (44-89) | Melanoma (includes 1 Uveal/1 Acral) | 10 (23.8) |
| Gender, n (%) | | Pancreatic cancer | 7 (16.7) |
| Male | 23 (55) | Head and neck squamous cell carcinoma | 5 (11.9) |
| Female | 19 (45) | Renal cell carcinoma | 5 (11.9) |
| Race, n (%) | | Colorectal cancer | 4 (9.5) |
| White | 34 (80.9) | Non-small cell lung cancer | 3 (7.1) |
| Asian | 3 (7.1) | Cutaneous squamous carcinoma | 3 (7.1) |
| Black | 2 (4.8) | Bladder cancer | 2 (4.8) |
| American Indian/Alaska native | 1 (2.4) | Other | 3 (7.1) |
| Other | 1 (2.4) | | |
| Unknown | 1 (2.4) | | |
| ECOG performance status, n (%) | | | |
| 0 | 22 (52) | | |
| 1 | 20 (48) | | |
| Mean number of prior | 3 (1-8) | | |
| therapies, n (range) | | | |

### Methods

Briefly, peripheral blood samples were taken prior to dosing on Day 1 and following dosing at 4 hours, and on Days 2, 3, 15, 29, 43 of cycle 1 and pre-dose/Day 29 on all other cycles. Dosing with AU-007 was done via intravenous dosing by weight-based dosing (mg/kg). Proleukin® (aldesleukin) was dosed subcutaneously by weight-based dose (IU/kg). Whole blood was stained for CD4+ T cells, CD8+ T cells, CD4+ Tregs, NK cells, B cells, and monocytes. Samples were analyzed by flow cytometry using TruCount™ for absolute cell counts. Changes from baseline values were determined off absolute counts. In addition, differential hematology counts were taken on Days 1, 15, and 29 of all cycles (or as needed per treating physician's decision) for safety evaluation and to examine eosinophil levels (eosinophils express the IL-2 trimeric receptor). Serum was taken prior to dosing on Day 1 and at 2 hours, 6 hours, and Days 2, 3, 15 pre/post, and 6 hours, 29 pre/post, 43 pre/post, and subsequent Day 1 of each cycle at pre/post. Samples were analyzed for INF-γ (LOQ 31 fg/ml), IL-2 (LOQ 31 fg/ml), and sCD25 (10 fg/ml) using the ECL Mesoscale Dynamics platform.

FIG. 1 graphically presents the administration and dosage schemes of BDG17.069 (AU-007)Q2W Monotherapy (left-1A dosages of 0.5 mg/kg, 1.5 mg/kg, 4.5 mg/kg, 9.0 mg/kg, and 12 mg/kg), BDG17.069 (AU-007)+a single IL-2 loading dose (center 1B dosages of 4.5 mg/kg AU-007+15K IU/Kg IL-2, 4.5 mg/kg AU-007+45K IU/Kg IL-2, 4.5 mg/kg AU-007+135K IU/Kg IL-2, and 4.5 mg/kg AU-007+270K IU/Kg IL-2), and combination therapy BDG17.069 (AU-007)+IL-2 (right 1C dosages of 4.5 mg/kg AU-007+15K IU/Kg IL-2, 4.5 mg/kg AU-007+45K IU/Kg IL-2, 4.5 mg/kg AU-007+135K IU/Kg IL-2, and 9.0 mg/kg AU-007+270K IU/Kg IL-2).

Recombinant human IL-2 (aldesleukin) was administered subcutaneously. Note that the doses and frequency are much lower and less frequent than the approved regimen of intravenously administered aldesleukin.

As described in Example 1, FIG. 1, the terms “3+3” and “1+2” refer to the size of the cohort at a given dose level. It means in the case of 3+3 that the first 3 enrolled patients were or will be administered BDG 17.069 at that dose level. If no dose-limiting toxicities (DLTs) are seen, then administration may escalate to the next highest dose level. However, if any of the first 3 patients do have a dose-limiting toxicity that is drug-related, then an additional three more patients need to receive treatment at that same dose level to see if they have DLTs before escalating. 1+2 follows the same principle—start with one patient, if DLTs are observed, add two more, or if no DLTs are observed in the first single patient, then dosages can be escalated. The trial continues to progress and the fourth dose escalation Cohort of Arm 1C has opened wherein AU-007 is being evaluated in the range of 4.5-12 mg/kg and with IL-2 at 270×10³ IU/kg. Future dose escalations of IL-2 up to about 500×10³ IU/kg may be evaluated.

The DLT evaluation period is the first 28 days of the 1^{st} cycle. Tumor assessment by computed tomography scan occurs with each 8-week cycle. The AU-007 and aldesleukin dose and schedule for Phase 2 expansion, will be based on safety, efficacy, pharmacokinetics (PK), and pharmacodynamics (PD).

Peripheral blood samples were taken at pre dose followed at 4 hours, and on days 2, 3, 15, 29, 43, of cycle 1 and pre-dose/day 29 on all other cycles. Whole blood was stained for CD4+ T cells, CD8+ T cells, CD4+ Tregs, NK cells, B cells, and monocytes. In addition, differential hematology counts were taken on days 1, 15 and 29 of all cycles (or ad hoc as per treating physician's needs) to examine eosinophil levels. Serum was taken at pre dose and at 2 hrs, 6 hrs and days 2, 3, 15 pre/post and 6 hours, 29 pre/post, 43 pre/post, and subsequent day one of each cycle at pre/post. Samples were analyzed for INF-gamma (LOQ 31 fg/ml), IL-2 (LOQ 31 fg/ml), and sCD25 (10 fg/ml) using the ECL Mesoscale Dynamics platform.

### Results:

FIG. 18 presents updated data from 15 patients (as of Oct. 13, 2023) enrolled in 5 cohorts of Arm 1A (0.5 mg/kg, 1.5 mg/kg, 4.5 mg/kg, 9.0 mg/kg, and 12 mg/kg). For patients in cohorts 1-4, AU-007 was well tolerated with no DLTs and all treatment-related adverse effects were Grade 1 (FIG. 20A). For Arm 1A, a best response of stable disease was noted in several patients (FIG. 20A) and three patients continued treatment. Three (3) patients in Arm 1A received a single aldesleukin dose following progression: NSCLC, uterine leiomyosarcoma, and uveal melanoma. The NSCLC and leiomyosarcoma patients had tumor scans following aldesleukin administration and are evaluated with the AU-007+aldesleukin cohorts for efficacy. Other patients were discontinued with either objective progression or clinical progression.

FIG. 19A presents the current data from 12 patients enrolled in the 4 cohorts of Arm 1B (4.5 mg/kg AU-007+15K IU/kg IL-2 or 45K IU/kg IL-2 or 135 IU/kg IL-2 or 270 IU/kg IL-2). FIG. 19B presents the current data from 15 patients enrolled in the first 3 cohorts of Arm 1C (4.5 mg/kg AU-007+15K IU/kg IL-2 or 45K IU/kg IL-2 or 135 IU/kg IL-2). 27 total patients enrolled into Arms 1B and 1C: Q2W AU-007+1 loading dose of aldesleukin and Q2W AU-007+Q2W aldesleukin, respectively.

For the patients in the first 2 cohorts of Arm 1B and 2 cohorts of Arm 1C, AU-007 was well tolerated with no DLTs and all but one treatment-related adverse effects were Grade 1 (FIGS. 21A and 21B). All five patients continued treatment in Arm 1B, the two patients continued treatment in Arm 1C, and additional patients joined Arm 1B and Arm 1C. additional patients are targeted to join Arm 1C and are in screening.

A summary of all adverse effects for Arms 1A, 1B, and 1C is presented in FIG. 20B and FIG. 20C. All drug-related Adverse Events (AEs) were Grade 1 or 2 except for 3 patients receiving AU-007+aldesleukin with transient (3-7 days) Grade 3 or 4 lymphopenia that were not associated with adverse outcomes. Transient lymphopenia is a known effect of IL-2 treatment. No patients discontinued for a drug-related AE; no dose-limiting toxicities (DLTs) were observed.

FIGS. 22A-22C and FIGS. 23A and 23B present blots of percent change of tumor burden vs. baseline in view of treatment regime and tumor type (FIGS. 22A-22C) or over time (FIGS. 23A-23B). The clinical details indicate that two patients (Patient AU01-0009—NSCLC with No Response to Chemo+anti-PD-L1 (dosage AU-007 1.5 mg/kg; See below) and Patient AU06-0014—Melanoma with No Response to PD-1+CTLA-4 (dosage AU-007 4.5 mg/kg+15K IU/kg)) who were unresponsive to checkpoint inhibitor therapy, are not only continuing in the present trial but their tumors have shown a negative percent change from baseline (reduced size) Two patients (NSCLC and uterine leiomyosarcoma) began therapy on AU-007 monotherapy (Arm 1A) and received one aldesleukin dose, 15K and 45K IU/kg respectively, after having progressed (stars on the graph; FIG. 23B). The velocity of tumor growth decreased in both patients after receiving aldesleukin with AU-007.

At this point in early development (Oct. 13, 2023), the greatest anti-tumor activity is observed with AU-007 in combination with aldesleukin, in patients with tumors known to be sensitive to immune-modulating drugs. This subset of patients is shown in the waterfall plot where the G.I. cancers are excluded (FIG. 22C). A best response of stable disease was observed in 9 of 33 (27%) evaluable for response. Specifically, 1 patient with bladder cancer had Non-target lesions (NTL) disease only and has stable disease (SD) through 3 cycles, with tumor reduction observed after Cycle 1. In a melanoma patient, there was a 40% decrease in target tumors that had progressed/not responded when treated with anti-CTLA-4+anti-PD-1 therapy; brain metastasis noted after Cycle 2 and treated with radiation, and the patient continues on treatment.

Patient AU01-0009: NSCLC with No Response to Chemo+Anti-PD-L1

The patient Cancer History: 83 year old man diagnosed with stage 3 squamous NSCLC in 2020. In January 2021 he received Chemo+anti-PD-L1. In June 2021 progression of the disease was observed in the opposite lung, wherein a watch and wait strategy was taken. By July 2022 further progression in lungs was observed. This subject received was enrolled in Arm 1A and received his first dose of AU-007 (1.5 mg/kg) Aug. 15, 2022. Table 16 presents clinical details and tumor size measurements.

TABLE 16

| | Pre- | | 8 Week | 16 Week | 24 Week | 32 Week | 40 Week |
|---|---|---|---|---|---|---|---|
| Target | treatment | Baseline | Scan | Scan | Scan | Scan* | Scan |
| Lesions | Jul. 14, | Aug. 9, | Oct. 7, | Dec. 5, | Jan. 30, | Apr. 5, | Jun. 5, |
| (mm) | 2022 | 2022 | 2022 | 2022 | 2023 | 2023 | 2023 |
| LUL | 47 | 58 | 53 | 55 | 74 | 93 | 95 |
| Lung | | | | | | | |
| L | Not | 20 | 17 | 17 | 13 | 16 | 16 |
| Adrenal | mentioned | | | | | | |
| R | Not | 23 | 17 | 16 | 15 | 17 | 16 |
| Adrenal | mentioned | | | | | | |
| Sum | 47 | 101 | 87 | 88 | 102 | 126 | 127 |

Progressive disease was noted due to the earlier positive trend and in consultation with medical staff, this patient received: 1 aldesleukin dose (15K IU/kg) at Week 32 and AU-007 dose was increased to 4.5 mg/kg.

Patient AU06-0014: Melanoma with No Response to Anti-PD-1/CTLA4; Significant Shrinkage in Two Lesions

The patient Cancer History: 2020, diagnosed with stage 3C melanoma: BRAF/NRAS WT

On July 2020, L arm lesion excised with lymph node biopsy. Between August 2020-April 2021 9 cycles adjuvant Nivolumab (anti-PD-1) were administered. On April 2021 recurrence L axillary node was noted and on May 2021 the axillary LN was excised. Between October-November 2021 the subject received adjuvant radiation and between September-November 2022 the patient received 4 Cycles Ipilimumab (anti-CTLA4)+Nivolumab (anti-PD-1). December 2022 progressive disease (PD) in the liver.

This patient was enrolled in Arm 1B and received a first dose of AU-007 (4.5 mg/kg)+Proleukin® (aldesleukin) (15K IU/kg) on Feb. 6, 2023. Table 17 presents clinical details and tumor size measurements.

TABLE 17

| | | 8 Week | 16 Week | 24 Week |
|---|---|---|---|---|
| | Baseline | Scan | Scan | Scan |
| | Jan. 17, | Apr. 3, | May 29, | Jul. 24, |
| Target Lesions | 2023 | 2023 | 2023 | 2023 |
| Liver Seg VII | 34 | 19 | 13 | 8 |
| Liver Seg IVA-B | 32 | 18 | 16 | 11 |
| LN-Portacaval | 17 | 34 | 33 | 31 |
| Sum | 83 | 71 | 62 | 50 |
| | | −14% | −25% | −40% |

The increase in lymph node (LN) may be driven by T cell infiltration, while a 14% total decrease in target lesions was observed.

FIGS. 24A, 24B, and 24C present the Baseline and 8-week scans. Portacaval lymph node: initial growth (with development of necrotic center) followed by stabilization may represent lymphocytic tumor infiltration (pseudo progression). Considering the lack of response to the former therapies, the scans showing 40% shrinkage in the target lesions of this melanoma patient demonstrate a significant improvement and therapeutic outcome. A new brain lesion was noted at the end of the 2nd cycle (16 weeks) and treated with radiation therapy. The patient continues on treatment.

Renal Cell Carcinoma (RCC) Patient, 68-Year-Old Man, Who Progressed on Prior Anti-PD-1 Treatment with Objective Progression in Mediastinal and Hilar LNs, June 2022.

Treatment of this RCC patient was initiated July 2023 at AU-007 (4.5 mg/kg)+15K IU/kg Q2W aldesleukin doses. Rapid shrinkage in the lymph node (LN) lesions was observed on initial scan (8 weeks). Comparison of baseline and 8-week scans showed 20% shrinkage in the Target Lesions (data not shown). The primary renal cancer remains in situ and was stable with no change in dimensions on the 8-week scan (not shown). A new cervical bone metastasis was noted at the end of the 1st cycle and will be surgically excised. The patient will continue on treatment following surgery.

### AU007 Pharmacokinetic (PK) Data Demonstrates IgG1 Therapeutic Characteristics

FIG. 25 presents PK data showing the pharmacokinetic data of patients based on dosage and regime. This data continues to demonstrate AU-007 concentration is dose proportionality and has PK characteristics similar to a standard IgG1 therapeutic human monoclonal antibody. Thus, AU-007 demonstrates low potential for immunogenicity.

Table 18 presents Cmax and step close to that predicted.

TABLE 18

| | est Cmax | | | Calculated |
|---|---|---|---|---|
| Dose | (70 kg) | Step | Actual Cmax | Step |
| 0.5 | 14 ug/ml | | 9.9 +/− 3 | |
| 1.5 | 42 ug/ml | 3 | 28.7 +/− 4 | 2.89 |
| 4.5 | 126 ug/ml | 3 | 91.9 +/− 29 | 3.2 |
| 9 | 252 ug/ml | 2 | 187.8 +/− 6 | 2 |

Table 19 presents data showing AU-007 PK and IL-2 Coverage (For Binding and Redirecting to Dimeric Receptors on Effector Cells)

TABLE 19

| | | | | Coverage of |
|---|---|---|---|---|
| | | | | Highest IL-2 Dose |
| AU-007 | | Serum | | (Proleukin ® |
| Dose | | AU-007 | Serum IL-2 | (aldesleukin) 270 |
| mg/kg | Time Point | ug/ml | Coverage pM | IU/kg) |
| 0.5 | Initial Peak | 12 | 82192 | 205 X |
| | Initial Trough | 4 | 27397 | 68 X |
| | Steady State | 20 | 136986 | 342 X |
| 1.5 | Initial Peak | 29 | 198630 | 496 X |
| | Initial Trough | 12 | 82192 | 205 X |
| | Steady State | 55 | 376712 | 941 X |
| 4.5 | Initial Peak | 110 | 753425 | 1883 X |
| | 50 hours | 90 | 616438 | 1541 X |
| | Steady State | | Data not yet available | Data not yet available |
| 9 | Initial Peak | 200 | 1369863 | 3424 X |
| | 50 hours | 150 | 1027397 | 2568 X |
| | Steady State | | Data not yet available | Data not yet available |
| 12 | Initial Peak | | Data not yet available | Data not yet available |
| | 50 hours | | Data not yet available | Data not yet available |
| | Steady State | | Data not yet available | Data not yet available |

### AU-007 PD Continues to Demonstrate Decrease in Tregs

FIGS. 26A-26B, 28A-28B, 29, 30A-30B, 31, 32A-32B, and 33-33B graphically present PD data showing the effect of AU-007 and the mechanism of action, including percent change in the absolute number of circulating regulatory T cells. Regulatory T cells were defined as CD3+CD4+CD25+CD127lo CD45+ cells. Consistent with the mechanism of action of inhibiting IL-2 from interacting with the trimeric receptor, regulatory T cells decreased in the peripheral circulation. This was observed in both the monotherapy arm and the arms which also included aldesleukin (Proleukin®) and was consistent among patients. FIG. 26B: Cohorts receiving only AU-007; FIG. 27: Cohorts with both AU-007 and at least 1 dose of aldesleukin. Percent change in the absolute number of circulating (FIGS. 28B and 29) CD8+ T cells and (FIGS. 30B and 31) NK cells. Increases in circulating effector cells were observed over time with monotherapy and lowest doses of IL-2 showing lower levels of peripheral effector cell expansion. Expansions were observed the longer the patient stayed on study. This is consistent with the mechanism of action of AU-007 stabilizing low levels of IL-2 and the requirement to build to an activation threshold of IL-2. In the presence of higher levels of IL-2, both CD8 and NK cells increased higher and earlier. Higher doses of IL-2 are being further explored and results are expected to be consistent with the present findings.

FIGS. 26A-26B show flow cytometry characterization of circulating Tregs (CD3+CD4+CD25+CD127 dim, Foxp3+) demonstrating an overall trend toward decreasing percentage of circulating Tregs in patients as measured either individually (FIG. 26A) or by group averages (FIG. 26B) for all dosed groups. FIG. 27 shows changes in peripheral blood Tregs for cohorts receiving AU-007+Proleukin® (aldesleukin). FIGS. 28A and 28B show the changes in absolute peripheral blood CD8 cells per individual patient (FIG. 28A) or based on group averages based on dose and regime (FIG. 28B). FIG. 29 presents the change in peripheral blood CD8 for cohorts receiving AU-007+Proleukin® (aldesleukin). When comparing this data to knowledge in the art is known that IL-2 causes initial dose dependent decreases in lymphocytes (likely migration into tissue) (Todd et al. (2016) Regulatory T Cell Responses in Participants with Type 1 Diabetes after a Single Dose of Interleukin2: A Non-Randomised, Open Label, Adaptive Dose Finding Trial. PLoS Med 13(10): e1002139.) FIGS. 30A and 30B show changes in Absolute Peripheral Blood NK cells per individual patient (FIG. 30A) or as a group average based on dose and regime (FIG. 30B), while FIG. 31 shows change in peripheral blood NK cells for cohorts receiving AU-007+Proleukin® (aldesleukin). As demonstrated in FIGS. 28A, 28B, 29, 30A, 30B, and 31, increases in circulating effector cells were observed over time with monotherapy and lowest doses of IL-2 showing lower levels of peripheral effector cell expansion. Expansions were observed the longer the patient stayed on study. This is consistent with the mechanism of action of AU-007 stabilizing low levels of IL-2 and the requirement to build to an activation threshold of IL-2. In the presence of higher levels of IL-2, both CD8+ T cells and NK cells increased higher and earlier. Higher doses of IL-2 are being further explored and results will be reported at a later date.

FIGS. 32A and 32B show that even though eosinophils express the same trimeric, CD25+ IL-2 receptor as Tregs and vascular endothelium, no increase in the absolute eosinophil counts were observed (FIG. 32A—all available data; FIG. 32B AU-007 and Proleukin® (aldesleukin) dosed patients), with patients having decreasing trends.

FIGS. 33A-33C show change in the ratio of CD8+ T cells to Tregs observed in the periphery, wherein the decrease in Tregs and changes in CD8+ T cells resulted in an increase in the CD8/Treg ratio when measured per patient (FIG. 33A), by dose group averages (FIG. 33B), or in patients receiving Proleukin® (aldesleukin) (FIG. 33C). Consistent with the observations observed with the changes in Tregs and CD8s, there is an observed trend to an increase in the CD8+/Treg ratio with monotherapy. In the presence of aldesleukin, an increase in the CD8+/Treg ratio was observed, particularly at higher doses of aldesleukin. Consistent with the mechanism of action, higher doses (of low dose IL2) and longer exposure trend to higher CD8/Treg ratios with no observed drug related toxicity. It is anticipated that increasing doses of aldesleukin will further enhance the peripheral response.

FIGS. 34A and 34B are heat maps showing the changes in circulating interferon gamma (IFN-g). Changes greater than 5-fold are represented by a solid box. FIG. 34A are patients receiving AU-007 and FIG. 34B are patients receiving AU-007 and Proleukin® (aldesleukin). Overall, the longer patients stay on trial or higher doses of AU-007, or addition of Proleukin® (aldesleukin) appears to be associated with more circulating IFN-g.

### Summary and Conclusion

Ongoing PK data demonstrates dose proportional AU-007 serum concentrations with typical characteristics of an IgG1 human mAb. Current available PD data demonstrates overall decreasing Tregs (% change and absolute) and eosinophils, which both express the trimeric IL-2 receptor. AU-007 monotherapy at AU-007 doses up to 9.0 mg/kg and combination therapy (with IL-2 low dose up to 45K IU/kg) is safe and well tolerated, with initial signs of immune modulation consistent with AU-007's mechanism of action. The clinical trial is continuing with higher doses of AU-007 up to 12 mg/kg, and IL-2 up to 270K IU/kg, with similar positive results expected.

This preliminary pharmacodynamic data from the AU-007 Phase 1 trial in multiple solid tumors show treatment with AU-007 as monotherapy or in combination with Proleukin® induces changes consistent with the mechanism of action (i.e., redirecting of IL-2 away from regulatory T cells and toward effectors) and consistent with an increase in the overall inflammatory profile of patients. The overall effect increases with time on therapy or with the addition of higher levels of Proleukin®. These changes were not associated with any drug-related events (data not shown). AU-007 treatment, with or without Proleukin®, led to decreases in eosinophils. Proleukin® administered as a single agent is known to raise eosinophil counts, and such increases have been associated with an adverse event profile. No changes were observed in circulating sCD25 levels (data not shown). IL-2 levels are still being investigated.

The preliminary data presented here support the overall mechanism of action for AU-007. The observed decreases in circulating regulatory T cells and increases in IFN-γ in the presence of the low doses of IL-2 assessed in this trial administered with AU-007 counters what is typically observed if low doses of IL-2 are given in the absence of AU-007. Low dose IL-2 given as a single agent increases Tregs and decreases IFN-γ in circulation and hence is being investigated as a treatment for autoimmune diseases. Proleukin® given as a single agent also has a well-characterized adverse event of increasing circulating levels of eosinophils, and lung toxicity can ensue from the eosinophilia. IL-2 interacting with the trimeric receptor on the eosinophils is thought to be the major contributor of this increase. Here, again consistent with the mechanism of action of AU-007 redirecting IL-2 away from the trimeric receptor, AU-007 or AU-007+Proleukin® produce decreases in circulating levels of eosinophils, supporting the proposed activity of the antibody. While the overall number of patients per group is small and each cohort has multiple cancer types, the trends observed here with AU-007 and increasing Proleukin® doses demonstrate favorable pharmacodynamic effects, including reductions in regulatory T cells, increases in CD8+ T cells and NK cells, increases in CD8+/Treg ratios, and increases in IFN-γ, in a broad range of cancer types. Further investigations with higher doses of Proleukin® administered with AU-007 are currently being investigated. Efficacy and safety data for the study are to be reported at a later date.

The ongoing investigation shows that AU-007 Q2W has a tolerable and manageable safety profile as a monotherapy evaluated up to 12 mg/kg, in combination with one aldesleukin loading dose evaluated up to 4.5 mg/kg AU-007+270K IU/kg aldesleukin, and in combination with aldesleukin Q2W evaluated up to 4.5K mg/kg AU-007+135K IU/kg aldesleukin.

Preliminary evidence of anti-tumor activity has now been observed in multiple heavily pre-treated patients, including progression through checkpoint inhibitors in patients with melanoma (anti-PD-1/CTLA-4), RCC (anti-PD-1), and NSCLC (anti-PD-1).

Trends toward decreasing Treg cells with concordant increases in CD8:Treg ratio, initial interferon-gamma (IFN-γ) increases, and absolute eosinophil decreases are consistent with the novel mechanism of action of AU-007.

Enrollment continued in Arm 1C (4.5 mg/kg AU-007+270K IU/kg aldesleukin Q2W). Further development of AU-007 continued in combination with aldesleukin, and Phase 2 expansion cohorts are planned at least in melanoma, RCC, and NSCLC patients.

### Example 8

### Phase 1 Dose Escalation Portion of Ongoing Phase 1/2 Clinical Trial of AU-007

Objective: Continued evaluation of anti-IL2 antibody clone BDG17.069 (AU-007) in subjects with cancer.

### Study Design—Updates

This example provides updated data of the single dose (1B) and dose escalation (1C) studies of the Clinical Phase 1/2 Trial of the anti-IL-2 antibody in combination with IL-2, described in Example 7. During the trial, pharmacodynamic markers in the periphery and tumor biopsies were collected to investigate the activity of AU-007 or AU-007+IL-2. Provided here is data from the ongoing studies performed, evaluating anti-IL-2 antibody clone BDG17.069 (AU-007) with both BDG17.069 and aldesleukin (FIG. 35; Arms 1B and 1C).

Recombinant human IL-2 (aldesleukin) was/and is being administered subcutaneously, at much lower doses and much less frequently than the approved regimen (approved regimen: 600,000 IU/kg every 8 hours for up to 14 administrations) of intravenously administered aldesleukin. In these studies, human IL-2 (aldesleukin) was/and is being administered subcutaneously either as a single dose or every two weeks, at the doses shown in FIG. 35 i.e., at 15K IU/kg, 45K IU/kg, 135K IU/kg, or 270K IU/kg,

### Results:

FIG. 36 presents an updated AU-007+Aldesleukin Waterfall Plot: Best % Change vs. Baseline. FIG. 37 presents an updated AU-007+Aldesleukin Spider Plot: % Change vs. Baseline Over Time. FIG. 38 presents an updated AU-007+Aldesleukin Waterfall Plot: Best % Change in non-G.I. Immune Sensitive Tumors (RCC, NSCLC, HNSCC, Acral Melanoma, Uterine-lyo (Leiomyosarcoma in the uterus), Bladder, Nasopharyngeal, and melanoma).

As can be observed in FIGS. 36-38, significant anti-tumor activity is observed with AU-007 in combination with aldesleukin, in patients suffering from bladder cancer (AU-007+aldesleukin (1 Dose), NSCLC (AU-007+aldesleukin (1 Dose), melanoma (AU-007+aldesleukin (1 Dose), RCC (AU-007+aldesleukin (Q2W)), and nasopharyngeal cancer (AU-007+aldesleukin (Q2W)). The subject suffering from RCC patient had to terminate the study secondary to complications due to the underlying cancer, necessitating rapid surgical intervention due to a lesion that was threatening to impinge on his spinal cord. At the time of his having to leave the study, there was ~20% tumor shrinkage. In particular, FIG. 37 shows a greater than 30% reduction of cancer (reduction of tumor size) in a nasopharyngeal patient, while FIGS. 37 and 38 show that the patient suffering from melanoma has around a 40% tumor shrinkage.

Computed tomography scans of tumor lesions at baseline 24-weeks show a 40% shrinkage in the target lesions of a melanoma patient whose tumors progressed through prior anti-PD-1+CTLA4 therapy (FIG. 39; See, Example 7 and FIGS. 24A-24C, update for Patient AU06-0014). The patient initially presented as a 62-year-old man with progression in the liver, December 2022. By February 2023, he entered the study wherein an initial Q2W AU-007 (4.5 mg/kg) dose+one (and only) 15K IU/kg aldesleukin dose were administered. Initial portacaval lymph node (LN) growth with necrotic center was followed by stabilization, which may represent pseudoprogression.

Scans of tumor lesions at based line and 20-weeks in an RCC patient show 20% shrinkage in the first 8 weeks in the target lesions. The RCC Patient's tumors has previously progressed through prior Anti-PD-1 therapy (FIG. 40; See, Example 7, update for RCC Patient). The patient initially presented as a 68-year-old man who had progressed on anti-PD-1 treatment June 2022. July 2023, he entered the study with initial AU-007 (4.5 mg/kg)+15K IU/kg Q2W aldesleukin doses. The primary renal cancer remains in situ and was stable.

Summary: With the continued positive trends, these studies are continuing. Anti-tumor activity has been observed in heavily pre-treated patients with several types of cancer. Further development of AU-007 therapy in combination with aldesleukin (IL-2), and Phase 2 expansion cohorts are planned at least in patients suffering from melanoma, RCC, and NSCLC.

### Example 9

Objective: Continued Evaluation of Anti-IL2 Antibody Clone BDG17.069 (AU-007) in Subject with Cancer.

### Study Design—Updates

This example provides updated results of the phase 1/2 study of AU-007, a monoclonal antibody (mAb) that binds to IL-2 and inhibits CD25 binding, in patients (pts) with advanced solid tumors. This example provides updates of studies described at least in Examples 7 and 8.

As presented previously, AU-007 is a human mAb that binds IL-2 on its CD25 binding epitope. AU-007 bound IL-2 cannot bind trimeric (CD25, CD122, CD132) IL-2 receptors (IL-2R) on regulatory T cells (Tregs), vascular endothelium, or eosinophils, but IL-2's binding to dimeric IL-2R (CD122, CD132) on T effector (T eff) and NK cells is unhindered. AU-007 thus redirects IL-2 towards T eff and NK cell activation, while diminishing Treg activation and vascular leak. AU-007 uniquely redirects IL-2 generated from T eff cell expansion, converting a Treg-mediated autoinhibitory loop into an immune stimulating loop.

Methods: As described previously, the study consists of 3 dose escalation arms followed by cohort expansion. Arm 1A evaluates escalating doses (0.5-12 mg/kg) of AU-007 (IV once every 2 weeks [Q2W]). Arm 1B evaluates AU-007 Q2W+escalating low doses (15K-270K IU/kg) of 1 aldesleukin subcutaneous (SC) dose. Arm 1C evaluates AU-007+escalating low doses of SC aldesleukin, both Q2W. There are at least nineteen solid tumor types allowed in escalation. Cohort expansion Arm 2B evaluates 9 mg/kg AU-007+one 135K IU/kg aldesleukin dose. Tumor assessments occur with each 8-week cycle.

### Results:

Fifty-three pts were enrolled as of 23 Jan. 2024: 15 in Arm 1A, 12 in Arm 1B, 25 in Arm 1C, and 1 in cohort expansion. AU-007 (+/−aldesleukin) was well-tolerated, with no dose limiting toxicities through all Arm 1A and 1B cohorts and the 3rd cohort (of 4) in 1C. Enrollment is ongoing in the final planned Arm 1C cohort and in Arm 2B expansion.

All drug related adverse events (AE) were Grade 1 or 2 except for 4 pts with transient lymphopenia (Grade 3 and 4) and 1 pt with Grade 3 anemia. The most common drug-related AEs were pyrexia (18%), fatigue (16%), nausea (14%), lymphopenia (8%), and chills (6%). Two transient Grade 2 drug-related serious AEs occurred: pyrexia (Arm 1B pt) and cytokine release syndrome (Arm 1C pt); both pts continued therapy.

A confirmed partial response (32% decrease) occurred in a nasopharyngeal carcinoma pt in Arm 1C who had progressed on anti-PD-1 therapy. Tumor shrinkage occurred in non-small cell lung, bladder, head and neck, colorectal (CRC), and renal cancer. In the CRC pt, target lesion diameter measurements by CT scan showed 27% reduction after 8 weeks of treatment with AU-007+aldesleukin (IL-2). A melanoma pt (Arm 1B) who had progressed on anti-CTLA-4+anti-PD-1 therapy had a 48% decrease in target tumors; a small brain metastasis was found at week 16 and irradiated. The patient remains on treatment. A pt with microsatellite stable CRC had a 26% tumor size reduction after the first cycle (Arm 1C) and continues on trial. Serum Tregs and eosinophils decreased in pts while NK and CD8 cell counts trended upwards. The CD8:Treg ratio trends upward in all cohorts.

### Conclusion

The mild toxicity profile and promising early efficacy observed in dose escalation across multiple tumor types in heavily pretreated pts, along with initial pharmacodynamic data, warrant continued evaluation of AU-007+low dose aldesleukin in the Ph 2 expansion cohorts of the study.

### Example 10

AU-007, a Human Monoclonal Antibody (mAb) that Binds to IL-2 and Inhibits CD25 Binding, Plus Low-Dose Aldesleukin: Phase 2 Update on Melanoma and Non-Small Cell Lung Cancer (NSCLC)

Background: AU-007 is a monoclonal antibody (mAb) that binds to the interleukin-2 (IL-2) cytokine at an epitope inhibiting its interaction with CD25 on the IL-2 receptor while allowing engagement with the dimeric receptor complex, CD122/CD132. Thus, AU-007 bound IL-2 expands Teff and NK cell populations (dimeric IL-2R) without expanding Tregs (trimeric IL-2R).

### Methods

The study consists of Phase (Ph) 1 dose escalation and four ongoing Ph2 expansion cohorts evaluating 8-week cycles of the recommended Ph2 dose (RP2D) of 9 mg/kg AU-007 IV Q2W+one subcutaneous (SC) 135K IU/kg aldesleukin dose on Day 1. Ph2 Cohorts 1 and 4 evaluate melanoma at RP2D±nivolumab (+nivolumab not reported); Cohorts 2 and 3 evaluate PD-L1-positive (≥1%) NSCLC that has progressed on anti-PD-1/L1±chemotherapy at RP2D, avelumab.

### Results

Ninety-eight patients were enrolled as of 30 May 2025 in all cohorts. AU-007+aldesleukin was well-tolerated in Ph1 with no dose-limiting toxicity. Twelve melanoma patients enrolled in Ph2 at the RP2D and two melanoma patients received AU-007 and single dose aldesleukin below the RP2D in Ph1. Two melanoma patients refractory to anti-CTLA-4 and PD-1 therapy had tumor reductions of 48% (14 months on treatment) and 100% (continues treatment at 16 months). A melanoma patient refractory to anti-PD-1+relatlimab therapy had a 58% reduction and continues treatment at 13 months.

Early NSCLC anti-tumor activity was seen in a Ph1 patient who failed prior anti-PD-L1 therapy and received AU-007 Q2W monotherapy with a 14% tumor reduction. In Ph2, two NSCLC patients have received the RP2D; one patient is ongoing with stable disease (SD) at five months.

The most common treatment-related adverse events were Grade 1/2 fatigue, chills, pyrexia, infusion-related reaction, and nausea.

### Conclusions

AU-007+low-dose SC aldesleukin exhibits a manageable toxicity profile with deep tumor reductions and durability in melanoma, and early signs of activity in the more recently opened NSCLC cohorts. Ph2 cohorts evaluating melanoma (±nivolumab) and NSCLC are ongoing.

### Example 11

Imneskibart (AU-007), a Human Monoclonal Antibody (mAb) that Binds IL-2 and Prevents CD25 Binding+Low-Dose Subcutaneous IL-2: Phase 2 Update on CPI-Refractory Melanoma

Background: Treatments activating effector cells against tumors are undermined by an autoinhibitory loop caused by endogenous IL-2 secreted from activated effector T cells (Teffs). Advantageously, Imneskibart (AU-007) can transform the IL-2 negative feedback loop into a positive feedback loop. In contrast, re-engineered IL-2 therapeutics cannot address the negative feedback loop, resulting in endogenous IL-2 stimulating regulatory T cell (Treg) expansion, and limiting efficacy.

Methods: This is an ongoing Phase 1/2 open label dose escalation and expansion study—Phase 1 has been completed.

The recommended Phase 2 dose (RP2D) of imneskibart and low-dose subcutaneous (SQ) aldesleukin (IL-2), imneskibart 9 mg/kg of a subject's body weight every two weeks (Q2W) intravenously (IV)+SQ aldesleukin loading dose of 135K IU/kg of a subject's body weight, is/was currently being evaluated in Phase 2 expansion in at least patients with cutaneous melanoma and patients with NSCLC.

Cutaneous melanoma cohorts evaluate the RP2D regimen of imneskibart+SQ aldesleukin and the RP2D regimen plus nivolumab 480 mg once every 4 weeks (Q4W) with a safety run-in (RP2D-1, aldesleukin 45K IU/kg) followed by cohort expansion. This study is examining unresectable locally advanced or metastatic cutaneous melanoma (including acral melanoma). Subject must have objective progression after receiving at least two cycles of prior doublet therapy (anti-PD-1+anti-CTLA-4 or anti-PD-1+anti-LAG-3), and confirmation of radiographic progression ≥4 weeks prior to Cycle 1 Day 1, to rule out late response to the most recent therapy administered. Patients with BRAF mutations must either be ineligible for or have refused a BRAF+MEK inhibitor therapy.

NSCLC cohort evaluates the RP2D regimen of imneskibart+SQ aldesleukin with a safety run-in (RP2D-1, aldesleukin 45K IU/kg) followed by cohort expansion. NSCLC patient subjects have PD-L1 positive (PD-L1+) (tumor proportion score ≥1%) NSCLC, which has failed to beneficially respond to prior checkpoint inhibitor (CPI) therapy±chemotherapy.

Efficacy is/was based on pharmacodynamic (PD) markers of immune stimulation and objective response; tumor assessments occur at the end of each 8-week cycle. Melanoma and NSCLC patients can receive an additional SQ aldesleukin dose (135 IU/kg aldesleukin) at the end of each 8-week cycle based on tumor growth kinetics observed on end-of-cycle scans.

### Enrolling Phase 2 in Post-CPI-Doublet Failed Melanoma and Post-CPI+/− Chemo-Failed, PD-L1 Positive Non-Small Cell Lung Cancer

Melanoma and NSCLC patients were/are being administered RP2D of 9 mg/kg imneskibart IV Q2W+one 135K IU/kg SQ aldesleukin loading dose. A boosting IL-2 dosing (135K IU/kg SQ aldesleukin) may be provided if tumor size is unchanged or increasing.

Enrollment goals and status for melanoma cohorts: (1) RP2D Imneskibart+SQ Aldesleukin Post CPI Melanoma n=20; 13 patients enrolled/6 ongoing. (2) Nivolumab+RP2D Imneskibart+SQ Aldesleukin Post Doublet CPI Melanoma n=20; 3 patients enrolled in the safety run-in administering 45K IU/kg of subject's body weight aldesleukin—safety run-in completed, all 3 patients ongoing; 3 patients enrolled at the IL-2 RP2D: 135K IU/kg of subject's body weight aldesleukin; and 2 patients ongoing. Currently there are 5 ongoing patients in the nivolumab combination cohort, 3 at the RP2D-1 and 2 at the RP2D+nivolumab.

Enrollment goals and status for NSCLC cohort: RP2D Imneskibart+SQ Aldesleukin Post PD-L1+NSCLC n=10; 4 patients enrolled/1 ongoing.

Patients tolerating study drug without confirmed objective pharmacodynamics (PD) can receive additional cycles of study drug until any one of the following conditions are met:
Patient meets criteria for objective progression by modified RECIST v1.1, i.e., progression that is confirmed by a second scan after receiving one more cycle of AU-007*.
Occurrence of a DLT*.
Sponsor, Investigator, or regulatory agency terminates the study.
Withdrawal of patient due to an AE or SAE.
Patient experiences medical condition that requires treatment with prohibited concomitant medication.
Withdrawal of patient consent.
Completion of protocol-defined therapy.
Pregnancy.
   * Patients meeting these criteria but otherwise experiencing substantial clinical benefit with acceptable toxicity based on Investigator opinion may continue therapy on a case-by-case basis.

### Results

Clear Anti-Tumor Activity in Melanoma with Imneskibart+Low-Dose IL-2 without Checkpoint Inhibitor (CPI) in Phase 1 and 2

FIG. 41A presents the best response in the target lesions of melanoma patients who progressed on prior CPI therapy, now treated with Imneskibart+low dose subcutaneous (SQ) IL-2 (loading dose) (RP2D); n=14. The Y-axis of FIG. 41A represents the best change in target lesion measurements from baseline.

Patients with BRAF/MEK mutations were eligible only if not treated with prior BRAF/MEK inhibitor. Measurements having an asterisk within the “bar” are patients who progressed on prior doublet checkpoint inhibitors: anti-CTLA-4+anti-PD-1 and/or anti-PD-1+anti-Lag-3. The two phase 1 patients are indicated by the downward arrow (administered RP2D). The 6 patients continuing in the trial are indicated by a filled circle.

The spider plot of FIG. 41B presents the percentage change over time vs. baseline in the target lesions of melanoma patients who progressed on prior CPI therapy and now treated with imneskibart+low dose SQ IL-2 (loading dose); n=14. The blue downward arrows indicate administration of an IL-2 booster dose (6 patients).

The results shown in FIGS. 41A and 41B demonstrate a strong signal of anti-tumor activity with administration of imneskibart+SQ loading dose aldesleukin, with ongoing deep and durable tumor reductions in patients who had previously progressed after receiving doublet checkpoint inhibitor therapy. Three patients with the deepest tumor reductions in target lesions continued treatment beyond one year: −48% reduction (patient was on study for 13 months), −58% reduction (patient continues on study for 16+ months), and a complete response (−100% reduction; patient continues on study for 20+ months).

Early Signs of Anti-Tumor Activity in Melanoma Patients Who Progressed on Prior Doublet CPI Therapy Now Treated with Nivolumab+Imneskibart+Low Dose SQ IL-2

An initial safety run-in evaluating a lower IL-2 loading dose of 45K IU/kg aldesleukin+480 mg once every 4 weeks (Q4W) nivolumab+9 mg/kg Q2W imneskibart was completed and the RP2D aldesleukin dose of 135K IU/kg was/is now being administered in combination with nivolumab and imneskibart to all newly enrolled patients. To be eligible, patients must have objective progression after receiving at least two cycles of prior doublet CPI therapy (anti-CTLA-4 and/or anti-PD-1+anti-LAG-3). Radiographic progression must have been demonstrated 4 weeks prior to Cyle 1 Day 1, to rule out late response to most recent therapy.

No dose limiting toxicity (DLT) was observed in the safety run-in. Initial signs of anti-tumor activity in very early clinical data in the safety run-in with the lower aldesleukin dose (45K IU/kg) were observed (Data not shown).

The best objective responses for the patients receiving the RP2D are shown in FIGS. 42A and 42B. Two patients treated at the RP2D-1 showed an increase of 24% and 2%; two patients treated at the RP2D had stable disease with 0 change in tumor measurement, one of whom continues treatment. One patient treated at the RP2D-1 showed a decrease in the best objective response of 22%. Three patients are continuing in the study as indicated by the circle, and two patients received an IL-2 boost, indicated by the arrow.

Early Evidence of Activity with Imneskibart+IL-2 in Phase 2 in NSCLC Patients Who Failed Prior (Checkpoint Inhibitor) CPI±Chemotherapy

FIGS. 43A and 43B show initial signals of antitumor activity with AU-007+SQ loading dose of aldesleukin for NSCLC patients treated at the RP2D. The Y-axis of FIG. 43A represents the best change in target lesion measurements from baseline. The NSCLC patient showing −24% decrease in target lesion size (best change from baseline) is continuing on in the clinical trial.

Patients with Target Tumor Reduction Other than Melanoma

Table 20 presents the best objective response target lesions treated with imneskibart+SQ aldesleukin.

TABLE 20
Best Objective Response to HNSCC, CRC, Bladder, and RCC Target Lesions.*

| | | | Best | Number | Best | |
|---|---|---|---|---|---|---|
| | Dose/Regimen | Dose/Regimen | Response | of Prior | Objective | Time on |
| | Imneskibart | Aldesleukin | on Prior | Cancer | Response | Treatment |
| Tumor | (mg/kg) | (IU/kg) | CPI | Regimens | (% Decrease) | (Months) |
| HNSCC¹ | 4.5 Q2W | 45K Q2W | PR (anti- | 4 | −100% | 28.5+³ |
| | | | PD-1) | | (Confirmed | |
| | | | | | CR) | |
| CRC | 9.0 Q2W | 135K Q2W | Not | 3 | −27% | 3.3 |
| (MSS) | | | applicable | | | |
| Bladder | 4.5 Q2W | 45K loading | PD (anti- | 1 | Metabolic | 32.5+³ |
| | | dose | PD-L1) | | CR² and | |
| | | | | | ctDNA | |
| | | | | | negative | |
| Bladder | 4.5 Q2W | 270K loading | PD (anti- | 4 | −13% | 4.7 |
| | | dose | PD-1) | | | |
| RCC | 9.0 Q2W | 135K loading | PD (anti- | 2 | −24% | 20.5+³ |
| | | dose | PD-1) | | | |
| RCC | 9.0 Q2W | 135K loading | PD (anti- | 6 | −12% | 14+³ |
| | | dose | PD-1) | | | |
| RCC | 9.0 Q2W | 135K loading | Not | 0 | −46% | 16.5+³ |
| | | dose | applicable | | | |

*
¹Head and neck nasopharyngeal histology.
²Patient with non-target lesion disease only with highly metabolically active tumors at baseline on PET scan that became negative at Cycle 7 (14M on study)
³Patient continues on study therapy.

### Adverse Event Summary in Phase 1 and 2: Mild and Tolerable Profile

Table 21 and Table 22 provide a summary of adverse events for patients treated with AU-007+IL-2 or AU-007+IL-2+nivolumab.

TABLE 21
Low Rate of Drug Related SAEs and Grade 3/4 AEs

| | Imneskibart + IL-2 | Nivolumab Combo |
|---|---|---|
| Event (n, %) | N = 77 | N = 5 |
| Any AE | 72 (93) | 5 (100) |
| Drug-Related AEs | 63 (82) | 3 (60) |
| Drug-Related SAEs | 5 (6) | 1 (20) |
| CRS | 3 | 1 |
| Infusion-related reaction | 1 | 0 |
| Pyrexia | 1 | 0 |
| Adrenal insufficiency | 0 | 0 |
| Injection site rash | 1 | 0 |
| Drug-Related Grade 3 or | 11 (15) | 0 |
| 4 AEs | | |
| Lymphopenia | 6 | 0 |
| CRS | 1 | 0 |
| Anemia | 2 | 0 |
| Lipase elevation | 1 | 0 |
| Neutropenia | 1 | 0 |
| Maculopapular rash | 1 | 0 |
| Hypertension | 0 | 0 |
| Autoimmune encephalitis | 1 | 0 |

TABLE 22
Most Drug Related AEs Tolerable and Easily Managed
Drug-Related Adverse Events in >10% of Patients

| | Imneskibart + IL-2¹ | Nivolumab Combo² |
|---|---|---|
| Adverse Event (n, %) | N = 77 | N = 5 |
| Pyrexia | 19 (25) | 0 |
| Fatigue | 17 (22) | 0 |
| Chills | 16 (21) | 1 (20) |
| Infusion-related reaction | 11 (14) | 0 |
| Nausea | 10 (13) | 1 (20) |
| Injection site reaction | 9 (12) | 2 (40) |
| Cytokine release syndrome | 7 (9) | 2 (40) |
| Anemia | 6 (8) | 0 |
| Diarrhea | 2 (2) | 0 |
| Hypotension | 1 (1) | 0 |
| Fall | 0 | 1 (20) |
| Dermatitis acneiform | 0 | 1 (20) |

¹Imneskibart Q2W + IL-2 loading dose or IL-2 Q2W.
²Imneskibart Q2W + IL-2 loading dose + CPI (nivolumab)

Most drug-related adverse events (AEs) were mild, Grade 1 or 2, with imneskibart+SQ loading dose aldesleukin alone or in combination with nivolumab. The low rate of drug related Grade 3 and 4 AEs observed with imneskibart+SQ loading dose aldesleukin continues with mature data. The addition of nivolumab to imneskibart+SQ loading dose aldesleukin did not increase the rate of drug related Grade 3 and 4 AEs in the emerging data. The most common Grade 3 or 4 AE was transient (3-7 days) lymphopenia that was not associated with adverse outcomes in any patient. Transient lymphopenia is a known effect of IL-2 treatment as lymphocytes traffic out of blood and into tissue One patient receiving imneskibart+SQ loading dose aldesleukin experienced Grade 4 CRS. It was resolved without tocilizumab using steroids, IV fluids, and brief vascular pressor support. This patient was noted retrospectively to have subclinical elevated IL-6 (5κ) serum levels likely due to an active case of gout at baseline.

### Pharmacodynamic Profile: Durable Decreases in Tregs Cells and Corresponding Increases CD8/Treg Ratios in Melanoma

FIGS. 44A and 44B show the absolute change in cell count of Tregs per μl of blood (FIG. 44A) and CD8/Treg ratio (FIG. 44B) based on ratio of counts per μl of blood. The mean peripheral blood Treg absolute cell counts continued to decrease after dosing with imneskibart+aldesleukin (RP2D). The addition of nivolumab to imneskibart and one-third RP2D SQ aldesleukin dose (RP2D-1; 45K IU/kg for a 3-patient safety-in) did not affect the decrease in the peripheral Treg population in the emerging data (FIG. 44A).

Increases in the peripheral blood CD8/Treg ratio were observed with the mean increases exceeding 2-fold over baseline with imneskibart+aldesleukin. Early data demonstrates an increasing CD8/Treg ratio with the addition of nivolumab to imneskibart and SQ aldesleukin (45K IU/kg) (FIG. 44B). Updated data shows that imneskibart led to a decrease in peripheral Tregs and increase in CD8 cells leading to CD8/Treg ratio increases up to 2-3 fold over BL. Initial pharmacodynamic data demonstrate that decreases in Tregs and increases in the CD8+/Treg ratio observed with nivolumab in combination with imneskibart and aldesleukin are similar in magnitude to those observed with imneskibart and aldesleukin alone.

Higher Peripheral Blood CD8/Treg Ratio Associated with Better Efficacy Vs Low CD8/Treg Ratio in Melanoma Patients

Longer durations of overall survival (OS), progression free survival (PFS), and time on treatment were observed in melanoma patients who achieved at least a 2-fold increase over their baseline CD8/Treg ratio (FIGS. 45A, 45B, and 45C). FIG. 45A presents the time of patients on treatment over the time of the ongoing study; FIG. 45B presents the PFS over the time of the ongoing study; and FIG. 45C presents the OS over the time of the ongoing study.

### Conclusions

The data presented here is from an ongoing study (Phase 2 clinical Trial). The most recent results show that Imneskibart+low-dose SQ aldesleukin (loading dose) possess a unique mechanism of action by reducing Tregs while increasing natural killer cells (NK) and CD 8 cells and thus increasing the Teff/Treg ratio, resulting in clinically meaningful tumor regressions with a manageable safety profile (low incidence of drug-related Grade 3/4 adverse events); therefore treatment can be safely administered in an outpatient setting.

Melanoma patients with primary resistance to prior doublet immune checkpoint inhibitors had durable, deep tumor reductions when treated with imneskibart+low-dose SQ IL-2 without CPI. Emerging data in melanoma patients receiving imneskibart+low-dose aldesleukin and nivolumab demonstrates an early signal of activity and mild safety profile in melanoma patients who progressed on prior doublet checkpoint inhibitor therapy.

Pharmacodynamic data demonstrated the continued durable peripheral Treg decreases with imneskibart+low-dose IL-2. Significantly, higher CD8/Treg ratio is associated with longer OS, PFS and time on treatment.

Early signal of activity was observed in an NSCLC patient whose tumor progressed on prior anti-PD-1/L1 therapy±chemotherapy receiving imneskibart+low-dose aldesleukin.

In addition to the activity observed in melanoma and NSCLC, two long-lasting complete responses were achieved in patients with bladder cancer and head and neck cancer, whose tumors progressed on prior checkpoint inhibitors.

### Example 12

Imneskibart (AU-007), a Human Monoclonal Antibody (mAb) that Binds IL-2 and Prevents CD25 Binding+Low-Dose Subcutaneous IL-2±Nivolumab in Patients with CPI-Refractory Cutaneous Melanoma

Objective: Continued results from an ongoing Phase 1/2 Study.

This example provides updated results of the phase 1/2 study of AU-007 in patients (pts) with CPI-Refractory cutaneous melanoma. This example provides updates of studies described at least in Examples 7-11.

Background: Imneskibart (AU-007) redirects IL-2 to Effector T Cells (Teff)/Natural Killer Cells (NK) and away from regulatory T Cells (Tregs) and Vascular Endothelium. Imneskibart is a human IgG1 monoclonal antibody designed by leveraging artificial intelligence. Imneskibart binds interleukin-2 (IL-2) with pM affinity and completely inhibits its binding to CD25, without hindering its binding to CD122/CD132.

### Study Design:

This is a Phase 1/2 open label dose escalation and expansion study. Enrollment is complete in Phase 2 evaluating imneskibart 9 mg/kg every two weeks (Q2W) intravenously (IV)+low-dose, SC aldesleukin (IL-2) loading dose of 135K IU/kg±checkpoint inhibitor (CPI) in patients with cutaneous melanoma and non-small cell lung cancer (NSCLC). A safety run-in cohort evaluated an aldesleukin loading dose of 45K IU/kg plus imneskibart and CPI as defined above.

The ongoing melanoma cohort evaluates imneskibart+loading dose SC aldesleukin as defined above and nivolumab 480 mg once every 4 weeks (04W).
Unresectable locally advanced or metastatic cutaneous melanoma (including acral melanoma).
Must have objective progression after receiving at least two cycles of prior doublet therapy (anti-PD-1+anti-CTLA-4 or anti-PD-1+anti-LAG-3).
Confirmation of radiographic progression 4 weeks prior to the first dose of study drug to rule out late response to most recent therapy.
Patients with BRAF mutations must either be ineligible for or have refused a BRAF+MEK inhibitor.
Efficacy is based on pharmacodynamic (PD) markers of immune stimulation and objective response based on Response Evaluation Criteria in Solid Tumors (RECIST v1.1); tumor assessments occur at the end of each cycle. Patients can receive an additional SC aldesleukin dose at the end of each cycle based on tumor growth kinetics observed on end-of-cycle scans.
Patients are treated in 8-week cycles and may continue additional cycles until discontinuation criteria as defined above are met.

Boost IL-2 dosing on Day 1 of each Cycle (Q8W) is allowed if tumor size is unchanged or increasing or if objective response not yet attained, i.e. if a patient has not met REQIST criteria for partial response (>/=30% decrease in target lesions) or complete response (100% decrease in target lesions).

Patient enrollment status: Phase 2 melanoma expansion cohorts: the imneskibart+SC aldesleukin cohort in patients with post CPI melanoma includes n=15, of which 14 were evaluable for efficacy. The nivolumab and imneskibart+SC aldesleukin cohort in patients with post doublet CPI melanoma includes n=21.

Table 23 presents melanoma patient characteristics.

TABLE 23
Melanoma Patient Characteristics

| | Imneskibart + | Nivolumab | |
|---|---|---|---|
| | IL-2 | Combo | Total |
| Patient Characteristics | N = 31* | N = 17 | N = 48 |
| Mean age, years (range) | 64.1 (34-89) | 64.3 (28-86) | 64.2 (28-89) |
| Gender, n (%) | | | |
| Male | 15 (48.4) | 15 (88.2) | 30 (62.5) |
| Female | 16 (51.6) | 2 (11.8) | 18 (37.5) |
| Race, n (%) | 29 (94) | 15 (88) | 44 (92) |
| White | 1 (3) | 0 | 1 (2) |
| Black | 0 | 0 | 0 |
| Asian | 1 (3) | 0 | 1 (2) |
| American Indian/Alaska native | 0 | 1 | 1 (2) |
| Other | 0 | 1 | 1 (2) |
| Not reported/missing | | | |
| ECOG performance | | | |
| Status, n (%)** | | | |
| 0 | 12 (38.7) | 10 (58.8) | 22 (45.8) |
| 1 | 19 (61.3) | 7 (41.2) | 26 (54.2) |
| Mean number of prior | 2.5 (1-7) | 2.7 (1-6) | 2.5 (1-7) |
| therapies n (range) | | | |

*Includes Phase 1 melanoma patients
Data cutoff as of Apr. 6, 2026
**ECOG Performance Status refers to a standardized clinical scale developed by the Eastern Cooperative Oncology Group for assessing a subject's level of functioning, including the ability to perform daily activities, self-care, and physical tasks. The ECOG Performance Status provides a numerical score that reflects the extent to which a disease affects the subject's daily living abilities and overall functional capacity. The ECOG Performance Status is typically measured on a discrete scale from 0 to 5, wherein lower scores indicate higher levels of functioning and higher scores indicate greater functional impairment. The ECOG Performance Status is widely used in clinical practice and clinical trials to characterize patient populations, monitor changes in functional status over time, and assess suitability for therapeutic interventions. Performance Status is defined as follows: 0-Fully active; able to carry on all pre-disease activities without restriction; 1-Restricted in physically strenuous activity but ambulatory and able to carry out light or sedentary work; 2-Ambulatory and capable of all self-care but unable to carry out work activities; up and about more than 50% of waking hours; 3-Capable of only limited self-care; confined to bed or chair more than 50% of waking hours; 4-Completely disabled; unable to carry out any self-care; totally confined to bed or chair; 5-Dead. Protocol inclusion criteria required patients to have an ECOG Performance Status of 0 or 1 to be eligible.

The results below show the activity after treatment with imneskibart+Low-Dose IL-2 without nivolumab in Phase 1 and 2 in Melanoma patients.

FIGS. 46A and 46B present the response of melanoma patients to administration of RP2D AU-007+IL-2 loading dose without nivolumab. Patients with BRAF/MEK mutations were eligible if not treated with prior BRAF/MEK inhibitor. FIG. 46A shows best response in the target lesions of melanoma patients who progressed on prior CPI therapy (anti-CTLA-4+anti-PD- and/or anti-PD-1+anti-LAG-3) treated with imneskibart (AU-007)+low dose SQ IL-2 (n=14). All patients failed prior doublet CPI (marked with an asterisk within each bar).

FIG. 46B shows the percentage change over time (months) vs. baseline in the target lesions of the same melanoma patient population (n=14). The arrows indicate administration of a low-dose SQ IL-2 boost (135,000 IU/kg) for a patient. The solid circles (in FIG. 46A) and [symbol] solid head arrows ( [symbol] ; in FIG. 46B) indicate patients continuing in the clinical trial. These results are updated results of the data presented in FIGS. 41A and 41B.

The results show the ability to slow or reverse tumor growth with boost IL-2. Accordingly, boost IL-2 every 8 weeks (Q8W) was administered until at least RECIST PR (−30% reduction) is achieved. In future trials, every 8-week IL-2 dosing (e.g., of 135,000 IU/kg) may be evaluated.

The results below show the activity in Melanoma Patients who progressed on prior doublet CPI therapy treated with Nivolumab+Imneskibart+Low-Dose, SC IL-2.

FIGS. 47A and 47B present clear anti-tumor activity in 12 melanoma patients who progressed on prior doublet CPI therapy, and were then treated with the RP2D-1 (3 patients) or RP2D (9 patients): nivolumab+imneskibart+low dose SQ IL-2. Patients with BRAF/MEK mutations were eligible only if not treated with prior BRAF/MEK inhibitor. Patients had progressed on prior doublet checkpoint inhibitors: anti-CTLA-4+anti-PD-1 and/or anti-PD-1+anti-Lag-3. These results are updated results of the data presented in FIGS. 42A and 42B.

FIG. 47A shows best response in the target lesions of melanoma patients who had progressed on prior doublet CPI therapy (n=12); patients that received RP2D-1 exhibited changes in target lesions of +24%, +2%, and −65% (partial response). Among patients treated at the RP2D, 3 obtained a partial response with 52%, 74%, and 77% decrease in target lesions. The solid circles in FIG. 47A indicate patients continuing in the clinical trial. The patient exhibiting a decrease in target lesions of 52% is in follow-up but is not receiving further treatment. Overall, 4/12 patients (33%) obtained a partial responses, 3 confirmed and 1 awaiting confirmation, with a 67% disease control rate (partial response+stable disease).

FIG. 47B shows percentage change over time vs. baseline in the target lesions of these melanoma patients who progressed on prior doublet CPI therapy. The arrows in FIG. 47B indicate administration of a low-dose SQ IL-2 boost (135,000 IU/kg). The solid head arrows ( [symbol] in FIG. 47B) indicate patients continuing in the clinical trial. The lowest graph indicates the patient that is under follow-up but is not receiving any treatment.

The results show the ability to slow or reverse tumor growth with a boost IL-2. Accordingly, a boost IL-2 Q8W until at least RECIST PR (−30% reduction) is achieved was implemented. Future trials may include every 8-week IL-2 dosing.

The results below show an Adverse Event Summary: Mild and Tolerable Profile. The results are an update of data appearing in Table 21 and Table 22 presented above.

Table 24 and Table 25 provide a summary of Adverse Events (AEs) and Serious Adverse Events (SAEs) for patients treated with imneskibart+IL-2 or imneskibart+IL-2+nivolumab combination.

TABLE 24
Low Rate of Drug-Related SAEs and Grade 3/4 AEs

| | Imneskibart + IL-2¹ | Nivolumab Combo² |
|---|---|---|
| Event (n, %) | N = 83 | N = 17 |
| Any AE | 79 (95) | 16 (94) |
| Drug-Related AEs | 68 (82) | 11 (65) |
| Drug-Related SAEs | 5 (6) | 4 (24) |
| CRS | 3 | 2 |
| Infusion-related reaction | 1 | 1 |
| Pyrexia | 1 | 0 |
| Adrenal insufficiency | 0 | 0 |
| Injection site rash | 1 | 0 |
| Pneumonitis | 0 | 1 |
| Drug-Related Grade 3 or 4 AEs | 12 (15) | 1 (6) |
| Lymphopenia | 7 | 0 |
| Anemia | 2 | 0 |
| CRS | 1 | 0 |
| Fatigue | 1 | 0 |
| Lipase elevation | 1 | 0 |
| Neutropenia | 1 | 0 |
| Maculopapular rash | 1 | 0 |
| Autoimmune encephalitis | 1 | 0 |
| Pneumonitis | 0 | 1 |

¹Imneskibart Q2W + IL-2 loading dose or IL-2 Q2W-all tumor types
²Imneskibart Q2W + IL-2 loading dose and nivolumab-melanoma

TABLE 25
Most Drug-Related AEs Tolerable and Easily Managed
Drug-Related Adverse Events in >10% of Patients

| | Imneskibart + IL-21 | Nivolumab Combo2 |
|---|---|---|
| Adverse Event (n, %) | N = 83 | N = 17 |
| Pyrexia | 21 (25) | 1 (6) |
| Fatigue | 20 (24) | 2 (12) |
| Chills | 18 (21) | 2 (12) |
| Nausea | 12 (15) | 2 (12) |
| Infusion-related | 11 (13) | 1 (6) |
| reaction | | |
| Injection site reaction | 11 (13) | 4 (24) |
| CRS | 7 (9) | 5 (29) |

1Imneskibart Q2W + IL-2 loading dose or IL-2 Q2W-all tumor types
2Imneskibart Q2W + IL-2 loading dose and nivolumab-melanoma

The results are consistent with the data reported and shown in Table 21 and Table 22, showing that most drug-related adverse events (AEs) were mild, Grade 1 or 2, with imneskibart+SC loading dose aldesleukin alone or in combination with nivolumab. The results show well-tolerated outpatient regimen with no treatment discontinuations due to drug-related adverse events.

The addition of nivolumab to imneskibart+SQ loading dose aldesleukin does not increase the rate of drug-related Grade 3/4 AEs in the emerging data.

The most common Grade 3 or 4 AE was transient (3-7 days) lymphopenia that was not associated with adverse outcomes in any patient. Transient lymphopenia is a known effect of IL-2 treatment as lymphocytes traffic out of blood and into tissue.

One patient receiving imneskibart+SQ loading dose aldesleukin experienced Grade 4 cytokine release syndrome (CRS). It was resolved without tocilizumab using steroids, IV fluids, and brief vascular pressor support. This patient was noted retrospectively to have subclinical elevated IL-6 (5×) serum levels likely due to an active case of gout at baseline.

FIG. 48A-FIG. 48D show the CT scans of a patient whose tumor previously progressed through prior anti-PD-1+anti CTLA-4 doublet therapy. The patient is a 28-year-old man with melanoma metastatic to the mediastinal lymph nodes and lung. The patient received prior neoadjuvant and then adjuvant pembrolizumab followed by ipilimumab+nivolumab. In September 2025, the patient began treatment with the RP2D imneskibart+aldesleukin loading dose at 135K IU/kg in combination with nivolumab 480 mg Q4W.

FIG. 48A-FIG. 48D present tumor assessments by computed tomography scans from the melanoma patient, where the cancer did not respond to prior checkpoint inhibitors anti-PD-1 and anti-CTLA-4 doublet therapy. FIG. 48A—presents the baseline CT scan, wherein bilateral lung and lymph node metastases are shown (circled in black); FIG. 48B—presents 8-month CT scan; FIG. 48C—presents the baseline L lower lobe lung metastases circled in black; FIG. 48D—presents the 8-month CT scan.

The results show a confirmed partial response with 32%, 35%, 42%, and 77% tumor size decrease at end of Cycles 1, 2, 3, and 4, respectively. The 77% reduction level was observed at 8 months in the target lesions of the patient.

FIG. 48A-FIG. 48D show the CT scans of a patient whose tumor previously progressed through prior anti-PD-1+anti LAG-3 doublet therapy. The patient is a 69-year-old man with melanoma metastatic to the left upper lobe of the lung, L subcutaneous thorax, colon, and peritoneum. The patient received prior ipilimumab, nivolumab+relatlimab, TIL therapy, and talimogene laherparepvec (T-VEC) therapy. December 2025, the patient began treatment with the RP2D imneskibart+aldesleukin in combination with nivolumab 480 mg Q4W.

FIG. 49A-FIG. 49D present tumor assessments by CT scan from the melanoma patient, where the cancer did not respond to checkpoint inhibitors anti-PD-1 and anti-LAG-3 doublet therapy. FIG. 49A presents the baseline CT scan, wherein L thorax subcutaneous metastases are shown (circled in black); FIG. 49B presents 4-month CT scan; FIG. 49C presents the baseline L upper lobe lung metastasis; FIG. 49D presents the 4-month scan.

The results show a confirmed partial response with 36% and 74% target tumor size decrease at end of Cycles 1 and 2, respectively. The 74% reduction level was observed at 4 months in the target lesions of the patient.

Analysis of the prior doublet checkpoint inhibitor cancer therapy and time on imneskibart study treatment is shown in FIG. 50. RECIST evaluable melanoma patients in Phase 1 and 2 who received 9 mg/kg Q2W imneskibart combined with the single loading dose of 135,000 IU/kg aldesleukin. Prior doublet treatment included: ipilimumab/nivolumab or nivolumab/relatlimab.

FIGS. 51A and 51B show intratumoral effector CD8 and NK cell increases after one 8-week treatment cycle with imneskibart+aldesleukin. Tumor biopsies were obtained during the study screening period prior to the initial study drug administration, and at the end of the initial 8-week treatment cycle. The tumors histologies were melanoma, NSCLC, and renal cell carcinoma (RCC). The initial data demonstrate a trend toward greater CD8 and NK cell counts in the on-treatment tumor samples compared to pre-treatment samples.

FIGS. 52A and 52B present Higher peripheral blood CD8/Treg Ratio Associated With Better Anti-Tumor Activity.

FIG. 52A presents the PFS over the time of the ongoing study. The results are updated data set of FIG. 45B. Longer durations of progression-free survival (PFS) were observed in melanoma patients who achieved a ≥2.16-fold increase over their baseline CD8/Treg ratio. Results are calculated by deriving the median greatest Treg decrease across all patients (excluding the first 2 days on treatment) and dividing the patients who were ≤ the median or > than the median for CD8/Treg ratio fold change versus baseline.

The median PFS and duration on treatment were 7.6 and 9 months (M) respectively (9.3 M median follow up); 6 patients continue treatment. These results are obtained from patients treated with imneskibart 9 mg/kg+135K IU/kg IL-2 in melanoma patients (n=14).

FIG. 52B shows the Progression Status at 16 Weeks. The Maximum CD8/Treg Fold Change vs. Baseline by Week 16 Progression Status. Patients divided by progression status after 16 weeks (2 treatment Cycles) on treatment and the individual patient maximum CD8/Treg ratio fold increase over baseline was plotted for each patient.

Boxplots summarize maximum CD8/Treg ratio fold increase by progression status group showing median (line in each box) with lower and upper box boundaries corresponding to the 25th and 75th percentile. A trend toward higher maximum CD8/Treg ratio fold increase was observed in patients without progression at 16 weeks compared to those with progression, suggesting a potential association between enhanced effector-to-regulatory T cell balance and clinical outcome.

### SUMMARY

The results show clinically meaningful activity observed with imneskibart triplet of imneskibart+low-dose, SC aldesleukin and nivolumab in doublet CPI- (post anti-PD-1+CTLA-4 or PD-1+LAG-3) refractory melanoma where late responses to prior therapy are effectively excluded: 4/12 patients with partial responses (33%), 3 confirmed and 1 awaiting confirmation, and 67% disease control rate (DCR).

Compelling durability and depth emerging: Responses are deepening and ongoing, consistent with de novo anti-tumor immunity and immune memory formation.

By “De novo anti-tumor immunity” it is meant the generation or induction of a new or newly functional anti-tumor immune response in a subject, which was not previously present or was not functionally active, including through activation, expansion, or recruitment of immune effector cells capable of mediating tumor cell recognition and/or killing.

By “Immune memory formation” it is meant the development of long-lived immune cells capable of mediating a more rapid and/or enhanced anti-tumor response upon subsequent exposure to the tumor, including, but not limited to, cells capable of recognizing tumor-associated antigens.

Imneskibart and low-dose, subcutaneous aldesleukin, with or without nivolumab, exhibit unique pharmacodynamic (PD), biological and pharmacokinetic (PK) effects in the IL-2 class, with a higher peripheral blood CD8/Treg ratio correlating with increased survival.

Pharmacodynamic analyses demonstrated decreased peripheral Tregs, increased CD8+ T cells and NK cells, and increased CD8/Treg ratio, supporting selective immune effector cell expansion.

PD data support hypothesis of selective effector cell expansion, validating imneskibart's mechanism of action of redirecting interleukin-2 (IL-2) away from trimeric IL-2 receptors (expressed on Tregs and vasculature) and toward dimeric IL-2 receptors (on CD8+ T effector and natural killer cells).

The data show that imneskibart demonstrates durable activity coupled with a persistent reduction in Tregs and a higher CD8/Treg ratio that is associated with longer OS, PFS and time on treatment for patients.

The emerging safety profile of the triplet regimen is well-tolerated and consistent with the known safety profiles of nivolumab and imneskibart+aldesleukin, with no new toxicity signals observed.

PK data demonstrate sustained, selective signaling with a half-life of about 19.5 days), enabling potent immune activation without Treg expansion or high-dose IL-2 toxicity, such as vascular leak syndrome pulmonary edema.

The results show synergy with anti-PD-1.

Data in the doublet-CPI refractory melanoma patient population strongly support continued clinical development with the triplet regimen of imneskibart+low-dose, SC aldesleukin and nivolumab.

Strong signal of anti-tumor activity in imneskibart doublet regimen, with ongoing deep and durable tumor reductions in patients who progressed after receiving doublet CPI therapy (prior anti-PD-1 and anti-CTLA-4 and/or anti-PD-1 and anti-LAG-3). Fourteen (14) evaluable patients received the imneskibart doublet regimen. Three patients with the deepest target tumor reductions continued treatment beyond one year: one patient with a 48% tumor reduction continued on treatment for 13 months; another patient with a 58% reduction in measurable non-target tumors continued on treatment for 18.5 months; and a third patient with a complete response (100% reduction) in the target lesions continues on treatment for more than 25 months.

The imneskibart+low dose, subcutaneous (SC) aldesleukin, with or without nivolumab, regimen may provide a treatment option for patients who are not eligible for, or do not receive, tumor infiltrating lymphocyte (TIL) therapy. TIL therapy requires the harvesting and ex vivo expansion of sufficient tumor derived lymphocytes, which may not be feasible in all patients. In addition, patients must undergo preparative regimens, including IL 2 administration and lymphodepletion, the associated toxicities may preclude treatment. In contrast, the present regimen does not rely on cell harvesting or expansion and may be administered without such preconditioning, thereby expanding treatment options for patients ineligible for TIL therapy.

What is claimed is:

1. A method of treating an unresectable locally advanced or metastatic solid cancer in a subject, said method comprising administering to said subject multiple doses of an anti-IL-2 antibody or a pharmaceutical composition thereof, a loading dose of IL-2 or a pharmaceutical composition thereof, and at least one booster dose of IL-2 or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3,
wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5;
wherein said loading dose and booster dose of IL-2 are subcutaneously administered at a dose of between about 15,000 IU/kg of said subject's body weight and 500,000 IU/kg of said subject's body weight;
wherein said pharmaceutical composition(s) further comprises a pharmaceutically acceptable carrier,
thereby treating said unresectable locally advanced or metastatic solid cancer in said subject.

2. The method according to claim 1, wherein said at least one booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, until at least RECIST Partial Response is achieved, or any combination thereof.

3. The method according to claim 1, wherein said anti-IL-2 antibody is (a) administered at a dose of between about 0.5 mg/kg of a subject's body weight and 12 mg/kg of said subject's body weight; between about 4.5 mg/kg of a subject's body weight and 12 mg/kg of said subject's body weight; or between about 9 mg/kg of a subject's body weight and 12 mg/kg of said subject's body weight; (b) administered once every two weeks; (c) administered intravenously; or (d) any combination thereof.

4. The method according to claim 1, wherein the administration of said loading dose of IL-2 is prior to, concurrent with, or following the administration of said anti-IL-2 antibody.

5. The method according to claim 1, wherein said loading dose of IL-2 or said least one booster dose of IL-2 is administered at a dose of between about 45,000 IU/kg of a subject's body weight and 270,000 IU/kg of said subject's body weight or between about 45,000 IU/kg of a subject's body weight and 135,000 IU/kg of said subject's body weight.

6. The method according to claim 1, wherein said anti-IL-2 antibody is administered at a dose of 9 mg/kg of said subject's body weight, the IL-2 loading dose is administered at a dose of 135,000 IU/kg of said subject's body weight, and the at least one booster dose of IL-2 is administered at a dose of 135,000 IU/kg of said subject's body weight.

7. The method according to claim 1, wherein said method further comprises administering a checkpoint inhibitor or a pharmaceutical composition thereof, said pharmaceutical composition further comprising a pharmaceutically acceptable carrier, wherein the checkpoint inhibitor comprises:
a. a PD-L1 checkpoint inhibitor, said PD-L1 checkpoint inhibitor optionally comprising atezolizumab, durvalumab, sugemalimab, envafolimab, or cosibelimab; or
b. a PD-1 checkpoint inhibitor, said PD-1 checkpoint inhibitor optionally comprising nivolumab, pembrolizumab, cemiplimab, camrelizumab, zimberelimab, tislelizumab, sintilimab, teriprizumab, prolgolimab, penpulimab, dostarlimab, genolimzumab, or retifanlimab
and wherein said checkpoint inhibitor is administered prior to, concurrent with, or following the administration of said anti-IL-2 antibody, said loading dose or booster dose of IL-2, or both.

8. The method according to claim 1, wherein said unresectable locally advanced or metastatic cancer comprises a cutaneous melanoma, a melanoma, a renal cell carcinoma (RCC), a non-small cell lung cancer (NSCLC), a squamous NSCLC, a non-squamous NSCLC, a head and neck carcinoma, a head and neck squamous cell carcinoma (HNSCC), a nasopharyngeal carcinoma, an esophageal carcinoma, a gastric carcinoma, a gastric or gastro-esophageal cancer, a gastroesophageal junction carcinoma, an esophageal squamous cell carcinoma, a cutaneous squamous cell carcinoma (cSCC), a hepato-cellular carcinoma (HCC), a colorectal cancer (CRC), a microsatellite instability high (MSHi) CRC, a microsatellite stable (MSHs) CRC, a cervical carcinoma, an endometrial carcinoma, a urothelial cancer, a bladder cancer, a ureteral cancer, a renal pelvis cancer, a malignant mesothelioma, a malignant pleural mesothelioma, a malignant peritoneal mesothelioma, a classical Hodgkin Lymphoma (cHL), a biliary tract carcinoma (BTC), a MSHi or mismatch repair deficient cancer, a Tumor Mutational Burden-High (TMB-H) cancer, Triple-negative breast cancer (TNBC), or a Merkel cell carcinoma (MCC), or a combination thereof.

9. The method according to claim 1, wherein said cancer progressed after prior checkpoint inhibitor therapy in said subject.

10. The method according to claim 1, wherein said method comprises a first line treatment, a second line treatment, or a third line treatment.

11. The method according to claim 1, wherein said method of treating comprises (i) reducing the size of the tumor, (ii) inhibiting or reducing growth of the tumor, (iii) inhibiting or reducing metastases of said tumor, (iv) inhibiting the appearance of new lesions, (v) decreasing or shrinking target lesions, (vi) eliminating target lesions, or (vii) any combination thereof.

12. The method according to claim 1, wherein (a) the amino acid sequence of the full length heavy chain is set forth in SEQ ID NO: 8 and the amino acid sequence of the full length light chain is set forth in SEQ ID NO: 9, (b) the amino acid sequence of the VH comprises the amino acid sequence of SEQ ID NO:6, and the VL comprises the amino acid sequence of SEQ ID NO:7, or (c) a combination thereof.

13. The method according to claim 1, wherein said antibody comprises an IgG, IgA, IgM, IgE, IgD, a Fv, a scFv, a Fab, a F(ab′)₂, a minibody, a diabody, or a triabody or wherein said antibody comprises a heavy chain comprising a mutation that reduces binding to fragment crystallizable gamma receptors (FcγRs), wherein reduced binding is compared with said antibody lacking the mutation in the heavy chain that affects FcγRs binding, and wherein optionally said mutation comprises L234A, L235A (LALA) mutations, or a combination thereof.

14. A combination therapy comprising an anti-IL-2 antibody or a pharmaceutical composition thereof, a loading dose of IL-2 or a pharmaceutical composition thereof, and nivolumab or a pharmaceutical composition thereof,
wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3,
wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5;
wherein said anti-IL-2 antibody is formulated for administration at a dose of 9 mg/kg of a subject's body weight, the loading dose of IL-2 is formulated for subcutaneous administration at a dose of 135,000 IU/kg of a subject's body weight, and said nivolumab is formulated for administration at a dose of 480 mg or 240 mg; and
wherein said pharmaceutical composition(s) further comprises a pharmaceutically acceptable carrier.

15. The combination therapy according to claim 14, wherein (a) the amino acid sequence of the VH comprises the amino acid sequence of SEQ ID NO:6 and the amino acid sequence of VL comprises the amino acid sequence of SEQ ID NO:7; (b) wherein the amino acid sequence of the full length heavy chain is set forth in SEQ ID NO:8 and the amino acid sequence of the full length light chain is set forth in SEQ ID NO:9; or (c) any combination thereof.

16. The combination therapy according to claim 14, wherein the antibody comprises an IgG, IgA, IgM, IgE, IgD, a Fv, a scFv, a Fab, a F(ab′)₂, a minibody, a diabody, or a triabody or wherein said antibody comprises a heavy chain comprising a mutation that reduces binding to a fragment crystallizable gamma receptors (FcγRs), wherein reduced binding is compared with said antibody lacking the mutation in the heavy chain that affects FcγRs binding, and wherein optionally said mutation comprises L234A, L235A (LALA) mutations.

17. A method of treating an unresectable locally advanced or metastatic solid cancer in a subject, said method comprising administering to said subject an anti-IL-2 antibody at a dose of 9 mg/kg of said subject's body weight or a pharmaceutical composition thereof, a loading dose of IL-2 at a dose of 135,000 IU/kg of said subject's body weight or a pharmaceutical composition thereof, and nivolumab at a dose of 480 mg or 240 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3,
wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5,
wherein said pharmaceutical composition(s) further comprises a pharmaceutically acceptable carrier,
thereby treating said unresectable locally advanced or metastatic solid cancer in said subject.

18. The method according to claim 17, wherein said anti-IL-2 antibody, said nivolumab, or both are administered intravenously to said subject and wherein said IL-2 loading dose is administered subcutaneously.

19. The method according to claim 17, wherein the administration of the loading dose IL-2 is prior to, concurrent with, or following the administration of said anti-IL-2 antibody, said nivolumab, or both.

20. The method according to claim 17, further comprising the step of administering one or more (a) additional doses of said anti-IL-2 antibody or a pharmaceutical composition thereof, (b) additional doses of said nivolumab or a pharmaceutical composition thereof, or (c) booster doses of IL-2 or a pharmaceutical composition thereof, or (d) any combination thereof, to said subject, wherein said pharmaceutical composition(s) further comprise(s) a pharmaceutically acceptable carrier.

21. The method according to claim 20, wherein
(a) said additional doses of said anti-IL-2 antibody or a pharmaceutical composition thereof are administered once every two weeks; or
(b) said additional doses of said nivolumab or a pharmaceutical composition thereof are administered once every four weeks; or
(c) any combination thereof.

22. The method according to claim 20, wherein said booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, until at least RECIST Partial Response is achieved, or any combination thereof, and wherein said IL-2 booster dose is administered subcutaneously.

23. The method according to claim 20, wherein the administration of said one or more booster doses of IL-2 is prior to, concurrent with, or following the administration of said one or more additional doses of said anti-IL-2 antibody, one or more additional doses of said nivolumab, or both, optionally wherein the at least one IL-2 booster dose is administered at a dose of 135,000 IU/kg of said subject's body weight.

24. The method according to claim 17, wherein said method of treating comprises (i) reducing the size of the tumor, (ii) inhibiting or reducing growth of the tumor, (iii) inhibiting or reducing metastases of said tumor, (iv) inhibiting the appearance of new lesions, (v) decreasing or shrinking target lesions, (vi) eliminating target lesions, or (vii) any combination thereof.

25. The method according to claim 17, wherein said solid cancer progressed after prior checkpoint inhibitor therapy in said subject.

26. The method according to claim 17, wherein said unresectable locally advanced or metastatic solid cancer comprises a cutaneous melanoma, a melanoma, a renal cell carcinoma (RCC), a head and neck carcinoma, a head and neck squamous cell carcinoma (HNSCC), a nasopharyngeal carcinoma, an esophageal carcinoma, a gastric carcinoma, a gastric or gastro-esophageal cancer, a gastroesophageal junction carcinoma, an esophageal squamous cell carcinoma, a cutaneous squamous cell carcinoma (cSCC), a hepato-cellular carcinoma (HCC), a colorectal cancer (CRC), a microsatellite instability high (MSHi) CRC, a microsatellite stable (MSHs) CRC, a cervical carcinoma, an endometrial carcinoma, a urothelial cancer, a bladder cancer, a ureteral cancer, a renal pelvis cancer, a malignant mesothelioma, a malignant pleural mesothelioma, a malignant peritoneal mesothelioma, a classical Hodgkin Lymphoma (cHL), a biliary tract carcinoma (BTC), a MSHi or mismatch repair deficient cancer, a Tumor Mutational Burden-High (TMB-H) cancer or a Merkel cell carcinoma (MCC).

27. The method according to claim 26, wherein said method of treating comprises a first line treatment, a second line treatment, or a third line treatment.

28. The method according to claim 17, wherein said unresectable locally advanced or metastatic solid cancer comprises a non-small cell lung cancer (NSCLC), a squamous NSCLC, or a non-squamous NSCLC, and wherein said method consists of a first line treatment.
